SEQUENCE LISTING (518 sequences)

SEQ ID NO: 1 - protein, 165 aa, synthetic construct [sequence omitted]

SEQ ID NO: 2 - protein, 4 aa, synthetic construct
  REGION 1..4: note = Synthetic
GGGS

SEQ ID NO: 3 - protein, 219 aa, synthetic construct
  REGION 1..219: note = Synthetic
CPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 4 - protein, 226 aa, synthetic construct
  REGION 1..226: note = Synthetic
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 5 - protein, 8 aa, synthetic construct
  REGION 1..8: note = Synthetic
LSGRSDNH

SEQ ID NO: 6 - protein, 8 aa, synthetic construct
  REGION 1..8: note = Synthetic
TGRGPSWV

SEQ ID NO: 7 - protein, 8 aa, synthetic construct
  REGION 1..8: note = Synthetic
PLTGRSGG

SEQ ID NO: 8 - protein, 8 aa, synthetic construct
  REGION 1..8: note = Synthetic
TARGPSFK

SEQ ID NO: 9 - protein, 12 aa, synthetic construct
  REGION 1..12: note = Synthetic
NTLSGRSENHSG

SEQ ID NO: 10 - protein, 12 aa, synthetic construct
  REGION 1..12: note = Synthetic
NTLSGRSGNHGS

SEQ ID NO: 11 - protein, 12 aa, synthetic construct
  REGION 1..12: note = Synthetic
TSTSGRSANPRG

SEQ ID NO: 12 - protein, 8 aa, synthetic construct
  REGION 1..8: note = Synthetic
TSGRSANP

SEQ ID NO: 13 - protein, 10 aa, synthetic construct
  REGION 1..10: note = Synthetic
VHMPLGFLGP

SEQ ID NO: 14 - protein, 8 aa, synthetic construct
  REGION 1..8: note = Synthetic
AVGLLAPP

SEQ ID NO: 15 - protein, 8 aa, synthetic construct
  REGION 1..8: note = Synthetic
AQNLLGMV

SEQ ID NO: 16 - protein, 8 aa, synthetic construct
  REGION 1..8: note = Synthetic
QNQALRMA

SEQ ID NO: 17 - protein, 8 aa, synthetic construct
  REGION 1..8: note = Synthetic
LAAPLGLL

SEQ ID NO: 18 - protein, 8 aa, synthetic construct
  REGION 1..8: note = Synthetic
STFPFGMF

SEQ ID NO: 19 - protein, 8 aa, synthetic construct
  REGION 1..8: note = Synthetic
ISSGLLSS

SEQ ID NO: 20 - protein, 8 aa, synthetic construct
  REGION 1..8: note = Synthetic
PAGLWLDP

SEQ ID NO: 21 - protein, 8 aa, synthetic construct
  REGION 1..8: note = Synthetic
VAGRSMRP

SEQ ID NO: 22 - protein, 8 aa, synthetic construct
  REGION 1..8: note = Synthetic
VVPEGRRS

SEQ ID NO: 23 - protein, 8 aa, synthetic construct
  REGION 1..8: note = Synthetic
ILPRSPAF

SEQ ID NO: 24 - protein, 8 aa, synthetic construct
  REGION 1..8: note = Synthetic
MVLGRSLL

SEQ ID NO: 25 - protein, 8 aa, synthetic construct
  REGION 1..8: note = Synthetic
QGRAITFI

SEQ ID NO: 26 - protein, 8 aa, synthetic construct
  REGION 1..8: note = Synthetic
SPRSIMLA

SEQ ID NO: 27 - protein, 8 aa, synthetic construct
  REGION 1..8: note = Synthetic
SMLRSMPL

SEQ ID NO: 28 - protein, 13 aa, synthetic construct
  REGION 1..13: note = Synthetic
ISSGLLSGRSDNH

SEQ ID NO: 29 - protein, 18 aa, synthetic construct
  REGION 1..18: note = Synthetic
AVGLLAPPGGLSGRSDNH

SEQ ID NO: 30 - protein, 22 aa, synthetic construct
  REGION 1..22: note = Synthetic
ISSGLLSSGGSGGSLSGRSDNH

SEQ ID NO: 31 - protein, 8 aa, synthetic construct
  REGION 1..8: note = Synthetic
LSGRSGNH

SEQ ID NO: 32 - protein, 9 aa, synthetic construct
  REGION 1..9: note = Synthetic
SGRSANPRG

SEQ ID NO: 33 - protein, 8 aa, synthetic construct
  REGION 1..8: note = Synthetic
LSGRSDDH

SEQ ID NO: 34 - protein, 8 aa, synthetic construct
  REGION 1..8: note = Synthetic
LSGRSDIH

SEQ ID NO: 35 - protein, 8 aa, synthetic construct
  REGION 1..8: note = Synthetic
LSGRSDQH

SEQ ID NO: 36 - protein, 8 aa, synthetic construct
  REGION 1..8: note = Synthetic
LSGRSDTH

SEQ ID NO: 37 - protein, 8 aa, synthetic construct
  REGION 1..8: note = Synthetic
LSGRSDYH

SEQ ID NO: 38 - protein, 8 aa, synthetic construct
  REGION 1..8: note = Synthetic
LSGRSDNP

SEQ ID NO: 39 - protein, 8 aa, synthetic construct
  REGION 1..8: note = Synthetic
LSGRSANP

SEQ ID NO: 40 - protein, 8 aa, synthetic construct
  REGION 1..8: note = Synthetic
LSGRSANI

SEQ ID NO: 41 - protein, 8 aa, synthetic construct
  REGION 1..8: note = Synthetic
LSGRSDNI

SEQ ID NO: 42 - protein, 8 aa, synthetic construct
  REGION 1..8: note = Synthetic
MIAPVAYR

SEQ ID NO: 43 - protein, 8 aa, synthetic construct
  REGION 1..8: note = Synthetic
RPSPMWAY

SEQ ID NO: 44 - protein, 8 aa, synthetic construct
  REGION 1..8: note = Synthetic
WATPRPMR

SEQ ID NO: 45 - protein, 8 aa, synthetic construct
  REGION 1..8: note = Synthetic
FRLLDWQW

SEQ ID NO: 46 - protein, 5 aa, synthetic construct
  REGION 1..5: note = Synthetic
ISSGL

SEQ ID NO: 47 - protein, 7 aa, synthetic construct
  REGION 1..7: note = Synthetic
ISSGLLS

SEQ ID NO: 48 - protein, 6 aa, synthetic construct
  REGION 1..6: note = Synthetic
ISSGLL

SEQ ID NO: 49 - protein, 15 aa, synthetic construct
  REGION 1..15: note = Synthetic
ISSGLLSGRSANPRG

SEQ ID NO: 50 - protein, 18 aa, synthetic construct
  REGION 1..18: note = Synthetic
AVGLLAPPTSGRSANPRG

SEQ ID NO: 51 - protein, 17 aa, synthetic construct
  REGION 1..17: note = Synthetic
AVGLLAPPSGRSANPRG

SEQ ID NO: 52 - protein, 13 aa, synthetic construct
  REGION 1..13: note = Synthetic
ISSGLLSGRSDDH

SEQ ID NO: 53 - protein, 13 aa, synthetic construct
  REGION 1..13: note = Synthetic
ISSGLLSGRSDIH

SEQ ID NO: 54 - protein, 13 aa, synthetic construct
  REGION 1..13: note = Synthetic
ISSGLLSGRSDQH

SEQ ID NO: 55 - protein, 13 aa, synthetic construct
  REGION 1..13: note = Synthetic
ISSGLLSGRSDTH

SEQ ID NO: 56 - protein, 13 aa, synthetic construct
  REGION 1..13: note = Synthetic
ISSGLLSGRSDYH

SEQ ID NO: 57 - protein, 13 aa, synthetic construct
  REGION 1..13: note = Synthetic
ISSGLLSGRSDNP

SEQ ID NO: 58 - protein, 13 aa, synthetic construct
  REGION 1..13: note = Synthetic
ISSGLLSGRSANP

SEQ ID NO: 59 - protein, 13 aa, synthetic construct
  REGION 1..13: note = Synthetic
ISSGLLSGRSANI

SEQ ID NO: 60 - protein, 18 aa, synthetic construct
  REGION 1..18: note = Synthetic
AVGLLAPPGGLSGRSDDH

SEQ ID NO: 61 - protein, 18 aa, synthetic construct
  REGION 1..18: note = Synthetic
AVGLLAPPGGLSGRSDIH

SEQ ID NO: 62 - protein, 18 aa, synthetic construct
  REGION 1..18: note = Synthetic
AVGLLAPPGGLSGRSDQH

SEQ ID NO: 63 - protein, 18 aa, synthetic construct
  REGION 1..18: note = Synthetic
AVGLLAPPGGLSGRSDTH

SEQ ID NO: 64 - protein, 18 aa, synthetic construct
  REGION 1..18: note = Synthetic
AVGLLAPPGGLSGRSDYH

SEQ ID NO: 65 - protein, 18 aa, synthetic construct
  REGION 1..18: note = Synthetic
AVGLLAPPGGLSGRSDNP

SEQ ID NO: 66 - protein, 18 aa, synthetic construct
  REGION 1..18: note = Synthetic
AVGLLAPPGGLSGRSANP

SEQ ID NO: 67 - protein, 18 aa, synthetic construct
  REGION 1..18: note = Synthetic
AVGLLAPPGGLSGRSANI

SEQ ID NO: 68 - protein, 13 aa, synthetic construct
  REGION 1..13: note = Synthetic
ISSGLLSGRSDNI

SEQ ID NO: 69 - protein, 18 aa, synthetic construct
  REGION 1..18: note = Synthetic
AVGLLAPPGGLSGRSDNI

SEQ ID NO: 70 - protein, 19 aa, synthetic construct
  REGION 1..19: note = Synthetic
GLSGRSDNHGGAVGLLAPP

SEQ ID NO: 71 - protein, 21 aa, synthetic construct
  REGION 1..21: note = Synthetic
GLSGRSDNHGGVHMPLGFLGP

SEQ ID NO: 72 - protein, 20 aa, synthetic construct
  REGION 1..20: note = Synthetic
LSGRSDNHGGVHMPLGFLGP

SEQ ID NO: 73 - protein, 7 aa, synthetic construct
  REGION 1..7: note = Synthetic
ISSGLSS

SEQ ID NO: 74 - protein, 8 aa, synthetic construct
  REGION 1..8: note = Synthetic
PVGYTSSL

SEQ ID NO: 75 - protein, 8 aa, synthetic construct
  REGION 1..8: note = Synthetic
DWLYWPGI

SEQ ID NO: 76 - protein, 8 aa, synthetic construct
  REGION 1..8: note = Synthetic
LKAAPRWA

SEQ ID NO: 77 - protein, 8 aa, synthetic construct
  REGION 1..8: note = Synthetic
GPSHLVLT

SEQ ID NO: 78 - protein, 8 aa, synthetic construct
  REGION 1..8: note = Synthetic
LPGGLSPW

SEQ ID NO: 79 - protein, 8 aa, synthetic construct
  REGION 1..8: note = Synthetic
MGLFSEAG

SEQ ID NO: 80 - protein, 8 aa, synthetic construct
  REGION 1..8: note = Synthetic
SPLPLRVP

SEQ ID NO: 81 - protein, 8 aa, synthetic construct
  REGION 1..8: note = Synthetic
RMHLRSLG

SEQ ID NO: 82 - protein, 8 aa, synthetic construct
  REGION 1..8: note = Synthetic
LLAPSHRA

SEQ ID NO: 83 - protein, 7 aa, synthetic construct
  REGION 1..7: note = Synthetic
GPRSFGL

SEQ ID NO: 84 - protein, 6 aa, synthetic construct
  REGION 1..6: note = Synthetic
GPRSFG

SEQ ID NO: 85 - protein, 8 aa, synthetic construct
  REGION 1..8: note = Synthetic
SARGPSRW

SEQ ID NO: 86 - protein, 8 aa, synthetic construct
  REGION 1..8: note = Synthetic
GGWHTGRN

SEQ ID NO: 87 - protein, 8 aa, synthetic construct
  REGION 1..8: note = Synthetic
HTGRSGAL

SEQ ID NO: 88 - protein, 8 aa, synthetic construct
  REGION 1..8: note = Synthetic
AARGPAIH

SEQ ID NO: 89 - protein, 8 aa, synthetic construct
  REGION 1..8: note = Synthetic
RGPAFNPM

SEQ ID NO: 90 - protein, 8 aa, synthetic construct
  REGION 1..8: note = Synthetic
SSRGPAYL

SEQ ID NO: 91 - protein, 8 aa, synthetic construct
  REGION 1..8: note = Synthetic
RGPATPIM

SEQ ID NO: 92 - protein, 4 aa, synthetic construct
  REGION 1..4: note = Synthetic
RGPA

SEQ ID NO: 93 - protein, 10 aa, synthetic construct
  REGION 1..10: note = Synthetic
GGQPSGMWGW

SEQ ID NO: 94 - protein, 10 aa, synthetic construct
  REGION 1..10: note = Synthetic
FPRPLGITGL

SEQ ID NO: 95 - protein, 8 aa, synthetic construct
  REGION 1..8: note = Synthetic
SPLTGRSG

SEQ ID NO: 96 - protein, 8 aa, synthetic construct
  REGION 1..8: note = Synthetic
SAGFSLPA

SEQ ID NO: 97 - protein, 9 aa, synthetic construct
  REGION 1..9: note = Synthetic
LAPLGLQRR

SEQ ID NO: 98 - protein, 8 aa, synthetic construct
  REGION 1..8: note = Synthetic
SGGPLGVR

SEQ ID NO: 99 - protein, 4 aa, synthetic construct
  REGION 1..4: note = Synthetic
PLGL

SEQ ID NO: 100 - protein, 7 aa, synthetic construct
  REGION 1..7: note = Synthetic
SGRSDNI

SEQ ID NO: 101 - protein, 165 aa, synthetic construct
  REGION 1..165: note = Synthetic
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 102 - protein, 169 aa, synthetic construct
  REGION 1..169: note = Synthetic
CDLPHTHNLRNKRAFTLLAQMRRLSPVSCLKDRKDFGFPLEKVDGQQIQKAQAIPVLHELTQQILSLFTSKESSTAWDASLLDSFCNDLQQQLSGLQACLMQQVGVQESPLTQEDSLLAVREYFHRITVYLREKKHSPCAWEVVRAEVWRALSSSANLLGRLREERNES

SEQ ID NO: 103 - protein, 167 aa, synthetic construct
  REGION 1..167: note = Synthetic
CDLPHTYNLRNKRALKVLAQMRRLPFLSCLKDRQDFGFPLEKVDNQQIQKAQAIPVLRDLTQQTLNLFTSKASSAAWNATLLDSFCNDLHQQLNDLQTCLMQQVGVQEPPLTQEDALLAVRKYFHRITVYLREKKHSPCAWEVVRAEVWRALSSSVNLLPRLSEEKE

SEQ ID NO: 104 - protein, 165 aa, synthetic construct [sequence omitted]

SEQ ID NO: 105 - protein, 495 aa, synthetic construct
  REGION 1..495: note = Synthetic
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKECDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMNEDSILAVRKYFQRITLYLKEKKYSPCAWEVVRAEIMRSFSLSTNLQESLRSKECDLPQTHSLGSRRTLMLLAQMRRISLFSCLKDRRDFGFPQEEFGNQFQKAETIPVLHEMIQQIFNLFSTKDSSAAWDETLLDKFYTELYQQLNDLEACVIQGVGVTETPLMNEDSILAVRKYFQRITLYLKEKKYSPCAWEVVRAEIMRSFSLSTNLQESLRSKE

SEQ ID NO: 106 - protein, 166 aa, synthetic construct
  REGION 1..166: note = Synthetic
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 107 - protein, 165 aa, synthetic construct
  REGION 1..165: note = Synthetic
SYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 108 - protein, 182 aa, synthetic construct
  REGION 1..182: note = Synthetic
MNNRWILHAAFLLCFSTTALSINYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN

SEQ ID NO: 109 - protein, 184 aa, synthetic construct
  REGION 1..184: note = Synthetic
MANRWTLHIAFLLCFSTTALSIDYKQLQFRQSTSIRTCQKLLRQLNGRLNLSYRTDFKIPMEVMHPSQMEKSYTAFAIQVMLQNVFLVFRSNFSSTGWNETIVESLLDELHQQTELLEIILKEKQEERLTWVTSTTTLGLKSYYWRVQRYLKDKKYNSYAWMVVRAEVFRNFSIILRLNRNFQN

SEQ ID NO: 110 - protein, 173 aa, synthetic construct
  REGION 1..173: note = Synthetic
MCDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 111 - protein, 271 aa, synthetic construct
  REGION 1..271: note = Synthetic
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 112 - protein, 270 aa, synthetic construct
  REGION 1..270: note = Synthetic
MAKVPDLFEDLKNCYSENEDYSSAIDHLSLNQKSFYDASYGSLHETCTDQFVSLRTSETSKMSNFTFKESRVTVSATSSNGKILKKRRLSFSETFTEDDLQSITHDLEETIQPRSAPYTYQSDLRYKLMKLVRQKFVMNDSLNQTIYQDVDKHYLSTTWLNDLQQEVKFDMYAYSSGGDDSKYPVTLKISDSQLFVSAQGEDQPVLLKELPETPKLITGSETDLIFFWKSINSKNYFTSAAYPELFIATKEQSRVHLARGLPSMTDFQIS

SEQ ID NO: 113 - protein, 269 aa, synthetic construct
  REGION 1..269: note = Synthetic
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 114 - protein, 269 aa, synthetic construct
  REGION 1..269: note = Synthetic
MATVPELNCEMPPFDSDENDLFFEVDGPQKMKGCFQTFDLGCPDESIQLQISQQHINKSFRQAVSLIVAVEKLWQLPVSFPWTFQDEDMSTFFSFIFEEEPILCDSWDDDDNLLVCDVPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSS

SEQ ID NO: 115 - protein, 177 aa, synthetic construct
  REGION 1..177: note = Synthetic
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 116 - protein, 178 aa, synthetic construct
  REGION 1..178: note = Synthetic
MEICWGPYSHLISLLLILLFHSEAACRPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 117 - protein, 193 aa, synthetic construct
  REGION 1..193: note = Synthetic
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 118 - protein, 192 aa, synthetic construct
  REGION 1..192: note = Synthetic
MAAMSEDSCVNFKEMMFIDNTLYFIPEENGDLESDNFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 119 - protein, 150 aa, synthetic construct
  REGION 1..150: note = Synthetic
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIIS

SEQ ID NO: 120 - protein, 169 aa, synthetic construct
  REGION 1..169: note = Synthetic
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 121 - protein, 153 aa, synthetic construct [sequence omitted]

SEQ ID NO: 122 - protein, 140 aa, synthetic construct
  REGION 1..140: note = Synthetic
MGLNPQLVVILLFFLECTRSHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 123 - protein, 177 aa, synthetic construct
  REGION 1..177: note = Synthetic
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 124 - protein, 154 aa, synthetic construct
  REGION 1..154: note = Synthetic
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 125 - protein, 144 aa, synthetic construct
  REGION 1..144: note = Synthetic
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 126 - protein, 144 aa, synthetic construct
  REGION 1..144: note = Synthetic
MLVTYILASVLLFSSVLGQRCSTTWGIRDTNYLIENLKDDPPSKCSCSGNVTSCLCLSVPTDDCTTPCYREGLLQLTNATQKSRLLPVFHRVKRIVEVLKNITCPSFSCEKPCNQTMAGNTLSFLKSLLGTFQKTEMQRQKSRP

SEQ ID NO: 127 - protein, 146 aa, synthetic construct [sequence omitted]

SEQ ID NO: 128 - protein, 131 aa, synthetic construct
  REGION 1..131: note = Synthetic
MALWVTAVLALACLGGLAAPGPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPF

SEQ ID NO: 129 - protein, 162 aa, synthetic construct
  REGION 1..162: note = Synthetic
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 130 - protein, 162 aa, synthetic construct
  REGION 1..162: note = Synthetic
MKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 131 - protein, 152 aa, synthetic construct
  REGION 1..152: note = Synthetic
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 132 - protein, 166 aa, synthetic construct
  REGION 1..166: note = Synthetic
MVLASSTTSIHTMLLLLLMLFHLGLQASISGRDTHRLTRTLNCSSIVKEIIGKLPEPELKTDDEGPSLRNKSFRRVNLSKFVESQGEVDPEDRYVIKSNLQKLNCCLPTSANDSALPGVFIRDLDDFRKKLRFYMVHLNDLETVLTSRPPQPASGSVSPNRGTVEC

SEQ ID NO: 133 - protein, 134 aa, synthetic construct
  REGION 1..134: note = Synthetic
MRMLLHLSLLALGAAYVYAIPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 134 - protein, 133 aa, synthetic construct
  REGION 1..133: note = Synthetic
MRRMLLHLSVLTLSCVWATAMEIPMSTVVKETLTQLSAHRALLTSNETMRLPVPTHKNHQLCIGEIFQGLDILKNQTVRGGTVEMLFQNLSLIKKYIDRQKEKCGEERRRTRQFLDYLQEFLGVMSTEWAMEG

SEQ ID NO: 135 - protein, 144 aa, synthetic construct
  REGION 1..144: note = Synthetic
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 136 - protein, 141 aa, synthetic construct
  REGION 1..141: note = Synthetic
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 137 - protein, 212 aa, synthetic construct
  REGION 1..212: note = Synthetic
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 138 - protein, 211 aa, synthetic construct
  REGION 1..211: note = Synthetic
MKFLSARDFHPVAFLGLMLVTTTAFPTSQVRRGDFTEDTTPNRPVYTTSQVGGLITHVLWEIVEMRKELCNGNSDCMNNDDALAENNLKLPEIQRNDGCYQTGYNQEICLLKISSGLLEYHSYLEYMKNNLKDNKKDKARVLQRDTETLIHIFNQEVKDLHKIVLPTPISNALLTDKLESQKEWLRTKTIQFILKSLEEFLKVTLRSTRQT

SEQ ID NO: 139 - protein, 199 aa, synthetic construct
  REGION 1..199: note = Synthetic
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 140 - protein, 199 aa, synthetic construct
  REGION 1..199: note = Synthetic
MNCVCRLVLVVLSLWPDRVVAPGPPAGSPRVSSDPRADLDSAVLLTRSLLADTRQLAAQMRDKFPADGDHSLDSLPTLAMSAGTLGSLQLPGVLTRLRVDLMSYLRHVQWLRRAGGPSLKTLEPELGALQARLERLLRRLQLLMSRLALPQAAPDQPVIPLGPPASAWGSIRAAHAILGGLHLTLDWAVRGLLLLKTRL

SEQ ID NO: 141 - protein, 207 aa, synthetic construct
  REGION 1..207: note = Synthetic
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 142 - protein, 208 aa, synthetic construct
  REGION 1..208: note = Synthetic
MAQLSAQRRMKLMALQLLLWQSALWSGREAVPLVTVSALPPSLPLPRSFLLKSLEQVRKIQASGSVLLEQLCATYKLCHPEELVLLGHSLGIPKASLSGCSSQALQQTQCLSQLHSGLCLYQGLLQALSGISPALAPTLDLLQLDVANFATTIWQQMENLGVAPTVQPTQSAMPAFTSAFQRRAGGVLAISYLQGFLETARLALHHLA

SEQ ID NO: 143 - protein, 219 aa, synthetic construct
  REGION 1..219: note = Synthetic
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 144 - protein, 328 aa, synthetic construct
  REGION 1..328: note = Synthetic
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 145 - protein, 335 aa, synthetic construct
  REGION 1..335: note = Synthetic
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 146 - protein, 215 aa, synthetic construct
  REGION 1..215: note = Synthetic
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 147 - protein, 202 aa, synthetic construct
  REGION 1..202: note = Synthetic
MKVLAAGVVPLLLVLHWKHGAGSPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

SEQ ID NO: 148 - protein, 203 aa, synthetic construct [sequence omitted]

SEQ ID NO: 149 - protein, 252 aa, synthetic construct
  REGION 1..252: note = Synthetic
MGVLLTQRTLLSLVLALLFPSMASMAAIGSCSKEYRVLLGQLQKQTDLMQDTSRLLDPYIRIQGLDVPKLREHCRERPGAFPSEETLRGLGRRGFLQTLNATLGCVLHRLADLEQRLPKAQDLERSGLNIEDLEKLQMARPNILGLRNNIYCMAQLLDNSDTAEPTKAGRGASQPPTPTPASDAFQRKLEGCRFLHGYHRFMHSVGRVFSKWGESPNRSRRHSPHQALRKGVRRTRPSRKGKRLMTRGQLPR

SEQ ID NO: 150 - protein, 263 aa, synthetic construct
  REGION 1..263: note = Synthetic
MQTRLLRTLLSLTLSLLILSMALANRGCSNSSSQLLSQLQNQANLTGNTESLLEPYIRLQNLNTPDLRAACTQHSVAFPSEDTLRQLSKPHFLSTVYTTLDRVLYQLDALRQKFLKTPAFPKLDSARHNILGIRNNVFCMARLLNHSLEIPEPTQTDSGASRSTTTPDVFNTKIGSCGFLWGYHRFMGSVGRVFREWDDGSTRSRRQSPLRARRKGTRRIRVRHKGTRRIRVRRKGTRRIWVRRKGSRKIRPSRSTQSPTTRA

SEQ ID NO: 151 - protein, 178 aa, synthetic construct
  REGION 1..178: note = Synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 152 - protein, 178 aa, synthetic construct
  REGION 1..178: note = Synthetic
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 153 - protein, 176 aa, synthetic construct
  REGION 1..176: note = Synthetic
MKASSLAFSLLSAAFYLLWTPSTGLKTLNLGSCVIATNLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEETE

SEQ ID NO: 154 - protein, 176 aa, synthetic construct
  REGION 1..176: note = Synthetic
MKGFGLAFGLFSAVGFLLWTPLTGLKTLHLGSCVITANLQAIQKEFSEIRDSVQAEDTNIDIRILRTTESLKDIKSLDRCCFLRHLVRFYLDRVFKVYQTPDHHTLRKISSLANSFLIIKKDLSVCHSHMACHCGEEAMEKYNQILSHFIELELQAAVVKALGELGILLRWMEEML

SEQ ID NO: 155 - protein, 546 aa, synthetic construct
  REGION 1..546: note = Synthetic
MKNQDKKNGAAKQSNPKSSPGQPEAGPEGAQERPSQAAPAVEAEGPGSSQAPRKPEGAQARTAQSGALRDVSEELSRQLEDILSTYCVDNNQGGPGEDGAQGEPAEPEDAEKSRTYVARNGEPEPTPVVNGEKEPSKGDPNTEEIRQSDEVGDRDHRRPQEKKKAKGLGKEITLLMQTLNTLSTPEEKLAALCKKYAELLEEHRNSQKQMKLLQKKQSQLVQEKDHLRGEHSKAVLARSKLESLCRELQRHNRSLKEEGVQRAREEEEKRKEVTSHFQVTLNDIQLQMEQHNERNSKLRQENMELAERLKKLIEQYELREEHIDKVFKHKDLQQQLVDAKLQQAQEMLKEAEERHQREKDFLLKEAVESQRMCELMKQQETHLKQQLALYTEKFEEFQNTLSKSSEVFTTFKQEMEKMTKKIKKLEKETTMYRSRWESSNKALLEMAEEKTVRDKELEGLQVKIQRLEKLCRALQTERNDLNKRVQDLSAGGQGSLTDSGPERRPEGPGAQAPSSPRVTEAPCYPGAPSTEASGQTGPQEPTSARA

SEQ ID NO: 156 - protein, 554 aa, synthetic construct
  REGION 1..554: note = Synthetic
MKNQDKKNGPAKHSNSKGSPGQREAGPEGAHGRPRQTAPGAEAEGSTSQAPGKTEGARAKAAQPGALCDVSEELSRQLEDILSTYCVDNNQGGPAEEGAQGEPTEPEDTEKSRTYAARNGEPEPGIPVVNGEKETSKGEPGTEEIRASDEVGDRDHRRPQEKKKAKGLGKEITLLMQTLNTLSTPEEKLAALCKKYAELLEEHRNSQKQMKLLQKKQSQLVQEKDHLRGEHSKAVLARSKLESLCRELQRHNRSLKEEGVQRAREEEEKRKEVTSHFQVTLNDIQLQMEQHNERNSKLRQENMELAERLKKLIEQYELREEHIDKVFKHKDLQQQLVDAKLQQAQEMLKEAEERHQREKEFLLKEAVESQRMCELMKQQETHLKQQLALYTEKFEEFQNTLSKSSEVFTTFKQEMEKMTKKIKKLEKETTMYRSRWESSNKALLEMAEEKTVRDKELEGLQVKIQRLEKLCRALQTERNDLNKRVQDLTAGGITDIGSERRPEATTASKEQGVESPGAQPASSPRATDAPCCSGAPSTGTAGQTGPGEPTPATA

SEQ ID NO: 157 - protein, 1331 aa, synthetic construct
  REGION 1..1331: note = Synthetic
MESHSRAGKSRKSAKFRSISRSLMLCNAKTSDDGSSPDEKYPDPFEISLAQGKEGIFHSSVQLADTSEAGPSSVPDLALASEAAQLQAAGNDRGKTCRRIFFMKESSTASSREKPGKLEAQSSNFLFPKACHQRARSNSTSVNPYCTREIDFPMTKKSAAPTDRQPYSLCSNRKSLSQQLDCPAGKAAGTSRPTRSLSTAQLVQPSGGLQASVISNIVLMKGQAKGLGFSIVGGKDSIYGPIGIYVKTIFAGGAAAADGRLQEGDEILELNGESMAGLTHQDALQKFKQAKKGLLTLTVRTRLTAPPSLCSHLSPPLCRSLSSSTCITKDSSSFALESPSAPISTAKPNYRIMVEVSLQKEAGVGLGIGLCSVPYFQCISGIFVHTLSPGSVAHLDGRLRCGDEIVEISDSPVHCLTLNEVYTILSHCDPGPVPIIVSRHPDPQVSEQQLKEAVAQAVENTKFGKERHQWSLEGVKRLESSWHGRPTLEKEREKNSAPPHRRAQKVMIRSSSDSSYMSGSPGGSPGSGSAEKPSSDVDISTHSPSLPLAREPVVLSIASSRLPQESPPLPESRDSHPPLRLKKSFEIVRKPMSSKPKPPPRKYFKSDSDPQKSLEERENSSCSSGHTPPTCGQEARELLPLLLPQEDTAGRSPSASAGCPGPGIGPQTKSSTEGEPGWRRASPVTQTSPIKHPLLKRQARMDYSFDTTAEDPWVRISDCIKNLFSPIMSENHGHMPLQPNASLNEEEGTQGHPDGTPPKLDTANGTPKVYKSADSSTVKKGPPVAPKPAWFRQSLKGLRNRASDPRGLPDPALSTQPAPASREHLGSHIRASSSSSSIRQRISSFETFGSSQLPDKGAQRLSLQPSSGEAAKPLGKHEEGRFSGLLGRGAAPTLVPQQPEQVLSSGSPAASEARDPGVSESPPPGRQPNQKTLPPGPDPLLRLLSTQAEESQGPVLKMPSQRARSFPLTRSQSCETKLLDEKTSKLYSISSQVSSAVMKSLLCLPSSISCAQTPCIPKEGASPTSSSNEDSAANGSAETSALDTGFSLNLSELREYTEGLTEAKEDDDGDHSSLQSGQSVISLLSSEELKKLIEEVKVLDEATLKQLDGIHVTILHKEEGAGLGFSLAGGADLENKVITVHRVFPNGLASQEGTIQKGNEVLSINGKSLKGTTHHDALAILRQAREPRQAVIVTRKLTPEAMPDLNSSTDSAASASAASDVSVESTAEATVCTVTLEKMSAGLGFSLEGGKGSLHGDKPLTINRIFKGAASEQSETVQPGDEILQLGGTAMQGLTRFEAWNIIKALPDGPVTIVIRRKSLQSKETTAAGDS

SEQ ID NO: 158 - protein, 1322 aa, synthetic construct
  REGION 1..1322: note = Synthetic
MEPHGHSGKSRKSTKFRSISRSLILCNAKTSDDGSSPDEKYPDPFETSLCQGKEGFFHSSMQLADTFEAGLSNIPDLALASDSAQLAAAGSDRGKHCRKMFFMKESSSTSSKEKSGKPEAQSSSFLFPKACHQRTRSNSTSVNPYSAGEIDFPMTKKSAAPTDRQPYSLCSNRKSLSQQLDYPILGTARPTRSLSTAQLGQLSGGLQASVISNIVLMKGQAKGLGFSIVGGKDSIYGPIGIYVKSIFAGGAAAADGRLQEGDEILELNGESMAGLTHQDALQKFKQAKKGLLTLTVRTRLTTPPSLCSHLSPPLCRSLSSSTCGAQDSSPFSLESPASPASTAKPNYRIMVEVSLKKEAGVGLGIGLCSIPYFQCISGIFVHTLSPGSVAHLDGRLRCGDEIVEINDSPVHCLTLNEVYTILSHCDPGPVPIIVSRHPDPQVSEQQLKEAVAQAVEGVKFGKDRHQWSLEGVKRLESSWHGRPTLEKEREKHSAPPHRRAQKIMVRSSSDSSYMSGSPGGSPCSAGAEPQPSEREGSTHSPSLSPGEEQEPCPGVPSRPQQESPPLPESLERESHPPLRLKKSFEILVRKPTSSKPKPPPRKYFKNDSEPQKKLEEKEKVTDPSGHTLPTCSQETRELLPLLLQEDTAGRAPCTAACCPGPAASTQTSSSTEGESRRSASPETPASPGKHPLLKRQARMDYSFDITAEDPWVRISDCIKNLFSPIMSENHSHTPLQPNTSLGEEDGTQGCPEGGLSKMDAANGAPRVYKSADGSTVKKGPPVAPKPAWFRQSLKGLRNRAPDPRRPPEVASAIQPTPVSRDPPGPQPQASSSIRQRISSFENFGSSQLPDRGVQRLSLQPSSGETTKFPGKQDGGRFSGLLGQGATVTAKHRQTEVESMSTTFPNSSEVRDPGLPESPPPGQRPSTKALSPDPLLRLLTTQSEDTQGPGLKMPSQRARSFPLTRTQSCETKLLDEKASKLYSISSQLSSAVMKSLLCLPSSVSCGQITCIPKERVSPKSPCNNSSAAEGFGEAMASDTGFSLNLSELREYSEGLTEPGETEDRNHCSSQAGQSVISLLSAEELEKLIEEVRVLDEATLKQLDSIHVTILHKEEGAGLGFSLAGGADLENKVITVHRVFPNGLASQEGTIQKGNEVLSINGKSLKGATHNDALAILRQARDPRQAVIVTRRTTVEATHDLNSSTDSAASASAASDISVESKEATVCTVTLEKTSAGLGFSLEGGKGSLHGDKPLTINRIFKGTEQGEMVQPGDEILQLAGTAVQGLTRFEAWNVIKALPDGPVTIVIRRTSLQCKQTTASADS

SEQ ID NO: 159 - protein, 155 aa, synthetic construct
  REGION 1..155: note = Synthetic
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 160 - protein, 158 aa, synthetic construct
  REGION 1..158: note = Synthetic
MSPGRASSVSLMLLLLLSLAATVKAAAIIPQSSACPNTEAKDFLQNVKVNLKVFNSLGAKVSSRRPSDYLNRSTSPWTLHRNEDPDRYPSVIWEAQCRHQRCVNAEGKLDHHMNSVLIQQEILVLKREPESCPFTFRVEKMLVGVGCTCVASIVRQAA

SEQ ID NO: 161 - protein, 261 aa, synthetic construct
  REGION 1..261: note = Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 162 - protein, 260 aa, synthetic construct
  REGION 1..260: note = Synthetic
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 163 - protein, 244 aa, synthetic construct
  REGION 1..244: note = Synthetic
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 164 - protein, 306 aa, synthetic construct
  REGION 1..306: note = Synthetic
MGTRGLQGLGGRPQGRGCLLLAVAGATSLVTLLLAVPITVLAVLALVPQDQGRRVEKIIGSGAQAQKRLDDSKPSCILPSPSSLSETPDPRLHPQRSNASRNLASTSQGPVAQSSREASAWMTILSPAADSTPDPGVQQLPKGEPETDLNPELPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVG

SEQ ID NO: 165 - protein, 232 aa, synthetic construct
  REGION 1..232: note = Synthetic
STESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 166 - protein, 138 aa, synthetic construct
  REGION 1..138: note = Synthetic
NHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 167 - protein, 205 aa, synthetic construct
  REGION 1..205: note = Synthetic
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 168 - protein, 254 aa, synthetic construct
  REGION 1..254: note = Synthetic
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 169 - protein, 309 aa, synthetic construct
  REGION 1..309: note = Synthetic
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 170 - protein, 146 aa, synthetic construct
  REGION 1..146: note = Synthetic
AVLTQKQKKQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 171 - protein, 241 aa, synthetic construct
  REGION 1..241: note = Synthetic
MPASSPGHMGGSVREPALSVALWLSWGAVLGAVTCAVALLIQQTELQSLRREVSRLQRSGGPSQKQGERPWQSLWEQSPDVLEAWKDGAKSRRRRAVLTQKHKKKHSVLHLVPVNITSKADSDVTEVMWQPVLRRGRGLEAQGDIVRVWDTGIYLLYSQVLFHDVTFTMGQVVSREGQGRRETLFRCIRSMPSDPDRAYNSCYSAGVFHLHQGDIITVKIPRANAKLSLSPHGTFLGFVKL

SEQ ID NO: 172 - protein, 193 aa, synthetic construct
  REGION 1..193: note = Synthetic
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 173 - protein, 195 aa, synthetic construct
  REGION 1..195: note = Synthetic
MPEEGRPCPWVRWSGTAFQRQWPWLLLVVFITVFCCWFHCSGLLSKQQQRLLEHPEPHTAELQLNLTVPRKDPTLRWGAGPALGRSFTHGPELEEGHLRIHQDGLYRLHIQVTLANCSSPGSTLQHRATLAVGICSPAAHGISLLRGRFGQDCTVALQRLTYLVHGDVLCTNLTLPLLPSRNADETFFGVQWICP

SEQ ID NO: 174 - protein, 234 aa, synthetic construct
  REGION 1..234: note = Synthetic
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 175 - protein, 239 aa, synthetic construct
  REGION 1..239: note = Synthetic
MEPGLQQAGSCGAPSPDPAMQVQPGSVASPWRSTRPWRSTSRSYFYLSTTALVCLVVAVAIILVLVVQKKDSTPNTTEKAPLKGGNCSEDLFCTLKSTPSKKSWAYLQVSKHLNNTKLSWNEDGTIHGLIYQDGNLIVQFPGLYFIVCQLQFLVQCSNHSVDLTLQLLINSKIKKQTLVTVCESGVQSKNIYQNLSQFLLHYLQVNSTISVRVDNFQYVDTNTFPLDNVLSVFLYSSSD

SEQ ID NO: 176 - protein, 281 aa, synthetic construct
  REGION 1..281: note = Synthetic
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 177 - protein, 279 aa, synthetic construct
  REGION 1..279: note = Synthetic
MQQPMNYPCPQIFWVDSSATSSWAPPGSVFPCPSCGPRGPDQRRPPPPPPPVSPLPPPSQPLPLPPLTPLKKKDHNTNLWLPVVFFMVLVALVGMGLGMYQLFHLQKELAELREFTNQSLKVSSFEKQIANPSTPSEKKEPRSVAHLTGNPHSRSIPLEWEDTYGTALISGVKYKKGGLVINETGLYFVYSKVYFRGQSCNNQPLNHKVYMRNSKYPEDLVLMEEKRLNYCTTGQIWAHSSYLGAVFNLTSADHLYVNISQLSLINFEESKTFFGLYKL

SEQ ID NO: 178 - protein, 199 aa, synthetic construct
  REGION 1..199: note = Synthetic
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 179 - protein, 173 aa, synthetic construct
  REGION 1..173: note = Synthetic
MEEMPLRESSPQRAERCKKSWLLCIVALLLMLLCSLGTLIYTSLKPTAIESCMVKFELSSSKWHMTSPKPHCVNTTSDGKLKILQSGTYLIYGQVIPVDKKYIKDNAPFVVQIYKKNDVLQTLMNDFQILPIGGVYELHAGDNIYLKFNSKDHIQKTNTYWGIILMPDLPFIS

SEQ ID NO: 180 - protein, 240 aa, synthetic construct
  REGION 1..240: note = Synthetic
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 181 - protein, 239 aa, synthetic construct
  REGION 1..239: note = Synthetic
MESVVQPSVFVVDGQTDIPFRRLEQNHRRRRCGTVQVSLALVLLLGAGLATQGWFLLRLHQRLGDIVAHLPDGGKGSWEKLIQDQRSHQANPAAHLTGANASLIGIGGPLLWETRLGLAFLRGLTYHDGALVTMEPGYYYVYSKVQLSGVGCPQGLANGLPITHGLYKRTSRYPKELELLVSRRSPCGRANSSRVWWDSSFLGGVVHLEAGEEVVVRVPGNRLVRPRDGTRSYFGAFMV

SEQ ID NO: 182 - protein, 183 aa, synthetic construct
  REGION 1..183: note = Synthetic
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 183 - protein, 197 aa, synthetic construct
  REGION 1..197: note = Synthetic
MEGEGVQPLDENLENGSRPRFKWKKTLRLVVSGIKGAGMLLCFIYVCLQLSSSPAKDPPIQRLRGAVTRCEDGQLFISSYKNEYQTMEVQNNSVVIKCDGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVP

SEQ ID NO: 184 - protein, 285 aa, synthetic construct
  REGION 1..285: note = Synthetic
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 185 - protein, 249 aa, synthetic construct
  REGION 1..249: note = Synthetic
MAMAFCPKDQYWDSSRKSCVSCALTCSQRSQRTCTDFCKFINCRKEQGRYYDHLLGACVSCDSTCTQHPQQCAHFCEKRPRSQANLQPELGRPQAGEVEVRSDNSGRHQGSEHGPGLRLSSDQLTLYCTLGVCLCAIFCCFLVALASFLRRRGEPLPSQPAGPRGSQANSPHAHRPVTEACDEVTASPQPVETCSFCFPERSSPTQESAPRSLGIHGFAGTAAPQPCMRATVGGLGVLRASTGDARPAT

SEQ ID NO: 186 - protein, 281 aa, synthetic construct [sequence omitted]

SEQ ID NO: 187 - protein, 291 aa, synthetic construct
  REGION 1..291: note = Synthetic
MPSSGALKDLSFSQHFRMMVICIVLLQVLLQAVSVAVTYMYFTNEMKQLQDNYSKIGLACFSKTDEDFWDSTDGEILNRPCLQVKRQLYQLIEEVTLRTFQDTISTVPEKQLSTPPLPRGGRPQKVAAHITGITRRSNSALIPISKDGKTLGQKIESWESSRKGHSFLNHVLFRNGELVIEQEGLYYIYSQTYFRFQEAEDASKMVSKDKVRTKQLVQYIYKYTSYPDPIVLMKSARNSCWSRDAEYGLYSIYQGGLFELKKNDRIFVSVTNEHLMDLDQEASFFGAFLIN

SEQ ID NO: 188 - protein, 249 aa, synthetic construct
  REGION 1..249: note = Synthetic
MAARRSQRRRGRRGEPGTALLVPLALGLGLALACLGLLLAVVSLGSRASLSAQEPAQEELVAEEDQDPSELNPQTEESQDPAPFLNRLVRPRRSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 189 - protein, 129 aa, synthetic construct
  REGION 1..129: note = Synthetic
MASAWPRSLPQILVLGFGLVLMRAAAGEQAPGTSPCSSGSSWSADLDKCMDCASCPARPHSDFCLGCAAAPPAHFRLLWPILGGALSLVLVLALVSSFLVWRRCRRREKFTTPIEETGGEGCPGVALIQ

SEQ ID NO: 190 - protein, 317 aa, synthetic construct
  REGION 1..317: note = Synthetic
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 191 - protein, 316 aa, synthetic construct
  REGION 1..316: note = Synthetic
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENADLQDSTLESEDTLPDSCRRMKQAFQGAVQKELQHIVGPQRFSGAPAMMEGSWLDVAQRGKPEAQPFAHLTINAASIPSGSHKVTLSSWYHDRGWAKISNMTLSNGKLRVNQDGFYYLYANICFRHHETSGSVPTDYLQLMVYVVKTSIKIPSSHNLMKGGSTKNWSGNSEFHFYSINVGGFFKLRAGEEISIQVSNPSLLDPDQDATYFGAFKVQDID

SEQ ID NO: 192 - protein, 390 aa, synthetic construct
  REGION 1..390: note = Synthetic
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 193 - protein, 390 aa, synthetic construct
  REGION 1..390: note = Synthetic
MPPSGLRLLPLLLPLPWLLVLTPGRPAAGLSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 194 - protein, 414 aa, synthetic construct
  REGION 1..414: note = Synthetic
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 195 - protein, 414 aa, synthetic construct
  REGION 1..414: note = Synthetic
MHYCVLSTFLLLHLVPVALSLSTCSTLDMDQFMRKRIEAIRGQILSKLKLTSPPEDYPEPDEVPPEVISIYNSTRDLLQEKASRRAAACERERSDEEYYAKEVYKIDMPSHLPSENAIPPTFYRPYFRIVRFDVSTMEKNASNLVKAEFRVFRLQNPKARVAEQRIELYQILKSKDLTSPTQRYIDSKVVKTRAEGEWLSFDVTDAVQEWLHHKDRNLGFKISLHCPCCTFVPSNNYIIPNKSEELEARFAGIDGTSTYASGDQKTIKSTRKKTSGKTPHLLLMLLPSYRLESQQSSRRKKRALDAAYCFRNVQDNCCLRPLYIDFKRDLGWKWIHEPKGYNANFCAGACPYLWSSDTQHTKVLSLYNTINPEASASPCCVSQDLEPLTILYYIGNTPKIEQLSNMIVKSCKCS

SEQ ID NO: 196 - protein, 412 aa, synthetic construct
  REGION 1..412: note = Synthetic
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 197 - protein, 410 aa, synthetic construct
  REGION 1..410: note = Synthetic
MHLQRALVVLALLNLATISLSLSTCTTLDFGHIKKKRVEAIRGQILSKLRLTSPPEPSVMTHVPYQVLALYNSTRELLEEMHGEREEGCTQETSESEYYAKEIHKFDMIQGLAEHNELAVCPKGITSKVFRFNVSSVEKNGTNLFRAEFRVLRVPNPSSKRTEQRIELFQILRPDEHIAKQRYIGGKNLPTRGTAEWLSFDVTDTVREWLLRRESNLGLEISIHCPCHTFQPNGDILENVHEVMEIKFKGVDNEDDHGRGDLGRLKKQKDHHNPHLILMMIPPHRLDSPGQGSQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 198 - protein, 193 aa, synthetic construct
  REGION 1..193: note = Synthetic
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 199 - protein, 192 aa, synthetic construct
  REGION 1..192: note = Synthetic
MGVPERPTLLLLLSLLLIPLGLPVLCAPPRLICDSRVLERYILEAKEAENVTMGCAEGPRLSENITVPDTKVNFYAWKRMEVEEQAIEVWQGLSLLSEAILQAQALLANSSQPPETLQLHIDKAISGLRSLTSLLRVLGAQKELMSPPDTTPPAPLRTLTVDTFCKLFRVYANFLRGKLKLYTGEVCRRGDR

SEQ ID NO: 200 - protein, 353 aa, synthetic construct
  REGION 1..353: note = Synthetic
MELTELLLVVMLLLTARLTLSSPAPPACDLRVLSKLLRDSHVLHSRLSQCPEVHPLPTPVLLPAVDFSLGEWKTQMEETKAQDILGAVTLLLEGVMAARGQLGPTCLSSLLGQLSGQVRLLLGALQSLLGTQLPPQGRTTAHKDPNAIFLSFQHLLRGKVRFLMLVGGSTLCVRRAPPTTAVPSRTSLVLTLNELPNRTSGLLETNFTASARTTGSGLLKWQQGFRAKIPGLLNQTSRSLDQIPGYLNRIHELLNGTRGLFPGPSRRTLGAPDISSGTSDTGSLPPNLQPGYSPSPTHPPTGQYTLFPLPPTLPTPVVQLHPLLPDPSAPTPTPTSPLLNTSYTHSQNLSQEG

SEQ ID NO: 201 - protein, 356 aa, synthetic construct
  REGION 1..356: note = Synthetic
MELTDLLLAAMLLAVARLTLSSPVAPACDPRLLNKLLRDSHLLHSRLSQCPDVDPLSIPVLLPAVDFSLGEWKTQTEQSKAQDILGAVSLLLEGVMAARGQLEPSCLSSLLGQLSGQVRLLLGALQGLLGTQLPLQGRTTAHKDPNALFLSLQQLLRGKVRFLLLVEGPTLCVRRTLPTTAVPSSTSQLLTLNKFPNRTSGLLETNFSVTARTAGPGLLSRLQGFRVKITPGQLNQTSRSPVQISGYLNRTHGPVNGTHGLFAGTSLQTLEASDISPGAFNKGSLAFNLQGGLPPSPSLAPDGHTPFPPSPALPTTHGSPPQLHPLFPDPSTTMPNSTAPHPVTMYPHPRNLSQET

SEQ ID NO: 202 - protein, 235 aa, synthetic construct
  REGION 1..235: note = Synthetic
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 203 - protein, 232 aa, synthetic construct
  REGION 1..232: note = Synthetic
MTVLAPAWSPNSSLLLLLLLLSPCLRGTPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPRPRQLLLLLLLLLPLTLVLLAAAWGLRWQRARRRGELHPGVPLPSHP

SEQ ID NO: 204 - protein, 273 aa, synthetic construct
  REGION 1..273: note = Synthetic
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 205 - protein, 273 aa, synthetic construct
  REGION 1..273: note = Synthetic
MKKTQTWIITCIYLQLLLFNPLVKTKEICGNPVTDNVKDITKLVANLPNDYMITLNYVAGMDVLPSHCWLRDMVIQLSLSLTTLLDKFSNISEGLSNYSIIDKLGKIVDDLVLCMEENAPKNIKESPKRPETRSFTPEEFFSIFNRSIDAFKDFMVASDTSDCVLSSTLGPEKDSRVSVTKPFMLPPVAASSLRNDSSSSNRKAAKAPEDSGLQWTAMALPALISLVIGFAFGALYWKKKQSSLTRAVENIQINEEDNEISMLQQKEREFQEV

SEQ ID NO: 206 - protein, 554 aa, synthetic construct
  REGION 1..554: note = Synthetic
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSFSPQLQESVFHLLVPSVILVLLAVGGLLFYRWRRRSHQEPQRADSPLEQPEGSPLTQDDRQVELPV

SEQ ID NO: 207 - protein, 552 aa, synthetic construct
  REGION 1..552: note = Synthetic
MTARGAAGRCPSSTWLGSRLLLVCLLMSRSIAKEVSEHCSHMIGNGHLKVLQQLIDSQMETSCQIAFEFVDQEQLDDPVCYLKKAFFLVQDIIDETMRFKDNTPNANATERLQELSNNLNSCFTKDYEEQNKACVRTFHETPLQLLEKIKNFFNETKNLLEKDWNIFTKNCNNSFAKCSSRDVVTKPDCNCLYPKATPSSDPASASPHQPPAPSMAPLAGLAWDDSQRTEGSSLLPSELPLRIEDPGSAKQRPPRSTCQTLESTEQPNHGDRLTEDSQPHPSAGGPVPGVEDILESSLGTNWVLEEASGEASEGFLTQEAKFSPSTPVGGSIQAETDRPRALSASPFPKSTEDQKPVDITDRPLTEVNPMRPIGQTQNNTPEKTDGTSTLREDHQEPGSPHIATPNPQRVSNSATPVAQLLLPKSHSWGIVLPLGELEGKRSTRDRRSPAELEGGSASEGAARPVARFNSIPLTDTGHVEQHEGSSDPQIPESVFHLLVPGIILVLLTVGGLLFYKWKWRSHRDPQTLDSSVGRPEDSSLTQDEDRQVELPV

SEQ ID NO: 208 - protein, 711 aa, synthetic construct
  REGION 1..711: note = Synthetic
MGWLPLLLLLTQCLGVPGQRSPLNDFQVLRGTELQHLLHAVVPGPWQEDVADAEECAGRCGPLMDCRAFHYNVSSHGCQLLPWTQHSPHTRLRRSGRCDLFQKKDYVRTCIMNNGVGYRGTMATTVGGLPCQAWSHKFPNDHKYTPTLRNGLEENFCRNPDGDPGGPWCYTTDPAVRFQSCGIKSCREAACVWCNGEEYRGAVDRTESGRECQRWDLQHPHQHPFEPGKFLDQGLDDNYCRNPDGSERPWCYTTDPQIEREFCDLPRCGSEAQPRQEATTVSCFRGKGEGYRGTANTTTAGVPCQRWDAQIPHQHRFTPEKYACKDLRENFCRNPDGSEAPWCFTLRPGMRAAFCYQIRRCTDDVRPQDCYHGAGEQYRGTVSKTRKGVQCQRWSAETPHKPQFTFTSEPHAQLEENFCRNPDGDSHGPWCYTMDPRTPFDYCALRRCADDQPPSILDPPDQVQFEKCGKRVDRLDQRRSKLRVVGGHPGNSPWTVSLRNRQGQHFCGGSLVKEQWILTARQCFSSCHMPLTGYEVWLGTLFQNPQHGEPSLQRVPVAKMVCGPSGSQLVLLKLERSVTLNQRVALICLPPEWYVVPPGTKCEIAGWGETKGTGNDTVLNVALLNVISNQECNIKHRGRVRESEMCTEGLLAPVGACEGDYGGPLACFTHNCWVLEGIIIPNRVCARSRWPAVFTRVSVFVDWIHKVMRLG

SEQ ID NO: 209 - protein, 1378 aa, synthetic construct
  REGION 1..1378: note = Synthetic
MGLPLPLLQSSLLLMLLLRLSAASTNLNWQCPRIPYAASRDFSVKYVVPSFSAGGRVQATAAYEDSTNSAVFVATRNHLHVLGPDLQFIENLTTGPIGNPGCQTCASCGPGPHGPPKDTDTLVLVMEPGLPALVSCGSTLQGRCFLHELEPRGKALHLAAPACLFSANNNKPEACTDCVASPLGTRVTVVEQGHASYFYVASSLDPELAASFSPRSVSIRRLKSDTSGFQPGFPSLSVLPKYLASYLIKYVYSFHSGDFVYFLTVQPISVTSPPSALHTRLVRLNAVEPEIGDYRELVLDCHFAPKRRRRGAPEGTQPYPVLQAAHSAPVDAKLAVELSISEGQEVLFGVFVTVKDGGSGMGPNSVVCAFPIYHLNILIEEGVEYCCHSSNSSSLLSRGLDFFQTPSFCPNPPGGEASGPSSRCHYFPLMVHASFTRVDLFNGLLGSVKVTALHVTRLGNVTVAHMGTVDGRVLQVEIARSLNYLLYVSNFSLGSSGQPVHRDVSRLGNDLLFASGDQVFKVPIQGPGCRHFLTCWRCLRAQRFMGCGWCGDRCDRQKECPGSWQQDHCPPEISEFYPHSGPLRGTTRLTLCGSNFYLRPDDVVPEGTHQITVGQSPCRLLPKDSSSPRPGSLKEFIQELECELEPLVTQAVGTTNISLVITNMPAGKHFRVEGISVQEGFSFVEPVLTSIKPDFGPRAGGTYLTLEGQSLSVGTSRAVLVNGTQCRLEQVNEEQILCVTPPGAGTARVPLHLQIGGAEVPGSWTFHYKEDPIVLDISPKCGYSGSHIMIHGQHLTSAWHFTLSFHDGQSTVESRCAGQFVEQQQRRCRLPEYVVRNPQGWATGNLSVWGDGAAGFTLPGFRFLPPPSPLRAGLVELKPEEHSVKVEYVGLGAVADCVTVNMTVGGEVCQHELRGDVVICPLPPSLQLGKDGVPLQVCVDGGCHILSQVVRSSPGRASQRILLIALLVLILLVAVLAVALIFNSRRRKKQLGAHSLSPTTLSDINDTASGAPNHEESSESRDGTSVPLLRTESIRLQDLDRMLLAEVKDVLIPHEQVVIHTDQVIGKGHFGVVYHGEYTDGAQNQTHCAIKSLSRITEVQEVEAFLREGLLMRGLHHPNILALIGIMLPPEGLPRVLLPYMRHGDLLHFIRSPQRNPTVKDLVSFGLQVACGMEYLAEQKFVHRDLAARNCMLDESFTVKVADFGLARGVLDKEYYSVRQHRHARLPVKWMALESLQTYRFTTKSDVWSFGVLLWELLTRGAPPYPHIDPFDLSHFLAQGRRLPQPEYCPDSLYHVMLRCWEADPAARPTFRALVLEVKQVVASLLGDHYVQLTAAYVNVGPRAVDDGSVPPEQVQPSPQHCRSTSKPRPLSEPPLPT

SEQ ID NO: 210 - protein, 11 aa, synthetic construct
  REGION 1..11: note = Synthetic
GSSGGSGGSGG

SEQ ID NO: 211 - protein, 8 aa, synthetic construct
  REGION 1..8: note = Synthetic
GGGSGGGS

SEQ ID NO: 212 - protein, 12 aa, synthetic construct
  REGION 1..12: note = Synthetic
GGGSGGGSGGGS

SEQ ID NO: 213 - protein, 15 aa, synthetic construct
  REGION 1..15: note = Synthetic
GGGGSGGGGSGGGGS

SEQ ID NO: 214 - protein, 25 aa, synthetic construct
  REGION 1..25: note = Synthetic
GGGGSGGGGSGGGGSGGGGSGGGGS

SEQ ID NO: 215 - protein, 10 aa, synthetic construct
  REGION 1..10: note = Synthetic
GGGGSGGGGS

SEQ ID NO: 216 - protein, 5 aa, synthetic construct
  REGION 1..5: note = Synthetic
GGGGS

SEQ ID NO: 217 - protein, 7 aa, synthetic construct
  REGION 1..7: note = Synthetic
GGGGSGS

SEQ ID NO: 218 - protein, 17 aa, synthetic construct
  REGION 1..17: note = Synthetic
GGGGSGGGGSGGGGSGS

SEQ ID NO: 219 - protein, 12 aa, synthetic construct
  REGION 1..12: note = Synthetic
GGSLDPKGGGGS

SEQ ID NO: 220 - protein, 20 aa, synthetic construct
  REGION 1..20: note = Synthetic
PKSCDKTHTCPPCPAPELLG

SEQ ID NO: 221 - protein, 17 aa, synthetic construct
  REGION 1..17: note = Synthetic
SKYGPPCPPCPAPEFLG

SEQ ID NO: 222 - protein, 12 aa, synthetic construct
  REGION 1..12: note = Synthetic
GKSSGSGSESKS

SEQ ID NO: 223 - protein, 14 aa, synthetic construct
  REGION 1..14: note = Synthetic
GSTSGSGKSSEGKG

SEQ ID NO: 224 - protein, 18 aa, synthetic construct
  REGION 1..18: note = Synthetic
GSTSGSGKSSEGSGSTKG

SEQ ID NO: 225 - protein, 18 aa, synthetic construct
  REGION 1..18: note = Synthetic
GSTSGSGKPGSGEGSTKG

SEQ ID NO: 226 - protein, 16 aa, synthetic construct
  REGION 1..16: note = Synthetic
GSTSGSGKPGSSEGST

SEQ ID NO: 227 - protein, 5 aa, synthetic construct
  REGION 1..5: note = Synthetic
GSGGS

SEQ ID NO: 228 - protein, 4 aa, synthetic construct
  REGION 1..4: note = Synthetic
GGGS

SEQ ID NO: 229 - protein, 4 aa, synthetic construct
  REGION 1..4: note = Synthetic
GGSG

SEQ ID NO: 230 - protein, 5 aa, synthetic construct
  REGION 1..5: note = Synthetic
GGSGG

SEQ ID NO: 231 - protein, 5 aa, synthetic construct
  REGION 1..5: note = Synthetic
GSGSG

SEQ ID NO: 232 - protein, 5 aa, synthetic construct
  REGION 1..5: note = Synthetic
GSGGG

SEQ ID NO: 233 - protein, 5 aa, synthetic construct
  REGION 1..5: note = Synthetic
GGGSG

SEQ ID NO: 234 - protein, 5 aa, synthetic construct
  REGION 1..5: note = Synthetic
GSSSG

SEQ ID NO: 235-244 - skipped (10 entries)

SEQ ID NO: 245 - protein, 8 aa, synthetic construct
  REGION 1..8: note = Synthetic
GPQGTAGQ

SEQ ID NO: 246-249 - skipped (4 entries)

SEQ ID NO: 250 - protein, 7 aa, synthetic construct
  REGION 1..7: note = Synthetic
YGAGLGW

SEQ ID NO: 251-262 - skipped (12 entries)

SEQ ID NO: 263 - protein, 11 aa, synthetic construct
  REGION 1..11: note = Synthetic
APRSALAHGLF

SEQ ID NO: 264 - protein, 8 aa, synthetic construct
  REGION 1..8: note = Synthetic
AQNLLGMY

SEQ ID NO: 265 - protein, 18 aa, synthetic construct
  REGION 1..18: note = Synthetic
LSGRSDNHGGAVGLLAPP

SEQ ID NO: 266 - protein, 20 aa, synthetic construct
  REGION 1..20: note = Synthetic
VHMPLGFLGPGGLSGRSDNH

SEQ ID NO: 267 - protein, 20 aa, synthetic construct
  REGION 1..20: note = Synthetic
LSGRSDNHGGVHMPLGFLGP

SEQ ID NO: 268 - protein, 22 aa, synthetic construct
  REGION 1..22: note = Synthetic
LSGRSDNHGGSGGSISSGLLSS

SEQ ID NO: 269 - protein, 22 aa, synthetic construct
  REGION 1..22: note = Synthetic
ISSGLLSSGGSGGSLSGRSGNH

SEQ ID NO: 270 - protein, 22 aa, synthetic construct
  REGION 1..22: note = Synthetic
LSGRSDNHGGSGGSQNQALRMA

SEQ ID NO: 271 - protein, 22 aa, synthetic construct
  REGION 1..22: note = Synthetic
QNQALRMAGGSGGSLSGRSDNH

SEQ ID NO: 272 - protein, 22 aa, synthetic construct
  REGION 1..22: note = Synthetic
LSGRSGNHGGSGGSQNQALRMA

SEQ ID NO: 273 - protein, 22 aa, synthetic construct
  REGION 1..22: note = Synthetic
QNQALRMAGGSGGSLSGRSGNH

SEQ ID NO: 274 - protein, 13 aa, synthetic construct
  REGION 1..13: note = Synthetic
ISSGLLSGRSGNH

SEQ ID NO: 275 - protein, 22 aa, synthetic construct
  REGION 1..22: note = Synthetic
AVGLLAPPGGTSTSGRSANPRG

SEQ ID NO: 276 - protein, 22 aa, synthetic construct
  REGION 1..22: note = Synthetic
TSTSGRSANPRGGGAVGLLAPP

SEQ ID NO: 277 - protein, 24 aa, synthetic construct
  REGION 1..24: note = Synthetic
VHMPLGFLGPGGTSTSGRSANPRG

SEQ ID NO: 278 - protein, 24 aa, synthetic construct
  REGION 1..24: note = Synthetic
TSTSGRSANPRGGGVHMPLGFLGP

SEQ ID NO: 279 - protein, 22 aa, synthetic construct
  REGION 1..22: note = Synthetic
LSGRSGNHGGSGGSISSGLLSS

SEQ ID NO: 280 - protein, 8 aa, synthetic construct
  REGION 1..8: note = Synthetic
PRFKIIGG

SEQ ID NO: 281 - protein, 8 aa, synthetic construct
  REGION 1..8: note = Synthetic
PRFRIIGG

SEQ ID NO: 282 - protein, 9 aa, synthetic construct
  REGION 1..9: note = Synthetic
SSRHRRALD

SEQ ID NO: 283 - protein, 14 aa, synthetic construct
  REGION 1..14: note = Synthetic
RKSSIIIRMRDVVL

SEQ ID NO: 284 - protein, 15 aa, synthetic construct
  REGION 1..15: note = Synthetic
SSSFDKGKYKKGDDA

SEQ ID NO: 285 - protein, 15 aa, synthetic construct
  REGION 1..15: note = Synthetic
SSSFDKGKYKRGDDA

SEQ ID NO: 286 - protein, 4 aa, synthetic construct
  REGION 1..4: note = Synthetic
IEGR

SEQ ID NO: 287 - protein, 4 aa, synthetic construct
  REGION 1..4: note = Synthetic
IDGR

SEQ ID NO: 288 - protein, 7 aa, synthetic construct
  REGION 1..7: note = Synthetic
GGSIDGR

SEQ ID NO: 289 - protein, 6 aa, synthetic construct
  REGION 1..6: note = Synthetic
PLGLWA

SEQ ID NO: 290 - protein, 8 aa, synthetic construct
  REGION 1..8: note = Synthetic
GPQGIAGQ

SEQ ID NO: 291 - protein, 8 aa, synthetic construct
  REGION 1..8: note = Synthetic
GPQGLLGA

SEQ ID NO: 292 - protein, 5 aa, synthetic construct
  REGION 1..5: note = Synthetic
GIAGQ

SEQ ID NO: 293 - protein, 8 aa, synthetic construct
  REGION 1..8: note = Synthetic
GPLGIAGI

SEQ ID NO: 294 - protein, 8 aa, synthetic construct
  REGION 1..8: note = Synthetic
GPEGLRVG

SEQ ID NO: 295 - protein, 8 aa, synthetic construct
  REGION 1..8: note = Synthetic
YGAGLGVV

SEQ ID NO: 296 - protein, 8 aa, synthetic construct
  REGION 1..8: note = Synthetic
AGLGVVER

SEQ ID NO: 297 - protein, 8 aa, synthetic construct
  REGION 1..8: note = Synthetic
AGLGISST

SEQ ID NO: 298 - protein, 8 aa, synthetic construct
  REGION 1..8: note = Synthetic
EPQALAMS

SEQ ID NO: 299 - protein, 8 aa, synthetic construct
  REGION 1..8: note = Synthetic
QALAMSAI

SEQ ID NO: 300 - protein, 8 aa, synthetic construct
  REGION 1..8: note = Synthetic
AAYHLVSQ

SEQ ID NO: 301 - protein, 8 aa, synthetic construct
  REGION 1..8: note = Synthetic
MDAFLESS

SEQ ID NO: 302 - protein, 8 aa, synthetic construct
  REGION 1..8: note = Synthetic
ESLPVVAV

SEQ ID NO: 303 - protein, 8 aa, synthetic construct
  REGION 1..8: note = Synthetic
SAPAVESE

SEQ ID NO: 304 - protein, 8 aa, synthetic construct
  REGION 1..8: note = Synthetic
DVAQFVLT

SEQ ID NO: 305 - protein, 7 aa, synthetic construct
  REGION 1..7: note = Synthetic
VAQFVLT

SEQ ID NO: 306 - protein, 8 aa, synthetic construct
  REGION 1..8: note = Synthetic
VAQFVLTE

SEQ ID NO: 307 - protein, 8 aa, synthetic construct
  REGION 1..8: note = Synthetic
AQFVLTEG

SEQ ID NO: 308 - protein, 8 aa, synthetic construct
  REGION 1..8: note = Synthetic
PVQPIGPQ

SEQ ID NO: 309 - protein, 422 aa, synthetic construct
  REGION 1..422: note = Synthetic
METDTLLLWVLLLWVPGSTGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGRSDNIGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 310 - DNA, 1269 bp, synthetic construct
  misc_feature 1..1269: note = Synthetic
  source 1..1269: mol_type = other DNA
atggaaaccgacacactgctgctgtgggtgctgcttttgtgggtgccaggatccacaggctgtgatctgcctcaaacgcattcattggggtccaggcgcacgcttatgttgcttgcacagatgaggagaatatcacttttctcttgcttgaaggaccgccacgattttggctttccgcaggaagagttcggtaaccagttccaaaaggcagagacaatccccgttttgcatgagatgatccaacagatctttaacctgttttcaaccaaggatagcagcgcagcgtgggatgagacactgcttgacaagttttacaccgagctctatcagcaacttaatgatctcgaagcctgcgtaattcaaggagtaggcgttacagagacacctttgatgaaggaggattccatccttgcagtaagaaaatacttccagaggatcaccctctacctcaaagaaaagaaatactccccatgcgcgtgggaagtagtgcgagctgaaataatgcggagcttttctttgtcaactaatctccaagaatctctgagaagcaaggagtcaggtaggtctgataatatcgggggaggttctgaatctaagtacggccctccttgtcctccatgtcctgctccagagtttctcggaggcccctccgtgttcctgtttcctccaaagcctaaggacaccctgatgatcagcagaacccctgaagtgacctgcgtggtggtcgacgtttcacaagaggaccccgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagggaaccccaggtttacacactgccacctagccaagaggaaatgaccaagaaccaggtgtccctgacctgcctggtcaagggcttttacccctccgatatcgccgtggaatgggagagcaatggccagcctgagaacaactacaagaccacacctcctgtgctggacagcgacggctcattcttcctgtacagcagactgaccgtggacaagagcagatggcagcagggcaacgtgttcagctgcagcgtgatgcacgaggccctgcacaaccactacacccagaagtctctgagcctgagctga

SEQ ID NO: 311 - protein, 428 aa, synthetic construct
  REGION 1..428: note = Synthetic
METDTLLLWVLLLWVPGSTGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEISSGLLSGRSDNIGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 312 - DNA, 1287 bp, synthetic construct
  misc_feature 1..1287: note = Synthetic
  source 1..1287: mol_type = other DNA
atggaaaccgacacactgctgctgtgggtgctgcttttgtgggtgccaggatccacaggctgtgatctgcctcaaacgcattcattggggtccaggcgcacgcttatgttgcttgcacagatgaggagaatatcacttttctcttgcttgaaggaccgccacgattttggctttccgcaggaagagttcggtaaccagttccaaaaggcagagacaatccccgttttgcatgagatgatccaacagatctttaacctgttttcaaccaaggatagcagcgcagcgtgggatgagacactgcttgacaagttttacaccgagctctatcagcaacttaatgatctcgaagcctgcgtaattcaaggagtaggcgttacagagacacctttgatgaaggaggattccatccttgcagtaagaaaatacttccagaggatcaccctctacctcaaagaaaagaaatactccccatgcgcgtgggaagtagtgcgagctgaaataatgcggagcttttctttgtcaactaatctccaagaatctctgagaagcaaggagattagttctggcctgctgtcaggtaggtctgataatatcgggggaggttctgaatctaagtacggccctccttgtcctccatgtcctgctccagagtttctcggaggcccctccgtgttcctgtttcctccaaagcctaaggacaccctgatgatcagcagaacccctgaagtgacctgcgtggtggtcgacgtttcacaagaggaccccgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagggaaccccaggtttacacactgccacctagccaagaggaaatgaccaagaaccaggtgtccctgacctgcctggtcaagggcttttacccctccgatatcgccgtggaatgggagagcaatggccagcctgagaacaactacaagaccacacctcctgtgctggacagcgacggctcattcttcctgtacagcagactgaccgtggacaagagcagatggcagcagggcaacgtgttcagctgcagcgtgatgcacgaggccctgcacaaccactacacccagaagtctctgagcctgagctga

SEQ ID NO: 313 - protein, 391 aa, synthetic construct
  REGION 1..391: note = Synthetic
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGRSDNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 314 - protein, 393 aa, synthetic construct
  REGION 1..393: note = Synthetic
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGRSDNICPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 315 - protein, 221 aa, synthetic construct
  REGION 1..221: note = Synthetic
CPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 316 - protein, 221 aa, synthetic construct
  REGION 1..221: note = Synthetic
CPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLG

SEQ ID NO: 317 - protein, 4 aa, synthetic construct
  REGION 1..4: note = Synthetic
SDNI

SEQ ID NO: 318 - protein, 6 aa, synthetic construct
  REGION 1..6: note = Synthetic
GSSGGS

SEQ ID NO: 319 - protein, 4 aa, synthetic construct
  REGION 1..4: note = Synthetic
ESKY

SEQ ID NO: 320 - protein, 407 aa, synthetic construct
  REGION 1..407: note = Synthetic
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLSSGGGGSGRSDNIGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 321 - protein, 4 aa, synthetic construct
  REGION 1..4: note = Synthetic
SGGG

SEQ ID NO: 322 - protein, 225 aa, synthetic construct
  REGION 1..225: note = Synthetic
SDNICPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLG

SEQ ID NO: 323 - protein, 392 aa, synthetic construct
  REGION 1..392: note = Synthetic
CDLPQTHSLGSRRTLMLLAQMRKISLFSCLKDRHDFGFPQEEFGNQFQKAETIPVLHEMIQQIFNLFTTKDSSAAWDEDLLDKFCTELYQQLNDLEACVMQEERVGETPLMNVDSILAVKKYFRRITLYLTEKKYSPCAWEVVRAEIMRSLSLSTNLQERLRRKELSGRSDNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 324 - protein, 165 aa, synthetic construct
  REGION 1..165: note = Synthetic
CDLPQTHSLGSRRTLMLLAQMRKISLFSCLKDRHDFGFPQEEFGNQFQKAETIPVLHEMIQQIFNLFTTKDSSAAWDEDLLDKFCTELYQQLNDLEACVMQEERVGETPLMNVDSILAVKKYFRRITLYLTEKKYSPCAWEVVRAEIMRSLSLSTNLQERLRRKE

SEQ ID NO: 325 - protein, 166 aa, synthetic construct; identical to SEQ ID NO: 106
  REGION 1..166: note = Synthetic

SEQ ID NO: 326 - protein, 203 aa, synthetic construct
  REGION 1..203: note = Synthetic
MTANHQSPGMHSILLLLLLPALTTTFSCNHLRHQDANFSWKSLQLLQNTAPPPPQPCPQQDVTFPFPETLLKSKDKKQAAITTLRILQHLFNMLSSPHTPKHWIDRTRHSLLNQIQHYIHHLEQCFVNQGTRSQRRGPRNAHLSINKYFRSIHNFLQHNNYSACTWDHVRLQARDCFRHVDTLIQWMKSRAPLTASSKRLNTQ

SEQ ID NO: 327 - protein, 187 aa, synthetic construct
  REGION 1..187: note = Synthetic
MALPCSFSVALVLLSCHSLCCLACHLPDTHSLRNWRVLTLLGQMRRLSASSCDHYTTDFAFPKELFDGQRLQEAQALSVVHVMTQKVFHLFCTNTSSAPWNMTLLEELCSGLSEQLDDLDACPLQEAGLAETPLMHEDSTLRTYFQRISLYLQDRNHSPCAWEMVRAEIGRSFFSLTILQERVRRRK

SEQ ID NO: 328 - protein, 191 aa, synthetic construct
  REGION 1..191: note = Synthetic
MPGPSAPPPPAIYSALALLLLLTPPANAFSCSPLRLHDSAFAWDSLQLLRNMAPSPTQPCPQQHAPCSFPDTLLDTNDTQQAAHTALHLLQHLFDTLSSPSTPAHWLHTARHDLLNQLQHHIHHLERCFPADAARLHRRGPRNLHLSINKYFGCIQHFLQNHTYSPCAWDHVRLEAHACFQRIHRLTRTMR

SEQ ID NO: 329 - protein, 189 aa, synthetic construct
  REGION 1..189: note = Synthetic
MAPAWSFLLALLLLSCNAICSLGCHLPHTHSLPNRRVLTLLRQLRRVSPSSCLQDRNDFAFPQEALGGSQLQKAQAISVLHEVTQHTFQLFSTEGSAAAWDESLLDKLRAALDQQLTDLQACLRQEEGLRGAPLLKEDASLAVRKYFHRLTLYLREKRHNPCAWEVVRAEVMRAFSSSTNLQERFRRKD

SEQ ID NO: 330 - protein, 193 aa, synthetic construct
  REGION 1..193: note = Synthetic
MAVPASPQHPRGYGILLLTLLLKALATTASACNHLRPQDATFSHDSLQLLRDMAPTLPQLCPQHNASCSFNDTILDTSNTRQADKTTHDILQHLFKILSSPSTPAHWNDSQRQSLLNRIHRYTQHLEQCLDSSDTRSRTRWPRNLHLTIKKHFSCLHTFLQDNDYSACAWEHVRLQARAWFLHIHNLTGNTRT

SEQ ID NO: 331 - protein, 194 aa, synthetic construct
  REGION 1..194: note = Synthetic
MALPSSFLVALVALGCNSVCSLGCDLPQTHGLLNRRALTLLGQMRRLPASSCQKDRNDFAFPQDVFGGDQSHKAQALSVVHVTNQKIFHFFCTEASSSAAWNTTLLEEFCTGLDRQLTRLEACVLQEVEEGEAPLTNEDIHPEDSILRNYFQRLSLYLQEKKYSPCAWEIVRAEIMRSLYYSSTALQKRLRSEK

SEQ ID NO: 332 - protein, 186 aa, synthetic construct
  REGION 1..186: note = Synthetic
MANKCILQIALLMCFSTTALSMSYDVLRYQQRSSNLACQKLLGQLPGTPQYCLEDRMNFEVPEEIMQPPQFQKEDAVLIIHEMLQQIFGILRRNFSSTGWNETVIKTILVELDGQMDDLETILEEIMEEENFPRGDMTILHLKKYYLSILQYLKSKEYRSCAWTVVQVEILRNFSFLNRLTDYLRN

SEQ ID NO: 333 - protein, 186 aa, synthetic construct
  REGION 1..186: note = Synthetic
MTHRCLLQMVLLLCFSTTALSRSYSLLRFQQRRSLALCQKLLRQLPSTPQHCLEARMDFQMPEEMKQAQQFQKEDAILVIYEMLQQIFNILTRDFSSTGWSETIIEDLLEELYEQMNHLEPIQKEIMQKQNSTMGDTTVLHLRKYYFNLVQYLKSKEYNRCAWTVVRVQILRNFSFLTRLTGYLRE

SEQ ID NO: 334 - protein, 165 aa, synthetic construct
  REGION 1..165: note = Synthetic
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMNEDSILAVRKYFQRITLYLKEKKYSPCAWEVVRAEIMRSFSLSTNLQESLRSKE

SEQ ID NO: 335 - protein, 5 aa, synthetic construct
  REGION 1..5: note = Synthetic
SGGGG

SEQ ID NO: 336 - protein, 396 aa, synthetic construct
  REGION 1..396: note = Synthetic
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGSGRSDNICPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 337 - protein, 399 aa, synthetic construct
  REGION 1..399: note = Synthetic
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGSGRSDNIGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 338 - protein, 403 aa, synthetic construct
  REGION 1..403: note = Synthetic
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGSGRSDNIESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 339 - protein, 407 aa, synthetic construct
  REGION 1..407: note = Synthetic
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGGGSGRSDNIGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 340 - protein, 237 aa, synthetic construct
  REGION 1..237: note = Synthetic
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGRSDNIGPPCPPCPAPEFLGGPSVFLFPPKPKDTLMISRTPEVTCVVVDVSQEDPEVQFNWYVDGVEVHNA

SEQ ID NO: 341 - protein, 398 aa, synthetic construct
  REGION 1..398: note = Synthetic
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGRSDNIESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 342 - protein, 402 aa, synthetic construct
  REGION 1..402: note = Synthetic
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGRSDNIGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 343 - protein, 17 aa, synthetic construct
  REGION 1..17: note = Synthetic
MRAWIFFLLCLAGRALA

SEQ ID NO: 344 - protein, 23 aa, synthetic construct
  REGION 1..23: note = Synthetic
MALTFALLVALLVLSCKSSCSVG

SEQ ID NO: 345 - protein, 20 aa, synthetic construct
  REGION 1..20: note = Synthetic
METDTLLLWVLLLWVPGSTG

SEQ ID NO: 346-503 - skipped (158 entries)

SEQ ID NO: 504 - protein, 6 aa, synthetic construct
  REGION 1..6: note = Synthetic
QGQSGS

SEQ ID NO: 505 - protein, 5 aa, synthetic construct
  REGION 1..5: note = Synthetic
GQSGS

SEQ ID NO: 506 - protein, 4 aa, synthetic construct
  REGION 1..4: note = Synthetic
QSGS

SEQ ID NO: 507 - protein, 7 aa, synthetic construct
  REGION 1..7: note = Synthetic
QGQSGQG

SEQ ID NO: 508 - protein, 6 aa, synthetic construct
  REGION 1..6: note = Synthetic
GQSGQG

SEQ ID NO: 509 - protein, 5 aa, synthetic construct
  REGION 1..5: note = Synthetic
QSGQG

SEQ ID NO: 510 - protein, 4 aa, synthetic construct
  REGION 1..4: note = Synthetic
SGQG

SEQ ID NO: 511 - protein, 6 aa, synthetic construct
  REGION 1..6: note = Synthetic
QGQSGQ

SEQ ID NO: 512 - protein, 5 aa, synthetic construct
  REGION 1..5: note = Synthetic
GQSGQ

SEQ ID NO: 513 - protein, 4 aa, synthetic construct
  REGION 1..4: note = Synthetic
QSGQ

SEQ ID NO: 514 - protein, 5 aa, synthetic construct
  REGION 1..5: note = Synthetic
QGQSG

SEQ ID NO: 515 - protein, 4 aa, synthetic construct
  REGION 1..4: note = Synthetic
QGQS

SEQ ID NO: 516 - protein, 10 aa, synthetic construct
  REGION 1..10: note = N-terminal deletion sequence in an Fc
EPKSCDKTHT

SEQ ID NO: 517 - protein, 12 aa, synthetic construct
  REGION 1..12: note = N-terminal deletion in an Fc
ELKTPLGDTTHT

SEQ ID NO: 518 - protein, 7 aa, synthetic construct
  REGION 1..7: note = N-terminal deletion sequence in an Fc
ESKYGPP

(19) United States
(12) Patent Application Publication
(10) Pub. No.: US 2025/0179141 A1
(43) Pub. Date: Jun. 5, 2025

(54) ACTIVATABLE CYTOKINE CONSTRUCTS AND RELATED COMPOSITIONS AND METHODS

(71) Applicant: CytomX Therapeutics, Inc., South San Francisco, CA (US)

(72) Inventors: Sayantan Mitra, Mountain View, CA (US); Nicole G. Lapuyade, San Francisco, CA (US); Hikmat Haizar Assi, San Pablo, CA (US); Madan M. Paidhungat, San Francisco, CA (US); Dylan L. Daniel, San Francisco, CA (US); Erwan Le Scolan, San Francisco, CA (US); Walter A. Bogdanoff, Rio Vista, CA (US); Na Cai, San Mateo, CA (US); Hsin WANG, San Mateo, CA (US)

(73) Assignee: CytomX Therapeutics, Inc., South San Francisco, CA (US)

(21) Appl. No.: 18/806,635

(22) Filed: Aug. 15, 2024

Related U.S. Application Data
(63) Continuation of application No. 17/824,294, filed on May 25, 2022, now Pat. No. 12,091,442.
Application No. 17/824,294 is a continuation of application No. 17/227,017, filed on Apr. 9, 2021, now Pat. No. 11,365,233.
(60) Provisional application No. 63/008,542, filed on Apr. 10, 2020.
(60) Provisional application No. 63/161,889, filed on Mar. 16, 2021.
(60) Provisional application No. 63/164,849, filed on Mar. 23, 2021.

(51) Int. Cl. C07K 14/56

(52) U.S. Cl. CPC C07K 14/56; C07K 2319/02; C07K 2319/30

(57) ABSTRACT

Provided herein are activatable cytokine constructs that include: (a) a first monomer construct comprising a first mature cytokine protein (CP1), a first cleavable moiety (CM1), and a first dimerization domain (DD1), wherein the CM1 is positioned between the CP1 and the DD1; and (b) a second monomer construct comprising a second mature cytokine protein (CP2), a second cleavable moiety (CM2), and a second dimerization domain (DD2), where the CM2 is positioned between the CP2 and the DD2, where: the CM1 and the CM2 function as a substrate for a protease; the DD1 and the DD2 bind each other; and where the ACC is characterized by a reduction in at least one activity of the CP1 and/or CP2 as compared to a control level of the at least one activity of the CP1 and/or CP2.

### CROSS-REFERENCE TO RELATED APPLICATIONS

This application is a continuation of U.S. application Ser. No. 17/824,294, filed May 25, 2022, which is a continuation of Ser. No. 17/227,017, filed Apr. 9, 2021, now U.S. Pat. No. 11,365,233, issued Jun. 21, 2022, which claims priority benefit of U.S. provisional application Nos. 63/008,542, filed Apr. 10, 2020, 63/161,889, filed Mar. 16, 2021, and 63/164,849, filed Mar. 23, 2021, the entire contents of which are incorporated herein by reference.

### REFERENCE TO SEQUENCE LISTING

The instant application contains a Sequence Listing which has been submitted electronically in XML format and is hereby incorporated by reference in its entirety. Said XML copy, created on Aug. 14, 2024 is named “4862-105US6.xml” and is 491,520 bytes in size.

### TECHNICAL FIELD

The present disclosure relates to the field of biotechnology, and more specifically, to activatable cytokine constructs.

### BACKGROUND

Cytokines are a family of naturally-occurring small proteins and glycoproteins produced and secreted by most nucleated cells in response to viral infection and/or other antigenic stimuli. Interferons are a subclass of cytokines. Interferons are presently grouped into three major classes: interferon type I, interferon type II, and interferon type III. Interferons exert their cellular activities by binding to specific membrane receptors on a cell surface.

Interferon therapy has many clinical benefits. For example, interferons are known to up-regulate the immune system and also to have antiviral and anti-proliferative properties. These biological properties have led to the clinical use of interferons as therapeutic agents for the treatment of viral infections and malignancies. Further, interferons are useful for recruiting a patient's innate immune system to identify and attack cancer cells. Accordingly, interferon therapy has been extensively used in cancer and antiviral therapy, including for the treatment of hepatitis, Kaposi sarcoma, hairy cell leukemia, chronic myeloid leukemia (CML), follicular lymphoma, renal cell cancer (RCC), melanoma, and other disease states. However, systemic administration of interferons is accompanied by dose-dependent toxicities, including strong flu-like symptoms, neurological symptoms, hepatotoxicity, bone marrow suppression, and arrhythmia, among others. In a Melanoma patient study, the combination of Pembrolizumab and Pegylated IFNa led to an ORR of 60.5%. The combination treatment was also associated with 49% of G3/G4 adverse events which required dose reduction of Pegylated IFNa (Davar et al., J. Clin. Oncol., 2018). These undesired side-effects have limited the dosage of interferon therapies and sometimes leads to discontinuation or delay of interferon treatment.

Interleukins are another subclass of cytokines. Interleukins regulate cell growth, differentiation, and motility. They are particularly important in stimulating immune responses, such as inflammation. Interleukins have been used for treatment of cancer, autoimmune disorders, and other disorders. For example, interleukin-2 (IL2) is indicated for treatment of melanoma, graft-versus-host disease (GVHD), neuroblastoma, renal cell cancer (RCC), and is also considered useful for conditions including acute coronary syndrome, acute myeloid syndrome, atopic dermatitis, autoimmune liver diseases, basal cell carcinoma, bladder cancer, breast cancer, candidiasis, colorectal cancer, cutaneous T-cell lymphoma, endometriomas, HIV invention, ischemic heart disease, rheumatoid arthritis, nasopharyngeal adenocarcinoma, non-small cell lung cancer (NSCLC), ovarian cancer, pancreatic cancer, systemic lupus erythematosus, tuberculosis, and other disorders. Other interleukins, such as IL-6, IL-7, IL-12, and IL-21, among others, are potential treatments for cancers and other disorders. Interleukin therapy is often accompanied by undesired side effects, including flu-like symptoms, nausea, vomiting, diarrhea, low blood pressure, and arrhythmia, among others.

Thus, the need and desire for improved specificity and selectivity of cytokine therapy to the desired target is of great interest. Increased targeting of cytokine therapeutics to the disease site could reduce systemic mechanism-based toxicities and lead to broader therapeutic utility.

### SUMMARY

The present disclosure provides activatable cytokine constructs (ACCs) that include: (a) a first monomer comprising a first mature cytokine protein (CP1), a first cleavable moiety (CM1), and a first dimerization domain (DD1), wherein the CM1 is positioned between the CP1 and the DD1; and (b) a second monomer comprising a second mature cytokine protein (CP2), a second cleavable moiety (CM2), and a second dimerization domain (DD2), wherein the CM2 is positioned between the CP2 and the DD2, where: the CM1 and the CM2 function as a substrate for a protease; the DD1 and the DD2 bind each other; and where the ACC is characterized by a reduction in at least one activity of the CP1 and/or CP2 as compared to a control level of the at least one activity of the CP1 and/or CP2. The protease(s) that cleave the CM1 and CM2 may be over-expressed in diseased tissue (e.g., tumor tissue) relative to healthy tissue. The ACC may be activated upon cleavage of the CM1 and/or CM2 so that the cytokine may exert its activity in the diseased tissue (e.g., in a tumor microenvironment) while the cytokine activity is attenuated in the context of healthy tissue. Thus, the ACCs provided herein may provide reduced toxicity relative to traditional cytokine therapeutics, enable higher effective dosages of cytokine, and/or increase the therapeutic window for the cytokine.

Provided herein are activatable cytokine constructs (ACC) that include a first monomer construct and a second monomer construct, wherein: (a) the first monomer construct comprises a first mature cytokine protein (CP1), a first cleavable moiety (CM1), and a first dimerization domain (DD1), wherein the CM1 is positioned between the CP1 and the DD1; and (b) the second monomer construct comprises a second mature cytokine protein (CP2), a second cleavable moiety (CM2), and a second dimerization domain (DD2), wherein the CM2 is positioned between the CP2 and the DD2; wherein the DD1 and the DD2 bind each other thereby forming a dimer of the first monomer construct and the second monomer construct; and wherein the ACC is characterized by having a reduced level of at least one CP1 and/or CP2 activity as compared to a control level of the at least one CP1 and/or CP2 activity.

The present disclosure provides activatable cytokine constructs (ACCs) that include: (a) a first monomer comprising a first mature cytokine protein (CP1), a first dimerization domain (DD1); and (b) a second monomer comprising a second mature cytokine protein (CP2), a cleavable moiety (CM), and a second dimerization domain (DD2), wherein the CM is positioned between the CP2 and the DD2, where: the CM functions as a substrate for a protease; the DD1 and the DD2 bind each other; and where the ACC is characterized by a reduction in at least one activity of the CP1 and/or CP2 as compared to a control level of the at least one activity of the CP1 and/or CP2.

The present disclosure provides activatable cytokine constructs (ACCs) that include: (a) a first monomer comprising a first mature cytokine protein (CP1), a cleavable moiety (CM), and a first dimerization domain (DD1), wherein the CM is positioned between the CP1 and the DD1; and (b) a second monomer comprising a second mature cytokine protein (CP2), and a second dimerization domain (DD2), where: the CM functions as a substrate for a protease; the DD1 and the DD2 bind each other; and where the ACC is characterized by a reduction in at least one activity of the CP1 and/or CP2 as compared to a control level of the at least one activity of the CP1 and/or CP2.

The present disclosure provides activatable cytokine constructs (ACCs) that include: (a) a first monomer comprising a first mature cytokine protein (CP1), and a first dimerization domain (DD1); and (b) a second monomer comprising a second mature cytokine protein (CP2), and a second dimerization domain (DD2), wherein the CP1, the CP2, or both CP1 and CP2 include(s) an amino acid sequence that functions as a substrate for a protease; the DD1 and the DD2 bind each other; and where the ACC is characterized by a reduction in at least one activity of the CP1 and/or CP2 as compared to a control level of the at least one activity of the CP1 and/or CP2.

The ACCs of the present disclosure are characterized in that CP1 and CP2 are not connected to peptide masks, for example, affinity masking moieties.

In some embodiments, the first monomer construct comprises a first polypeptide that comprises the CP1, the CM1, and the DD1. In some embodiments, the second monomer construct comprises a second polypeptide that comprises the CP2, the CM2, and the DD2. In some embodiments, the DD1 and the DD2 are a pair selected from the group consisting of: a pair of Fc domains, a sushi domain from an alpha chain of human IL-15 receptor (IL15Rα) and a soluble IL-15; barnase and barnstar; a protein kinase A (PKA) and an A-kinase anchoring protein (AKAP); adapter/docking tag modules based on mutated RNase I fragments; an epitope and single domain antibody (sdAb); an epitope and single chain variable fragment (scFv); and soluble N-ethyl-maleimide sensitive factor attachment protein receptors (SNARE) modules based on interactions of the proteins syntaxin, synaptotagmin, synaptobrevin, and SNAP25, an antigen-binding domain and an epitope.

In some embodiments, the DD1 and the DD2 are a pair of Fc domains. In some embodiments, the pair of Fc domains is a pair of human Fc domains. In some embodiments, the human Fc domains are human IgG1 Fc domains, human IgG2 Fc domains, human IgG3 Fc domains, or human IgG4 Fc domains. In some embodiments, the human Fc domains are human IgG4 Fc domains. In some embodiments, the human Fc domains comprise a sequence that is at least 80% identical to SEQ ID NO: 3. In some embodiments, the human Fc domains each comprise a sequence that is at least 90%, 95%, 96%, 97%, 98%, or 99% identical to SEQ ID NO: 3. In some embodiments, the human Fc domains each comprise SEQ ID NO: 3. In some embodiments, the DD1 and the DD2 are the same. For example, DD1 and the DD2 may be a pair of identical human IgG4 Fc domains. In some embodiments, the dimerization domains have amino acid sequences of SEQ ID NOs: 315 and 316, respectively. In some embodiments, the human Fc domains include mutations to eliminate glycosylation and/or to reduce Fc-gamma receptor binding. In some embodiments, the human Fc domains comprise the mutation N297Q, N297A, or N297G; in some embodiments the human Fc domains comprise a mutation at position 234 and/or 235, for example L235E, or L234A and L235A (in IgG1), or F234A and L235A (in IgG4); in some embodiments the human Fc domains are IgG2 Fc domains that comprise the mutations V234A, G237A, P238S, H268Q/A, V309L, A330S, or P331S, or a combination thereof (all according to EU numbering).

Additional examples of engineered human Fc domains are known to those skilled in the art. Examples of Ig heavy chain constant region amino acids in which mutations in at least one amino acid leads to reduced Fc function include, but are not limited to, mutations in amino acid 228, 233, 234, 235, 236, 237, 239, 252, 254, 256, 265, 270, 297, 318, 320, 322, 327, 329, 330, and 331 of the heavy constant region (according to EU numbering). Examples of combinations of mutated amino acids are also known in the art, such as, but not limited to a combination of mutations in amino acids 234, 235, and 331, such as L234F, L235E, and P331S or a combination of amino acids 318, 320, and 322, such as E318A, K320A, and K322A.

Further examples of engineered Fc domains include F243L/R292P/Y300L/V305I/P396 IgG1; S239D/I332E IgG1; S239D/I332E/A330L IgG1; S298A/E333A/K334A; in one heavy chain, L234Y/L235Q/G236W/S239M/H268D/D270E/S298A IgG1, and in the opposing heavy chain, D270E/K326D, A330M/K334E IgG; G236A/S239D/I332E IgG1; K326W/E333S IgG1; S267E/H268F/S324T IgG1; E345R/E430G/S440Y IgG1; N297A or N297Q or N297G IgG1; L235E IgG1; L234A/L235A IgG1; F234A/L235A IgG4; H268Q/V309L/A330S/P331S IgG2; V234A/G237A/P238S/H268A/V309L/A330S/P331S IgG2; M252Y/S254T/T256E IgG1; M428L/N434S IgG1; S267E/L328F IgG1; N325S/L328F IgG1, and the like. In some embodiments, the engineered Fc domain comprises one or more substitutions selected from the group consisting of N297A IgG1, N297Q IgG1, and S228P IgG4.

In some embodiments, DD1 comprises an antigen-binding domain and DD2 comprises a corresponding epitope. In some embodiments, the antigen-binding domain is an anti-His tag antigen-binding domain and wherein the DD2 comprises a His tag. In some embodiments, the antigen-binding domain is a single chain variable fragment (scFv). In some embodiments, the antigen-binding domain is a single domain antibody (sdAb). In some embodiments, at least one of DD1 and DD2 comprises a dimerization domain substituent selected from the group consisting of a non-polypeptide polymer and a small molecule. In some embodiments, DD1 and DD2 comprise non-polypeptide polymers covalently bound to each other. In some embodiments, the non-polypeptide polymer is a sulfur-containing polyethylene glycol, and wherein DD1 and DD2 are covalently bound to each other via one or more disulfide bonds. In some embodiments, at least one of DD1 and DD2 comprises a small molecule. In some embodiments, the small molecule is biotin. In some embodiments, DD1 comprises biotin and DD2 comprises an avidin.

In some embodiments, the CP1 and the CP2 are mature cytokines. In some embodiments, each of the CP1 and the CP2 comprise a mature cytokine sequence and further comprise a signal peptide (also referred to herein as a “signal sequence”). In some embodiments, the CP1 and/or the CP2 is/are each individually selected from the group consisting of: an interferon, an interleukin, GM-CSF, G-CSF, LIF, OSM, CD154, LT-β, TNF-α, TNF-β, 4-1BBL, APRIL, CD70, CD153, CD178, GITRL, LIGHT, OX40L, TALL-1, TRAIL, TWEAK, TRANCE, TGF-β1, TGF-β1, TGF-β3, Epo, Tpo, Flt-3L, SCF, M-CSF, and MSP. The CP1 and/or CP2 may be a wild-type human or non-human animal sequence, a mutant sequence, a truncated sequence, a hybrid sequence, or sequence comprising insertions. In some embodiments, the CP1 and the CP2 are the same. In some embodiments, the CP1 and the CP2 are different and this disclosure includes selection and combination of any two of the cytokine proteins listed herein. In some embodiments, the CP1 and/or the CP2 is/are an interferon. In some embodiments, the CP1 and the CP2 both are an interferon. In some embodiments, the CP1 and the CP2 are different interferons. In some embodiments, the CP1 and the CP2 are the same interferon. In some embodiments, the CP1 or the CP2 is an interferon. In some embodiments, one of the CP1 and the CP2 is an interferon, and the other of CP1 or CP2 is a cytokine other than an interferon. In some aspects, one or both cytokines are monomeric cytokines. In some aspects, one or both interferons are monomeric interferons. In some aspects, either CP1 or CP2 is a monomeric interferon and the other CP1 or CP2 is a different cytokine. In some aspects, the CP1 and/or the CP2 include a mutant cytokine sequence. In some aspects, the CP1 and/or the CP2 include a universal cytokine sequence. In some aspects, the CP1 and/or the CP2 include a truncated sequence that retains cytokine activity.

In some embodiments, the interferon(s) is/are a human wildtype mature interferon. In some embodiments, the interferon(s) may be type I and type II interferons, for example including, but not limited to interferon-alpha, interferon-beta, interferon-omega, interferon-gamma, and interferon-tau. In some embodiments, the interferons is/are an interferon-alpha. In some embodiments, the interferon(s) is/are selected from the group consisting of: interferon alpha-2a, interferon alpha-2b, and interferon alpha-n3. In some embodiments, the interferon(s) is/are interferon alpha-2b. In some embodiments, the interferon(s) is/are a mutant interferon. In some embodiments, the interferon(s) is/are a mutant interferon wherein an endogenous protease cleavage site has been rendered disfunctional by substitution, deletion, or insertion of one or more amino acids. In some embodiments, the interferon(s) is/are a universal cytokine molecule, e.g., having a hybrid sequence of different cytokine subtypes or a chimeric cytokine sequence or a humanized cytokine sequence. In some embodiments, the interferon(s) is/are a universal interferon molecule. In some embodiments, the interferon(s) is/are a universal interferon alpha, e.g., a hybrid of interferon alpha 1 and interferon alpha 2b. In some embodiments, the CP1 and/or CP2 comprises a sequence that is at least 80% identical to SEQ ID NO: 1. In some embodiments, the CP1 and/or CP2 comprises a sequence that is at least 90%, 95%, 96%, 97%, 98%, or 99% identical to SEQ ID NO: 1. In some embodiments, the CP1 and/or CP2 comprises a sequence of SEQ ID NO: 1. In some embodiments, the interferon is an interferon beta. In some embodiments, the interferon beta is selected from the group consisting of interferon beta-1a, and interferon beta-1b. In some embodiments, the CP1 and/or the CP2 comprises an IFab domain. In some embodiments, the CP1 and/or the CP2 comprises an interleukin. In some embodiments, the interleukin is selected from the group consisting of IL-1α, IL-1β, IL-1RA, IL-18, IL-2, IL-4, IL-7, IL-9, IL-13, IL-15, IL-3, IL-5, IL-6, IL-11, IL-12, IL-10, IL-20, IL-14, IL-16, and IL-17.

In some embodiments, the CM1 and/or the CM2 comprise a total of about 3 amino acids to about 15 amino acids. In some embodiments, the CM1 and the CM2 comprise substrates for different proteases. In some embodiments, the CM1 and the CM2 are of the same length and comprise the same amino acid sequence. In some embodiments, wherein the CM1 and the CM2 comprise substrates for the same protease. In some embodiments, the protease(s) is/are selected from the group consisting of: ADAM8, ADAM9, ADAM10, ADAM12, ADAM15, ADAM17/TACE, ADAMDEC1, ADAMTS1, ADAMTS4, ADAMTS5, BACE, Renin, Cathepsin D, Cathepsin E, Caspase 1, Caspase 2, Caspase 3, Caspase 4, Caspase 5, Caspase 6, Caspase 7, Caspase 8, Caspase 9, Caspase 10, Caspase 14, Cathepsin B, Cathepsin C, Cathepsin K, Cathespin L, Cathepsin S, Cathepsin V/L2, Cathepsin X/Z/P, Cruzipain, Legumain, Otubain-2, KLK4, KLK5, KLK6, KLK7, KLK8, KLK10, KLK11, KLK13, KLK14, Meprin, Neprilysin, PSMA, BMP-1, matrix metalloproteinases (e.g., MMP-1, MMP-2, MMP-3, MMP-7, MMP-9, MMP-10, MMP-11, MMP-12, MMP-13, MMP-14, MMP-15, MMP-16, MMP-17, MMP-19, MMP-20, MMP-23, MMP-24, MMP-26, MMP-27), activated protein C, cathepsin A, cathepsin G, Chymase, FVIIa, FIXa, FXa, FXIa, FXIIa, Elastase, Granzyme B, Guanidinobenzoatase, HtrA1, human neutrophil lyase, lactoferrin, marapsin, NS3/4A, PACE4, Plasmin, PSA, tPA, thrombin, tryptase, uPA, DESC1, DPP-4, FAP, Hepsin, Matriptase-2, MT-SP1/Matripase, TMPRSS2, TMPRSS3, and TMPRSS4. In some embodiments, the protease(s) is/are selected from the group consisting of: uPA, legumain, MT-SP1, ADAM17, BMP-1, TMPRSS3, TMPRSS4, MMP-2, MMP-9, MMP-12, MMP-13, and MMP-14.

Suitable cleavable moieties have been disclosed in WO 2010/081173, WO 2015/048329, WO 2015/116933, WO 2016/118629, and WO 2020/118109, the disclosures of which are incorporated herein by reference in their entireties.

In some embodiments, the CM1 and/or the CM2 comprise a sequence selected from the group consisting of: LSGRSDNH (SEQ ID NO: 5), TGRGPSWV (SEQ ID NO: 6), PLTGRSGG (SEQ ID NO: 7), TARGPSFK (SEQ ID NO: 8), NTLSGRSENHSG (SEQ ID NO: 9), NTLSGRSGNHGS (SEQ ID NO: 10), TSTSGRSANPRG (SEQ ID NO: 11), TSGRSANP (SEQ ID NO: 12), VHMPLGFLGP (SEQ ID NO: 13), AVGLLAPP (SEQ ID NO: 14), AQNLLGMV (SEQ ID NO: 15), QNQALRMA (SEQ ID NO: 16), LAAPLGLL (SEQ ID NO: 17), STFPFGMF (SEQ ID NO: 18), ISSGLLSS (SEQ ID NO: 19), PAGLWLDP (SEQ ID NO: 20), VAGRSMRP (SEQ ID NO: 21), VVPEGRRS (SEQ ID NO: 22), ILPRSPAF (SEQ ID NO: 23), MVLGRSLL (SEQ ID NO: 24), QGRAITFI (SEQ ID NO: 25), SPRSIMLA (SEQ ID NO: 26), SMLRSMPL (SEQ ID NO: 27), ISSGLLSGRSDNH (SEQ ID NO: 28), AVGLLAPPGGLSGRSDNH (SEQ ID NO: 29), ISSGLLSSGGSGGSLSGRSDNH (SEQ ID NO: 30), LSGRSGNH (SEQ ID NO: 31), SGRSANPRG (SEQ ID NO: 32), LSGRSDDH (SEQ ID NO: 33), LSGRSDIH (SEQ ID NO: 34), LSGRSDQH (SEQ ID NO: 35), LSGRSDTH (SEQ ID NO: 36), LSGRSDYH (SEQ ID NO: 37), LSGRSDNP (SEQ ID NO: 38), LSGRSANP (SEQ ID NO: 39), LSGRSANI (SEQ ID NO: 40), LSGRSDNI (SEQ ID NO: 41), MIAPVAYR (SEQ ID NO: 42), RPSPMWAY (SEQ ID NO: 43), WATPRPMR (SEQ ID NO: 44), FRLLDWQW (SEQ ID NO: 45), ISSGL (SEQ ID NO: 46), ISSGLLS (SEQ ID NO: 47), ISSGLL (SEQ ID NO: 48), ISSGLLSGRSANPRG (SEQ ID NO: 49), AVGLLAPPTSGRSANPRG (SEQ ID NO: 50), AVGLLAPPSGRSANPRG (SEQ ID NO: 51), ISSGLLSGRSDDH (SEQ ID NO: 52), ISSGLLSGRSDIH (SEQ ID NO: 53), ISSGLLSGRSDQH (SEQ ID NO: 54), ISSGLLSGRSDTH (SEQ ID NO: 55), ISSGLLSGRSDYH (SEQ ID NO: 56), ISSGLLSGRSDNP (SEQ ID NO: 57), ISSGLLSGRSANP (SEQ ID NO: 58), ISSGLLSGRSANI (SEQ ID NO: 59), AVGLLAPPGGLSGRSDDH (SEQ ID NO: 60), AVGLLAPPGGLSGRSDIH (SEQ ID NO: 61), AVGLLAPPGGLSGRSDQH (SEQ ID NO: 62), AVGLLAPPGGLSGRSDTH (SEQ ID NO: 63), AVGLLAPPGGLSGRSDYH (SEQ ID NO: 64), AVGLLAPPGGLSGRSDNP (SEQ ID NO: 65), AVGLLAPPGGLSGRSANP (SEQ ID NO: 66), AVGLLAPPGGLSGRSANI (SEQ ID NO: 67), ISSGLLSGRSDNI (SEQ ID NO: 68), AVGLLAPPGGLSGRSDNI (SEQ ID NO: 69), GLSGRSDNHGGAVGLLAPP (SEQ ID NO: 70), GLSGRSDNHGGVHMPLGFLGP (SEQ ID NO: 71), LSGRSDNHGGVHIMIPLGFLGP (SEQ ID NO: 72), ISSGLSS (SEQ ID NO: 73), PVGYTSSL (SEQ ID NO: 74), DWLYWPGI (SEQ ID NO: 75), LKAAPRWA (SEQ ID NO: 76), GPSHLVLT (SEQ ID NO: 77), LPGGLSPW (SEQ ID NO: 78), MGLFSEAG (SEQ ID NO: 79), SPLPLRVP (SEQ ID NO: 80), RMILRSLG (SEQ ID NO: 81), LLAPSHRA (SEQ ID NO: 82), GPRSFGL (SEQ ID NO: 83), GPRSFG (SEQ ID NO: 84), SARGPSRW (SEQ ID NO: 85), GGWHTGRN (SEQ ID NO: 86), HTGRSGAL (SEQ ID NO: 87), AARGPAIH (SEQ ID NO: 88), RGPAFNPM (SEQ ID NO: 89), SSRGPAYL (SEQ ID NO: 90), RGPATPIM (SEQ ID NO: 91), RGPA (SEQ ID NO: 92), GGQPSGMWGW (SEQ ID NO: 93), FPRPLGITGL (SEQ ID NO: 94), SPLTGRSG (SEQ ID NO: 95), SAGFSLPA (SEQ ID NO: 96), LAPLGLQRR (SEQ ID NO: 97), SGGPLGVR (SEQ ID NO: 98), PLGL (SEQ ID NO: 99), and SGRSDNI (SEQ ID NO: 100). In some embodiments, the CM comprises a sequence selected from the group consisting of: ISSGLLSGRSDNH (SEQ ID NO: 28), LSGRSDDH (SEQ ID NO: 33), ISSGLLSGRSDQH (SEQ ID NO: 54), SGRSDNI (SEQ ID NO: 100), and ISSGLLSGRSDNI (SEQ ID NO: 68). In some embodiments, the protease(s) is/are produced by a tumor in the subject, e.g., the protease(s) are produced in greater amounts in the tumor than in healthy tissues of the subject. In some embodiments, the subject has been diagnosed or identified as having a cancer.

In some embodiments, the CP1 and the CM1 directly abut each other in the first monomer construct. In some embodiments, the CM1 and the DD1 directly abut each other in the first monomer construct. In some embodiments, the CP2 and the CM2 directly abut each other in the second monomer construct. In some embodiments, the CM2 and the DD2 directly abut each other in the second monomer construct. In some embodiments, the first monomer construct comprises the CP1 directly abutting the CM1, and the CM1 directly abutting the DD1, wherein the CM1 comprises a sequence that is selected from the group consisting of SEQ ID Nos 5-100. In some embodiments, the second monomer construct comprises the CP2 directly abutting the CM2, and the CM2 directly abutting the DD2, wherein the CM2 comprises a sequence that is selected from the group consisting of SEQ ID Nos 5-100. In some embodiments, the first monomer construct comprises the CP1 directly abutting the CM1, and the CM1 directly abutting the DD1, wherein the CM1 comprises a sequence that is no more than 13, 12, 11, 10, 9, 8, 7, 6, 5 or 4 amino acids in length. In some embodiments, the second monomer construct comprises the CP2 directly abutting the CM2, and the CM2 directly abutting the DD2, wherein the CM2 comprises a sequence that is no more than 13, 12, 11, 10, 9, 8, 7, 6, 5 or 4 amino acids in length. In some embodiments, the first and second monomer construct each are configured such that the cytokine (CM1 and CM2, respectively) directly abuts a cleavable moiety (CM1 and CM2, respectively) that is no more than 10, 9, 8, 7, 6, 5, or 4 amino acids in length, and the cleavable moiety directly abuts a dimerization domain (DD1 and DD2, respectively) that is the Fc region of a human IgG, wherein the N-terminus of the Fc region is the first cysteine residue in the hinge region reading in the N- to C-direction (e.g., Cysteine 226 of human IgG1, using EU numbering). In some aspects, the dimerization domain is an IgG Fc region wherein the upper hinge residues have been deleted. For example, the Fc is a variant wherein N-terminal sequences EPKSCDKTHT (SEQ ID NO: 516), ERK, ELKTPLGDTTHT (SEQ ID NO: 517), or ESKYGPP (SEQ ID NO: 518) have been deleted.

In some embodiments, the first monomer construct comprises at least one linker. In some embodiments, the at least one linker is a linker L1 disposed between the CP1 and the CM1 and/or a linker L2 disposed between the CM1 and the DD1. In some embodiments, the second monomer construct comprises at least one linker. In some embodiments, the at least one linker is a linker L3 disposed between the CP2 and the CM2 and/or a linker L4 disposed between the CM2 and the DD2. In some embodiments, the first monomer construct comprises a linker L1 and the second monomer construct comprises a linker L3. In some embodiments, L1 and L3 are the same. In some embodiments, the first monomer construct comprises a linker L2 and the second monomer construct comprises a linker L4. In some embodiments, L2 and L4 are the same. In some embodiments, each linker has a total length of 1 amino acid to about 15 amino acids. In some embodiments, each linker has a total length of at least 5 amino acids. As used herein, the term “linker” refers to a peptide, the amino acid sequence of which is not a substrate for a protease.

In some embodiments, the first monomer construct comprises at least one linker, wherein each linker is independently selected from from the group consisting of GSSGGSGGSGG (SEQ ID NO: 210); GGGS (SEQ ID NO: 2); GGGSGGGS (SEQ ID NO: 211); GGGSGGGSGGGS (SEQ ID NO: 212); GGGGSGGGGSGGGGS (SEQ ID NO: 213); GGGGSGGGGSGGGGSGGGGSGGGGS (SEQ ID NO: 214); GGGGSGGGGS (SEQ ID NO: 215); GGGGS (SEQ ID NO: 216); GS; GGGGSGS (SEQ ID NO: 217); GGGGSGGGGSGGGGSGS (SEQ ID NO: 218); GGSLDPKGGGGS (SEQ ID NO: 219); PKSCDKTHTCPPCPAPELLG (SEQ ID NO: 220); SKYGPPCPPCPAPEFLG (SEQ ID NO: 221); GKSSGSGSESKS (SEQ ID NO: 222); GSTSGSGKSSEGKG (SEQ ID NO: 223); GSTSGSGKSSEGSGSTKG (SEQ ID NO: 224); GSTSGSGKPGSGEGSTKG (SEQ ID NO: 225); GSTSGSGKPGSSEGST (SEQ ID NO: 226); (GS)n, (GGS)n, (GSGGS)n (SEQ ID NO: 227), (GGGS)n (SEQ ID NO: 228), (GGGGS)n (SEQ ID NO: 216), wherein each n is an integer of at least one; GGSG (SEQ ID NO: 229); GGSGG (SEQ ID NO: 230); GSGSG (SEQ ID NO: 231; GSGGG (SEQ ID NO: 232); GGGSG (SEQ ID NO: 233); GSSSG (SEQ ID NO: 234); GGGGSGGGGSGGGGS (SEQ ID NO: 213); GGGGSGGGGSGGGGSGGGGS (SEQ ID NO: 214); and GSTSGSGKPGSSEGST (SEQ ID NO: 226). In some embodiments, the linker comprises a sequence of GGGS (SEQ ID NO: 2).

As used herein, the term “spacer” refers herein to an amino acid residue or a peptide incorporated at a free terminus of the mature ACC, for example between the signal peptide and the N-terminus of the mature ACC. In some aspects, a spacer (or “header”) may contain glutamine (Q) residues. In some aspects, residues in the spacer minimize aminopeptidase and/or exopeptidase action to prevent cleavage of N-terminal amino acids. Illustrative and non-limiting spacer amino acid sequences may comprise or consist of any of the following exemplary amino acid sequences: QGQSGS (SEQ ID NO: 504); GQSGS (SEQ ID NO: 505); QSGS (SEQ ID NO: 506); SGS; GS; S; QGQSGQG (SEQ ID NO: 507); GQSGQG (SEQ ID NO: 508); QSGQG (SEQ ID NO: 509); SGQG (SEQ ID NO: 510); GQG; QG; G; QGQSGQ (SEQ ID NO: 511); GQSGQ (SEQ ID NO: 512); QSGQ (SEQ ID NO: 513); QGQSG (SEQ ID NO: 514); QGQS (SEQ ID NO: 515); SGQ; GQ; and Q. In some embodiments, spacer sequences may be omitted.

In some embodiments, the first monomer construct, comprises in a N- to C-terminal direction, the CP1, the CM1, and, linked directly or indirectly to the C-terminus of the CM1, the DD1. In some embodiments, the first polypeptide comprises in a C- to N-terminal direction, the CP1, the CM1, and, linked directly or indirectly to the N-terminus of the CM1, the DD1. In some embodiments, the second polypeptide comprises in a N- to C-terminal direction, the CP2, CM2, and, linked directly or indirectly to the C-terminus of the CM2, the DD2. In some embodiments, the second polypeptide comprises in a C- to N-terminal direction, the CP2, CM2, and, linked directly or indirectly to the CM2, the DD2.

In some embodiments, the first monomer construct comprises in an N- to C-terminal direction, the CP1, an optional linker, the CM1, an optional linker, and the DD1, wherein DD1 is an Fc region of an IgG, wherein the N-terminus of the Fc region is the first cysteine residue in the hinge region reading in the N- to C-direction (e.g., Cysteine 226 of human IgG1 or IgG4, using EU numbering), and wherein the CM1 and any linker(s) interposed between the CP1 and the N-terminal cysteine of the DD1 have a combined total length of no more than 15, 14, 13, 12, 11, 10, 9, 8, 7, 6, 5, or 4 amino acids, preferably no more than 10 amino acids, especially preferably no more than 7 amino acids. In some embodiments, the second monomer construct comprises in an N- to C-terminal direction, the CP2, an optional linker, the CM2, an optional linker, and the DD2, wherein DD2 is an Fc region of an IgG, wherein the N-terminus of the Fc region is the first cysteine residue in the hinge region reading in the N- to C-direction (e.g., Cysteine 226 of human IgG1 or IgG4, using EU numbering), and wherein the CM2 and any linker(s) interposed between the CP2 and the N-terminal cysteine of the DD2 have a combined total length of no more than 15, 14, 13, 12, 11, 10, 9, 8, 7, 6, 5, or 4 amino acids, preferably no more than 10 amino acids, especially preferably no more than 7 amino acids.

In some embodiments, the ACC is a homodimer in which the first monomer construct and the second monomer construct are identical and comprise the amino acid sequence of SEQ ID NO: 313. In some embodiments, the first monomer construct and the second monomer construct each comprise an amino acid sequence that is at least 90%, 95%, 96%, 97%, 98%, or 99% identical to SEQ ID NO: 313. In some embodiments, the first monomer construct and the second monomer construct each comprise, in an N- to C-terminal direction, SEQ ID NO:1; a CM comprising an amino acid sequence selected from the group consisting of SEQ ID NO: 41, SEQ ID NO: 68, and SEQ ID NO: 100; and a dimerization domain.

In some embodiments, the at least one CP1 and/or CP2 activity is a binding affinity (K_{D}) of the CP1 and/or the CP2 for its cognate receptor as determined using surface plasmon resonance. For example, where the CP1 or CP2 is an interferon, the cognate receptor may be the interferon-alpha/beta receptor (IFNAR). In some embodiments, the at least one CP1 and/or CP2 activity is a level of proliferation of lymphoma cells. In some embodiments, the at least one CP1 and/or CP2 activity is the level of JAK/STAT/ISGF3 pathway activation in a lymphoma cell. In some embodiments, the at least one activity is a level of secreted alkaline phosphatase (SEAP) production in a lymphoma cell. In some embodiments, the ACC (prior to exposure to proteases) is characterized by at least a 2-fold reduction in at least one CP1 and/or CP2 activity as compared to the control level. In some embodiments, the ACC is characterized by at least a 5-fold reduction in at least one CP1 and/or CP2 activity as compared to the control level. In some embodiments, the ACC is characterized by at least a 10-fold reduction in at least one activity of the CP1 and/or CP2 as compared to the control level. In some embodiments, the ACC is characterized by at least a 20-fold, 50-fold, 100-fold, 200-fold, 300-fold, 400-fold, 500-fold, 600-fold, 700-fold, 800-fold, 900-fold, 1000-fold, 1100-fold, 1200-fold, 1300-fold, 1400-fold, 1500-fold, 1600-fold, 1700-fold, 1800-fold, 1900-fold, 2000-fold reduction in at least one CP1 and/or CP2 activity as compared to the control level. In some embodiments, the control level of the at least one activity of the CP1 and/or CP2, is the activity of the CP1 and/or CP2 in the ACC following exposure of the ACC to the protease(s). In some embodiments, the control level of the at least one CP1 and/or CP2, is the corresponding CP1 and/or CP2 activity of a corresponding wildtype mature cytokine.

In some embodiments, the ACC is characterized by generating a cleavage product following exposure to the protease(s), wherein the cleavage product comprises the at least one activity of the CP1 and/or CP2. In some embodiments, the at least one activity of the CP1 and/or CP2 is anti-proliferation activity. In some embodiments, the control level is an EC50 value of the wildtype mature cytokine, and wherein ratio of EC50 (cleavage product) to EC50 (wildtype control level) is less than about 10, or less than about 9, or less than about 8, or less than about 7, or less than about 6, or less than about 5, or less than about 4, or less than about 3, or less than about 2, or less than about 1.5, or equal to about 1. In some embodiments, the EC50 of the cleavage product is approximately the same as the EC50 of the wildtype mature cytokine, demonstrating that the following cleavage, the activity of the CP1 and/or CP2 is fully recovered, or nearly fully recovered.

Provided herein are compositions comprising any one of the ACCs described herein. In some embodiments, the composition is a pharmaceutical composition. Also provided herein are kits comprising at least one dose of any one of the compositions described herein.

Provided herein are methods of treating a subject in need thereof comprising administering to the subject a therapeutically effective amount of any one of the ACCs described herein or any one of the compositions described herein. In some embodiments, the subject has been identified or diagnosed as having a cancer. In some non-limiting embodiments, the cancer is Kaposi sarcoma, hairy cell leukemia, chronic myeloid leukemia (CML), follicular lymphoma, renal cell cancer (RCC), melanoma, neuroblastoma, basal cell carcinoma, bladder cancer, breast cancer, colorectal cancer, cutaneous T-cell lymphoma, nasopharyngeal adenocarcinoma, non-small cell lung cancer (NSCLC), ovarian cancer, pancreatic cancer. In some non-limiting embodiments, the cancer is a lymphoma. In some non-limiting embodiments, the lymphoma is Burkitt's lymphoma.

Provided herein are nucleic acids encoding a polypeptide that comprises the CP1 and CM1 of any one of the ACCs described herein. In some embodiments, the polypeptide further comprises any one of the DD1 described herein. Also provided herein are nucleic acids encoding a polypeptide that comprises the CP2 and CM2 of any one of the ACCs described herein. When the monomers are identical, then the present disclosure provides a single nucleic acid encoding the monomer that dimerizes to form ACC. In some embodiments, the polypeptide further comprises any one of the DD2 described herein. Also provided herein are vectors comprising any one of the nucleic acids described herein. In some embodiments, the vector is an expression vector. Also provided herein are cells comprising any one of the nucleic acids described herein or any one of the vectors described herein.

Provided herein are pairs of nucleic acids that together encode a polypeptide that comprises the CP1 and CM1 of the first monomer construct and a polypeptide that comprises the CP2 and CM2 of the second monomer construct of any one of the ACCs described herein. Also provided herein are pairs of vectors that together comprise any of one of the pair of nucleic acids described herein. In some embodiments, the pair of vectors is a pair of expression vectors. Also provided herein are cells comprising any one of the pairs of nucleic acids described herein or any one of the pairs of vectors described herein. In other embodiments, the present invention provides a vector comprising the pair of vectors.

Provided herein are methods of producing an ACC comprising: culturing any one of the cells described herein in a liquid culture medium under conditions sufficient to produce the ACC; and recovering the ACC from the cell or the liquid culture medium. In some embodiments, the method further comprises: isolating the ACC recovered from the cell or the liquid culture medium. In some embodiments, the method further comprises: formulating isolated ACC into a pharmaceutical composition.

Provided herein are ACCs produced by any one of the methods described herein. Also provided herein are compositions comprising any one the ACCs described herein. Also provided herein are compositions of any one of the compositions described herein, wherein the composition is a pharmaceutical composition. Also provided herein are kits comprising at least one dose of any one of the compositions described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

The term “a” and “an” refers to one or more (i.e., at least one) of the grammatical object of the article. By way of example, “a cell” encompasses one or more cells.

As used herein, the terms “about” and “approximately,” when used to modify an amount specified in a numeric value or range, indicate that the numeric value as well as reasonable deviations from the value known to the skilled person in the art. For example ±20%, ±10%, or ±5%, are within the intended meaning of the recited value where appropriate.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of,” as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” or “consisting of” would find direct support due to this definition for any elements disclosed throughout this disclosure. Based on this definition, any element disclosed herein or incorporated by reference may be included in or excluded from the claimed invention.

As used herein, a plurality of compounds, elements, or steps may be presented in a common list for convenience. However, these lists should be construed as though each member of the list is individually identified as a separate and unique member. Thus, no individual member of such list should be construed as a defacto equivalent of any other member of the same list solely based on their presentation in a common group without indications to the contrary.

Furthermore, certain molecules, constructs, compositions, elements, moieties, excipients, disorders, conditions, properties, steps, or the like may be discussed in the context of one specific embodiment or aspect or in a separate paragraph or section of this disclosure. It is understood that this is merely for convenience and brevity, and any such disclosure is equally applicable to and intended to be combined with any other embodiments or aspects found anywhere in the present disclosure and claims, which all form the application and claimed invention at the filing date. For example, a list of constructs, molecules, method steps, kits, or compositions described with respect to a construct, composition, or method is intended to and does find direct support for embodiments related to constructs, compositions, formulations, and methods described in any other part of this disclosure, even if those method steps, active agents, kits, or compositions are not re-listed in the context or section of that embodiment or aspect.

Unless otherwise specified, a “nucleic acid sequence encoding a protein” includes all nucleotide sequences that are degenerate versions of each other and thus encode the same amino acid sequence.

The term “N-terminally positioned” when referring to a position of a first domain or sequence relative to a second domain or sequence in a polypeptide primary amino acid sequence means that the first domain or sequence is located closer to the N-terminus of the polypeptide primary amino acid sequence than the second domain or sequence. In some embodiments, there may be additional sequences and/or domains between the first domain or sequence and the second domain or sequence.

The term “C-terminally positioned” when referring to a position of a first domain or sequence relative to a second domain or sequence in a polypeptide primary amino acid sequence means that the first domain or sequence is located closer to the C-terminus of the polypeptide primary amino acid sequence than the second domain or sequence. In some embodiments, there may be additional sequences and/or domains between the first domain or sequence and the second domain or sequence.

The term “exogenous” refers to any material introduced from or originating from outside a cell, a tissue, or an organism that is not produced by or does not originate from the same cell, tissue, or organism in which it is being introduced.

The term “transduced,” “transfected,” or “transformed” refers to a process by which an exogenous nucleic acid is introduced or transferred into a cell. A “transduced,” “transfected,” or “transformed” cell (e.g., mammalian cell) is one that has been transduced, transfected, or transformed with exogenous nucleic acid (e.g., a vector) that includes an exogenous nucleic acid encoding any of the activatable cytokine constructs described herein.

The term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the nucleic acids described herein, the nucleic acid is DNA. In some embodiments of any of the nucleic acids described herein, the nucleic acid is RNA.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include: amino acids with acidic side chains (e.g., aspartate and glutamate), amino acids with basic side chains (e.g., lysine, arginine, and histidine), non-polar amino acids (e.g., alanine, valine, leucine, isoleucine, proline, phenylalanine, methionine, and tryptophan), uncharged polar amino acids (e.g., glycine, asparagine, glutamine, cysteine, serine, threonine and tyrosine), hydrophilic amino acids (e.g., arginine, asparagine, aspartate, glutamine, glutamate, histidine, lysine, serine, and threonine), hydrophobic amino acids (e.g., alanine, cysteine, isoleucine, leucine, methionine, phenylalanine, proline, tryptophan, tyrosine, and valine). Other families of amino acids include: aliphatic-hydroxy amino acids (e.g., serine and threonine), amide family (e.g., asparagine and glutamine), aliphatic family (e.g., alanine, valine, leucine and isoleucine), aromatic family (e.g., phenylalanine, tryptophan, and tyrosine).

As used herein the phrase “specifically binds,” or “immunoreacts with” means that the activatable antigen-binding protein complex reacts with one or more antigenic determinants of the desired target antigen and does not react with other polypeptides, or binds at much lower affinity, e.g., about or greater than 10⁻⁶ M.

The term “treatment” refers to ameliorating at least one symptom of a disorder. In some embodiments, the disorder being treated is a cancer and to ameliorate at least one symptom of a cancer.

### BRIEF DESCRIPTION OF DRAWINGS

FIG. 1A is a schematic of an illustrative activatable cytokine construct comprising a first and second monomer construct that bind to each other either covalently or non-covalently via first and second dimerization domains DD1 140 and DD2 190, respectively. The first monomer construct comprises, from N-terminus to C-terminus, a first mature cytokine protein CP1 100, a first optional linker 110, a first cleavable moiety CM1 120, a second optional linker 130, and a first dimerization domain DD1 140. The second monomer construct comprises, from N-terminus to C-terminus, a second mature cytokine protein CP2 150, a third optional linker 160, a second cleavable moiety CM2 170, a fourth optional linker 180, and a second dimerization domain DD2 190.

FIG. 1B is a schematic of an illustrative activatable cytokine construct comprising a first and second monomer construct that bind to each other either covalently or non-covalently via first and second dimerization domains DD1 200 and DD2 250, respectively. The first monomer construct comprises, from N-terminus to C-terminus, a first dimerization domain DD1 200, a second optional linker 210, a first cleavable moiety CM1 220, a first optional linker 230, and a first mature cytokine protein CP1 240. The second monomer construct comprises, from N-terminus to C-terminus, a second dimerization domain DD2 250, a fourth optional linker 260, a second cleavable moiety CM2 270, a third optional linker 280, and a second mature cytokine protein CP2 290.

FIG. 2A is a schematic of an illustrative activatable cytokine construct comprising a first and second monomer construct that bind to each other by non-covalent means via first and second dimerization domains DD1 340 and DD2 390, respectively. The first monomer construct comprises, from N-terminus to C-terminus, a first mature cytokine protein CP1 300, a first optional linker 310, a first cleavable moiety CM1 320, a second optional linker 330, and a first dimerization domain DD1 340. The second monomer construct comprises, from N-terminus to C-terminus, a second mature cytokine protein CP2 350, a third optional linker 360, a second cleavable moiety CM2 370, a fourth optional linker 380, and a second dimerization domain DD2 390.

FIG. 2B is a schematic of an illustrative activatable cytokine construct comprising a first and second monomer construct that bind to each other by non-covalent means via first and second dimerization domains DD1 400 and DD2 450, respectively. The first monomer construct comprises, from N-terminus to C-terminus, a first dimerization domain DD1 400, a second optional linker 410, a first cleavable moiety CM1 420, a first optional linker 430, and a first mature cytokine protein CP1 440. The second monomer construct comprises, from N-terminus to C-terminus, a second dimerization domain DD2 450, a fourth optional linker 460, a second cleavable moiety CM2 470, a third optional linker 480, and a second mature cytokine protein CP2 490.

FIG. 3 provides the amino acid sequence of an illustrative activatable cytokine construct IFN-α2b-1204dL-hIgG4 (SEQ ID NO:309), wherein the first and second monomer constructs have an identical amino acid sequence. From N-terminus to C-terminus, the amino acid sequence of the first and second monomer constructs encodes: a mouse signal peptide (italicized text, not bolded); mature human interferon-α2b (underscored text); cleavable moiety 1204dL (bolded text); a linker (italicized and bolded text); and a human IgG4 Fc domain (text that is not italicized, bolded, or underscored).

FIG. 4 provides the amino acid sequence of an illustrative activatable cytokine construct IFN-α2b-1490DNI-hIgG4 (SEQ ID NO:311), wherein the first and second monomer constructs have an identical amino acid sequence. From N-terminus to C-terminus, the amino acid sequence encodes: a mouse signal peptide (italicized text, not bolded); mature human interferon-α2b (underscored text); cleavable moiety 1490DNI (bolded text, not italicized); a linker (italicized and bolded text); and a human IgG4 Fc domain (text that is not italicized, bolded, or underscored).

FIG. 5 depicts the cleavage reaction of activatable cytokine construct IFNα-2b-hIgG4 Fc (with either cleavable moiety 1204dL or cleavable moiety 1490) and a protease (either uPA or MT-SP1), which generates monomeric mature IFNα-2b.

FIG. 6 is image of a gel loaded with: (1) ACC with cleavable moiety 1204 (1204); (2) product of protease membrane type serine protease 1 (MT-SP1) and ACC IFNα-2b-hIgG4 Fc with cleavable moiety 1204 (1204 MT-SP1); (3) product of ACC IFNα-2b-hIgG4 Fc with cleavable moiety 1204 and protease uPA (1204 uPA); (4) ACC IFNα-2b-hIgG4 Fc with cleavable moiety 1204 fused to a 5 amino acid linker (1204+1); (5) product of IFNα-2b-hIgG4 Fc 1204+1 and MT-SP1 (1204+1 MT-SP1); (6) ACC IFNα-2b-hIgG4 Fc with cleavable moiety 1490; (7) product of MT-SP1 and ACC IFNα-2b-hIgG4 Fc with cleavable moiety 1490; product of uPA and ACC IFNα-2b-hIgG4 Fc with cleavable moiety 1490 (1490 uPA).

FIG. 7 provides the results from an HEK293 cell-based reporter assay to assess interferon-α2b activity of Sylatron® (peginterferon alfa-2b) and various interferon α-2b (IFNa2b) fusions: human IgG4 N-terminally fused to IFNa2b (IFNa2b NhG4); Human IgG4 N-terminally fused to IFNa2b via a five amino acid linker (IFNa2b 5AA NhG4); activatable cytokine construct IFN-α2b-1204dL-hIgG4 (IFNa2b 1204DNIdL NhG4); an activatable cytokine construct that includes the same components as IFN-α2b-1204dL-hIgG4, but which also has a 5 amino acid linker positioned between the mature cytokine protein component and the cleavable moiety (IFNa2b 5AA 1204DNIdL NhG4); and activatable cytokine construct IFN-α2b-1490DNI-hIgG4 (IFNa2b 1490DNI NhG4).

FIG. 8A depicts the effect of length of a flexible linker in an interferon-α2b-Fc fusion on EC50 as determined by an HEK293 cell-based reporter assay. FIG. 8B depicts the effect of length of a Linking Region (LR) in an interferon-α2b-Fc fusion on EC50 as determined by an HEK293 cell-based reporter assay.

FIG. 9 provides the results of a Daudi apoptosis assay to determine the anti-proliferative activity of Sylatron® and various IFNa2b fusions: human IgG4 N-terminally fused to IFNa2b (IFNa2b NhG4); Human IgG4 N-terminally fused to IFNa2b via a five amino acid linker (IFNa2b 5AA NhG4); activatable cytokine construct IFN-α2b-1204dL-hIgG4 (IFNa2b 1204DNIdL NhG4); an activatable cytokine construct that includes the same components as IFN-α2b-1204dL-hIgG4, but which also has a 5 amino acid linker positioned between the mature cytokine protein component and the cleavable moiety (IFNa2b 5AA 1204DNIdL NhG4); and activatable cytokine construct IFN-α2b-1490DNI-hIgG4 (IFNa2b 1490DNI NhG4).

FIG. 10A depicts the effect of length of a linker in an interferon-α2b-Fc fusion protein on EC50 as determined from a Daudi apoptosis assay. FIG. 10B depicts the effect of length of a Linking Region (LR) in an interferon-α2b-Fc fusion on EC50 as determined from an Daudi apoptosis assay.

FIG. 11 provides the results of a Daudi lymphoma cell-based assay for measuring the anti-proliferation activity of an ACC (IFNa2b 1204DNIdL NhG4); protease-treated ACC (IFNa2b 1204DNIdL NhG4+uPA); and the recombinant parental cytokine (IFNa2b). The results indicated that, following treatment of the ACC with a protease, the activity of the cytokine in the ACC could be restored to a level comparable to the recombinant parental cytokine.

FIG. 12 depicts the results of an HEK293 cell-based reporter assay to assess the activity of an ACC (IFNa2b 1204DNIdL NhG4); a protease-treated (activated) ACC (IFNa-2b 1204DNIdL NhG4+uPA); Sylatron®; and the recombinant parental cytokine (IFNa2b). The results indicated that, following treatment of the ACC with a protease, the activity of the cytokine in the ACC could be restored to a level comparable to the recombinant parental cytokine.

FIG. 13 depicts the results of a Daudi lymphoma cell-based assay for measuring the anti-proliferation activity (top) and the results of an HEK293 cell-based reporter assay for measuring the activity (bottom) of an ACC (ProC440), a protease-treated ACC (ProC440+uPA), and stem cell IFNa2b. The results indicated that activity was reduced 1000× by making the ACC structure of the present disclosure and, following treatment of the ACC with a protease, the activity of the cytokine in the ACC was restored to a level comparable to the recombinant parental cytokine.

FIG. 14A depicts the structure of ProC440, and shows that cleavage with uPa at the expected site in the CM was confirmed by Mass spectrometry analysis. In addition to sensitivity to uPa activation, ProC440 is cleaved by MMP4. FIG. 14B shows the analysis by Mass spectrometry identified a MMP14 cleavage site at the C-terminal extremity of IFNa (at L161) near the cleavable moiety. Protease activation with MMP14 restored activity to a level that is comparable to the recombinant cytokine.

FIG. 15 depicts the structures of ProC440 and ProC657 (N IFNa2b 0AA 1204DNIdL 0AA IgG4 KiHSS). The activities of the ACCs ProC440 and ProC657, a protease-treated ACC (ProC440+uPA), and stem cell IFNa2b were tested using IFN-responsive HEK293 cells. The results indicated that activity of ProC657 was reduced as compared to Stem Cell IFNα-2b or uPa-activated ProC440 but increased as compared to ProC440.

FIG. 16 (top) depicts anti-proliferative effects of ACC ProC440 in vivo using the Daudi xenograft tumor model. ACC ProC440 induced complete tumor regression at a dose as low as 0.1 mg/kg and slowed-down tumor growth at a dose of 0.02 mg/kg. FIG. 16 (bottom) depicts anti-proliferative effects of Sylatron® in vivo using the Daudi xenograft tumor model.

FIG. 17A depicts the structure of ProC286 and the activity of ProC286 compared to the activity of Sylatron® in the Daudi apotosis assay. ProC286 and Sylatron® showed similar levels of activity indicating that ProC286 could be used as surrogate Sylatron® control to evaluate the tolerability of IFNα-2b in the hamster study. FIG. 17B depicts the structure of ProC291 and the activity of ProC291 compared to the activity of Sylatron® in the Daudi apotosis assay. ProC291 showed significantly reduced activity compared to Sylatron® and ProC286.

FIG. 18 depicts the specific activity of IFNa-con (recombinant interferon alpha, a non-naturally occurring type-I interferon), ProC440+uPA, PEG-IFNa2b (Sylatron), and ProC440 and anticipated toxic doses in a dose-escalation study in vivo, e.g., at escalating doses of 0.08, 0.4, 2, 10, 15 mg/kg (“mpk”).

FIG. 19 depicts the structure of ACC ProC859 universal interferon (top), the anti-proliferative effects of ACC ProC859 in a B16 mouse melanoma cell assay and the activity of ACC ProC859 in the IFN-responsive HEK293 assay.

FIG. 20A is a schematic of an illustrative activatable cytokine construct comprising a first and second monomer construct that bind to each other by non-covalent means via first and second dimerization domains DD1 540 and DD2 590, respectively. The first monomer construct comprises, from N-terminus to C-terminus, a first mature cytokine protein CP1 500, a first optional linker 510, a first cleavable moiety CM1 520, a second optional linker 530, and a first dimerization domain DD1 540. The second monomer construct comprises, from N-terminus to C-terminus, a second mature cytokine protein CP2 550, a third optional linker 560, and a second dimerization domain DD2 590.

FIG. 20B is a schematic of an illustrative activatable cytokine construct comprising a first and second monomer construct that bind to each other by non-covalent means via first and second dimerization domains DD1 600 and DD2 650, respectively. The first monomer construct comprises, from N-terminus to C-terminus, a first dimerization domain DD1 600, a first optional linker 630 and a first mature cytokine protein CP1 640. The second monomer construct comprises, from N-terminus to C-terminus, a second dimerization domain DD2 650, a second optional linker 660, a cleavable moiety CM 670, a third optional linker 680, and a second mature cytokine protein CP2 690.

FIG. 21A is a schematic of an illustrative activatable cytokine construct comprising a first and second monomer construct that bind to each other by non-covalent means via first and second dimerization domains DD1 740 and DD2 790, respectively. The first monomer construct comprises, from N-terminus to C-terminus, a first mature cytokine protein CP 700, a first optional linker 710, a first cleavable moiety CM1 720, a second optional linker 730, and a first dimerization domain DD1 740. The second monomer construct comprises, from N-terminus to C-terminus, a polypeptide or protein that lacks cytokine activity 780, and a second dimerization domain DD2 790. The polypeptide or protein that lacks cytokine activity 780 may, for example, be a truncated cytokine protein that lacks cytokine activity, a mutated cytokine protein that lacks cytokine activity, a stub sequence, or a polypeptide sequence that binds with high affinity to CP 700 and reduces the cytokine activity of the second moiety as compared to the control level of the second moiety. The DD1 740 and the DD2 790 may be the same or different.

FIG. 21B is a schematic of an illustrative activatable cytokine construct comprising a first and second monomer construct that bind to each other by non-covalent means via first and second dimerization domains DD1 800 and DD2 850, respectively. The first monomer construct comprises, from N-terminus to C-terminus, a first dimerization domain DD1 800 and a polypeptide or protein that lacks cytokine activity 830. The second monomer construct comprises, from N-terminus to C-terminus, a second dimerization domain DD2 850, a first optional linker 860, a cleavable moiety CM 870, a second optional linker 880, and a mature cytokine protein CP 890. The polypeptide or protein that lacks cytokine activity 830 may, for example, be a truncated cytokine protein that lacks cytokine activity, a mutated cytokine protein that lacks cytokine activity, a stub sequence, or a polypeptide sequence that binds with high affinity to CP 700 and reduces the cytokine activity of the second moiety as compared to the control level of the second moiety. The DD1 800 and the DD2 850 may be the same or different.

FIG. 22 shows the animal weight loss when dosed with 2 mpk, 10 mpk, and 15 mpk of control hIgG4, ProC286, or ProC440 over treatment periods in Syrian Gold Hamsters.

FIG. 23 shows the clinical chemistry outcomes (Alkaline phosphatase, Alanine transaminase, and Aspartate transaminase) in Syrian Gold Hamsters dosed with 2 mpk, 10 mpk, and 15 mpk of control hIgG4, ProC286, or ProC440.

FIG. 24 shows the hematology analysis outcomes (Reticulocyte, Neutrophil, and White Blood Cells (WBC) counts) in Syrian Gold Hamsters dosed with 2 mpk, 10 mpk, and 15 mpk of control hIgG4, ProC286, or ProC440.

FIG. 25 schematically shows an embodiment of an ACC denoting its Linking Region (LR).

### DETAILED DESCRIPTION

Provided herein are activatable cytokine constructs (ACCs) that exhibit a reduced level of at least one activity of the corresponding cytokine, but which, after exposure to an activation condition, yield a cytokine product having substantially restored activity. Activatable cytokine constructs of the present invention may be designed to selectively activate upon exposure to diseased tissue, and not in normal tissue. As such, these compounds have the potential for conferring the benefit of a cytokine-based therapy, with potentially less of the toxicity associated with certain cytokine-based therapies.

Also provided herein are related intermediates, compositions, kits, nucleic acids, and recombinant cells, as well as related methods, including methods of using and methods of producing any of the activatable cytokine constructs described herein.

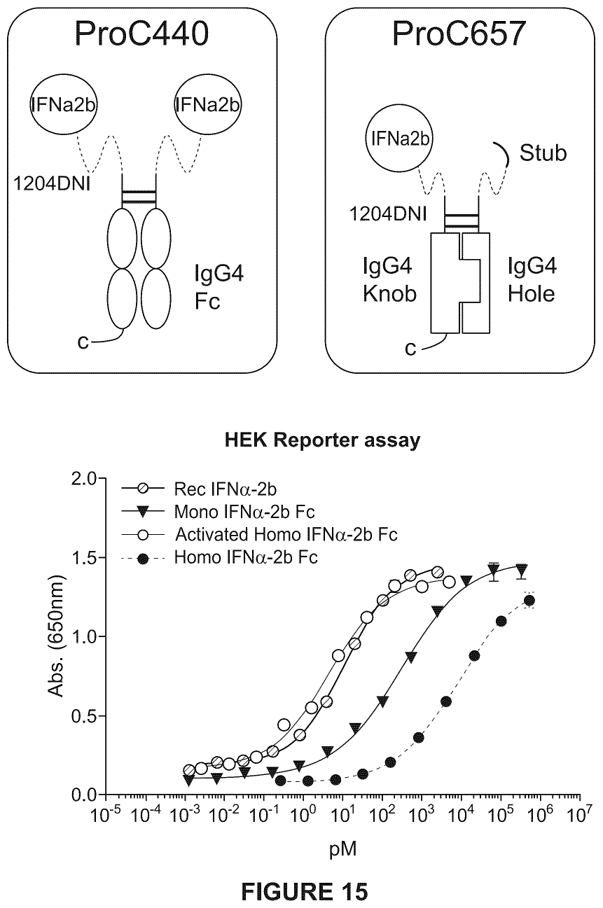

The inventors have surprisingly found that ACCs having the specific elements and structural orientations described herein appear potentially effective in improving the safety and therapeutic index of cytokines in therapy, particularly for treating cancers. While cytokines are regulators of innate and adaptive immune system and have broad anti-tumor activity in pre-clinical models, their clinical success has been limited by systemic toxicity and poor systemic exposure to target tissues. The inventors have surprisingly found that ACCs having the specific elements and structural orientations described herein appear to reduce the systemic toxicity associated with cytokine therapeutics and improve targeting and exposure to target issues. As such, the present disclosure provides a method of reducing target-mediated drug disposition (TMDD) of cytokine therapeutics by administering ACCs having the specific elements and structural orientations described herein to a subject. As such, the invention solves the problem of sequestration of a significant fraction of the administered cytokine dose by normal tissues, which is a problem that limits the fraction of the dose available in the systemic circulation to reach the target tissues, e.g., cancerous tissue, in conventional cytokine therapeutics. The present cytokine construct localizes target binding to tumor tissues, thereby maintaining potency, reducing side effects, enabling new target opportunities, improving the therapeutic window for validated targets, creating a therapeutic window for undruggable targets, and providing multiple binding modalities. The present disclosure enables safe and effective systemic delivery, thereby avoiding the dose-dependent toxicities of conventional systemic cytokine therapies, and also avoids a requirement for intra-tumoral injection. The present disclosure provides a means for imparting localized anti-viral activity, immunomodulatory activity, antiproliferative activity and pro-apoptotic activity. The inventors surprisingly found that dimerization of the first and second monomer constructs achieves high reduction of cytokine activity, particularly higher reduction than when a single cytokine is attached to a dimerization domain. See FIG. 15.

Additionally, the inventors have discovered that the degree of reduction of cytokine activity can be adjusted by varying the flexible linker length or the linking region length. The inventors surprisingly found that reduction of cytokine activity on the order of 1,000 fold or more can be achieved by attaching a cytokine via a short protease cleavable sequence to a sterically constrained dimerization domain (such as an Fc domain of a human IgG that is truncated at the first cysteine in the hinge region, e.g., Cys226 as numbered by EU numbering). Surprisingly, protease cleavage occurs despite the steric constraint, and full cytokine activity is regained upon cleavage of the cytokine from the dimerization domain.

Applicant's U.S. Provisional App. No. 63/008,542, filed Apr. 10, 2020, which describes certain activatable cytokine constructs, is incorporated herein by reference in its entirety.

### Activatable Cytokine Constructs

Activatable cytokine constructs of the present invention are dimer complexes comprising a first monomer construct and a second monomer construct. Dimerization of the monomeric components is facilitated by a pair of dimerization domains. In one aspect, each monomer construct includes a cytokine protein, a cleavable moiety, and a dimerization domain (DD). In one aspect, one monomer construct includes a cytokine protein, a cleavable moiety, and a DD, whereas the other monomer construct includes a cytokine protein and a DD, but not a cleavable moiety. In one aspect, one monomer construct includes a cytokine protein, a cleavable moiety, and a DD, whereas the other monomer construct includes a protein or peptide that lacks cytokine activity and a DD, but not a cleavable moiety. In a specific embodiment, the present invention provides an activatable cytokine construct (ACC) that includes a first monomer construct and a second monomer construct, wherein:
(a) the first monomer construct comprises a first mature cytokine protein (CP1), a first cleavable moiety (CM1), and a first dimerization domain (DD1),
   wherein the CM1 is positioned between the CP1 and the DD1; and
(b) the second monomer construct comprises a second mature cytokine protein (CP2), a second cleavable moiety (CM2), and a second dimerization domain (DD2),
   wherein the CM2 is positioned between the CP2 and the DD2;
wherein the DD1 and the DD2 bind each other thereby forming a dimer of the first monomer construct and the second monomer construct; and
wherein the ACC is characterized by having a reduced level of at least one CP1 and/or CP2 activity as compared to a control level of the at least one CP1 and/or CP2 activity.

The term “activatable” when used in reference to a cytokine construct, refers to a cytokine construct that exhibits a first level of one or more activities, whereupon exposure to a condition that causes cleavage of one or both cleavable moieties results in the generation of a cytokine construct that exhibits a second level of the one or more activities, where the second level of activity is greater than the first level of activity. Non-limiting examples of an activities include any of the exemplary activities of a cytokine described herein or known in the art.

The term “mature cytokine protein” refers herein to a cytokine protein that lacks a signal sequence. A cytokine protein (CP) may be a mature cytokine protein or a cytokine protein with a signal peptide. Thus, the ACCs of the present disclosure may include a mature cytokine protein sequence in some aspects. In some aspects, the ACCs of the present disclosure may include a mature cytokine protein sequence and, additionally, a signal sequence. In some aspects, the ACCs of the present disclosure may include sequences disclosed herein, including or lacking the signal sequences recited herein.

The terms “cleavable moiety” and “CM” are used interchangeably herein to refer to a peptide, the amino acid sequence of which comprises a substrate for a sequence-specific protease. Cleavable moieties that are suitable for use as CM1 and/or CM2 include any of the protease substrates that are known the art. Exemplary cleavable moieties are described in more detail below.

The terms “dimerization domain” and “DD” are used interchangeably herein to refer to one member of a pair of dimerization domains, wherein each member of the pair is capable of binding to the other via one or more covalent or non-covalent interactions. The first DD and the second DD may be the same or different. Exemplary DDs suitable for use as DD1 and or DD2 are described in more detail herein below.

As used herein, a polypeptide, such as a cytokine or an Fc domain, may be a wild-type polypeptide (e.g., a naturally-existing polypeptide) or a variant of the wild-type polypeptide. A variant may be a polypeptide modified by substitution, insertion, deletion and/or addition of one or more amino acids of the wild-type polypeptide, provided that the variant retains the basic function or activity of the wild-type polypeptide. In some examples, a variant may have altered (e.g., increased or decreased) function or activity comparing with the wild-type polypeptide. In some aspects, the variant may be a functional fragment of the wild-type polypeptide. The term “functional fragment” means that the sequence of the polypeptide (e.g., cytokine) may include fewer amino acids than the full-length polypeptide sequence, but sufficient polypeptide chain length to confer activity (e.g., cytokine activity).

The first and second monomer constructs may further comprise additional elements, such as, for example, one or more linkers, and the like. The additional elements are described below in more detail. The organization of the CP, CM, and DD components in each of the first and second monomer constructs may be arranged in the same order in each monomer construct. The CP1, CM1, and DD1 components may be the same or different as compared to the corresponding CP2, CM2, and DD2, in terms of, for example, molecular weight, size, amino acid sequence of the CP and CM components (and the DD components in embodiments where the DD components are polypeptides), and the like. Thus, the resulting dimer may have symmetrical or asymmetrical monomer construct components.

In some embodiments, the first monomer construct comprises, from N- to C-terminus of the CP and CM components, the CP1, the CM1, and, linked directly or indirectly (via a linker) to the C-terminus of the CM1, the DD1. In other embodiments, the first monomer construct comprises from C- to N-terminus of the CP and CM components, the CP1, the CM1, and, linked directly or indirectly (via a linker) to the N-terminus of the CM1, the DD1. In some embodiments, the second monomer construct comprises, from N- to C-terminal terminus of the CP and CM components, the CP2, the CM2, and, linked directly or indirectly (via a linker) to the C-terminus of the CM2, the DD2. In other embodiments, the second monomer construct comprises, from C- to N-terminus of the CP and CM components, the CP2, the CM2, and, linked directly or indirectly (via a linker) to the N-terminus of the CM2, the DD2.

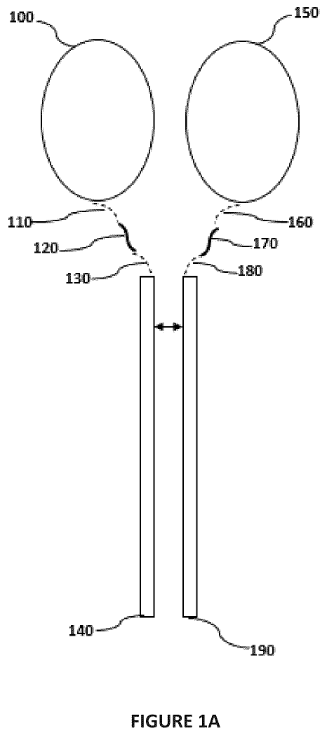

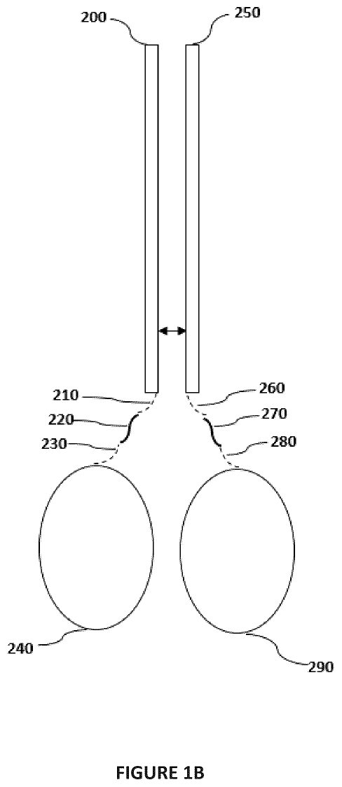

In certain embodiments, the first and second monomeric constructs are oriented such that the components in each member of the dimer are organized in the same order from N-terminus to C-terminus of the CP and CM components. A schematic of an illustrative ACC is provided in FIG. 1A. With reference to FIG. 1A, the ACC comprises, from N-terminus to C-terminus of the CP and CM components: (1) a first monomer construct having a CP1 100; a CM1 120 C-terminally positioned relative to the CP1 100; an optional linker 110, which, if present, is positioned between the C-terminus of the CP1 100 and the N-terminus of the CM1 120; a DD1 140; and an optional linker 130, which, if present, is positioned between the C-terminus of the CM1 120; and the DD1 140; (2) a second monomeric construct having a CP2 150; a CM2 170 that is C-terminally positioned relative to the CP2 150; an optional linker 160, which, if present, is positioned between the C-terminus of the CP2 150 and the N-terminus of the CM2 170; a DD2 190; and an optional linker 180, which, if present, is positioned between the C-terminus of the CM2 170 and the DD2 190; and (3) one or more covalent or non-covalent bonds (←→).

A schematic of a further illustrative ACC, with its components organized in the reverse orientation of the ACC is provided in FIG. 1B. With reference to FIG. 1B, the ACC comprises, from N-terminus to C-terminus of the CP and CM components: (1) a first monomeric construct having a DD1 200; a CM1 220; an optional linker 210, which, if present, is positioned between the DD1 200 and the N-terminus of the CM1 220; a CP1 240 C-terminally positioned relative to the CM1 220; and an optional linker 230, which, if present, is positioned between the C-terminus of the CM1 220 and the N-terminus of the CP1 240; (2) a second monomeric construct having a DD2 250; a CM2 270; an optional linker 260, which, if present, is positioned between the DD2 250 and the N-terminus of the CM2 270; a CP2 290 C-terminally positioned relative to the CM2 270; and an optional linker 280, which, if present, is positioned between the C-terminus of the CM2 290 and the N-terminus of the CP2 290; and (3) one or more covalent or non-covalent bonds (←→).

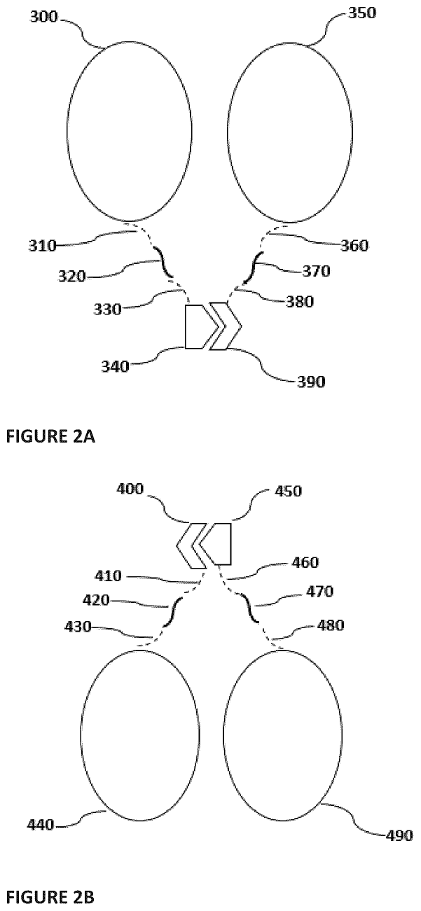

FIG. 2A is a schematic of an illustrative activatable cytokine construct comprising a first and second monomer construct that bind to each other by non-covalent means via first and second dimerization domains DD1 340 and DD2 390, respectively. The first monomer construct comprises, from N-terminus to C-terminus of the CP and CM components, a first mature cytokine protein CP1 300, a first optional linker 310, a first cleavable moiety CM1 320, a second optional linker 330, and a first dimerization domain DD1 340. The second monomer construct comprises, from N-terminus to C-terminus, a second mature cytokine protein CP2 350, a third optional linker 360, a second cleavable moiety CM2 370, a fourth optional linker 380, and a second dimerization domain DD2 390.

FIG. 2B is a schematic of an illustrative activatable cytokine construct comprising a first and second monomer construct that bind to each other by non-covalent means via first and second dimerization domains DD1 400 and DD2 450, respectively. The first monomer construct comprises, from N-terminus to C-terminus of the CP and CM components, a first dimerization domain DD1 400, a second optional linker 410, a first cleavable moiety CM1 420, a first optional linker 430, and a first mature cytokine protein CP1 440. The second monomer construct comprises, from N-terminus to C-terminus of the CP and CM components, a second dimerization domain DD2 450, a fourth optional linker 460, a second cleavable moiety CM2 470, a third optional linker 480, and a second mature cytokine protein CP2 490. In alternative aspects, one of the two moieties depicted as CP1 440 and CP2 490 is a truncated cytokine protein that lacks cytokine activity. For example, either CP1 or CP2 may be a truncated interferon alpha 2b having the first 151 amino acids of wild-type interferon alpha 2b. In alternative aspects, one of the two moieties depicted as CP1 440 and CP2 490 is a mutated cytokine protein that lacks cytokine activity. For example, either CP1 or CP2 may be a truncated interferon alpha 2b having a L130P mutation. In alternative aspects, one of the two moieties depicted as CP1 440 and CP2 490 is a polypeptide sequence that lacks cytokine activity, e.g., a signal moiety and/or a stub sequence. In alternative aspects, a first one of the two moieties depicted as CP1 440 and CP2 490 is a polypeptide sequence that binds with high affinity to a second one of the two moieties depicted as CP1 440 and CP2 490 and reduces the cytokine activity of the second moiety as compared to the control level of the second moiety.

The ACC structure was discovered to be highly effective at reducing activity of the mature cytokine protein components in a way that does not lead to substantially impaired cytokine activity after activation. The activation condition for the ACCs described herein is exposure to a protease that can cleave at least one of the cleavable moieties (CMs) in the ACC. As demonstrated in the Examples, activation of the ACC resulted in substantial recovery of cytokine activity. The results suggest that conformation of the cytokine components was not irreversibly altered within the context of the ACC. Significantly, the ACC does not rely on having to identify and utilize a peptide mask that has binding affinity for the cytokine protein component to achieve a masking effect. Thus, the ACC does not comprise a peptide mask having binding affinity for the cytokine protein component. The present inventors unexpectedly found that the ACC structure is sufficient to avoid off-target and undesired activity and/or toxic side effects of cytokines without use of any masking moieties having binding affinity for the cytokine protein component. Thus, the ACCs described herein are characterized in that they lack affinity masking moieties or peptide masking moieties.

The ACC may employ any of a variety of mature cytokine proteins, cleavable moieties, and DDs as the CP1, CP2, CM1, CM2, DD1, and DD2, respectively. For example, any of a variety of mature cytokine proteins that are known in the art or sequence and/or truncation variants thereof, may be suitable for use as either or both CP1 and CP2 components of the ACC. The mature cytokine proteins, CP1 and CP2 may be the same or different. In certain specific embodiments, CP1 and CP2 are the same. In other embodiments, CP1 and CP2 are different. The ACC may comprise additional amino acid residues at either or both N- and/or C-terminal ends of the CP1 and/or CP2.

In some embodiments, the CP1 and/or the CP2 may each independently comprise a mature cytokine protein selected from the group of: an interferon (such as, for example, an interferon alpha, an interferon beta, an interferon gamma, an interferon tau, and an interferon omega), an interleukin (such as, for example, IL-1α, IL-1β, IL-1RA, IL-18, IL-2, IL-4, IL-7, IL-9, IL-13, IL-15, IL-3, IL-5, GM-CSF, IL-6, IL-11, IL-21), G-CSF, IL-12, LIF, OSM, IL-10, IL-20, IL-14, IL-16, IL-17, CD154, LT-β, TNF-α, TNF-0, 4-1BBL, APRIL, CD70, CD153, CD178, GITRL, LIGHT, OX40L, TALL-1, TRAIL, TWEAK, TRANCE, TGF-β1, TGF-β1, TGF-β3, EPOo, TPO, Flt-3L, SCF, M-CSF, and MSP, and the like, as well as sequence and truncation variants thereof. For example, sequences of such proteins include those exemplified herein and additional sequences can be obtained from ncbi.nlm.nih.gov/protein. Truncation variants that are suitable for use in the ACCs of the present invention include any N- or C-terminally truncated cytokine that retains a cytokine activity. Exemplary truncation variants employed in the present invention include any of the truncated cytokine polypeptides that are known in the art (see, e.g., Slutzki et al., J. Mol. Biol. 360:1019-1030, 2006, and US 2009/0025106), as well as cytokine polypeptides that are N- and/or C-terminally truncated by 1 to about 40 amino acids, 1 to about 35 amino acids, 1 to about 30 amino acids, 1 to about 25 amino acids, 1 to about 20 amino acids, 1 to about 15 amino acids, 1 to about 10 amino acids, 1 to about 8 amino acids, 1 to about 6 amino acids, 1 to about 4 amino acids, that retain a cytokine activity. In some of the foregoing embodiments, the truncated CP is an N-terminally truncated CP. In other embodiments, the truncated CP is a C-terminally truncated CP. In certain embodiments, the truncated CP is a C- and an N-terminally truncated CP.

In some embodiments, the CP1 and/or the CP2 each independently comprise an amino acid sequence that is at least 80% identical (e.g., at least 82%, at least 84%, at least 86%, at least 88%, at least 90%, at least 92%, at least 94%, at least 95%, at least 96%, at least 97%, at least 98%, at least 99%, or 100% identical) to a cytokine reference sequence selected from the group consisting of: SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 12, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, and SEQ ID NO: 209. The percentage of sequence identity refers to the level of amino acid sequence identity between two or more peptide sequences when aligned using a sequence alignment program, e.g., the suite of BLAST programs, publicly available on the Internet at the NCBI website. See also Altschul et al., J. Mol. Biol. 215:403-10, 1990. In some aspects, the ACC includes an interferon alpha 2b mutant, for example, an interferon alpha 2b molecule having a mutation at position L130, e.g., L130P mutation, as either CP1 or CP2. In some aspects, the ACC includes an interferon alpha 2b mutant having a mutation at position I24, F64, I60, I63, F64, W76, I116, L117, F123, or L128, or a combination thereof. For example, the interferon alpha 2b mutant may include mutations I116 to T, N. or R; L128 to N, H, or R; I24 to P or Q; L117H; or L128T, or a combination thereof. In some aspects, the interferon alpha 2b mutant may include mutations I24Q, I60T, F64A, W76H, I116R, and L128N, or a subset thereof. In some aspects, the ACC includes as one of CP1 and CP2 a truncated interferon alpha 2b molecule that lacks cytokine activity. For example, the truncated interferon alpha 2b may consist of 151 or fewer amino acids of interferon alpha 2b, e.g., any one of amino acids in the wild-type interferon alpha 2b sequence from N to C-terminus: 1 to 151, 1 to 150, 1 to 149, 1 to 148, . . . 1 to 10, 1 to 9, 1 to 8, 1 to 7, 1 to 6, or 2 to 151, 3 to 151, 4 to 151, 5 to 150, 6 to 149, 7 to 148, 8 to 147, or any intervening sequence of amino acids or mutants thereof.

In certain specific embodiments, the CP1 and/or the CP2 comprise an interferon. Interferons that are suitable for use in the constructs of the present invention as CP1 and/or CP2 include, for example, an interferon-alpha, an interferon-beta, an interferon-omega, and an interferon-tau. In some embodiments, when the interferon is an interferon alpha, it may be an interferon alpha-2a, an interferon alpha-2b, or an interferon alpha-n3. Further examples of interferon alpha include interferon alpha-1, interferon alpha-4, interferon alpha-5, interferon alpha-6, interferon alpha-7, interferon alpha-8, interferon alpha-10, interferon alpha-13, interferon alpha-14, interferon alpha-16, interferon alpha-17, and interferon alpha-21. In some embodiments, the interferon is a recombinant or purified interferon alpha. In certain embodiments, when the interferon is an interferon-beta, it is selected from the group consisting of an interferon beta-1a and an interferon beta-1b. In some embodiments, the CP1 and/or the CP2 comprises an IFab domain of an interferon alpha or an interferon beta. The IFab domain is responsible for the cytokine release and antivirus functions of interferons. Exemplary IFab sequences are provided in SEQ ID Nos: 325-334.

In some embodiments, the CP1 and/or the CP2 exhibit(s) an interferon activity and include(s) an amino acid sequence that is at least 80% identical, at least 82% identical, at least 84% identical, at least 86% identical, at least 88% identical, at least 90% identical, at least 92% identical, at least 94% identical, at least 96% identical, at least 98% identical, or at least 99% identical, or 100% identical to an interferon alpha reference sequence selected from the group consisting of SEQ ID NO: 1, SEQ ID NO: 101, SEQ ID NO: 102, SEQ ID NO: 103, SEQ ID NO: 104, and SEQ ID NO: 105. In certain specific embodiments, the interferon alpha reference sequence is SEQ ID NO: 1 (human interferon alpha-2b). In some embodiments, the CP1 and/or the CP2 comprise a mature alpha interferon having an amino acid sequence selected from the group consisting of SEQ ID NO: 1, SEQ ID NO: 101, SEQ ID NO: 102, SEQ ID NO: 103, SEQ ID NO: 104, and SEQ ID NO: 105. In certain embodiments, the CP1 and/or the CP2 comprise a mature human alpha interferon having the amino acid sequence of SEQ ID NO: 1. In some of the above-described embodiments, the CP1 and the CP2 comprise the same amino acid sequence.

In other embodiments, the CP1 and/or the CP2 exhibit(s) an interferon activity and include(s) an amino acid sequence that is at least 80% identical, at least 82% identical, at least 84% identical, at least 86% identical, at least 88% identical, at least 90% identical, at least 92% identical, at least 94% identical, at least 96% identical, at least 98% identical, or at least 99% identical, or 100% identical to an interferon beta reference sequence selected from the group consisting of SEQ ID NO: 106, SEQ ID NO: 107, SEQ ID NO: 108, and SEQ ID NO: 109. In certain embodiments, the interferon beta reference sequence is a human interferon beta reference sequence selected from the group consisting of SEQ ID NO: 106 and SEQ ID NO: 107. In some embodiments, the CP1 and/or the CP2 comprise a mature beta interferon having an amino acid sequence selected from the group consisting of SEQ ID NO: 106, SEQ ID NO: 107, SEQ ID NO: 108, and SEQ ID NO: 109. In some of the above-described embodiments, the CP1 and the CP2 comprise the same amino acid sequence.

In some embodiments, the CP1 and/or CP2 exhibit(s) an interferon activity and include(s) an amino acid sequence that is at least 80% identical, at least 82% identical, at least 84% identical, at least 86% identical, at least 88% identical, at least 90% identical, at least 92% identical, at least 94% identical, at least 96% identical, at least 98% identical, or at least 99% identical, or 100% identical to an interferon omega reference sequence corresponding to SEQ ID NO: 110 (human interferon omega). In certain specific embodiments, the CP1 and/or CP2 comprise a mature human omega interferon having the amino acid sequence of SEQ ID NO: 110. In some of the above-described embodiments, the CP1 and the CP2 comprise the same amino acid sequence.

In some embodiments, the CP1 and/or the CP2 exhibit(s) an interleukin activity and include(s) an amino acid sequence that is at least 80% identical, at least 82%, at least 84%, at least 86%, at least 88%, at least 90%, at least 92%, at least 94%, at least 95%, at least 96%, at least 97%, at least 98%, or at least 99% identical or 100% identical to an interleukin reference sequence selected from the group consisting of: SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 12, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 143, SEQ ID NO: 144, SEQ ID NO: 145, SEQ ID NO: 146, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, and SEQ ID NO: 160. In some embodiments, CP1 and/or CP2 comprises a mature interleukin having an amino acid sequence selected from the group consisting of: SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 12, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 143, SEQ ID NO: 144, SEQ ID NO: 145, SEQ ID NO: 146, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, and SEQ ID NO: 160. In some of the above-described embodiments, the CP1 and the CP2 comprise the same amino acid sequence.

In some embodiments, CP1 and/or CP2 exhibit(s) an interleukin activity and include(s) an amino acid sequence that is at least 80% identical, at least 82%, at least 84%, at least 86%, at least 88%, at least 90%, at least 92%, at least 94%, at least 95%, at least 96%, at least 97%, at least 98%, or at least 99% identical to an interleukin reference sequence selected from the group consisting of SEQ ID NO: 111 (human IL-1 alpha), SEQ ID NO: 113 (human IL-1 beta), SEQ ID NO: 115 (human IL-1RA), SEQ ID NO: 117 (human IL-18), SEQ ID NO: 119 (human IL-2), SEQ ID NO: 121 (human IL-4), SEQ ID NO: 123 (human IL-7), SEQ ID NO: 125 (human IL-9), SEQ ID NO: 127 (human IL-13), SEQ ID NO: 129 (human IL-15), SEQ ID NO: 131 (human IL-3), SEQ ID NO: 133 (human IL-5), SEQ ID NO: 137 (human IL-6), SEQ ID NO: 139 (human IL-11), SEQ ID NO: 143 (human IL-12 alpha), SEQ ID NO: 144 (human IL-12 beta), SEQ ID NO: 151 (human IL-10), SEQ ID NO: 153 (human IL-20); SEQ ID NO: 155 (human IL-14), SEQ ID NO: 157 (human IL-16), and SEQ ID NO: 159 (human IL-17). In certain of these embodiments, CP1 and/or CP2 comprise an amino acid sequence from the group consisting of SEQ ID NO: 111 (human IL-1 alpha), SEQ ID NO: 113 (human IL-1 beta), SEQ ID NO: 115 (human IL-1RA), SEQ ID NO: 117 (human IL-18), SEQ ID NO: 119 (human IL-2), SEQ ID NO: 121, SEQ ID NO: 123 (human IL-7), SEQ ID NO: 125 (human IL-9), SEQ ID NO: 127 (human IL-13), SEQ ID NO: 129 (human IL-15), SEQ ID NO: 131 (human IL-3), SEQ ID NO: 133 (human IL-5), SEQ ID NO: 137 (human IL-6), SEQ ID NO: 139 (human IL-11), SEQ ID NO: 143 (human IL-12 alpha), SEQ ID NO: 144 (human IL-12 beta), SEQ ID NO: 151 (human IL-10), SEQ ID NO: 153 (human IL-20); SEQ ID NO: 155 (human IL-14), SEQ ID NO: 157 (human IL-16), and SEQ ID NO: 159 (human IL-17). In some of the above-described embodiments, the CP1 and the CP2 comprise the same amino acid sequence.

The number of amino acids in the sequence of the cytokine proteins employed may vary, depending on the specific cytokine protein employed. In some embodiments, the CP1 and/or the CP2 includes a total of about 10 amino acids to about 700 amino acids, about 10 amino acids to about 650 amino acids, about 10 amino acids to about 600 amino acids, about 10 amino acids to about 550 amino acids, about 10 amino acids to about 500 amino acids, about 10 amino acids to about 450 amino acids, about 10 amino acids to about 400 amino acids, about 10 amino acids to about 350 amino acids, about 10 amino acids to about 300 amino acids, about 10 amino acids to about 250 amino acids, about 10 amino acids to about 200 amino acids, about 10 amino acids to about 150 amino acids, about 10 amino acids to about 100 amino acids, about 10 amino acids to about 80 amino acids, about 10 amino acids to about 60 amino acids, about 10 amino acids to about 40 amino acids, about 10 amino acids to about 20 amino acids, about 20 amino acids to about 700 amino acids, about 20 amino acids to about 650 amino acids, about 20 amino acids to about 600 amino acids, about 20 amino acids to about 550 amino acids, about 20 amino acids to about 500 amino acids, about 20 amino acids to about 450 amino acids, about 20 amino acids to about 400 amino acids, about 20 amino acids to about 350 amino acids, about 20 amino acids to about 300 amino acids, about 20 amino acids to about 250 amino acids, about 20 amino acids to about 200 amino acids, about 20 amino acids to about 150 amino acids, about 20 amino acids to about 100 amino acids, about 20 amino acids to about 80 amino acids, about 20 amino acids to about 60 amino acids, about 20 amino acids to about 40 amino acids, about 40 amino acids to about 700 amino acids, about 40 amino acids to about 650 amino acids, about 40 amino acids to about 600 amino acids, about 40 amino acids to about 550 amino acids, about 40 amino acids to about 500 amino acids, about 40 amino acids to about 450 amino acids, about 40 amino acids to about 400 amino acids, about 40 amino acids to about 350 amino acids, about 40 amino acids to about 300 amino acids, about 40 amino acids to about 250 amino acids, about 40 amino acids to about 200 amino acids, about 40 amino acids to about 150 amino acids, about 40 amino acids to about 100 amino acids, about 40 amino acids to about 80 amino acids, about 40 amino acids to about 60 amino acids, about 60 amino acids to about 700 amino acids, about 60 amino acids to about 650 amino acids, about 60 amino acids to about 600 amino acids, about 60 amino acids to about 550 amino acids, about 60 amino acids to about 500 amino acids, about 60 amino acids to about 450 amino acids, about 60 amino acids to about 400 amino acids, about 60 amino acids to about 350 amino acids, about 60 amino acids to about 300 amino acids, about 60 amino acids to about 250 amino acids, about 60 amino acids to about 200 amino acids, about 60 amino acids to about 150 amino acids, about 60 amino acids to about 100 amino acids, about 60 amino acids to about 80 amino acids, about 80 amino acids to about 700 amino acids, about 80 amino acids to about 650 amino acids, about 80 amino acids to about 600 amino acids, about 80 amino acids to about 550 amino acids, about 80 amino acids to about 500 amino acids, about 80 amino acids to about 450 amino acids, about 80 amino acids to about 400 amino acids, about 80 amino acids to about 350 amino acids, about 80 amino acids to about 300 amino acids, about 80 amino acids to about 250 amino acids, about 80 amino acids to about 200 amino acids, about 80 amino acids to about 150 amino acids, about 80 amino acids to about 100 amino acids, about 100 amino acids to about 700 amino acids, about 100 amino acids to about 650 amino acids, about 100 amino acids to about 600 amino acids, about 100 amino acids to about 550 amino acids, about 100 amino acids to about 500 amino acids, about 100 amino acids to about 450 amino acids, about 100 amino acids to about 400 amino acids, about 100 amino acids to about 350 amino acids, about 100 amino acids to about 300 amino acids, about 100 amino acids to about 250 amino acids, about 100 amino acids to about 200 amino acids, about 100 amino acids to about 150 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 450 amino acids, about 150 amino acids to about 400 amino acids, about 150 amino acids to about 350 amino acids, about 150 amino acids to about 300 amino acids, about 150 amino acids to about 250 amino acids, about 150 amino acids to about 200 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 450 amino acids, about 200 amino acids to about 400 amino acids, about 200 amino acids to about 350 amino acids, about 200 amino acids to about 300 amino acids, about 200 amino acids to about 250 amino acids, about 250 amino acids to about 700 amino acids, about 250 amino acids to about 650 amino acids, about 250 amino acids to about 600 amino acids, about 250 amino acids to about 550 amino acids, about 250 amino acids to about 500 amino acids, about 250 amino acids to about 450 amino acids, about 250 amino acids to about 400 amino acids, about 250 amino acids to about 350 amino acids, about 250 amino acids to about 300 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 450 amino acids, about 300 amino acids to about 400 amino acids, about 300 amino acids to about 350 amino acids, about 350 amino acids to about 700 amino acids, about 350 amino acids to about 650 amino acids, about 350 amino acids to about 600 amino acids, about 350 amino acids to about 550 amino acids, about 350 amino acids to about 500 amino acids, about 350 amino acids to about 450 amino acids, about 350 amino acids to about 400 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 450 amino acids, about 450 amino acids to about 700 amino acids, about 450 amino acids to about 650 amino acids, about 450 amino acids to about 600 amino acids, about 450 amino acids to about 550 amino acids, about 450 amino acids to about 500 amino acids, about 500 amino acids to about 700 amino acids, about 500 amino acids to about 650 amino acids, about 500 amino acids to about 600 amino acids, about 500 amino acids to about 550 amino acids, about 550 amino acids to about 700 amino acids, about 550 amino acids to about 650 amino acids, about 550 amino acids to about 600 amino acids, about 600 amino acids to about 700 amino acids, about 600 amino acids to about 650 amino acids, or about 650 amino acids to about 700 amino acids. In some embodiments, CP1 and/or the CP2 is a mature wildtype human cytokine protein.

Each monomer construct of the ACC may employ any of a variety of dimerization domains. Suitable DDs include both polymeric (e.g., a synthetic polymer, a polypeptide, a polynucleotide, and the like) and small molecule (non-polymeric moieties having a molecular weight of less than about 1 kilodalton, and sometimes less than about 800 daltons) types of moieties. The pair of DDs may be any pair of moieties that are known in the art to bind to each other.

For example, in some embodiments, the DD1 and the DD2 are members of a pair selected from the group of: a sushi domain from an alpha chain of human IL-15 receptor (IL15Rα) and a soluble IL-15; barnase and barnstar; a PKA and an AKAP; adapter/docking tag molecules based on mutated RNase I fragments; a pair of antigen-binding domains (e.g., a pair of single domain antibodies); soluble N-ethyl-maleimide sensitive factor attachment protein receptors (SNARE) modules based on interactions of the proteins syntaxin, synaptotagmin, synaptobrevin, and SNAP25; a single domain antibody (sdAb) and corresponding epitope; an antigen-binding domain (e.g., a single chain antibody such as a single chain variable fragment (scFv), a single domain antibody, and the like) and a corresponding epitope; coiled coil polypeptide structions (e.g., Fos-Jun coiled coil structures, acid/base coiled-coil helices, Glu-Lys coiled coil helices, leucine zipper structures), small molecule binding pairs such as biotin and avidin or streptavidin, amine/aldehyde, lectin/carbohydrate; a pair of polymers that can bind each other, such as, for example, a pair of sulfur- or thiol-containing polymers (e.g., a pair of Fc domains, a pair of thiolized-human serum albumin polypeptides, and the like); and the like.

In some embodiments, the DD1 and DD2 are non-polypeptide polymers. The non-polypeptide polymers may covalently bound to each other. In some examples, the non-polypeptide polymers may be a sulfur-containing polymer, e.g., sulfur-containing polyethylene glycol. In such cases, the DD1 and DD2 may be covalently bound to each other via one or more disulfide bonds.

When the pair of DD1 and DD2 are members of a pair of epitope and antigen-binding domain, the epitope may be a naturally or non-naturally occurring epitope. Exemplary non-naturally occurring epitopes include, for example, a non-naturally occurring peptide, such as, for example, a poly-His peptide (e.g., a His tag, and the like).

In certain specific embodiments, the DD1 and the DD2 are a pair of Fc domains. As used herein, an “Fc domain” refers to a contiguous amino acid sequence of a single heavy chain of an immunoglobulin. A pair of Fe domains associate together to form an Fc region of an immunoglobulin.

In some embodiments, the pair of Fc domains is a pair of human Fc domains (e.g., a pair of wildtype human Fc domains). In some embodiments, the human Fc domains are human IgG1 Fc domains (e.g., wildtype human IgG1 Fc domains), human IgG2 Fc domains (e.g., wildtype human IgG2 Fc domains), human IgG3 Fc domains (e.g., wildtype human IgG3 Fc domains), or human IgG4 Fc domains (e.g., wildtype human IgG4 Fc domains). In some embodiments, the human Fc domains comprise a sequence that is at least 80% identical (e.g., at least 82%, at least 84%, at least 85%, at least 86%, at least 88%, at least 90%, at least 92%, at least 94%, at least 95%, at least 96%, at least 97%, at least 98%, at least 99%, or 100% identical) to SEQ ID NO: 3.

In some embodiments, the pair of Fc domains comprise a knob mutant and a hole mutant of a Fc domain. The knob and hole mutants may interact with each other to facilitate the dimerization. In some embodiments, the knob and hole mutants may comprise one or more amino acid modifications within the interface between two Fc domains (e.g., in the CH3 domain). In one example, the modifications comprise amino acid substitution T366W and optionally the amino acid substitution S354C in one of the antibody heavy chains, and the amino acid substitutions T366S, L368A, Y407V and optionally Y349C in the other one of the antibody heavy chains (numbering according to EU index of Kabat numbering system). Examples of the knob and hole mutants include Fc mutants of SEQ ID NOs: 315 and 316, as well as those described in U.S. Pat. Nos. 5,731,168; 7,695,936; and 10,683,368, which are incorporated herein by reference in their entireties. In some embodiments, the dimerization domains comprise a sequence that is at least 80% identical (e.g., at least 82%, at least 84%, at least 85%, at least 86%, at least 88%, at least 90%, at least 92%, at least 94%, at least 95%, at least 96%, at least 97%, at least 98%, at least 99%, or 100% identical) to SEQ ID NOs: 315 and 316, respectively.

In some embodiments, DD1 and/or DD2 can further include a serum half-life extending moiety (e.g., polypeptides that bind serum proteins, such as immunoglobulin (e.g., IgG) or serum albumin (e.g., human serum albumin (HSA)). Examples of half-life extending moieties include hexa-hat GST (glutathione S-transferase) glutathione affinity, Calmodulin-binding peptide (CBP), Strep-tag, Cellulose Binding Domain, Maltose Binding Protein, S-Peptide Tag, Chitin Binding Tag, Immuno-reactive Epitopes, Epitope Tags, E2Tag, HA Epitope Tag, Myc Epitope, FLAG Epitope, AUl and AU5 Epitopes, Glu-Glu Epitope, KT3 Epitope, IRS Epitope, Btag Epitope, Protein Kinase-C Epitope, and VSV Epitope.

In some embodiments, DD1 and/or DD2 each include a total of about 5 amino acids to about 250 amino acids, about 5 amino acids to about 200 amino acids, about 5 amino acids to about 180 amino acids, about 5 amino acids to about 160 amino acids, about 5 amino acids to about 140 amino acids, about 5 amino acids to about 120 amino acids, about 5 amino acids to about 100 amino acids, about 5 amino acids to about 80 amino acids, about 5 amino acids to about 60 amino acids, about 5 amino acids to about 40 amino acids, about 5 amino acids to about 20 amino acids, about 5 amino acids to about 10 amino acids, about 10 amino acids to about 250 amino acids, about 10 amino acids to about 200 amino acids, about 10 amino acids to about 180 amino acids, about 10 amino acids to about 160 amino acids, about 10 amino acids to about 140 amino acids, about 10 amino acids to about 120 amino acids, about 10 amino acids to about 100 amino acids, about 10 amino acids to about 80 amino acids, about 10 amino acids to about 60 amino acids, about 10 amino acids to about 40 amino acids, about 10 amino acids to about 20 amino acids, about 20 amino acids to about 250 amino acids, about 20 amino acids to about 200 amino acids, about 20 amino acids to about 180 amino acids, about 20 amino acids to about 160 amino acids, about 20 amino acids to about 140 amino acids, about 20 amino acids to about 120 amino acids, about 20 amino acids to about 100 amino acids, about 20 amino acids to about 80 amino acids, about 20 amino acids to about 60 amino acids, about 20 amino acids to about 40 amino acids, about 40 amino acids to about 250 amino acids, about 40 amino acids to about 200 amino acids, about 40 amino acids to about 180 amino acids, about 40 amino acids to about 160 amino acids, about 40 amino acids to about 140 amino acids, about 40 amino acids to about 120 amino acids, about 40 amino acids to about 100 amino acids, about 40 amino acids to about 80 amino acids, about 40 amino acids to about 60 amino acids, about 60 amino acids to about 250 amino acids, about 60 amino acids to about 200 amino acids, about 60 amino acids to about 180 amino acids, about 60 amino acids to about 160 amino acids, about 60 amino acids to about 140 amino acids, about 60 amino acids to about 120 amino acids, about 60 amino acids to about 100 amino acids, about 60 amino acids to about 80 amino acids, about 80 amino acids to about 250 amino acids, about 80 amino acids to about 200 amino acids, about 80 amino acids to about 180 amino acids, about 80 amino acids to about 160 amino acids, about 80 amino acids to about 140 amino acids, about 80 amino acids to about 120 amino acids, about 80 amino acids to about 100 amino acids, about 100 amino acids to about 250 amino acids, about 100 amino acids to about 200 amino acids, about 100 amino acids to about 180 amino acids, about 100 amino acids to about 160 amino acids, about 100 amino acids to about 140 amino acids, about 100 amino acids to about 120 amino acids, about 120 amino acids to about 250 amino acids, about 120 amino acids to about 200 amino acids, about 120 amino acids to about 180 amino acids, about 120 amino acids to about 160 amino acids, about 120 amino acids to about 140 amino acids, about 140 amino acids to about 250 amino acids, about 140 amino acids to about 200 amino acids, about 140 amino acids to about 180 amino acids, about 140 amino acids to about 160 amino acids, about 160 amino acids to about 250 amino acids, about 160 amino acids to about 200 amino acids, about 160 amino acids to about 180 amino acids, about 180 amino acids to about 250 amino acids, about 180 amino acids to about 200 amino acids, or about 200 amino acids to about 250 amino acids. In some embodiments, DD1 and DD2 are each an Fc domain that comprises a portion of the hinge region that includes two cysteine residues, a CH2 domain, and a CH3 domain. In some embodiments, DD1 and DD2 are each an Fc domain whose N-terminus is the first cysteine residue in the hinge region reading in the N- to C-direction (e.g., Cysteine 226 of human IgG1 or IgG4, using EU numbering).

In some aspects, positioned between the CP and the DD components, either directly or indirectly (e.g., via a linker), is a cleavable moiety that comprises a substrate for a protease. In some embodiments, the CM1 and CM2 may each independently comprise a substrate for a protease selected from the group consisting of ADAM8, ADAM9, ADAM10, ADAM12, ADAM15, ADAM17/TACE, ADEMDEC1, ADAMTS1, ADAMTS4, ADAMTS5, BACE, Renin, Cathepsin D, Cathepsin E, Caspase 1, Caspase 2, Caspase 3, Caspase 4, Caspase 5, Caspase 6, Caspase 7, Caspase 8, Caspase 9, Caspase 10, Caspase 14, Cathepsin A, Cathepsin B, Cathepsin C, Cathepsin G, Cathepsin K, Cathepsin L, Cathepsin S, Cathepsin V/L2, Cathepsin X/Z/P, Chymase, Cruzipain, DESC1, DPP-4, FAP, Legumain, Otubain-2, Elastase, FVIIa, FiXA, FXa, FXIa, FXIIa, Granzyme B, Guanidinobenzoatase, Hepsin, HtrA1, Human Neutrophil Elastase, KLK4, KLK5, KLK6, KLK7, KLK8, KLK10, KLK11, KLK13, KLK14, Lactoferrin, Marapsin, Matriptase-2, Meprin, MT-SP1/Matriptase, Neprilysin, NS3/4A, PACE4, Plasmin, PSMA, PSA, BMP-1, MMP1, MMP2, MMP3, MMP7, MMP8, MMP9, MMP10, MMP11, MMP12, MMP13, MMP14, MMP15, MMP16, MMPP17, MMP19, MMP20, MMP23, MMP24, MMP26, MMP27, TMPRSS2, TMPRSS3, TMPRSS4, tPA, Thrombin, Tryptase, and uPA.

In some embodiments of any of the ACCs described herein, the protease that cleaves any of the CMs described herein can be ADAM8, ADAM9, ADAM10, ADAM12, ADAM15, ADAM17/TACE, ADAMDEC1, ADAMTS1, ADAMTS4, ADAMTS5, BACE, Renin, Cathepsin D, Cathepsin E, Caspase 1, Caspase 2, Caspase 3, Caspase 4, Caspase 5, Caspase 6, Caspase 7, Caspase 8, Caspase 9, Caspase 10, Caspase 14, Cathepsin B, Cathepsin C, Cathepsin K, Cathespin L, Cathepsin S, Cathepsin V/L2, Cathepsin X/Z/P, Cruzipain, Legumain, Otubain-2, KLK4, KLK5, KLK6, KLK7, KLK8, KLK10, KLK11, KLK13, KLK14, Meprin, Neprilysin, PSMA, BMP-1, MMP-1, MMP-2, MMP-3, MMP-7, MMP-9, MMP-10, MMP-11, MMP-12, MMP-13, MMP-14, MMP-15, MMP-16, MMP-17, MMP-19, MMP-20, MMP-23, MMP-24, MMP-26, MMP-27, activated protein C, cathepsin A, cathepsin G, Chymase, FVIIa, FIXa, FXa, FXIa, FXIIa, Elastase, Granzyme B, Guanidinobenzoatase, HtrA1, human neutrophil lyase, lactoferrin, marapsin, NS3/4A, PACE4, Plasmin, PSA, tPA, thrombin, tryptase, uPA, DESC1, DPP-4, FAP, Hepsin, Matriptase-2, MT-SP1/Matripase, TMPRSS2, TMPRSS3, and TMPRSS4.

In some embodiments of any of the ACCs described herein, the protease is selected from the group of: uPA, legumain, MT-SP1, ADAM17, BMP-1, TMPRSS3, TMPRSS4, MMP-2, MMP-9, MMP-12, MMP-13, and MMP-14.

Increased levels of proteases having known substrates have been reported in a number of cancers. See, e.g., La Roca et al., British J. Cancer 90(7):1414-1421, 2004. Substrates suitable for use in the CM1 and/or CM2 components employed herein include those which are more prevalently found in cancerous cells and tissue. Thus, in certain embodiments, CM1 and/or CM2 each independently comprise a substrate for a protease that is more prevalently found in diseased tissue associated with a cancer. In some embodiments, the cancer is selected from the group of: gastric cancer, breast cancer, osteosarcoma, and esophageal cancer. In some embodiments, the cancer is breast cancer. In some embodiments, the cancer is a HER2-positive cancer. In some embodiments, the cancer is Kaposi sarcoma, hairy cell leukemia, chronic myeloid leukemia (CML), follicular lymphoma, renal cell cancer (RCC), melanoma, neuroblastoma, basal cell carcinoma, cutaneous T-cell lymphoma, nasopharyngeal adenocarcimoa, breast cancer, ovarian cancer, bladder cancer, BCG-resistant non-muscle invasive bladder cancer (NMIBC), endometrial cancer, pancreatic cancer, non-small cell lung cancer (NSCLC), colorectal cancer, esophageal cancer, gallbladder cancer, glioma, head and neck carcinoma, uterine cancer, cervical cancer, or testicular cancer, and the like. In some of the above-described embodiments, the CM components comprise substrates for protease(s) that is/are more prevalent in tumor tissue.

In some embodiments, CM1 and/or CM2 each independently include(s) a sequence selected from the group consisting of SEQ ID NO: 5 to SEQ ID NO: 100, as well as C-terminal and N-terminal truncation variants thereof.

In some embodiments, the CM includes a sequence selected from the group of: ISSGLLSGRSDNH (SEQ ID NO: 28), LSGRSDDH (SEQ ID NO: 33), ISSGLLSGRSDQH (SEQ ID NO: 54), and ISSGLLSGRSDNI (SEQ ID NO: 68).

In certain embodiments, CM1 and/or CM2 include(s) a sequence selected from the group of: APRSALAHGLF (SEQ ID NO: 263), AQNLLGMY (SEQ ID NO: 264), LSGRSDNHGGAVGLLAPP (SEQ ID NO: 265), VHMPLGFLGPGGLSGRSDNH (SEQ ID NO: 266), LSGRSDNHGGVHMPLGFLGP (SEQ ID NO: 267), LSGRSDNHGGSGGSISSGLLSS (SEQ ID NO: 268), ISSGLLSSGGSGGSLSGRSGNH (SEQ ID NO: 269), LSGRSDNHGGSGGSQNQALRMA (SEQ ID NO: 270), QNQALRMAGGSGGSLSGRSDNH (SEQ ID NO:271), LSGRSGNHGGSGGSQNQALRMA (SEQ ID NO: 272), QNQALRMAGGSGGSLSGRSGNH (SEQ ID NO: 273), ISSGLLSGRSGNH (SEQ ID NO: 274), as well as C-terminal and N-terminal truncation variants thereof. Examples of CM also include those described in U.S. Patent Application Publication Nos. 2016/0289324, 2019/0284283, and in publication numbers WO 2010/081173, WO 2015/048329, WO 2015/116933, WO 2016/118629, and WO 2020/118109, which are incorporated herein by reference in their entireties.

Truncation variants of the aforementioned amino acid sequences that are suitable for use in a CM1 and/or CM2 are any that retain the recognition site for the corresponding protease. These include C-terminal and/or N-terminal truncation variants comprising at least 3 contiguous amino acids of the above-described amino acid sequences, or at least 4, or at least 5, or at least 6, or at least 7 amino acids of the foregoing amino acid sequences that retain a recognition site for a protease. In certain embodiments, the truncation variant of the above-described amino acid sequences is an amino acid sequence corresponding to any of the above, but that is C- and/or N-terminally truncated by 1 to about 10 amino acids, 1 to about 9 amino acids, 1 to about 8 amino acids, 1 to about 7 amino acids, 1 to about 6 amino acids, 1 to about 5 amino acids, 1 to about 4 amino acids, or 1 to about 3 amino acids, and which: (1) has at least three amino acid residues; and (2) retains a recognition site for a protease. In some of the foregoing embodiments, the truncated CM is an N-terminally truncated CM. In some embodiments, the truncated CM is a C-terminally truncated CM. In some embodiments, the truncated C is a C- and an N-terminally truncated CM.

In some embodiments of any of the activatable cytokine constructs described herein, the CM1 and/or the CM2 comprise a total of about 3 amino acids to about 25 amino acids. In some embodiments, the CM1 and/or CM2 comprise a total of about 3 amino acids to about 25 amino acids, about 3 amino acids to about 20 amino acids, about 3 amino acids to about 15 amino acids, about 3 amino acids to about 10 amino acids, about 3 amino acids to about 5 amino acids, about 5 amino acids to about 25 amino acids, about 5 amino acids to about 20 amino acids, about 5 amino acids to about 15 amino acids, about 5 amino acids to about 10 amino acids, about 10 amino acids to about 25 amino acids, about 10 amino acids to about 20 amino acids, about 10 amino acids to about 15 amino acids, about 15 amino acids to about 25 amino acids, about 15 amino acids to about 20 amino acids, or about 20 amino acids to about 25 amino acids.

In some embodiments, the ACC may comprise multiple CMs that comprise substrates for different proteases. In some embodiments, the CM1 and the CM2 comprise substrates for different proteases. In some embodiments, the CM1 and the CM2 comprise substrates for the same protease.

The first and second monomer constructs may comprise one or more additional components including one or more linkers, and the like. In some embodiments, the first monomer can include a linker disposed between the CP1 and the CM1. In some embodiments, the CP1 and the CM1 directly abut each other in the first monomer. In some embodiments, the first monomer comprises a linker disposed between the CM1 and the DD1. In some embodiments, the linker has a total length of 1 amino acid to about 15 amino acids. In some embodiments, the CM1 and the DD1 directly abut each other in the first monomer. In some embodiments, the CM and any linkers disposed between the CP1 and DD1 have a combined total length of 3 to 15 amino acids, or 3 to 10 amino acids, or 3 to 7 amino acids.

In some embodiments, the second monomer comprises a linker disposed between the CP2 and the CM2. In some embodiments, the CP2 and the CM2 directly abut each other in the second monomer. In some embodiments, the second monomer comprises a linker disposed between the CM2 and the DD2. In some embodiments, the linker has a total length of 1 amino acid to about 15 amino acids. In some embodiments, the linker comprises a sequence of GGGS (SEQ ID NO: 2). In some embodiments, the CM2 (e.g., any of the cleavable moieties described herein) and the DD2 (e.g., any of the DDs described herein) directly abut each other in the second monomer. In some embodiments, the CM and any linkers disposed between the CP2 and DD2 have a combined total length of 3 to 15 amino acids, or 3 to 10 amino acids, or 3 to 7 amino acids.

In some embodiments, the first monomer and/or the second monomer can include a total of about 50 amino acids to about 800 amino acids, about 50 amino acids to about 750 amino acids, about 50 amino acids to about 700 amino acids, about 50 amino acids to about 650 amino acids, about 50 amino acids to about 600 amino acids, about 50 amino acids to about 550 amino acids, about 50 amino acids to about 500 amino acids, about 50 amino acids to about 450 amino acids, about 50 amino acids to about 400 amino acids, about 50 amino acids to about 350 amino acids, about 50 amino acids to about 300 amino acids, about 50 amino acids to about 250 amino acids, about 50 amino acids to about 200 amino acids, about 50 amino acids to about 150 amino acids, about 50 amino acids to about 100 amino acids, about 100 amino acids to about 800 amino acids, about 100 amino acids to about 750 amino acids, about 100 amino acids to about 700 amino acids, about 100 amino acids to about 650 amino acids, about 100 amino acids to about 600 amino acids, about 100 amino acids to about 550 amino acids, about 100 amino acids to about 500 amino acids, about 100 amino acids to about 450 amino acids, about 100 amino acids to about 400 amino acids, about 100 amino acids to about 350 amino acids, about 100 amino acids to about 300 amino acids, about 100 amino acids to about 250 amino acids, about 100 amino acids to about 200 amino acids, about 100 amino acids to about 150 amino acids, about 150 amino acids to about 800 amino acids, about 150 amino acids to about 750 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 450 amino acids, about 150 amino acids to about 400 amino acids, about 150 amino acids to about 350 amino acids, about 150 amino acids to about 300 amino acids, about 150 amino acids to about 250 amino acids, about 150 amino acids to about 200 amino acids, about 200 amino acids to about 800 amino acids, about 200 amino acids to about 750 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 450 amino acids, about 200 amino acids to about 400 amino acids, about 200 amino acids to about 350 amino acids, about 200 amino acids to about 300 amino acids, about 200 amino acids to about 250 amino acids, about 250 amino acids to about 800 amino acids, about 250 amino acids to about 750 amino acids, about 250 amino acids to about 700 amino acids, about 250 amino acids to about 650 amino acids, about 250 amino acids to about 600 amino acids, about 250 amino acids to about 550 amino acids, about 250 amino acids to about 500 amino acids, about 250 amino acids to about 450 amino acids, about 250 amino acids to about 400 amino acids, about 250 amino acids to about 350 amino acids, about 250 amino acids to about 300 amino acids, about 300 amino acids to about 800 amino acids, about 300 amino acids to about 750 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 450 amino acids, about 300 amino acids to about 400 amino acids, about 300 amino acids to about 350 amino acids, about 350 amino acids to about 800 amino acids, about 350 amino acids to about 750 amino acids, about 350 amino acids to about 700 amino acids, about 350 amino acids to about 650 amino acids, about 350 amino acids to about 600 amino acids, about 350 amino acids to about 550 amino acids, about 350 amino acids to about 500 amino acids, about 350 amino acids to about 450 amino acids, about 350 amino acids to about 400 amino acids, about 400 amino acids to about 800 amino acids, about 400 amino acids to about 750 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 450 amino acids, about 450 amino acids to about 800 amino acids, about 450 amino acids to about 750 amino acids, about 450 amino acids to about 700 amino acids, about 450 amino acids to about 650 amino acids, about 450 amino acids to about 600 amino acids, about 450 amino acids to about 550 amino acids, about 450 amino acids to about 500 amino acids, about 500 amino acids to about 800 amino acids, about 500 amino acids to about 750 amino acids, about 500 amino acids to about 700 amino acids, about 500 amino acids to about 650 amino acids, about 500 amino acids to about 600 amino acids, about 500 amino acids to about 550 amino acids, about 550 amino acids to about 800 amino acids, about 550 amino acids to about 750 amino acids, about 550 amino acids to about 700 amino acids, about 550 amino acids to about 650 amino acids, about 550 amino acids to about 600 amino acids, about 600 amino acids to about 800 amino acids, about 600 amino acids to about 750 amino acids, about 600 amino acids to about 700 amino acids, about 600 amino acids to about 650 amino acids, about 650 amino acids to about 800 amino acids, about 650 amino acids to about 750 amino acids, about 650 amino acids to about 700 amino acids, about 700 amino acids to about 800 amino acids, about 700 amino acids to about 750 amino acids, or about 750 amino acids to about 800 amino acids.

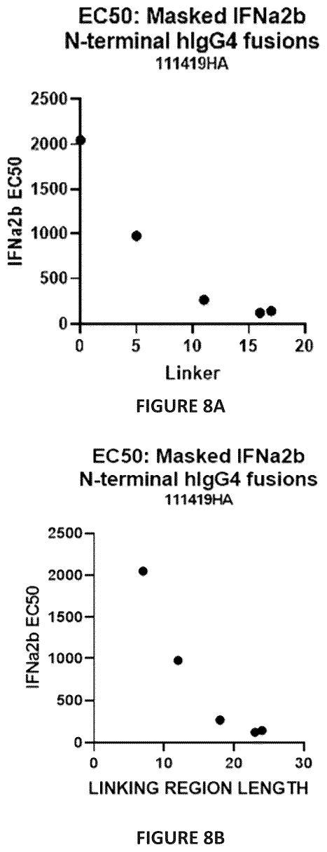

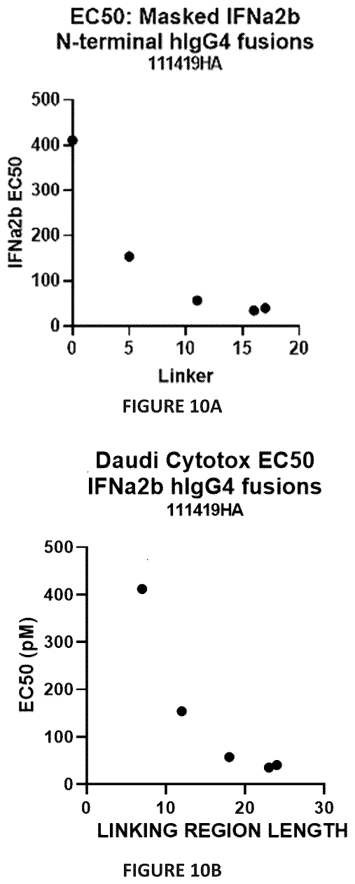

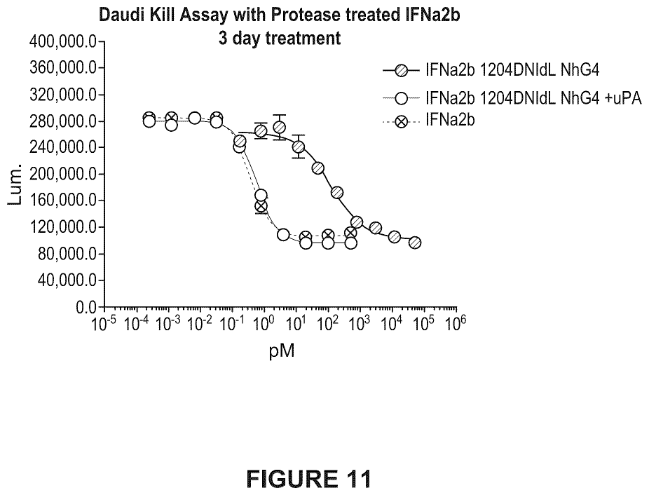

In some embodiments of any of the ACCs described herein, one or more linkers (e.g., flexible linkers) can be introduced into the activatable cytokine construct to provide flexibility at one or more of the junctions between domains, between moieties, between moieties and domains, or at any other junctions where a linker would be beneficial. In some embodiments, where the ACC is provided as a conformationally constrained construct, a flexible linker can be inserted to facilitate formation and maintenance of a structure in the uncleaved activatable cytokine construct. Any of the linkers described herein can provide the desired flexibility to facilitate the inhibition of the binding of a target (e.g., a receptor of a cytokine), or to facilitate cleavage of a CM by a protease. In some embodiments, linkers are included in the ACC that are all or partially flexible, such that the linker can include a flexible linker as well as one or more portions that confer less flexible structure to provide for a desired ACC. Some linkers may include cysteine residues, which may form disulfide bonds and reduce flexibility of the construct. In some embodiments, reducing the length of the linkers or Linking Region reduces the activity of the mature cytokine protein in the ACCs (see, e.g., FIGS. 8A-8B and 10A-10B). In most instances, linker length is determined by counting, in a N- to C-direction, the number of amino acids from the N-terminus of the linker adjacent to the C-terminal amino acid of the preceding component, to the C-terminus of the linker adjacent to the N-terminal amino acid of the following component (i.e., where the linker length does not include either the C-terminal amino acid of the preceding component or the N-terminal amino acid of the following component). In embodiments in which a linker is employed at the N-terminus of a DD that comprises an Fc domain, linker length is determined by counting the number of amino acids from the N-terminus of the linker adjacent to the C-terminal amino acid of the preceding component to C-terminus of the linker adjacent to the first cysteine of an Fc hinge region (i.e., where the linker length does not include the C-terminal amino acid of the preceding component or the first cysteine of the Fc hinge region).

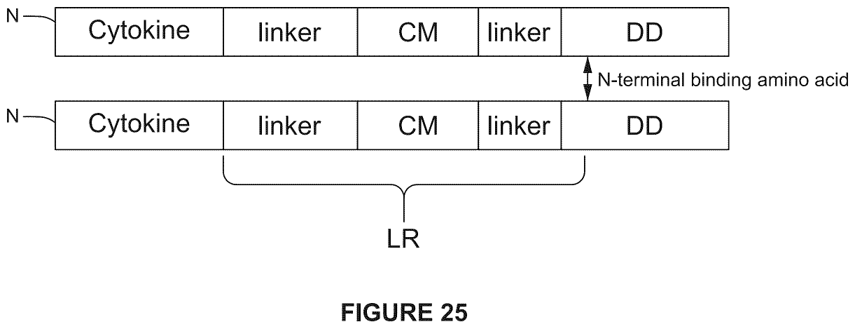

As apparent from the present disclosure and FIG. 25, ACCs of the present disclosure include a stretch of amino acids between the CP and the proximal point of interaction between the dimerization domains. That stretch of amino acids may be referred to as a Linking Region (LR). As used herein, the term “Linking Region” or “LR” refers to the stretch of amino acid residues between the C-terminus of the cytokine and the amino acid residue that is N-terminally adjacent to the proximal point of interaction between the dimerization domains (i.e., the linking region does not include the C-terminal amino acid of the cytokine or the N-terminal amino acid of the DD that forms the proximal point of interaction to the DD of the corresponding second monomer). For example, when the DDs are a pair of Fc domains, the linking region is the stretch of amino acid residues between the C-terminus of the cytokine and the first N-terminal cysteine residue that participates in the disulfide linkage of the Fc (e.g., Cysteine 226 of an IgG1 or IgG4 Fc domain, according to EU numbering). When the dimerization domain is not a peptide, then the linking region is the stretch of amino acid residues following the C-terminus of the cytokine until the last amino acid. For example, when the DDs are a biotin-streptavidin pair, the linking region of the biotin-containing monomer is the stretch of amino acid residues between the C-terminus of the cytokine and the biotin molecule, and the linking region of the streptavidin-containing monomer is the stretch of amino acid residues between the C-terminus of the cytokine and the streptavidin molecule. In some aspects, the Linking Region may comprise no more than 24, 18, 14, 12, 11, 10, 9, 8, 7, 6, 5, or 4 amino acids, e.g., 5 to 14, 7 to 12, or 8 to 11 amino acids.

In some embodiments, additional amino acid sequences may be positioned N-terminally or C-terminally to any of the domains of any of the ACCs. Examples include, but are not limited to, targeting moieties (e.g., a ligand for a receptor of a cell present in a target tissue) and serum half-life extending moieties (e.g., polypeptides that bind serum proteins, such as immunoglobulin (e.g., IgG) or serum albumin (e.g., human serum albumin (HSA)).

In some embodiments of any of the activatable cytokine constructs described herein, a linker can include a total of about 1 amino acid to about 25 amino acids (e.g., about 1 amino acid to about 24 amino acids, about 1 amino acid to about 22 amino acids, about 1 amino acid to about 20 amino acids, about 1 amino acid to about 18 amino acids, about 1 amino acid to about 16 amino acids, about 1 amino acid to about 15 amino acids, about 1 amino acid to about 14 amino acids, about 1 amino acid to about 12 amino acids, about 1 amino acid to about 10 amino acids, about 1 amino acid to about 8 amino acids, about 1 amino acid to about 6 amino acids, about 1 amino acid to about 5 amino acids, about 1 amino acid to about 4 amino acids, about 1 amino acid to about 3 amino acids, about 1 amino acid to about 2 amino acids, about 2 amino acids to about 25 amino acids, about 2 amino acids to about 24 amino acids, about 2 amino acids to about 22 amino acids, about 2 amino acids to about 20 amino acids, about 2 amino acids to about 18 amino acids, about 2 amino acids to about 16 amino acids, about 2 amino acids to about 15 amino acids, about 2 amino acids to about 14 amino acids, about 2 amino acids to about 12 amino acids, about 2 amino acids to about 10 amino acids, about 2 amino acids to about 8 amino acids, about 2 amino acids to about 6 amino acids, about 2 amino acids to about 5 amino acids, about 2 amino acids to about 4 amino acids, about 2 amino acids to about 3 amino acids, about 4 amino acids to about 25 amino acids, about 4 amino acids to about 24 amino acids, about 4 amino acids to about 22 amino acids, about 4 amino acids to about 20 amino acids, about 4 amino acids to about 18 amino acids, about 4 amino acids to about 16 amino acids, about 4 amino acids to about 15 amino acids, about 4 amino acids to about 14 amino acids, about 4 amino acids to about 12 amino acids, about 4 amino acids to about 10 amino acids, about 4 amino acids to about 8 amino acids, about 4 amino acids to about 6 amino acids, about 4 amino acids to about 5 amino acids, about 5 amino acids to about 25 amino acids, about 5 amino acids to about 24 amino acids, about 5 amino acids to about 22 amino acids, about 5 amino acids to about 20 amino acids, about 5 amino acids to about 18 amino acids, about 5 amino acids to about 16 amino acids, about 5 amino acids to about 15 amino acids, about 5 amino acids to about 14 amino acids, about 5 amino acids to about 12 amino acids, about 5 amino acids to about 10 amino acids, about 5 amino acids to about 8 amino acids, about 5 amino acids to about 6 amino acids, about 6 amino acids to about 25 amino acids, about 6 amino acids to about 24 amino acids, about 6 amino acids to about 22 amino acids, about 6 amino acids to about 20 amino acids, about 6 amino acids to about 18 amino acids, about 6 amino acids to about 16 amino acids, about 6 amino acids to about 15 amino acids, about 6 amino acids to about 14 amino acids, about 6 amino acids to about 12 amino acids, about 6 amino acids to about 10 amino acids, about 6 amino acids to about 8 amino acids, about 8 amino acids to about 25 amino acids, about 8 amino acids to about 24 amino acids, about 8 amino acids to about 22 amino acids, about 8 amino acids to about 20 amino acids, about 8 amino acids to about 18 amino acids, about 8 amino acids to about 16 amino acids, about 8 amino acids to about 15 amino acids, about 8 amino acids to about 14 amino acids, about 8 amino acids to about 12 amino acids, about 8 amino acids to about 10 amino acids, about 10 amino acids to about 25 amino acids, about 10 amino acids to about 24 amino acids, about 10 amino acids to about 22 amino acids, about 10 amino acids to about 20 amino acids, about 10 amino acids to about 18 amino acids, about 10 amino acids to about 16 amino acids, about 10 amino acids to about 15 amino acids, about 10 amino acids to about 14 amino acids, about 10 amino acids to about 12 amino acids, about 12 amino acids to about 25 amino acids, about 12 amino acids to about 24 amino acids, about 12 amino acids to about 22 amino acids, about 12 amino acids to about 20 amino acids, about 12 amino acids to about 18 amino acids, about 12 amino acids to about 16 amino acids, about 12 amino acids to about 15 amino acids, about 12 amino acids to about 14 amino acids, about 14 amino acids to about 25 amino acids, about 14 amino acids to about 24 amino acids, about 14 amino acids to about 22 amino acids, about 14 amino acids to about 20 amino acids, about 14 amino acids to about 18 amino acids, about 14 amino acids to about 16 amino acids, about 14 amino acids to about 15 amino acids, about 15 amino acids to about 25 amino acids, about 15 amino acids to about 24 amino acids, about 15 amino acids to about 22 amino acids, about 15 amino acids to about 20 amino acids, about 15 amino acids to about 18 amino acids, about 15 amino acids to about 16 amino acids, about 16 amino acids to about 25 amino acids, about 16 amino acids to about 24 amino acids, about 16 amino acids to about 22 amino acids, about 16 amino acids to about 20 amino acids, about 16 amino acids to about 18 amino acids, about 18 amino acids to about 25 amino acids, about 18 amino acids to about 24 amino acids, about 18 amino acids to about 22 amino acids, about 18 amino acids to about 20 amino acids, about 20 amino acids to about 25 amino acids, about 20 amino acids to about 24 amino acids, about 20 amino acids to about 22 amino acids, about 22 amino acid to about 25 amino acids, about 22 amino acid to about 24 amino acids, or about 24 amino acid to about 25 amino acids).

In some embodiments of any of the ACCs described herein, the linker includes a total of about 1 amino acid, about 2 amino acids, about 3 amino acids, about 4 amino acids, about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, about 20 amino acids, about 21 amino acids, about 22 amino acids, about 23 amino acids, about 24 amino acids, or about 25 amino acids.

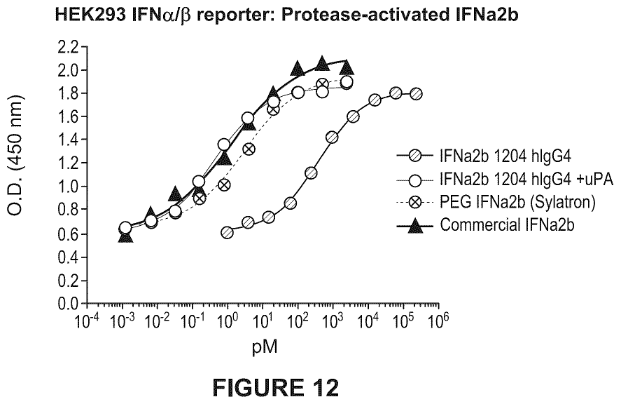

Surprisingly, the inventors have discovered that ACCs that do not comprise any linkers between the CP and the DD exhibit the most significant reduction in cytokine activity relative to the wildtype mature cytokine. See FIGS. 8A and 10A. Further, a configuration in which there are no linkers between the CP and the DD still allows effective cleavage of a CM positioned between the CP and the DD. See FIGS. 12-14. Thus, in some embodiments, the ACC does not comprise any linkers between the CP and the DD, and the CM between the CP and the DD comprises not more than 10, 9, 8, 7, 6, 5, 4, or 3 amino acids. In some embodiments the total number of amino acids in the LR comprises not more than 25 amino acids, e.g., not more than 25, 24, 23, 22, 21, 20, 19, 18, 17, 16, 15, 14, 13, 12, 11, 10, 9, 8, 7, 6, 5, 4, or 3 amino acids, or 3 to 10 amino acids or 5 to 15 amino acids, or 7 to 12 amino acids, or any range or specific number of amino acids selected from the range encompassed by 3 to 25 amino acids.

In some embodiments of any of the ACCs described herein, a linker can be rich in glycine (Gly or G) residues. In some embodiments, the linker can be rich in serine (Ser or S) residues. In some embodiments, the linker can be rich in glycine and serine residues. In some embodiments, the linker has one or more glycine-serine residue pairs (GS) (e.g., 1, 2, 3, 4, 5, 6, 7, 8, 9, or 10 or more GS pairs). In some embodiments, the linker has one or more Gly-Gly-Gly-Ser (GGGS) sequences (e.g., 1, 2, 3, 4, 5, 6, 7, 8, 9, or 10 or more GGGS sequences). In some embodiments, the linker has one or more Gly-Gly-Gly-Gly-Ser (GGGGS) sequences (e.g., 1, 2, 3, 4, 5, 6, 7, 8, 9, or 10 or more GGGGS sequences). In some embodiments, the linker has one or more Gly-Gly-Ser-Gly (GGSG) sequences (e.g., 1, 2, 3, 4, 5, 6, 7, 8, 9, or 10 or more GGSG sequences).

In some embodiments of any of the ACCs described herein, a linker includes any one of or a combination of one or more of: GSSGGSGGSGG (SEQ ID NO: 210), GGGS (SEQ ID NO: 2), GGGSGGGS (SEQ ID NO: 211), GGGSGGGSGGGS (SEQ ID NO: 212), GGGGSGGGGSGGGGS (SEQ ID NO: 213), GGGGSGGGGSGGGGSGGGGSGGGGS (SEQ ID NO: 214), GGGGSGGGGS (SEQ ID NO: 215), GGGGS (SEQ ID NO: 216), GS, GGGGSGS (SEQ ID NO: 217), GGGGSGGGGSGGGGSGS (SEQ ID NO: 218), GGSLDPKGGGGS (SEQ ID NO: 219), PKSCDKTHTCPPCPAPELLG (SEQ ID NO: 220), SKYGPPCPPCPAPEFLG (SEQ ID NO: 221), GKSSGSGSESKS (SEQ ID NO: 222), GSTSGSGKSSEGKG (SEQ ID NO: 223), GSTSGSGKSSEGSGSTKG (SEQ ID NO: 224), and GSTSGSGKPGSGEGSTKG (SEQ ID NO: 225).

Non-limiting examples of linkers can include a sequence that is at least 70% identical (e.g., at least 72%, at least 74%, at least 75%, at least 76%, at least 78%, at least 80%, at least 82%, at least 84%, at least 85%, at least 86%, at least 88%, at least 90%, at least 92%, at least 94%, at least 95%, at least 96%, at least 97%, at least 98%, at least 99%, or 100% identical) to GGGS (SEQ ID NO: 2), GSSGGSGGSGG (SEQ ID NO: 210), GGGGSGGGGSGGGGS (SEQ ID NO: 213), GGGGSGS (SEQ ID NO: 217), GGGGSGGGGSGGGGSGS (SEQ ID NO: 218), GGGGSGGGGSGGGGSGGGGS (SEQ ID NO: 214), GGSLDPKGGGGS (SEQ ID NO: 215), and GSTSGSGKPGSSEGST (SEQ ID NO: 226).

In some embodiments, the linker includes a sequence selected from the group of: GGSLDPKGGGGS (SEQ ID NO: 219), GGGGSGGGGSGGGGSGS (SEQ ID NO: 218), GGGGSGS (SEQ ID NO: 217), GS, (GS)n, (GGS)n, (GSGGS)n (SEQ ID NO: 227) and (GGGS)n (SEQ ID NO: 228), GGSG (SEQ ID NO: 229), GGSGG (SEQ ID NO: 230), GSGSG (SEQ ID NO: 231), GSGGG (SEQ ID NO: 232), GGGSG (SEQ ID NO: 233), GSSSG (SEQ ID NO: 234), GGGGSGGGGSGGGGS (SEQ ID NO: 213), GGGGSGGGGSGGGGSGGGGS (SEQ ID NO: 214), GSTSGSGKPGSSEGST (SEQ ID NO: 226), (GGGGS)n (SEQ ID NO: 216), wherein n is an integer of at least one. In some embodiments, the linker includes a sequence selected from the group consisting of: GGSLDPKGGGGS (SEQ ID NO: 219), GGGGSGGGGSGGGGSGS (SEQ ID NO: 218), GGGGSGS (SEQ ID NO: 217), and GS. In some embodiments of any of the ACCs described herein, the linker includes a sequence selected from the group of: GGGGSGGGGSGGGGS (SEQ ID NO: 213), GGGGSGGGGSGGGGSGGGGS (SEQ ID NO: 214), and GSTSGSGKPGSSEGST (SEQ ID NO: 226). In some embodiments of any of the activatable cytokine constructs described herein, the linker includes a sequence selected from the group of: GGGGSGGGGSGGGGS (SEQ ID NO: 213) or GGGGS (SEQ ID NO: 216). In some embodiments, the linker comprises a sequence of GGGS (SEQ ID NO: 2).

In some embodiments, an ACC can include one, two, three, four, five, six, seven, eight, nine, or ten linker sequence(s) (e.g., the same or different linker sequences of any of the exemplary linker sequences described herein or known in the art). In some embodiments, a linker comprises sulfo-SIAB, SMPB, and sulfo-SMPB, wherein the linkers react with primary amines sulfhydryls.

In some embodiments of any of the ACCs described herein, the ACC is characterized by a reduction in at least one activity of the CP1 and/or CP2 as compared to a control level of the at least one activity of the CP1 and/or CP2. In some embodiments, a control level can be the level of the activity for a recombinant CP1 and/or CP2 (e.g., a commercially available recombinant CP1 and/or CP2, a recombinant wildtype CP1 and/or CP2, and the like). In some embodiments, a control level can be the level of the activity of a cleaved (activated) form of the ACC. In certain embodiments, a control level can be the level of the activity of a pegylated CP1 and/or CP2.

In some embodiments, the at least one activity is the binding affinity (K_{D}) of the CP1 and/or the CP2 for its cognate receptor as determined using surface plasmon resonance (e.g., performed in phosphate buffered saline at 25° C.). In certain embodiments, the at least one activity is the level of proliferation of lymphoma cells. In other embodiments, the at least one activity is the level of JAK/STAT/ISGF3 pathway activation in a lymphoma cell. In some embodiments, the at least one activity is a level of SEAP production in a lymphoma cell. In a further embodiment, the at least one activity of the CP1 and/or CP2 is level of cytokine-stimulated gene induction using, for example RNAseq methods (see, e.g., Zimmerer et al., Clin. Cancer Res. 14(18):5900-5906, 2008; Hilkens et al., J. Immunol. 171:5255-5263, 2003).

In some embodiments, the ACC is characterized by at least a 2-fold reduction in at least one CP1 and/or CP2 activity as compared to the control level of the at least one CP1 and/or CP2 activity. In some embodiments, the ACC is characterized by at least a 5-fold reduction in at least one activity of the CP1 and/or CP2 as compared to the control level of the at least one activity of the CP1 and/or CP2. In some embodiments, the ACC is characterized by at least a 10-fold reduction in at least one activity of the CP1 and/or CP2 as compared to the control level of the at least one activity of the CP1 and/or CP2. In some embodiments, the ACC is characterized by at least a 20-fold reduction in at least one activity of the CP1 and/or CP2 as compared to the control level of the at least one activity of the CP1 and/or CP2. In some embodiments, the ACC is characterized by at least a 30-fold, 40-fold, 50-fold, 60-fold, 70-fold, 80-fold, 90-fold, 100-fold, 500-fold, or 1000-fold reduction in at least one activity of the CP1 and/or CP2 as compared to the control level of the at least one activity of the CP1 and/or CP2. In some embodiments, ACC is characterized by at least a 1- to 20-fold reduction, a 200- to 500-fold reduction, a 300- to 500-fold reduction, a 400- to 500-fold reduction, a 500- to 600-fold reduction, a 600- to 700-fold reduction, a 150- to 1000-fold reduction, a 100- to 1500-fold reduction, a 200- to 1500-fold reduction, a 300- to 1500-fold reduction, a 400- to 1500-fold reduction, a 500- to 1500-fold reduction, a 1000- to 1500-fold reduction, a 100- to 1000-fold reduction, a 200- to 1000-fold reduction, a 300- to 1000-fold reduction, a 400- to 1000-fold reduction, a 500- to 1000-fold reduction, a 100- to 500-fold reduction, a 20- to 50-fold reduction, a 30- to 50-fold reduction, a 40- to 50-fold reduction, a 100- to 400-fold reduction, a 200- to 400-fold reduction, or a 300- to 400-fold reduction, a 100- to 300-fold reduction, a 200- to 300-fold reduction, or a 100- to 200-fold reduction in at least one activity of the CP1 and/or CP2 as compared to the control level of the at least one activity of the CP1 and/or CP2.

In some embodiments, the control level of the at least one activity of the CP1 and/or CP2 is the activity of the CP1 and/or CP2 released from the ACC following cleavage of CM1 and CM2 by the protease(s) (the “cleavage product”). In some embodiments, the control level of the at least one activity of the CP1 and/or CP2 is the activity of a corresponding wildtype mature cytokine (e.g., recombinant wildtype mature cytokine).

In some embodiments, incubation of the ACC with the protease yields an activated cytokine product(s), where one or more activities of CP1 and/or CP2 of the activated cytokine product(s) is greater than the one or more activities of CP1 and/or CP2 of the intact ACC. In some embodiments, one or more activities of CP1 and/or CP2 of the activated cytokine product(s) is at least 1-fold greater than the one or more activities of CP1 and/or CP2 of the ACC. In some embodiments, one or more activities of CP1 and/or CP2 of the activated cytokine product(s) is at least 2-fold greater than the one or more activities of CP1 and/or CP2 of the ACC. In some embodiments, one or more activities of CP1 and/or CP2 of the activated cytokine product(s) is at least 5-fold greater than the one or more activities of CP1 and/or CP2 of the ACC. In some embodiments, one or more activities of CP1 and/or CP2 of the activated cytokine product(s) is at least 10-fold greater than the one or more activities of CP1 and/or CP2 of the ACC. In some embodiments, one or more activities of CP1 and/or CP2 of the activated cytokine product(s) is at least 20-fold greater than the one or more activities of CP1 and/or CP2 of the ACC. In some embodiments, one or more activities of CP1 and/or CP2 of the activated cytokine product(s) is at least 1- to 20-fold greater, 2- to 20-fold greater, 3- to 20-fold greater, 4- to 20-fold greater, 5- to 20-fold greater, 10- to 20-fold greater, 15- to 20-fold greater, 1- to 15-fold greater, 2- to 15-fold greater, 3- to 15-fold greater, 4- to 15-fold greater, 5- to 15-fold greater, 10- to 15-fold greater, 1- to 10-fold greater, 2- to 10-fold greater, 3- to 10-fold greater, 4- to 10-fold greater, 5- to 10-fold greater, 1- to 5-fold greater, 2- to 5-fold greater, 3- to 5-fold greater, 4- to 5-fold greater, 1- to 4-fold greater, 2- to 4-fold greater, 3- to 4-fold greater, 1- to 3-fold greater, 2- to 3-fold greater, or 1- to 2-fold greater than the one or more activities of CP1 and/or CP2 of the ACC.

In some embodiments, an ACC can include a sequence that is at least 80% (e.g., at least 82%, at least 84%, at least 86%, at least 88%, at least 90%, at least 92%, at least 94%, at least 96%, at least 98%, at least 99%, or 100%) identical to SEQ ID NO: 309 or 311. In some embodiments, an ACC can be encoded by a nucleic acid including a sequence that is at least 80% (e.g., at least 82%, at least 84%, at least 86%, at least 88%, at least 90%, at least 92%, at least 94%, at least 96%, at least 98%, at least 99%, or 100%) identical to SEQ ID NO: 310 or 312. In some aspects, an ACC may include such sequences but either without the signal sequences of those sequences. Signal sequences are not particularly limited. Some non-limiting examples of signal sequences include, e.g., residues 1-20 of SEQ ID NO: 309 and corresponding residues and nucleotides in the other sequences, or substituted with a signal sequence from another species or cell line. Other examples of signal sequences include MRAWIFFLLCLAGRALA (SEQ ID NO: 343) and MALTFALLVALLVLSCKSSCSVG (SEQ ID NO: 344).

Various exemplary aspects of these activatable cytokine constructs are described below and can be used in any combination in the methods provided herein without limitation. Exemplary aspects of the activatable cytokine constructs and methods of making activatable cytokine constructs are described below.

In some embodiments, the CM is selected for use with a specific protease. The protease may be one produced by a tumor cell (e.g., the tumor cell may express greater amounts of the protease than healthy tissues). In some embodiments, the CM is a substrate for at least one protease selected from the group of an ADAM 17, a BMP-1, a cysteine protease such as a cathepsin, a HtrA1, a legumain, a matriptase (MT-SP1), a matrix metalloprotease (MMP), a neutrophil elastase, a TMPRSS, such as TMPRSS3 or TMPRSS4, a thrombin, and a u-type plasminogen activator (uPA, also referred to as urokinase).

In some embodiments, a CM is a substrate for at least one matrix metalloprotease (MMP). Examples of MMPs include MMP1, MMP2, MMP3, MMP7, MMP8, MMP9, MMP10, MMPP11, MMP12, MMP13, MMP14, MMPP15, MMPP16, MMP17, MMP19, MMP20, MMP23, MMP24, MMP26, and MMP27. In some embodiments, the CM is a substrate for MMP9, MMP14, MMP1, MMP3, MMP13, MMP17, MMP11, and MMPP19. In some embodiments, the CM is a substrate for MMP7. In some embodiments, the CM is a substrate for MMP9. In some embodiments, the CM is a substrate for MMP14. In some embodiments, the CM is a substrate for two or more MMPs. In some embodiments, the CM is a substrate for at least MMP9 and MMP14. In some embodiments, the CM includes two or more substrates for the same MMP. In some embodiments, the CM includes at least two or more MMP9 substrates. In some embodiments, the CM includes at least two or more MMP14 substrates.

In some embodiments, a CM is a substrate for an MMP and includes the sequence ISSGLLSS (SEQ ID NO: 19); QNQALRMA (SEQ ID NO: 16); AQNLLGMV (SEQ ID NO: 15); STFPFGMF (SEQ ID NO: 18); PVGYTSSL (SEQ ID NO: 74); DWLYWPGI (SEQ ID NO: 75); MIAPVAYR (SEQ ID NO: 42); RPSPMWAY (SEQ ID NO: 43); WATPRPMR (SEQ ID NO: 44); FRLLDWQW (SEQ ID NO: 45); LKAAPRWA (SEQ ID NO: 76); GPSHLVLT (SEQ ID NO: 77); LPGGLSPW (SEQ ID NO: 78); MGLFSEAG (SEQ ID NO: 79); SPLPLRVP (SEQ ID NO: 80); RMHLRSLG (SEQ ID NO: 81); LAAPLGLL (SEQ ID NO: 17); AVGLLAPP (SEQ ID NO: 14); LLAPSHRA (SEQ ID NO: 82); PAGLWLDP (SEQ ID NO: 20); and/or ISSGLSS (SEQ ID NO: 73).

In some embodiments, a CM is a substrate for thrombin. In some embodiments, the CM is a substrate for thrombin and includes the sequence GPRSFGL (SEQ ID NO: 83) or GPRSFG (SEQ ID NO: 84).

In some embodiments, a CM includes an amino acid sequence selected from the group of NTLSGRSENHSG (SEQ ID NO: 9); NTLSGRSGNHGS (SEQ ID NO: 10); TSTSGRSANPRG (SEQ ID NO: 11); TSGRSANP (SEQ ID NO: 12); VAGRSMRP (SEQ ID NO: 21); VVPEGRRS (SEQ ID NO: 22); ILPRSPAF (SEQ ID NO: 23); MVLGRSLL (SEQ ID NO: 24); QGRAITFI (SEQ ID NO: 25); SPRSIMLA (SEQ ID NO: 26); and SMLRSMPL (SEQ ID NO: 27).

In some embodiments, a CM is a substrate for a neutrophil elastase. In some embodiments, a CM is a substrate for a serine protease. In some embodiments, a CM is a substrate for uPA. In some embodiments, a CM is a substrate for legumain. In some embodiments, the CM is a substrate for matriptase. In some embodiments, the CM is a substrate for a cysteine protease. In some embodiments, the CM is a substrate for a cysteine protease, such as a cathepsin.

In some embodiments, a CM includes a sequence of ISSGLLSGRSDNH (SEQ ID NO: 28); ISSGLLSSGGSGGSLSGRSDNH (SEQ ID NO: 30); AVGLLAPPGGTSTSGRSANPRG (SEQ ID NO: 275); TSTSGRSANPRGGGAVGLLAPP (SEQ ID NO: 276); VHMPLGFLGPGGTSTSGRSANPRG (SEQ ID NO: 277); TSTSGRSANPRGGGVHMPLGFLGP (SEQ ID NO: 278); AVGLLAPPGGLSGRSDNH (SEQ ID NO: 29); LSGRSDNHGGAVGLLAPP (SEQ ID NO: 70); VHUMIPLGFLGPGGLSGRSDNH (SEQ ID NO: 266); LSGRSDNHGGVHMPLGFLGP (SEQ ID NO: 267); LSGRSDNHGGSGGSISSGLLSS (SEQ ID NO: 268); LSGRSGNHGGSGGSISSGLLSS (SEQ ID NO: 279); ISSGLLSSGGSGGSLSGRSGNH (SEQ ID NO: 269); LSGRSDNHGGSGGSQNQALRMA (SEQ ID NO: 270); QNQALRMAGGSGGSLSGRSDNH (SEQ ID NO: 271); LSGRSGNHGGSGGSQNQALRMA (SEQ ID NO: 272); QNQALRMAGGSGGSLSGRSGNH (SEQ ID NO: 273), and/or ISSGLLSGRSGNH (SEQ ID NO: 274).

In some embodiments, the CM1 and/or the CM2 comprise a sequence selected from the group consisting of: SEQ ID NO: 5 through SEQ ID NO: 100. In some embodiments, the CM comprises a sequence selected from the group of: ISSGLLSGRSDNH (SEQ ID NO: 28), LSGRSDDH (SEQ ID NO: 33), ISSGLLSGRSDQH (SEQ ID NO: 54), SGRSDNI (SEQ ID NO: 100), and ISSGLLSGRSDNI (SEQ ID NO: 68).

In some aspects, the ACC includes a CP1 selected from SEQ ID Nos: 1 and 101-209, a CM1 selected from SEQ ID Nos: 5-100 and 263-308, and a DD1 dimerized with a CP2 selected from SEQ ID Nos: 1 and 101-209, a CM2 selected from SEQ ID Nos: 5-100 and 263-308, and a DD2. In some aspects, the ACC may include, between CP1 and CM1 and/or between CM1 and DD1, a linker selected from SEQ ID Nos: 2 and 210-234, 245, or 250, and between CP2 and CM2 and/or between CM2 and DD2, a linker selected from SEQ ID Nos: 2 and 210-234, 245, or 250. In some embodiments, the ACC includes a DD1 and/or a DD2 that has an amino acid sequence that is at least 80% identical (e.g., at least 82%, at least 84%, at least 85%, at least 86%, at least 88%, at least 90%, at least 92%, at least 94%, at least 95%, at least 96%, at least 97%, at least 98%, at least 99%, or 100% identical) to SEQ ID NO: 3 or SEQ ID NO: 4. In some embodiments, the ACC includes a DD1 that has an amino acid sequence that is at least 80% identical (e.g., at least 82%, at least 84%, at least 85%, at least 86%, at least 88%, at least 90%, at least 92%, at least 94%, at least 95%, at least 96%, at least 97%, at least 98%, at least 99%, or 100% identical) to SEQ ID NO: 315 or SEQ ID NO: 316. In some embodiments, the ACC includes a DD2 that has an amino acid sequence that is at least 80% identical (e.g., at least 82%, at least 84%, at least 85%, at least 86%, at least 88%, at least 90%, at least 92%, at least 94%, at least 95%, at least 96%, at least 97%, at least 98%, at least 99%, or 100% identical) to SEQ ID NO: 315 or SEQ ID NO: 316.

### Conjugation to Agents

This disclosure also provides methods and materials for including additional elements in any of the ACCs described herein including, for example, a targeting moiety to facilitate delivery to a cell or tissue of interest, an agent (e.g., a therapeutic agent, an antineoplastic agent), a toxin, or a fragment thereof.

In some embodiments of any of the ACCs described herein, the ACC can be conjugated to a cytotoxic agent, including, without limitation, a toxin (e.g., an enzymatically active toxin of bacterial, fungal, plant, or animal origin, or fragments thereof) or a radioactive isotope. In some embodiments of any of the ACCs described herein, the activatable cytokine construct can be conjugated to a cytotoxic agent including, without limitation, a toxin (e.g., an enzymatically active toxin of bacterial, fungal, plant, or animal origin, or fragments thereof), or a radioactive isotope.

Non-limiting exemplary cytotoxic agents that can be conjugated to any of the ACCs described herein include: dolastatins and derivatives thereof (e.g., auristatin E, AFP, monomethyl auristatin D (MMAD), monomethyl auristatin F (MMAF), monomethyl auristatin E (MMAE), desmethyl auristatin E (DMAE), auristatin F, desmethyl auristatin F (DMAF), dolastatin 16 (DmJ), dolastatin 16 (Dpv), auristatin derivatives (e.g., auristatin tyramine, auristatin quinolone), maytansinoids (e.g., DM-1, DM-4), maytansinoid derivatives, duocarmycin, alpha-amanitin, turbostatin, phenstatin, hydroxyphenstatin, spongistatin 5, spongistatin 7, halistatin 1, halistatin 2, halistatin 3, halocomstatin, pyrrolobenzimidazoles (PBI), cibrostatin6, doxaliform, cemadotin analogue (CemCH2-SH), Pseudomonas toxin A (PES8) variant, Pseudomonase toxin A (ZZ-PE38) variant, ZJ-101, anthracycline, doxorubicin, daunorubicin, bryostatin, camptothecin, 7-substituted campothecin, 10, 11-difluoromethylenedioxycamptothecin, combretastatins, debromoaplysiatoxin, KahaMide-F, discodermolide, and Ecteinascidins.

Non-limiting exemplary enzymatically active toxins that can be conjugated to any of the ACCs described herein include: diphtheria toxin, exotoxin A chain from Pseudomonas aeruginosa, ricin A chain, abrin A chain, modeccin A chain, alpha-sarcin, Aleuriies fordii proteins, dianfhin proteins, Phytoiaca Americana proteins (e.g., PAPI, PAPII, and PAP-8), Momordica charantia inhibitor, curcin, crotirs, Sapaonaria officinalis inhibitor, geionin, mitogeliin, restrictocin, phenomycin, neomycin, and tricothecenes.

Non-limiting exemplary anti-neoplastics that can be conjugated to any of the ACCs described herein include: adriamycin, cerubidine, bleomycin, alkeran, velban, oncovin, fluorouracil, methotrexate, thiotepa, bisantrene, novantrone, thioguanine, procarabizine, and cytarabine.

Non-limiting exemplary antivirals that can be conjugated to any of the ACCs described herein include: acyclovir, vira A, and symmetrel.

Non-limiting exemplary antifungals that can be conjugated to any of the ACCs described herein include: nystatin.

Non-limiting exemplary conjugatable detection reagents that can be conjugated to any of the ACCs described herein include: fluorescein and derivatives thereof, fluorescein isothiocyanate (FITC).

Non-limiting exemplary antibacterials that can be conjugated to any of the activatable cytokine constructs described herein include: aminoglycosides, streptomycin, neomycin, kanamycin, amikacin, gentamicin, and tobramycin.

Non-limiting exemplary 3beta,16beta,17alpha-trihydroxycholest-5-en-22-one 16-O-(2-O-4-methoxybenzoyl-beta-D-xylopyranosyl)-(1-->3)-(2-O-acetyl-alpha-L-arabinopyranoside) (OSW-1) that can be conjugated to any of the activatable cytokine constructs described herein include: s-nitrobenzyloxycarbonyl derivatives of 06-benzylguanine, toposisomerase inhibitors, hemiasterlin, cephalotaxine, homoharringionine, pyrrol obenzodiazepine dimers (PBDs), functionalized pyrrolobenzodiazepenes, calcicheamicins, podophyiitoxins, taxanes, and vinca alkoids.

Non-limiting exemplary radiopharmaceuticals that can be conjugated to any of the activatable cytokine constructs described herein include: ¹²³I, ⁸⁹Zr, ¹²⁵I, ¹³¹I, ⁹⁹mTc, ²⁰¹Tl, ⁶²Cu, ¹⁸F, ⁶⁸Ga, ¹³N, ¹⁵O, ³⁸K, ⁸²Rb, ¹¹¹In, ¹³³Xe, ¹¹C, and ⁹⁹mTc (Technetium).

Non-limiting exemplary heavy metals that can be conjugated to any of the ACCs described herein include: barium, gold, and platinum.

Non-limiting exemplary anti-mycoplasmals that can be conjugated to any of the ACCs described herein include: tylosine, spectinomycin, streptomycin B, ampicillin, sulfanilamide, polymyxin, and chloramphenicol.

Those of ordinary skill in the art will recognize that a large variety of possible moieties can be conjugated to any of the activatable cytokine constructs described herein. Conjugation can include any chemical reaction that will bind the two molecules so long as the ACC and the other moiety retain their respective activities. Conjugation can include many chemical mechanisms, e.g., covalent binding, affinity binding, intercalation, coordinate binding, and complexation. In some embodiments, the preferred binding is covalent binding. Covalent binding can be achieved either by direct condensation of existing side chains or by the incorporation of external bridging molecules. Many bivalent or polyvalent linking agents are useful in conjugating any of the activatable cytokine constructs described herein. For example, conjugation can include organic compounds, such as thioesters, carbodiimides, succinimide esters, glutaraldehyde, diazobenzenes, and hexamethylene diamines. In some embodiments, the activatable cytokine construct can include, or otherwise introduce, one or more non-natural amino acid residues to provide suitable sites for conjugation.

In some embodiments of any of the ACCs described herein, an agent and/or conjugate is attached by disulfide bonds (e.g., disulfide bonds on a cysteine molecule) to the antigen-binding domain. Since many cancers naturally release high levels of glutathione, a reducing agent, glutathione present in the cancerous tissue microenvironment can reduce the disulfide bonds, and subsequently release the agent and/or the conjugate at the site of delivery.

In some embodiments of any of the ACCs described herein, when the conjugate binds to its target in the presence of complement within the target site (e.g., diseased tissue (e.g., cancerous tissue)), the amide or ester bond attaching the conjugate and/or agent to the linker is cleaved, resulting in the release of the conjugate and/or agent in its active form. These conjugates and/or agents when administered to a subject, will accomplish delivery and release of the conjugate and/or the agent at the target site (e.g., diseased tissue (e.g., cancerous tissue)). These conjugates and/or agents are particularly effective for the in vivo delivery of any of the conjugates and/or agents described herein.

In some embodiments, the linker is not cleavable by enzymes of the complement system. For example, the conjugate and/or agent is released without complement activation since complement activation ultimately lyses the target cell. In such embodiments, the conjugate and/or agent is to be delivered to the target cell (e.g., hormones, enzymes, corticosteroids, neurotransmitters, or genes). Furthermore, the linker is mildly susceptible to cleavage by serum proteases, and the conjugate and/or agent is released slowly at the target site.

In some embodiments of any of the ACCs described herein, the conjugate and/or agent is designed such that the conjugate and/or agent is delivered to the target site (e.g., disease tissue (e.g., cancerous tissue)) but the conjugate and/or agent is not released.

In some embodiments of any of the ACCs described herein, the conjugate and/or agent is attached to an antigen-binding domain either directly or via a non-cleavable linker. Exemplary non-cleavable linkers include amino acids (e.g., D-amino acids), peptides, or other organic compounds that may be modified to include functional groups that can subsequently be utilized in attachment to antigen-binding domains by methods described herein.

In some embodiments of any of the ACCs described herein, an ACC includes at least one point of conjugation for an agent. In some embodiments, all possible points of conjugation are available for conjugation to an agent. In some embodiments, the one or more points of conjugation include, without limitation, sulfur atoms involved in disulfide bonds, sulfur atoms involved in interchain disulfide bonds, sulfur atoms involved in interchain sulfide bonds but not sulfur atoms involved in intrachain disulfide bonds, and/or sulfur atoms of cysteine or other amino acid residues containing a sulfur atom. In such cases, residues may occur naturally in the protein construct structure or may be incorporated into the protein construct using methods including, without limitation, site-directed mutagenesis, chemical conversion, or mis-incorporation of non-natural amino acids.

This disclosure also provides methods and materials for preparing an ACC for conjugation. In some embodiments of any of the ACCs described herein, an ACC is modified to include one or more interchain disulfide bonds. For example, disulfide bonds in the ACC can undergo reduction following exposure to a reducing agent such as, without limitation, TCEP, DTT, or β-mercaptoethanol. In some cases, the reduction of the disulfide bonds is only partial. As used herein, the term partial reduction refers to situations where an ACC is contacted with a reducing agent and a fraction of all possible sites of conjugation undergo reduction (e.g., not all disulfide bonds are reduced). In some embodiments, an activatable cytokine construct is partially reduced following contact with a reducing agent if less than 99%, (e.g., less than 98%, 97%, 96%, 95%, 90%, 85%, 80%, 75%, 70%, 65%, 60%, 55%, 50%, 45%, 40%, 35%, 30%, 25%, 20%, 15%, 10% or less than 5%) of all possible sites of conjugation are reduced. In some embodiments, the ACC having a reduction in one or more interchain disulfide bonds is conjugated to a drug reactive with free thiols.

This disclosure also provides methods and materials for conjugating a therapeutic agent to a particular location on an ACC. In some embodiments of any of the ACC described herein, an ACC is modified so that the therapeutic agents can be conjugated to the ACC at particular locations on the ACC. For example, an ACC can be partially reduced in a manner that facilitates conjugation to the ACC. In such cases, partial reduction of the ACC occurs in a manner that conjugation sites in the ACC are not reduced. In some embodiments, the conjugation site(s) on the ACC are selected to facilitate conjugation of an agent at a particular location on the protein construct. Various factors can influence the “level of reduction” of the ACC upon treatment with a reducing agent. For example, without limitation, the ratio of reducing agent to ACC, length of incubation, incubation temperature, and/or pH of the reducing reaction solution can require optimization in order to achieve partial reduction of the ACC with the methods and materials described herein. Any appropriate combination of factors (e.g., ratio of reducing agent to ACC, the length and temperature of incubation with reducing agent, and/or pH of reducing agent) can be used to achieve partial reduction of the ACC (e.g., general reduction of possible conjugation sites or reduction at specific conjugation sites).

An effective ratio of reducing agent to ACC can be any ratio that at least partially reduces the ACC in a manner that allows conjugation to an agent (e.g., general reduction of possible conjugation sites or reduction at specific conjugation sites). In some embodiments, the ratio of reducing agent to ACC will be in a range from about 20:1 to 1:1, from about 10:1 to 1:1, from about 9:1 to 1:1, from about 8:1 to 1:1, from about 7:1 to 1:1, from about 6:1 to 1:1, from about 5:1 to 1:1, from about 4:1 to 1:1, from about 3:1 to 1:1, from about 2:1 to 1:1, from about 20:1 to 1:1.5, from about 10:1 to 1:1.5, from about 9:1 to 1:1.5, from about 8:1 to 1:1.5, from about 7:1 to 1:1.5, from about 6:1 to 1:1.5, from about 5:1 to 1:1.5, from about 4:1 to 1:1.5, from about 3:1 to 1:1.5, from about 2:1 to 1:1.5, from about 1.5:1 to 1:1.5, or from about 1:1 to 1:1.5. In some embodiments, the ratio is in a range of from about 5:1 to 1:1. In some embodiments, the ratio is in a range of from about 5:1 to 1.5:1. In some embodiments, the ratio is in a range of from about 4:1 to 1:1. In some embodiments, the ratio is in a range from about 4:1 to 1.5:1. In some embodiments, the ratio is in a range from about 8:1 to about 1:1. In some embodiments, the ratio is in a range of from about 2.5:1 to 1:1.

An effective incubation time and temperature for treating an ACC with a reducing agent can be any time and temperature that at least partially reduces the ACC in a manner that allows conjugation of an agent to an ACC (e.g., general reduction of possible conjugation sites or reduction at specific conjugation sites). In some embodiments, the incubation time and temperature for treating an ACC will be in a range from about 1 hour at 37° C. to about 12 hours at 37° C. (or any subranges therein).

An effective pH for a reduction reaction for treating an ACC with a reducing agent can be any pH that at least partially reduces the ACC in a manner that allows conjugation of the ACC to an agent (e.g., general reduction of possible conjugation sites or reduction at specific conjugation sites).

When a partially-reduced ACC is contacted with an agent containing thiols, the agent can conjugate to the interchain thiols in the ACC. An agent can be modified in a manner to include thiols using a thiol-containing reagent (e.g., cysteine or N-acetyl cysteine). For example, the ACC can be partially reduced following incubation with reducing agent (e.g., TEPC) for about 1 hour at about 37° C. at a desired ratio of reducing agent to ACC. An effective ratio of reducing agent to ACC can be any ratio that partially reduces at least two interchain disulfide bonds located in the ACC in a manner that allows conjugation of a thiol-containing agent (e.g., general reduction of possible conjugation sites or reduction at specific conjugation sites).

In some embodiments of any of the ACCs described herein, an ACC is reduced by a reducing agent in a manner that avoids reducing any intrachain disulfide bonds. In some embodiments of any of the ACCs described herein, an ACC is reduced by a reducing agent in a manner that avoids reducing any intrachain disulfide bonds and reduces at least one interchain disulfide bond.

In some embodiments of any of the ACCs described herein, the ACC can also include an agent conjugated to the ACC. In some embodiments, the conjugated agent is a therapeutic agent.

In some embodiments, the agent (e.g., agent conjugated to an activatable cytokine construct) is a detectable moiety such as, for example, a label or other marker. For example, the agent is or includes a radiolabeled amino acid, one or more biotinyl moieties that can be detected by marked avidin (e.g., streptavidin containing a fluorescent marker or enzymatic activity that can be detected by optical or calorimetric methods), one or more radioisotopes or radionuclides, one or more fluorescent labels, one or more enzymatic labels, and/or one or more chemiluminescent agents. In some embodiments, detectable moieties are attached by spacer molecules.

In some embodiments, the agent (e.g., cytotoxic agent conjugated to an activatable cytokine construct) is linked to the ACC using a carbohydrate moiety, sulfhydryl group, amino group, or carboxylate group.

In some embodiments of any of the ACCs described herein conjugated to an agent, the agent (e.g., cytotoxic agent conjugated to an activatable cytokine construct) is conjugated to the ACC via a linker and/or a CM (also referred to as a cleavable sequence). In some embodiments, the agent (e.g., cytotoxic agent conjugated to an activatable cytokine construct) is conjugated to a cysteine or a lysine in the ACC. In some embodiments, the agent (e.g., cytotoxic agent conjugated to an activatable cytokine construct) is conjugated to another residue of the ACC, such as those residues disclosed herein. In some embodiments, the linker is a thiol-containing linker. In some embodiments, the linker is a non-cleavable linker. Some non-limiting examples of cleavable moieties and linkers are provided in Table 1.

TABLE 1

| Types of CMs | Amino Acid Sequence |
|---|---|
| Plasmin CMs | |
| Pro-urokinase | PRFKIIGG (SEQ ID NO: 280) |
| | PRFRIIGG (SEQ ID NO: 281) |
| TGFβ | SSRHRRALD (SEQ ID NO: 282) |
| Plasminogen | RKSSIIIRMRDVVL (SEQ ID NO: |
| | 283) |
| Staphylokinase | SSSFDKGKYKKGDDA (SEQ ID |
| | NO: 284) |
| | SSSFDKGKYKRGDDA (SEQ ID |
| | NO: 285) |
| Factor Xa CMs | IEGR (SEQ ID NO: 286) |
| | IDGR (SEQ ID NO: 287) |
| | GGSIDGR (SEQ ID NO: 288) |
| MMP CMs | |
| Gelatinase A | PLGLWA (SEQ ID NO: 289) |
| Collagenase CMs | |
| Calf skin collagen | GPQGIAGQ (SEQ ID NO: 290) |
| (α1(I) chain) | |
| Calf skin collagen | GPQGLLGA (SEQ ID NO: 291) |
| (α2(I) chain) | |
| Bovine cartilage | GIAGQ (SEQ ID NO: 292) |
| collagen (α1(II) | |
| chain) | |
| Human liver | GPLGIAGI (SEQ ID NO: 293) |
| collagen (α1(III) | |
| chain) | |
| Human α₂M | GPEGLRVG (SEQ ID NO: 294) |
| Human PZP | YGAGLGVV (SEQ ID NO: 295) |
| | AGLGVVER (SEQ ID NO: 296) |
| | AGLGISST (SEQ ID NO: 297) |
| Rat α₁M | EPQALAMS (SEQ ID NO: 298) |
| | QALAMSAI (SEQ ID NO: 299) |
| Rat α₂M | AAYHLVSQ (SEQ ID NO: 300) |
| | MDAFLESS (SEQ ID NO: 301) |
| Rat α₁I₃(2J) | ESLPVVAV (SEQ ID NO: 302) |
| Rat α₁I₃(27J) | SAPAVESE (SEQ ID NO: 303) |
| Human fibroblast | DVAQFVLT (SEQ ID NO: 304) |
| collagenase | |
| (autolytic | VAQFVLT (SEQ ID NO: 305) |
| cleavages) | |
| | VAQFVLTE (SEQ ID NO: 306) |
| | AQFVLTEG (SEQ ID NO: 307) |
| | PVQPIGPQ (SEQ ID NO: 308) |

Those of ordinary skill in the art will recognize that a large variety of possible moieties can be coupled to the ACCs of the disclosure. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference). In general, an effective conjugation of an agent (e.g., cytotoxic agent) to an ACC can be accomplished by any chemical reaction that will bind the agent to the ACC while also allowing the agent and the ACC to retain functionality.

In some embodiments of any of the ACCs conjugated to an agent, a variety of bifunctional protein-coupling agents can be used to conjugate the agent to the ACC including, without limitation, N-succinimidyl-3-(2-pyridyldithiol) propionate (SPDP), iminothiolane (IT), bifunctional derivatives of imidoesters (e.g., dimethyl adipimidate HCL), active esters (e.g., disuccinimidyl suberate), aldehydes (e.g., glutareldehyde), bis-azido compounds (e.g., bis (p-azidobenzoyl) hexanediamine), bis-diazonium derivatives (e.g., bis-(p-diazoniumbenzoyl)-ethylenediamine), diisocyanates (e.g., tolyene 2,6-diisocyanate), and bis-active fluorine compounds (e.g., 1,5-difluoro-2,4-dinitrobenzene). For example, a ricin immunotoxin can be prepared as described in Vitetta et al., Science 238: 1098 (1987). In some embodiments, a carbon-14-labeled 1-isothiocyanatobenzyl-3-methyldiethylene triaminepentaacetic acid (MX-DTPA) chelating agent can be used to conjugate a radionucleotide to the ACC. (See, e.g., WO94/11026).

Suitable linkers and CMs are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an ACC by way of an oligopeptide linker. In some embodiments, suitable linkers include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide]hexanoate (Pierce Chem. Co. Cat. #2165-G); and (v) sulfo-NHS (N-hydroxysulfo-succinimide: Pierce Chem. Co., Cat. #24510) conjugated to EDC. Additional linkers include, but are not limited to, SMCC, sulfo-SMCC, SPDB, or sulfo-SPDB.

The CMs and linkers described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically-hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

In some embodiments of any of the ACCs, an agent can be conjugated to the ACC using a modified amino acid sequence included in the amino acid sequence of the ACC. By inserting conjugation-enabled amino acids at specific locations within the amino acid sequence of the ACC, the protein construct can be designed for controlled placement and/or dosage of the conjugated agent (e.g., cytotoxic agent). For example, the ACC can be modified to include a cysteine amino acid residue at positions on the first monomer, the second monomer, the third monomer, and/or the fourth monomer that provide reactive thiol groups and does not negatively impact protein folding and/or assembly and does not alter antigen-binding properties. In some embodiments, the ACC can be modified to include one or more non-natural amino acid residues within the amino acid sequence of the ACC to provide suitable sites for conjugation. In some embodiments, the ACC can be modified to include enzymatically activatable peptide sequences within the amino acid sequence of the ACC.

### Nucleic Acids

Provided herein are nucleic acids including sequences that encode the first monomer construct (or the protein portion of the first monomer construct) (e.g., any of the first monomers constructs described herein) and the second monomer construct (or the protein portion of the second monomer construct) (e.g., any of the second monomer constructs described herein) of any of the ACCs described herein. In some embodiments, a pair of nucleic acids together encode the first monomer construct (or the protein portion of the first monomer construct) and the second monomer construct (or the protein portion of the second monomer construct). In some embodiments, the nucleic acid sequence encoding the first monomer construct (or the protein portion of the first monomer construct) is at least 70% identical (e.g., at least 72% identical, at least 74% identical, at least 76% identical, at least 78% identical, at least 80% identical, at least 82% identical, at least 84% identical, at least 86% identical, at least 88% identical, at least 90% identical, at least 92% identical, at least 94% identical, at least 96% identical, at least 98% identical, at least 99% identical, or 100% identical) to the nucleic acid sequence encoding the second monomer construct (or the protein portion of the second monomer construct).

In some embodiments, the nucleic acid encoding the protein portion of a first monomer construct encodes a polypeptide comprising the CP1 and CM1 moieties. In some embodiments, the nucleic acid encoding the protein portion of a second monomer encodes a polypeptide comprising the CP2 and CM2 moieties. In some embodiments, a pair of nucleic acids together encode the protein portion of a first monomer construct and the protein portion of the second monomer construct, wherein the protein portions are then conjugated to the DD1 and DD2 moieties, respectively (in a subsequent conjugation step).

In some embodiments, the nucleic acid encoding the first monomer construct encodes a polypeptide comprising the DD1 moiety. In some embodiments, the nucleic acid encoding the second monomer construct encodes a polypeptide comprising the DD2 moiety.

### Vectors

Provided herein are vectors and sets of vectors including any of the nucleic acids described herein. One skilled in the art will be capable of selecting suitable vectors or sets of vectors (e.g., expression vectors) for making any of the ACCs described herein, and using the vectors or sets of vectors to express any of the ACCs described herein. For example, in selecting a vector or a set of vectors, the cell must be considered because the vector(s) may need to be able to integrate into a chromosome of the cell and/or replicate in it. Exemplary vectors that can be used to produce an ACC are also described below.

As used herein, the term “vector” refers to a polynucleotide capable of inducing the expression of a recombinant protein (e.g., a first or second monomer) in a cell (e.g., any of the cells described herein). A “vector” is able to deliver nucleic acids and fragments thereof into a host cell, and includes regulatory sequences (e.g., promoter, enhancer, poly(A) signal). Exogenous polynucleotides may be inserted into the expression vector in order to be expressed. The term “vector” also includes artificial chromosomes, plasmids, retroviruses, and baculovirus vectors.

Methods for constructing suitable vectors that include any of the nucleic acids described herein, and suitable for transforming cells (e.g., mammalian cells) are well-known in the art. See, e.g., Sambrook et al., Eds. “Molecular Cloning: A Laboratory Manual,” 2^{nd} Ed., Cold Spring Harbor Press, 1989 and Ausubel et al., Eds. “Current Protocols in Molecular Biology,” Current Protocols, 1993.

Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. A vector can, for example, include sufficient cis-acting elements for expression; other elements for expression can be supplied by the host mammalian cell or in an in vitro expression system. Skilled practitioners will be capable of selecting suitable vectors and mammalian cells for making any of the ACCs described herein.

In some embodiments of any of the ACCs described herein, the ACC may be made biosynthetically using recombinant DNA technology and expression in eukaryotic or prokaryotic species.

In some embodiments, the vector includes a nucleic acid encoding the first monomer and the second monomer of any of the ACCs described herein. In some embodiments, the vector is an expression vector.

In some embodiments, a pair of vectors together include a pair of nucleic acids that together encode the first monomer and the second monomer of any of the ACCs described herein. In some embodiments, the pair of vectors is a pair of expression vectors.

### Cells

Also provided herein are host cells including any of the vector or sets of vectors described herein including any of the nucleic acids described herein.

Any of the ACCs described herein can be produced by any cell (e.g., a mammalian cell). In some embodiments, a host cell is a mammalian cell (e.g., a human cell), a rodent cell (e.g., a mouse cell, a rat cell, a hamster cell, or a guinea pig cell), or a non-human primate cell.

Methods of introducing nucleic acids and vectors (e.g., any of the vectors or any of the sets of vectors described herein) into a cell are known in the art. Non-limiting examples of methods that can be used to introducing a nucleic acid into a cell include: lipofection, transfection, calcium phosphate transfection, cationic polymer transfection, viral transduction (e.g., adenoviral transduction, lentiviral transduction), nanoparticle transfection, and electroporation.

In some embodiments, the introducing step includes introducing into a cell a vector (e.g., any of the vectors or sets of vectors described herein) including a nucleic acid encoding the monomers that make up any of the ACCs described herein.

In some embodiments of any of the methods described herein, the cell can be a eukaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. Non-limiting examples of mammalian cells include Chinese hamster ovary (CHO) cells and human embryonic kidney cells (e.g., HEK293 cells).

In some embodiments, the cell contains the nucleic acid encoding the first monomer and the second monomer of any one of the ACCs described herein. In some embodiments, the cell contains the pair of nucleic acids that together encode the first monomer and the second monomer of any of the ACCs described herein.

### Methods of Producing Activatable Cytokine Constructs

Provided herein are methods of producing any of the ACCs described herein that include: (a) culturing any of the recombinant host cells described herein in a liquid culture medium under conditions sufficient to produce the ACC; and (b) recovering the ACC from the host cell and/or the liquid culture medium.

Methods of culturing cells are well known in the art. Cells can be maintained in vitro under conditions that favor cell proliferation, cell differentiation and cell growth. For example, cells can be cultured by contacting a cell (e.g., any of the cells described herein) with a cell culture medium that includes the necessary growth factors and supplements sufficient to support cell viability and growth.

In some embodiments of any of the methods described herein, the method further includes isolating the recovered ACC. Non-limiting examples of methods of isolation include: ammonium sulfate precipitation, polyethylene glycol precipitation, size exclusion chromatography, ligand-affinity chromatography, ion-exchange chromatography (e.g., anion or cation), and hydrophobic interaction chromatography.

In some embodiments, the cells can produce a protein portion of a first monomer construct that includes the CP1 and the CM1, and a protein portion of a second monomer construct that includes the CP2 and the CM2, and then the protein portions are subsequently conjugated to the DD1 and DD2 moieties, respectively.

Compositions and methods described herein may involve use of non-reducing or partially-reducing conditions that allow disulfide bonds to form between the dimerization domains to form and maintain dimerization of the ACCs.

In some embodiments of any of the methods described herein, the method further includes formulating the isolated ACC into a pharmaceutical composition. Various formulations are known in the art and are described herein. Any of the isolated ACCs described herein can be formulated for any route of administration (e.g., intravenous, intratumoral, subcutaneous, intradermal, oral (e.g., inhalation), transdermal (e.g., topical), transmucosal, or intramuscular).

Also provided herein are ACCs produced by any of the methods described herein. Also provided are compositions (e.g., pharmaceutical compositions) that include any of the ACCs produced by any of the methods described herein. Also provided herein are kits that include at least one dose of any of the compositions (e.g., pharmaceutical compositions) described herein.

### Methods of Treatment

Provided herein are methods of treating a disease (e.g., a cancer (e.g., any of the cancers described herein)) in a subject including administering a therapeutically effective amount of any of the ACCs described herein to the subject.

As used herein, the term “subject” refers to any mammal. In some embodiments, the subject is a feline (e.g., a cat), a canine (e.g., a dog), an equine (e.g., a horse), a rabbit, a pig, a rodent (e.g., a mouse, a rat, a hamster or a guinea pig), a non-human primate (e.g., a simian (e.g., a monkey (e.g., a baboon, a marmoset), or an ape (e.g., a chimpanzee, a gorilla, an orangutan, or a gibbon)), or a human. In some embodiments, the subject is a human.

In some embodiments, the subject has been previously identified or diagnosed as having the disease (e.g., cancer (e.g., any of the cancers described herein)).

As used herein, the term “treat” includes reducing the severity, frequency or the number of one or more (e.g., 1, 2, 3, 4, or 5) symptoms or signs of a disease (e.g., a cancer (e.g., any of the cancers described herein)) in the subject (e.g., any of the subjects described herein). In some embodiments where the disease is cancer, treating results in reducing cancer growth, inhibiting cancer progression, inhibiting cancer metastasis, or reducing the risk of cancer recurrence in a subject having cancer.

In some embodiments of any of the methods described herein, the disease is a cancer. Also provided herein are methods of treating a subject in need thereof (e.g., any of the exemplary subjects described herein or known in the art) that include administering to the subject a therapeutically effective amount of any of the ACCs described herein or any of the compositions (e.g., pharmaceutical compositions) described herein.

In some embodiments of these methods, the subject has been identified or diagnosed as having a cancer. Non-limiting examples of cancer include: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, a lymphoma (e.g., B-cell lymphoma, B-cell non-Hodgkin's lymphoma, Hodgkin's lymphoma, cutaneous T-cell lymphoma), a leukemia (e.g., hairy cell leukemia, chronic lymphocytic leukemia (CLL), acute myeloid leukemia (AML), chronic myeloid leukemia (CML), acute lymphocytic leukemia (ALL)), myelodysplastic syndromes (MDS), Kaposi sarcoma, retinoblastoma, stomach cancer, urothelial carcinoma, lung cancer, renal cell carcinoma, gastric and esophageal cancer, pancreatic cancer, prostate cancer, brain cancer, colon cancer, bone cancer, lung cancer, breast cancer, colorectal cancer, ovarian cancer, nasopharyngeal adenocarcinoma, non-small cell lung carcinoma (NSCLC), squamous cell head and neck carcinoma, endometrial cancer, bladder cancer, cervical cancer, liver cancer, and hepatocellular carcinoma. In some embodiments, the cancer is a lymphoma. In some embodiments, the lymphoma is Burkitt's lymphoma. In some aspects, the subject has been identified or diagnosed as having familial cancer syndromes such as Li Fraumeni Syndrome, Familial Breast-Ovarian Cancer (BRCA1 or BRAC2 mutations) Syndromes, and others. The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine.

Exemplary cancers described by the National Cancer Institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

Further exemplary cancers include diffuse large B-cell lymphoma (DLBCL) and mantle cell lymphoma (MCL).

Metastases of the aforementioned cancers can also be treated or prevented in accordance with the methods described herein.

In some embodiments, these methods can result in a reduction in the number, severity, or frequency of one or more symptoms of the cancer in the subject (e.g., as compared to the number, severity, or frequency of the one or more symptoms of the cancer in the subject prior to treatment).

In some embodiments of any of the methods described herein, the methods further include administering to a subject an additional therapeutic agent (e.g., one or more of the therapeutic agents listed in Table 2).

TABLE 2
Additional Therapeutic Agents

| Antibody Trade Name | |
|---|---|
| (antibody name) | Target |
| Raptiva ™ (efalizumab) | CD11a |
| Arzerra ™ (ofatumumab) | CD20 |
| Bexxar ™ (tositumomab) | CD20 |
| Gazyva ™ (obinutuzumab) | CD20 |
| Ocrevus ™ (ocrelizumab) | CD20 |
| Rituxan ™ (rituximab) | CD20 |
| Zevalin ™ (ibritumomab tiuxetan) | CD20 |
| Adcetris ™ (brentuximab vedotin) | CD30 |
| Myelotarg ™ (gemtuzumab) | CD33 |
| Mylotarg ™ (gemtuzumab ozogamicin) | CD33 |
| (vadastuximab) | CD33 |
| (vadastuximab talirine) | CD33 |
| Campath ™ (alemtuzumab) | CD52 |
| Lemtrada ™ (alemtuzumab) | CD52 |
| Tactress ™ (tamtuvetmab) | CD52 |
| Soliris ™ (eculizumab) | Complement C5 |
| Ultomiris ™ (ravulizumab) | Complement C5 |
| (olendalizumab) | Complement C5 |
| Yervoy ™ (ipilimumab) | CTLA-4 |
| (tremelimumab) | CTLA-4 |
| Orencia ™ (abatacept) | CTLA-4 |
| Hu5c8 | CD40L |
| (letolizumab) | CD40L |
| Rexomun ™ (ertumaxomab) | CD3/Her2 |
| Erbitux ™ (cetuximab) | EGFR |
| Portrazza ™ (necitumumab) | EGFR |
| Vectibix ™ (panitumumab) | EGFR |
| CH806 | EGFR |
| (depatuxizumab) | EGFR |
| (depatuxizumab mafodotin) | EGFR |
| (futuximab:modotuximab) | EGFR |
| ICR62 (imgatuzumab) | EGFR |
| (laprituximab) | EGFR |
| (losatuxizumab) | EGFR |
| (losatuxizumab vedotin) | EGFR |
| mAb 528 | EGFR |
| (matuzumab) | EGFR |
| (nimotuzumab) | EGFR |
| (tomuzotuximab) | EGFR |
| (zalutumumab) | EGFR |
| MDX-447 | EGFR/CD64 |
| (adecatumumab) | EpCAM |
| Panorex ™ (edrecolomab) | EpCAM |
| Vicinium ™ | EpCAM |
| Synagis ™ (palivizumab) | F protein of RSV |
| ReoPro ™ (abiciximab) | Glycoprotein receptor IIb/IIIa |
| Herceptin ™ (trastuzumab) | Her2 |
| Herceptin ™ Hylecta (trastuzumab; | Her2 |
| Hyaluronidase) | |
| (trastuzumab deruxtecan) | Her2 |
| (hertuzumab verdotin) | Her2 |
| Kadcyla ™ (trastuzumab emtansine) | Her2 |
| (margetuximab) | Her2 |
| (timigutuzumab) | Her2 |
| Xolair ™ (omalizumab) | IgE |
| (ligelizumab) | IgE |
| (figitumumab) | IGF1R |
| (teprotumumab) | IGF1R |
| Simulect ™ (basiliximab) | IL2R |
| Zenapax ™ (daclizumab) | IL2R |
| Zinbryta ™ (daclizumab) | IL2R |
| Actemra ™ (tocilizumab) | IL-6 receptor |
| Kevzara ™ (sarilumab) | IL-6 receptor |
| (vobarilizumab) | IL-6 receptor |
| Stelara ™ (ustekinumab) | IL-12/IL-23 |
| Tysabri ™ (natalizumab) | Integrinα4 |
| (abrilumab) | Integrinα4 |
| | Jagged 1 or Jagged 2 |
| (fasinumab) | NGF |
| (fulranumab) | NGF |
| (tanezumab) | NGF |
| | Notch, e.g., Notch 1 |
| Pidilizumab | Delta like-1 (PD-1 pathway |
| | inhibitor) |
| Opdivo ® (nivolumab) | PD1 |
| Keytruda ® (pembrolizumab) | PD1 |
| Libtayo ® (cemiplimab) | PD1 |
| BGB-A317 (tislelizumab) | PD1 |
| PDR001 (spartalizumab) | PD1 |
| JNJ-63723283 (cetrelimab) | PD1 |
| TSR042 (dostarlimab) | PD1 |
| AGEN2034 (balstilimab) | PD1 |
| JS001 (toripalimab) | PD1 |
| IOBI308 (sintilimab) | PD1 |
| BCD100 (prolgolimab) | PD1 |
| CBT-501 (genolimzumab) | PD1 |
| ABBV181 (budigalimab) | PD1 |
| AK105 | PD1 |
| BI-754091 | PD1 |
| INCSHR-1210 | PD1 |
| MEDI0680 | PD1 |
| MGA012 | PD1 |
| SHR-1210 | PD1 |
| Imfinzi ™ (durvalumab) | PD-L1 |
| Tecentriq ® (atezolizumab) | PD-L1 |
| Bavencio ® (avelumab) | PD-L1 |
| KN035 (envafolimab) | PD-L1 |
| BMS936559 (MDX1105) | PD-L1 |
| BGBA 333 | PD-L1 |
| FAZ053 | PD-L1 |
| LY-3300054 | PD-L1 |
| SH-1316 | PD-L1 |
| AMP-224 | PD-L2 |
| (bavituximab) | Phosphatidylserine |
| huJ591 | PSMA |
| RAV12 | RAAG12 |
| Prolia ™ (denosumab) | RANKL |
| GC1008 (fresolimumab) | TGFbeta |
| Cimzia ™ (Certolizumab Pegol) | TNFα |
| Remicade ™ (infliximab) | TNFα |
| Humira ™ (adalimumab) | TNFα |
| Simponi ™ (golimumab) | TNFα |
| Enbrel ™ (etanercept) | TNF-R |
| (mapatumumab) | TRAIL-R1 |
| Avastin ™ (bevacizumab) | VEGF |
| Lucentis ™ (ranibizumab) | VEGF |
| (brolucizumab) | VEGF |
| (vanucizumab) | VEGF |

### Compositions/Kits

Also provided herein are compositions (e.g., pharmaceutical compositions) including any of the ACCs described herein and one or more (e.g., 1, 2, 3, 4, or 5) pharmaceutically acceptable carriers (e.g., any of the pharmaceutically acceptable carriers described herein), diluents, or excipients.

In some embodiments, the compositions (e.g. pharmaceutical compositions) that include any of the ACCs described herein can be disposed in a sterile vial or a pre-loaded syringe.

In some embodiments, the compositions (e.g. pharmaceutical compositions) that include any of the ACCs described herein can be formulated for different routes of administration (e.g., intravenous, subcutaneous, intramuscular, intraperitoneal, or intratumoral).

In some embodiments, any of the pharmaceutical compositions described herein can include one or more buffers (e.g., a neutral-buffered saline, a phosphate-buffered saline (PBS), amino acids (e.g., glycine), one or more carbohydrates (e.g., glucose, mannose, sucrose, dextran, or mannitol), one or more antioxidants, one or more chelating agents (e.g., EDTA or glutathione), one or more preservatives, and/or a pharmaceutically acceptable carrier (e.g., bacteriostatic water, PBS, or saline).

As used herein, the phrase “pharmaceutically acceptable carrier” refers to any and all solvents, dispersion media, coatings, antibacterial agents, antimicrobial agents, isotonic and absorption delaying agents, and the like, compatible with pharmaceutical administration. Suitable carriers include, but are not limited to: water, saline, ringer's solutions, dextrose solution, and about 5% human serum albumin.

In some embodiments of any of the pharmaceutical compositions described herein, any of the ACCs described herein are prepared with carriers that protect against rapid elimination from the body, e.g., sustained and controlled release formulations, including implants and microencapsulated delivery systems. Biodegradable, biocompatible polymers can be used, e.g., ethylene vinyl acetate, polyanhydrides, polyglycolic acid, collage, polyorthoesters, and polylactic acid. Methods for preparation of such pharmaceutical compositions and formulations are apparent to those skilled in the art.

Also provided herein are kits that include any of the ACCs described herein, any of the compositions that include any of the ACCs described herein, or any of the pharmaceutical compositions that include any of the ACCs described herein. Also provided are kits that include one or more second therapeutic agent(s) selected from Table 2 in addition to an ACC described herein. The second therapeutic agent(s) may be provided in a dosage administration form that is separate from the ACC. Alternatively, the second therapeutic agent(s) may be formulated together with the ACC.

Any of the kits described herein can include instructions for using any of the compositions (e.g., pharmaceutical compositions) and/or any of the ACCs described herein. In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein.

The present disclosure includes the following non-limiting aspects:
1. An activatable cytokine construct (ACC) that includes a first monomer construct and a second monomer construct, wherein:
   (a) the first monomer construct comprises a first mature cytokine protein (CP1), a first cleavable moiety (CM1), and a first dimerization domain (DD1),
      wherein the CM1 is positioned between the CP1 and the DD1; and
   (b) the second monomer construct comprises a second mature cytokine protein (CP2), a second cleavable moiety (CM2), and a second dimerization domain (DD2),
      wherein the CM2 is positioned between the CP2 and the DD2; or
   (a) the first monomer construct comprises a first mature cytokine protein (CP1), a first dimerization domain (DD1), and
   (b) the second monomer construct comprises a second mature cytokine protein (CP2), a cleavable moiety (CM), and a second dimerization domain (DD2), wherein the CM is positioned between the CP2 and the DD2, wherein the CM functions as a substrate for a protease; or
   (a) the first monomer construct comprises a first mature cytokine protein (CP1), a cleavable moiety (CM), and a first dimerization domain (DD1), wherein the CM is positioned between the CP1 and the DD1, and
   (b) the second monomer construct comprises a second mature cytokine protein (CP2), and a second dimerization domain (DD2),
   wherein the CM functions as a substrate for a protease; or
   (a) the first monomer construct comprises a first mature cytokine protein (CP1), and a first dimerization domain (DD1), and
   (b) the second monomer construct comprises a second mature cytokine protein (CP2), and a second dimerization domain (DD2), wherein the CP1, the CP2, or both CP1 and CP2 include(s) an amino acid sequence that functions as a substrate for a protease;
   wherein the DD1 and the DD2 bind each other thereby forming a dimer of the first monomer construct and the second monomer construct; and
   wherein the ACC is characterized by having a reduced level of at least one CP1 and/or CP2 activity as compared to a control level of the at least one CP1 and/or CP2 activity.
2. The ACC of aspect 1, wherein the first monomer construct comprises a first polypeptide that comprises the CP1, the CM1, and the DD1.
3. The ACC of any one or combination of aspect 1 or 2, wherein the second monomer construct comprises a second polypeptide that comprises the CP2, the CM2, and the DD2.
4. The ACC of any one or combination of aspects 1-3, wherein the DD1 and the DD2 are a pair selected from the group consisting of: a pair of Fc domains, a sushi domain from an alpha chain of human IL-15 receptor (IL15Rα) and a soluble IL-15; barnase and barnstar; a PKA and an AKAP; adapter/docking tag modules based on mutated RNase I fragments; an epitope and sdAb; an epitope and scFv; and SNARE modules based on interactions of the proteins syntaxin, synaptotagmin, synaptobrevin, and SNAP25, an antigen-binding domain and an epitope.
5. The ACC of aspect 4, wherein the DD1 and the DD2 are a pair of Fc domains.
6. The ACC of aspect 5, wherein the pair of Fc domains is a pair of human Fc domains.
7. The ACC of aspect 6, wherein the human Fc domains are human IgG1 Fc domains, human IgG2 Fc domains, human IgG3 Fc domains, or human IgG4 Fc domains.
8. The ACC of aspect 7, wherein the human Fc domains are human IgG4 Fc domains.
9. The ACC of aspect 8, wherein the human Fc domains comprise a sequence that is at least 80%, 85%, 90%, 95%, 96%, 97%, 98%, or 99% identical to SEQ ID NO: 3, SEQ ID NO: 315, or SEQ ID NO: 316.
10. The ACC of aspect 9, wherein the human Fc domains comprise a sequence that is at least 90% identical to SEQ ID NO: 3, SEQ ID NO: 315, or SEQ ID NO: 316.
11. The ACC of aspect 10, wherein the human Fc domains comprise SEQ ID NO: 3, SEQ ID NO: 315, or SEQ ID NO: 316.
12. The ACC of any one or combination of aspects 1-3 and 5-11, wherein the DD1 and the DD2 are the same.
13. The ACC of aspect 4, wherein DD1 comprises an antigen-binding domain and DD2 comprises a corresponding epitope.
14. The ACC of aspect 13, wherein the antigen-binding domain is an anti-His tag antigen-binding domain and wherein the DD2 comprises a His tag.
15. The ACC of aspect 13, wherein the antigen-binding domain is a single chain variable fragment (scFv).
16. The ACC of aspect 13, wherein the antigen-binding domain is a single domain antibody (sdAb).
17. The ACC of aspect 1, wherein at least one of DD1 and DD2 comprises a dimerization domain substituent selected from the group consisting of a non-polypeptide polymer and a small molecule.
18. The ACC of aspect 17, wherein DD1 and DD2 comprise non-polypeptide polymers covalently bound to each other.
19. The ACC of aspect 18, wherein the non-polypeptide polymer is a sulfur-containing polyethylene glycol, and wherein DD1 and DD2 are covalently bound to each other via one or more disulfide bonds.
20. The ACC of aspect 17, wherein at least one of DD1 and DD2 comprises a small molecule.
21. The ACC of aspect 20, wherein the small molecule is biotin.
22. he ACC of aspect 20, wherein DD1 comprises biotin and DD2 comprises an avidin.
23. The ACC of any one or combination of aspects 1-22, wherein the CP1 and/or the CP2 is/are each individually selected from the group consisting of: an interferon, an interleukin, GM-CSF, G-CSF, LIF, OSM, CD154, LT-β, TNF-α, TNF-β, 4-1BBL, APRIL, CD70, CD153, CD178, GITRL, LIGHT, OX40L, TALL-1, TRAIL, TWEAK, TRANCE, TGF-β1, TGF-β1, TGF-β3, Epo, Tpo, Flt-3L, SCF, M-CSF, and MSP.
24. The ACC of any one or combination of aspects 1-23, wherein the CP1 and the CP2 are the same.
25. The ACC of any one or combination of aspects 1-23 wherein the CP1 and the CP2 are different.
26. The ACC of any one or combination of aspects 1-23, wherein the CP1 and/or the CP2 is/are an interferon.
27. The ACC of aspect 26, wherein the CP1 and the CP2 are an interferon.
28. The ACC of aspect 26, wherein the CP1 and the CP2 are different interferons.
29. The ACC of aspect 26, wherein the CP1 and the CP2 are the same interferon.
30. The ACC of aspect 26, wherein the CP1 or the CP2 is an interferon.
31. The ACC of any one or combination of aspects 26-30, wherein the interferon(s) is/are a human wildtype mature interferon.
32. The ACC of any one or combination of aspects 26-31, wherein the interferon(s) is/are selected from the group consisting of: interferon-alpha, interferon-beta, interferon-omega, and interferon-tau.
33. The ACC of aspect 32, wherein the interferons is/are an interferon-alpha.
34. The ACC of aspect 33, wherein the interferon(s) is/are selected from the group consisting of: interferon alpha-2a, interferon alpha-2b, and interferon alpha-n3.
35. The ACC of aspect 34, wherein the interferon(s) is/are interferon alpha-2b.
36. The ACC of aspect 35, wherein the CP1 and/or CP2 comprises a sequence that is at least 80% identical to SEQ ID NO: 1.
37. The ACC of aspect 36, wherein the CP1 and/or CP2 comprises a sequence that is at least 90% identical to SEQ ID NO: 1.
38. The ACC of aspect 37, wherein the CP1 and/or CP2 comprises a sequence of SEQ ID NO: 1.
39. The ACC of aspect 32, wherein the interferon is an interferon beta.
40. The ACC of aspect 39, wherein the interferon beta is selected from the group consisting of interferon beta-1a, and interferon beta-1b.
41. The ACC of any one of aspects 1-40, wherein the CP1 and/or the CP2 comprises an IFab domain.
42. The ACC of aspect 41, wherein the CP1 and/or the CP2 comprises an interleukin.
43. The ACC of aspect 42, wherein the interleukin is selected from the group consisting of IL-1α, IL-1β, IL-1RA, IL-18, IL-2, IL-4, IL-7, IL-9, IL-13, IL-15, IL-3, IL-5, IL-6, IL-11, IL-12, IL-10, IL-20, IL-14, IL-16, and IL-17.
44. The ACC of any one or combination of aspects 1-43, wherein the CM1 and/or the CM2 comprise a total of about 3 amino acids to about 15 amino acids.
45. The ACC of any one or combination of aspects 1-44, wherein the CM1 and the CM2 comprise substrates for different proteases.
46. The ACC of any one or combination of aspects 1-44, wherein the CM1 and the CM2 comprise substrates for the same protease.
47. The ACC of any one or combination of aspects 1-46, wherein the protease(s) is/are selected from the group consisting of: ADAM8, ADAM9, ADAM10, ADAM12, ADAM15, ADAM17/TACE, ADAMDEC1, ADAMTS1, ADAMTS4, ADAMTS5, BACE, Renin, Cathepsin D, Cathepsin E, Caspase 1, Caspase 2, Caspase 3, Caspase 4, Caspase 5, Caspase 6, Caspase 7, Caspase 8, Caspase 9, Caspase 10, Caspase 14, Cathepsin B, Cathepsin C, Cathepsin K, Cathespin L, Cathepsin S, Cathepsin V/L2, Cathepsin X/Z/P, Cruzipain, Legumain, Otubain-2, KLK4, KLK5, KLK6, KLK7, KLK8, KLK10, KLK11, KLK13, KLK14, Meprin, Neprilysin, PSMA, BMP-1, MMP-1, MMP-2, MMP-3, MMP-7, MMP-9, MMP-10, MMP-11, MMP-12, MMP-13, MMP-14, MMP-15, MMP-16, MMP-17, MMP-19, MMP-20, MMP-23, MMP-24, MMP-26, MMP-27, activated protein C, cathepsin A, cathepsin G, Chymase, FVIIa, FIXa, FXa, FXIa, FXIIa, Elastase, Granzyme B, Guanidinobenzoatase, HtrA1, human neutrophil lyase, lactoferrin, marapsin, NS3/4A, PACE4, Plasmin, PSA, tPA, thrombin, tryptase, uPA, DESC1, DPP-4, FAP, Hepsin, Matriptase-2, MT-SP1/Matripase, TMPRSS2, TMPRSS3, and TMPRSS4.
48. The ACC of aspect 47, wherein the protease(s) is/are selected from the group consisting of: uPA, legumain, MT-SP1, ADAM17, BMP-1, TMPRSS3, TMPRSS4, MMP-2, MMP-9, MMP-12, MMP-13, and MMP-14.
49. The ACC of aspect 47, wherein the CM1 and/or the CM2 comprise a sequence selected from the group consisting of: LSGRSDNH (SEQ ID NO: 5), TGRGPSWV (SEQ ID NO: 6), PLTGRSGG (SEQ ID NO: 7), TARGPSFK (SEQ ID NO: 8), NTLSGRSENHSG (SEQ ID NO: 9), NTLSGRSGNHGS (SEQ ID NO: 10), TSTSGRSANPRG (SEQ ID NO: 11), TSGRSANP (SEQ ID NO: 12), VHMPLGFLGP (SEQ ID NO: 13), AVGLLAPP (SEQ ID NO: 14), AQNLLGMV (SEQ ID NO: 15), QNQALRMA (SEQ ID NO: 16), LAAPLGLL (SEQ ID NO: 17), STFPFGMF (SEQ ID NO: 18), ISSGLLSS (SEQ ID NO: 19), PAGLWLDP (SEQ ID NO: 20), VAGRSMRP (SEQ ID NO: 21), VVPEGRRS (SEQ ID NO: 22), ILPRSPAF (SEQ ID NO: 23), MVLGRSLL (SEQ ID NO: 24), QGRAITFI (SEQ ID NO: 25), SPRSIMLA (SEQ ID NO: 26), SMLRSMPL (SEQ ID NO: 27), ISSGLLSGRSDNH (SEQ ID NO: 28), AVGLLAPPGGLSGRSDNH (SEQ ID NO: 29), ISSGLLSSGGSGGSLSGRSDNH (SEQ ID NO: 30), LSGRSGNH (SEQ ID NO: 31), SGRSANPRG (SEQ ID NO: 32), LSGRSDDH (SEQ ID NO: 33), LSGRSDIH (SEQ ID NO: 34), LSGRSDQH (SEQ ID NO: 35), LSGRSDTH (SEQ ID NO: 36), LSGRSDYH (SEQ ID NO: 37), LSGRSDNP (SEQ ID NO: 38), LSGRSANP (SEQ ID NO: 39), LSGRSANI (SEQ ID NO: 40), LSGRSDNI (SEQ ID NO: 41), MIAPVAYR (SEQ ID NO: 42), RPSPMWAY (SEQ ID NO: 43), WATPRPMR (SEQ ID NO: 44), FRLLDWQW (SEQ ID NO: 45), ISSGL (SEQ ID NO: 46), ISSGLLS (SEQ ID NO: 47), ISSGLL (SEQ ID NO: 48), ISSGLLSGRSANPRG (SEQ ID NO: 49), AVGLLAPPTSGRSANPRG (SEQ ID NO: 50), AVGLLAPPSGRSANPRG (SEQ ID NO: 51), ISSGLLSGRSDDH (SEQ ID NO: 52), ISSGLLSGRSDIH (SEQ ID NO: 53), ISSGLLSGRSDQH (SEQ ID NO: 54), ISSGLLSGRSDTH (SEQ ID NO: 55), ISSGLLSGRSDYH (SEQ ID NO: 56), ISSGLLSGRSDNP (SEQ ID NO: 57), ISSGLLSGRSANP (SEQ ID NO: 58), ISSGLLSGRSANI (SEQ ID NO: 59), AVGLLAPPGGLSGRSDDH (SEQ ID NO: 60), AVGLLAPPGGLSGRSDIH (SEQ ID NO: 61), AVGLLAPPGGLSGRSDQH (SEQ ID NO: 62), AVGLLAPPGGLSGRSDTH (SEQ ID NO: 63), AVGLLAPPGGLSGRSDYH (SEQ ID NO: 64), AVGLLAPPGGLSGRSDNP (SEQ ID NO: 65), AVGLLAPPGGLSGRSANP (SEQ ID NO: 66), AVGLLAPPGGLSGRSANI (SEQ ID NO: 67), ISSGLLSGRSDNI (SEQ ID NO: 68), AVGLLAPPGGLSGRSDNI (SEQ ID NO: 69), GLSGRSDNHGGAVGLLAPP (SEQ ID NO: 70), GLSGRSDNHGGVHIMIPLGFLGP (SEQ ID NO: 71), LSGRSDNHGGVHIMIPLGFLGP (SEQ ID NO: 72), ISSGLSS (SEQ ID NO: 73), PVGYTSSL (SEQ ID NO: 74), DWLYWPGI (SEQ ID NO: 75), LKAAPRWA (SEQ ID NO: 76), GPSHLVLT (SEQ ID NO: 77), LPGGLSPW (SEQ ID NO: 78), MGLFSEAG (SEQ ID NO: 79), SPLPLRVP (SEQ ID NO: 80), RMILRSLG (SEQ ID NO: 81), LLAPSHRA (SEQ ID NO: 82), GPRSFGL (SEQ ID NO: 83), GPRSFG (SEQ ID NO: 84), SARGPSRW (SEQ ID NO: 85), GGWHTGRN (SEQ ID NO: 86), HTGRSGAL (SEQ ID NO: 87), AARGPAIH (SEQ ID NO: 88), RGPAFNPM (SEQ ID NO: 89), SSRGPAYL (SEQ ID NO: 90), RGPATPIM (SEQ ID NO: 91), RGPA (SEQ ID NO: 92), GGQPSGMWGW (SEQ ID NO: 93), FPRPLGITGL (SEQ ID NO: 94), SPLTGRSG (SEQ ID NO: 95), SAGFSLPA (SEQ ID NO: 96), LAPLGLQRR (SEQ ID NO: 97), SGGPLGVR (SEQ ID NO: 98), PLGL (SEQ ID NO: 99), and SGRSDNI (SEQ ID NO: 100).
50. The ACC of aspect 47, wherein the CM1 and/or the CM2 comprises a sequence selected from the group consisting of: ISSGLLSGRSDNH (SEQ ID NO: 28), LSGRSDDH (SEQ ID NO: 33), ISSGLLSGRSDQH (SEQ ID NO: 54), SGRSDNI (SEQ ID NO: 100), and ISSGLLSGRSDNI (SEQ ID NO: 68).
51. The ACC of any one or combination of aspects 1-50, wherein the protease(s) is/are produced by a tumor in a subject.
52. The ACC of aspect 51, wherein the subject has been diagnosed or identified as having a cancer.
53. The ACC of any one or combination of aspects 1-52, wherein the CP1 and the CM1 directly abut each other in the first monomer construct.
54. The ACC of any one or combination of aspects 1-53, wherein the CM1 and the DD1 directly abut each other in the first monomer construct.
55. The ACC of any one or combination of aspects 1-54, wherein the CP2 and the CM2 directly abut each other in the second monomer construct.
56. The ACC of any one or combination of aspects 1-55, wherein the CM2 and the DD2 directly abut each other in the second monomer construct.
57. The ACC of any one or combination of aspects 1-56, wherein the first monomer construct comprises at least one linker.
58. The ACC of aspect 57, wherein the at least one linker is a linker L1 disposed between the CP1 and the CM1 and/or a linker L2 disposed between the CM1 and the DD1.
59. The ACC of aspect 58, wherein the second monomer construct comprises at least one linker.
60. The ACC of aspect 59, wherein the at least one linker is a linker L3 disposed between the CP2 and the CM2 and/or a linker L4 disposed between the CM2 and the DD2.

The ACC of aspect 60, wherein the first monomer construct comprises a linker L1 and the second monomer construct comprises a linker L3.
61. The ACC of aspect 61, wherein L1 and L3 are the same.
62. The ACC of aspect 62, wherein the second monomer construct comprises a linker L2 and the second monomer construct comprises a linker L4.
63. The ACC of aspect 63, wherein L2 and L4 are the same.
64. The ACC of aspect 64, wherein each linker has a total length of 1 amino acid to about 15 amino acids.
65. The ACC of aspect 65, wherein each linker has a total length of at least 5 amino acids.
66. The ACC of any one or combination of aspects 1-66, wherein the first monomer construct comprises at least one linker, wherein each linker is independently selected from from the group consisting of GSSGGSGGSGG (SEQ ID NO: 210); GGGS (SEQ ID NO: 2); GGGSGGGS (SEQ ID NO: 211); GGGSGGGSGGGS (SEQ ID NO: 212); GGGGSGGGGSGGGGS (SEQ ID NO: 213); GGGGSGGGGSGGGGSGGGGSGGGGS (SEQ ID NO: 214); GGGGSGGGGS (SEQ ID NO: 215); GGGGS (SEQ ID NO: 216); GS; GGGGSGS (SEQ ID NO: 217); GGGGSGGGGSGGGGSGS (SEQ ID NO: 218); GGSLDPKGGGGS (SEQ ID NO: 219); PKSCDKTHTCPPCPAPELLG (SEQ ID NO: 220); SKYGPPCPPCPAPEFLG (SEQ ID NO: 221); GKSSGSGSESKS (SEQ ID NO: 222); GSTSGSGKSSEGKG (SEQ ID NO: 223); GSTSGSGKSSEGSGSTKG (SEQ ID NO: 224); GSTSGSGKPGSGEGSTKG (SEQ ID NO: 225); GSTSGSGKPGSSEGST (SEQ ID NO: 226); (GS)n, (GGS)n, (GSGGS)n (SEQ ID NO: 227), (GGGS)n (SEQ ID NO: 228), (GGGGS)n (SEQ ID NO: 216), wherein each n is an integer of at least one; GGSG (SEQ ID NO: 229); GGSGG (SEQ ID NO: 230); GSGSG (SEQ ID NO: 231; GSGGG (SEQ ID NO: 232); GGGSG (SEQ ID NO: 233); GSSSG (SEQ ID NO: 234); GGGGSGGGGSGGGGS (SEQ ID NO: 213); GGGGSGGGGSGGGGSGGGGS (SEQ ID NO: 214); and GSTSGSGKPGSSEGST (SEQ ID NO: 226).
67. The ACC of aspect 67, wherein the linker comprises a sequence of GGGS (SEQ ID NO: 2).
68. The ACC of any one or combination of aspects 1-68, wherein the first monomer construct, comprises in a N- to C-terminal direction, the CP1, the CM1, and, linked directly or indirectly to the C-terminus of the CM1, the DD1.
69. The ACC of any one or combination of aspects 1-69, wherein the first polypeptide comprises in a C- to N-terminal direction, the CP1, the CM1, and, linked directly or indirectly to the N-terminus of the CM1, the DD1.
70. The ACC of any one or combination of aspects 1-70, wherein the second polypeptide comprises in a N- to C-terminal direction, the CP2, CM2, and, linked directly or indirectly to the C-terminus of the CM2, the DD2.
71. The ACC of any one or combination of aspects 1-71, wherein the second polypeptide comprises in a C- to N-terminal direction, the CP2, CM2, and, linked directly or indirectly to the CM2, the DD2.
72. The ACC of aspect 69, wherein the first monomer construct comprises, in the N- to C-terminal direction, the CP1, the CM1, and the DD1, wherein the CP1 and the CM1 directly abut each other, wherein the CM1 and the DD1 directly abut each other, wherein the CM1 is a peptide of not more than 10 amino acids, wherein the second monomer construct is the same as the first monomer construct, and wherein the first and second monomer constructs are covalently bound to each other via at least two disulfide bonds.
73. The ACC of aspect 73, wherein CP1 is an interferon.
74. The ACC of aspect 74, wherein CP1 is an interferon-alpha.
75. The ACC of any one or combination of aspects 1-75, wherein the at least one CP1 and/or CP2 activity is a binding affinity (K_{D}) of the CP1 and/or the CP2 for its cognate receptor as determined using surface plasmon resonance.
76. The ACC of any one or combination of aspects 1-75, wherein the at least one CP1 and/or CP2 activity is a level of proliferation of lymphoma cells.
77. The ACC of any one or combination of aspects 1-75, wherein the at least one CP1 and/or CP2 activity is a level of JAK/STAT/ISGF3 pathway activation in a lymphoma cell.
78. The ACC of any one or combination of aspects 1-75, wherein the at least one activity is a level of SEAP production in a lymphoma cell.
79. The ACC of any one or combination aspects 1-79, wherein the ACC is characterized by at least a 20-fold reduction in at least one CP1 and/or CP2 activity as compared to the control level.
80. The ACC of aspect 80, wherein the ACC is characterized by at least a 50-fold reduction in at least one CP1 and/or CP2 activity as compared to the control level.
81. The ACC of aspect 81, wherein the ACC is characterized by at least a 100-fold reduction in at least one activity of the CP1 and/or CP2 as compared to the control level.
82. The ACC of aspect 82, wherein the ACC is characterized by at least a 500-fold reduction in at least one CP1 and/or CP2 activity as compared to the control level.
83. The ACC of any one or combination of aspects 1-83, wherein the control level of the at least one activity of the CP1 and/or CP2, is the activity of the CP1 and/or CP2 in the ACC following exposure of the ACC to the protease(s).
84. The ACC of any one or combination of aspects 1-83, wherein the control level of the at least one CP1 and/or CP2, is the corresponding CP1 and/or CP2 activity of a corresponding wildtype mature cytokine.
85. The ACC of any one or combination of aspects 1-85, wherein the ACC is characterized by generating a cleavage product following exposure to the protease(s), wherein the cleavage product comprises the at least one activity of the CP1 and/or CP2.
86. The ACC of aspect 86, wherein the at least one activity of the CP1 and/or CP2 is anti-proliferation activity.
87. The ACC of aspect 87, wherein the control level is an EC50 value, and wherein ratio of EC50 (cleavage product) to EC50 (control level) is less than about 10, or less than about 9, or less than about 8, or less than about 7, or less than about 6, or less than about 5, or less than about 4, or less than about 3, or less than about 2, or less than about 1.5.
88. A composition comprising an ACC of any one or combination of aspects 1-88.
89. The composition of aspect 89, wherein the composition is a pharmaceutical composition.
90. A container, vial, syringe, injector pen, or kit comprising at least one dose of the composition of aspect 89 or 90.
91. A method of treating a subject in need thereof comprising administering to the subject a therapeutically effective amount of the ACC of any one or combination of aspects 1-88 or the composition of aspects 89 or 90.
92. The method of aspect 92, wherein the subject has been identified or diagnosed as having a cancer.
93. The method of aspect 93, wherein the cancer is a lymphoma, solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, bladder cancer, stomach cancer, urothelial carcinoma, lung cancer, colon cancer, renal cell carcinoma, gastric and esophageal cancer, pancreatic cancer, prostate cancer, breast cancer, colorectal cancer, ovarian cancer, non-small cell lung carcinoma, squamous cell head and neck carcinoma, endometrial cancer, cervical cancer, liver cancer, or hepatocellular carcinoma.
94. The method of aspect 94, wherein the lymphoma is Burkitt's lymphoma.
95. A nucleic acid encoding a polypeptide that comprises the CP1 and CM1 of the ACC of any one or combination of aspects 1-88.
96. The nucleic acid of aspect 96, wherein the polypeptide further comprises a DD1 of any one or combination of aspects 1-16 or aspects 23-88.
97. A nucleic acid encoding a polypeptide that comprises the CP2 and CM2 of the ACC of any one or combination of aspects 1-88.
98. The nucleic acid of aspect 98, wherein the polypeptide further comprises the DD2 of any one or combination of aspects 1-16 or aspects 23-88.
99. A vector comprising the nucleic acid of any one or combination of aspects 96-99.
100. The vector of aspect 100, wherein the vector is an expression vector.
101. A cell comprising the nucleic acid of any one or combination of aspects 96-99 or the vector of aspect 100 or 101.
102. A pair of nucleic acids that together encode a polypeptide that comprises the CP1 and CM1 of the first monomer construct and a polypeptide that comprises the CP2 and CM2 of the second monomer construct of any one or combination of aspects 1-88.
103. A pair of vectors that together comprise the pair of nucleic acids of aspect 103.
104. The pair of vectors of aspect 104, wherein the pair of vectors is a pair of expression vectors.
105. A cell comprising the pair of nucleic acids of aspect 102 or the pair of vectors of aspects 104 or 105.
106. A method of producing an ACC comprising:
   culturing a cell of aspect 102 or 106 in a liquid culture medium under conditions sufficient to produce the ACC; and
   recovering the ACC from the cell or the liquid culture medium.
107. The method of aspect 107, further comprising:
   isolating the ACC recovered from the cell or the liquid culture medium.
108. The method of aspect 108, further comprising:
   formulating isolated ACC into a pharmaceutical composition.
109. An ACC produced by the method of aspect 107.
110. A composition comprising an ACC of aspect 110.
111. The composition of aspect 111, wherein the composition is a pharmaceutical composition.
112. A container, vial, syringe, injector pen, or kit comprising at least one dose of the composition of aspect 111 or 112.
113. An activatable cytokine construct (ACC) comprising a first monomer construct and a second monomer construct, wherein:
   (a) the first monomer construct comprises a first mature cytokine protein (CP1), a first cleavable moiety (CM1), and a first dimerization domain (DD1);
   (b) the second monomer construct comprises a second mature cytokine protein (CP2), a second cleavable moiety (CM2), and a second dimerization domain (DD2);
   (c) the first monomer construct is a polypeptide comprising, in an N- to C-terminal direction, the CP1, the CM1, and the DD1, further wherein:
      (i) the CP1 and the CM1 directly abut each other,
      (ii) the CM1 and the DD1 directly abut each other,
      (iii) the CM1 comprises no more than 10 amino acids and functions as a substrate for a protease, and
      (iv) the CP1 is a mature interferon;
   (d) further wherein:
      (i) the second monomer construct is the same as the first monomer construct,
      (ii) the first and second monomer constructs are covalently bound to each other via at least one disulfide bond, and
      (iii) the DD1 and the DD2 are a pair of human IgG Fc domains;
   (e) the DD1 and the DD2 bind each other thereby forming a dimer of the first monomer construct and the second monomer construct; and
   (f) the ACC is characterized by having a reduced level of interferon activity as compared to a corresponding control interferon.
114. The ACC of aspect 114, wherein the CP1 is a mature human interferon alpha.
115. The ACC of any one or combination of aspects 114-115, wherein the mature interferon is mature interferon alpha-2b.
116. The ACC of any one or combination of aspects 114-116, wherein the mature interferon is a truncated form of a wild type mature interferon alpha-2b.
117. The ACC of any one or combination of aspects 114-116, wherein the mature interferon comprises a sequence that is at least 95% identical to SEQ ID NO: 1.
118. The ACC of any one or combination of aspects 114-116, wherein the mature interferon alpha comprises the sequence of SEQ ID NO: 1.
119. The ACC of any one or combination of aspects 114-119, wherein the CM1 and the CM2 each comprises no more than 7 amino acids.
120. The ACC of any one or combination of aspects 114-120, wherein the CM1 and the CM2 each independently functions as a substrate of urokinase (uPa) and/or a matrix metalloproteinase (MMP).
121. The ACC of any one or combination of aspects 114-121, wherein the CM1 and the CM2 each independently functions as a substrate of urokinase (uPa) and/or MMP-14.
122. The ACC of any one or combination of aspects 114-122, wherein the CM1 and the CM2 each comprises a sequence that is at least 85% identical to SEQ ID NO: 100.

The ACC of any one or combination of aspects 114-123, wherein the CM1 and the CM2 each comprises a sequence selected from the group consisting of SEQ ID NO: 41, SEQ ID NO: 68, and SEQ ID NO: 100.

The ACC of any one or combination of aspects 114-124, wherein the DD1 and the DD2 are a pair of human IgG4 Fc domains.
123. The ACC of aspect 125, wherein the DD1 and the DD2 are a pair of human IgG4 Fc domains truncated at N-terminus to Cysteine 226 as numbered by EU numbering.

The ACC of aspect 125 or 126, wherein the human IgG4 Fc domains comprise a S228P mutation as numbered by EU numbering.

The ACC of any one or combination of aspects 114-127, wherein the DD1 and the DD2 each comprises a sequence that is at least 95% identical to SEQ ID NO: 3.

The ACC of any one or combination of aspects 114-128, wherein the DD1 and the DD2 each comprises a sequence of SEQ ID NO: 3.
124. The ACC of any one or combination of aspects 114-129, wherein the first and second monomer constructs are covalently bound to each other via at least two disulfide bonds.
125. The ACC of any one or combination of aspects 114-130, wherein the first and second monomer constructs are covalently bound to each other via at least three disulfide bonds.
126. The ACC of any one or combination of aspects 114-131, wherein the first and second monomer constructs are covalently bound to each other via at least four disulfide bonds.
127. The ACC of any one or combination of aspects 114-132, wherein the first monomer construct further comprises a signal sequence directly abutting the N-terminus of the CM1.
128. The ACC of aspect 133, wherein the signal sequence comprises a sequence that is at least 95% identical to SEQ ID NO: 345.
129. The ACC of aspect 133, wherein the signal sequence comprises the sequence of SEQ ID NO: 345.
130. The ACC of any one or combination of aspects 114-135 comprising a Linking Region comprising no more than 24 amino acids, no more than 18 amino acids, or no more than 12 amino acids.
131. The ACC of aspect 136, wherein the Linking Region comprises 7 to 12 amino acids.
132. The ACC of aspect 136, wherein the Linking Region comprises 7 amino acids.
133. The ACC of any one or combination of aspects 114-138, wherein the ACC is characterized by at least a 500-fold reduction in interferon activity as compared to a corresponding control interferon.
134. The ACC of any one or combination of aspects 114-139, wherein the CP1 is an interferon alpha and the control interferon is a recombinant interferon alpha.
135. The ACC of any one or combination of aspects 114-139, wherein the CP1 is an interferon alpha-2b and the control interferon is pegylated interferon alpha-2b.
136. The ACC of any one or combination of aspects 114-141, wherein the interferon activity is an anti-proliferation activity in lymphoma cells.
137. The ACC of any one or combination of aspects 114-141, wherein the interferon activity is induction of secreted embryonic alkaline phosphatase production in interferon-responsive HEK293 cells.
138. The ACC of any of aspects 114-143, wherein the ACC is further characterized by generating a cleavage product following exposure to the protease for which CM1 functions as a substrate, wherein the ratio of the interferon activity of the control interferon to the cleavage product is less than about 2, and wherein the control interferon is a corresponding recombinant wildtype interferon or a corresponding pegylated interferon.
139. The ACC of aspect 144, wherein the EC50 of the cleavage product is approximately the same as the EC50 of the corresponding recombinant wildtype interferon.
140. The ACC of aspect 114, wherein the first and second monomer constructs each comprises a sequence that is at least 95% identical to SEQ ID NO: 313.
141. The ACC of aspect 146, wherein the ACC is characterized by at least a 500-fold reduction in interferon activity as compared to wild type interferon alpha-2b, and wherein the ACC is further characterized by generating a cleavage product following exposure to uPA, wherein the cleavage product has approximately the same interferon activity as wildtype interferon alpha-2b, wherein interferon activity is measured in an anti-proliferation assay in lymphoma cells or in an assay of induction of secreted embryonic alkaline phosphatase production in interferon-responsive HEK293 cells.
142. The ACC of aspect 146, wherein the ACC exhibits lower toxicity in vivo compared to either wildtype interferon alpha-2b or PEGylated interferon alpha-2b.
143. An activatable cytokine construct (ACC) comprising a first monomer construct and a second monomer construct, wherein:
   (a) the first monomer construct comprises a first mature cytokine protein (CP1), a first cleavable moiety (CM1), and a first dimerization domain (DD1);
   (b) the second monomer construct comprises a second mature cytokine protein (CP2), a second cleavable moiety (CM2), and a second dimerization domain (DD2);
   (c) the first monomer construct is a polypeptide comprising, in an N- to C-terminal direction, the CP1, the CM1, and the DD1, further wherein:
      (i) the CP1 and the CM1 directly abut each other,
      (ii) the CM1 and the DD1 directly abut each other,
      (iii) the CP1 comprises a sequence that is at least 85% identical to SEQ ID NO: 1,
      (iv) the CM1 comprises a sequence that is at least 85% identical to SEQ ID: 100,
   (d) further wherein:
      (i) the second monomer construct is the same as the first monomer construct,
      (ii) the first and second monomer constructs are covalently bound to each other via at least one disulfide bond, and
      (iii) the DD1 and DD2 are a pair of human IgG4 Fc domains;
   (e) the DD1 and the DD2 bind each other thereby forming a dimer of the first monomer construct and the second monomer construct; and
   (f) the ACC is characterized by having a reduced level of interferon alpha activity as compared to the interferon alpha activity of PEGylated interferon alpha-2b.
144. A composition comprising the ACC of any one or combination of aspects 114-149.
145. The composition of aspect 150, where the composition is a pharmaceutical composition.
146. A container, vial, syringe, injector pen, or kit comprising at least one dose of the composition of aspect 150 or 151.
147. A method of treating a subject in need thereof comprising administering to the subject a therapeutically effective amount of the ACC of any one or combination of aspects 114-149 or the composition of aspect 150 or 151.
148. The method of aspect 153, wherein the subject has been identified or diagnosed as having a cancer.
149. A nucleic acid encoding a polypeptide that comprises the first monomer of the ACC of any one or combination of aspects 114-149.
150. A vector comprising the nucleic acid of aspect 155.
151. The vector of aspect 156, wherein the vector is an expression vector.
152. A mammalian cell comprising the nucleic acid of aspect 155 or the vector of aspect 156 or 157.
153. The mammalian cell of aspect 158, wherein the mammalian cell is an HEK293 cell or a CHO cell.
154. A method of manufacturing an ACC, the method comprising:
   a. expressing the ACC in the mammalian cell of aspect 158 or 159; and
   b. purifying the expressed ACC.
155. The ACC of any one or combination of aspects 114-149, wherein the CM1 functions as a substrate for a protease that is over-expressed in a tumor tissue.

### EXAMPLES

The invention is further described in the following examples, which do not limit the scope of the invention described in the claims.

### Example 1: Production of Activatable Cytokine Constructs

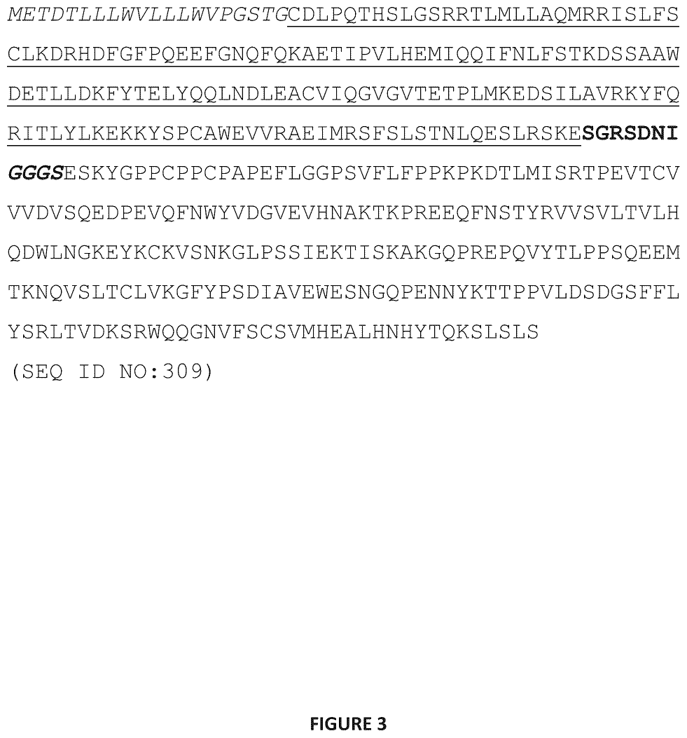

Activatable cytokine construct IFN-α2b-1204DNIdl-hIgG4 was prepared by recombinant methods. The 1^{st} and 2^{nd} monomer constructs of this ACC were identical, with each being a polypeptide having the amino acid sequence shown in FIG. 3 (SEQ ID NO:309). Each of the 1^{st} and 2^{nd} monomer constructs comprises, from N-terminus to C-terminus, a signal sequence from a mouse IgG kappa signal sequence (residues 1-20 of SEQ ID NO:309), a mature cytokine protein that corresponds to human interferon alpha-2b (SEQ ID NO:1), a cleavable moiety having the amino acid sequence of SEQ ID NO:99, a linker having the amino acid sequence, GGGS (SEQ ID NO:2), and a DD corresponding to human IgG Fc (SEQ ID NO:4). The polypeptide was prepared by transforming a host cell with a polynucleotide having the sequence of SEQ ID NO: 310, followed by cultivation of the resulting recombinant host cells. Dimerization of the resulting expressed polypeptides yielded activatable cytokine construct, IFN-α2b 1204DNIdl hIgG4.

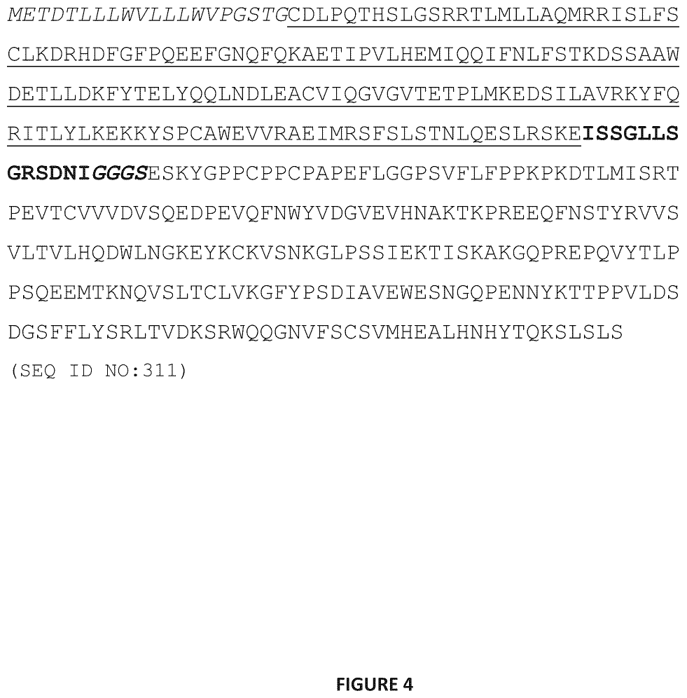

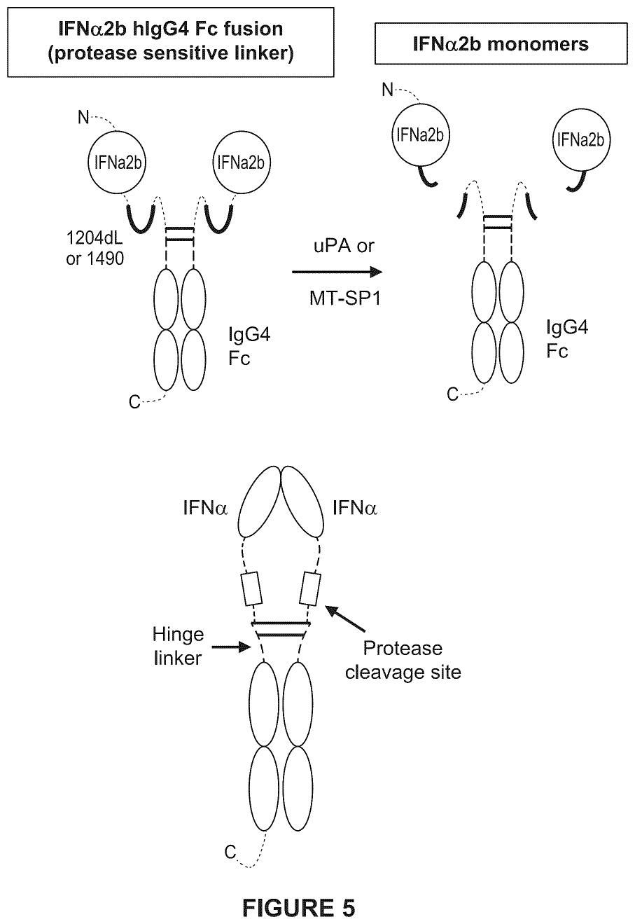

Activatable cytokine construct IFN-α-2b 1490DNI-hIgG4 was also prepared by recombinant methods. The 1^{st} and 2^{nd} monomer constructs of this ACC were also identical, with each being a polypeptide having the amino acid sequence shown in FIG. 4 (SEQ ID NO:311). Each of the 1^{st} and 2^{nd} monomer constructs of this ACC comprises, from N-terminus to C-terminus, a signal sequence from a mouse IgG kappa signal sequence (residues 1-20 of SEQ ID NO:309), a mature cytokine protein that corresponds to human interferon alpha-2b (SEQ ID NO:1), a cleavable moiety having the amino acid sequence of SEQ ID NO:68, a linker having the amino acid sequence, GGGS (SEQ ID NO:2), and a DD corresponding to human IgG Fc (SEQ ID NO:4). The polypeptide was prepared by transforming a host cell with a polynucleotide having the sequence of SEQ ID NO: 312, followed by cultivation of the resulting recombinant host cells. Dimerization of the resulting expressed polypeptides yielded activatable cytokine construct, IFN-α2b 1204dl hIgG4.

Additional activatable cytokine constructs were prepared that included an additional five amino acid residues in the linkers.

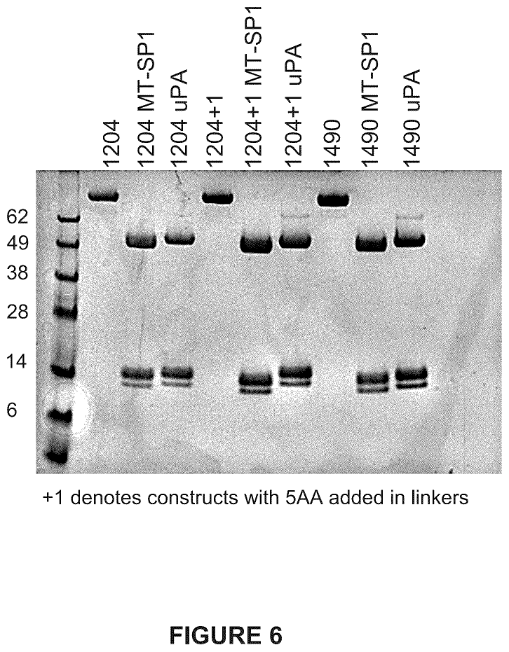

Electrophoresis was performed on the activatable cytokine constructs and protease-treated activatable cytokine constructs. FIG. 6 depicts the gel, which shows the results for (from left to right): (1) ACC IFN-α2b-1204DNIdl-hIgG4 (“1204”); (2) MT-SP1-treated IFN-α2b-1204DNIdl-hIgG4 (“1204 MT-SP1”); (3) uPA-treated IFN-α2b-1204DNIdl-hIgG4 (“1204 uPA”); (4) IFN-α2b-1204DNIdl-hIgG4 with five amino acid residues added to the linker (“1204+1”); (5) MT-SP1-treated IFN-α2b-1204DNIdl-hIgG4 (“1204+1 MT-SP1”); (6) uPA-treated IFN-α2b-1204DNIdl-hIgG4 (“1204+1 uPA”); (7) IFN-α-2b 1490DNI-hIgG4 (“1490”); (8) MT-SP1-treated IFN-α-2b 1490DNI-hIgG4 (“1490 MT-SP1”); and (9) uPA-treated IFN-α-2b 1490DNI-hIgG4 (“1490 uPA”). The results suggest that the proteases were effective at cleaving the cleavable moieties in the activatable cytokine constructs.

### Example 2. IFN-alpha-2b Activity of Activatable Cytokine Constructs

A cell-based reporter assay for human type I interferons was used to test the activity of the ACCs described in Example 1.

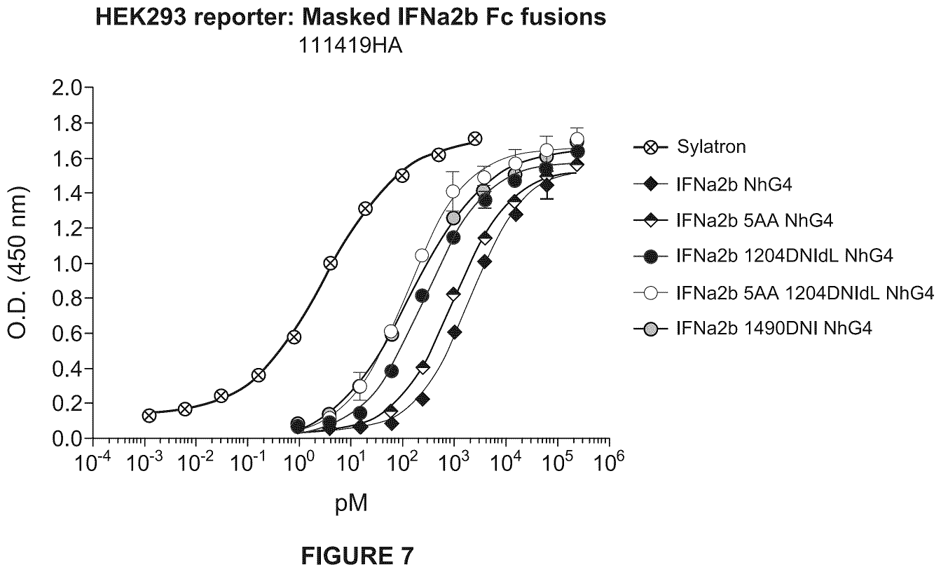

IFN-responsive HEK293 cells were generated by stable transfection with the human STAT2 and IRF9 genes to obtain a fully active type I IFN signaling pathway. The cells also feature an inducible SEAP (secreted embryonic alkaline phosphatase) reporter gene under the control of the IFNα/β inducible ISG54 promoter. To maintain transgene expression, cells were cultured in DMEM GlutaMax media supplemented with 10% FBS, Pen/Strep, 30 μg/mL of blasticidin, 100 μg/ml of zeocin and 100 μg/mL of normocin. The addition of type I IFN to these cells activates the JAK/STAT/ISGF3 pathway and subsequently induces the production of SEAP which can be readily assessed in the supernatant using Quanti-Blue solution, a colorimetric detection for alkaline phosphatase activity. Using this reporter assay, the activity of IFNα-2b containing ACCs was compared to the activity of Sylatron® (Peginterferon alfa-2b). The data in FIG. 7 show that IFNα-2b activity of the ACCs was significantly reduced as compared to the IFNα-2b activity of Sylatron® (Peginterferon alfa-2b).

Furthermore, the data in FIGS. 8A and 8B show that the activity of the (uncleaved) ACCs could be modulated by varying the length of the linker or Linking Region. The data in FIG. 8A-8B show the results of IFNa-2b-hIgG4 Fc fusion constructs with varying linker lengths, or without a linker between the IFNa-2b and the hIgG4 Fc as tested in the HEK293 reporter assay. The fusion proteins tested in this experiment include, in an N- to C-terminal direction, the mature IFNalpha-2b cytokine sequence, an optional linker and/or cleavable moiety, and the Fc domain of human IgG4 of SEQ ID NO: 4 (including the full hinge region such that the N-terminus of the Fc sequence begins with the amino acid sequence ESKYGPPCPPC . . . ). The first construct (Linking Region=7) has no linker or cleavable moiety; its sequence in the N- to C-terminal direction consists of SEQ ID NO: 1 fused to SEQ ID NO: 4. The second construct (Linking Region=12) has a 5 amino acid linker SGGGG (SEQ ID NO: 335); its sequence in the N- to C-terminal direction consists of SEQ ID NO: 1 fused to SEQ ID NO: 335 fused to SEQ ID NO: 4. The third construct (Linking Region=18) includes a 7 amino acid CM (SGRSDNI) and a 4 amino acid linker GGGS; its sequence in the N- to C-terminal direction consists of SEQ ID NO: 1 fused to SEQ ID NO: 100 fused to SEQ ID NO: 2 fused to SEQ ID NO: 4. The fourth construct (Linking Region=23) includes a 5 amino acid linker, a 7 amino acid CM, and a 4 amino acid linker; its sequence in the N- to C-terminal direction consists of SEQ ID NO: 1 fused to SEQ ID NO: 335 fused to SEQ ID NO: 100 fused to SEQ ID NO: 2 fused to SEQ ID NO: 4. The fifth construct (Linking Region=24) includes a 13 amino acid CM (ISSGLLSGRSDNI) and a 4 amino acid linker; its sequence in the N- to C-terminal direction consists of SEQ ID NO: 1 fused to SEQ ID NO: 68 fused to SEQ ID NO: 2 fused to SEQ ID NO: 4.

### Example 3: In Vitro Anti-Proliferative Effect of ACCs on Cancer Cells

The anti-proliferative effects of IFNα-2b and IFNα-2b-containing ACCs were tested in vitro using Daudi cells, a cell line of human B-cell lymphoblastic origin. Daudi cells were prepared at a concentration of 2×10⁵ cells/mL in RPMI-1640 media supplemented with 10% FBS and 50 μL aliquots were pipetted into wells of a white flat-bottom 96-well plate (10K/well). The tested ACCs or controls were diluted in RPMI 1640 media supplemented with 10% FBS. Duplicate five-fold serial dilutions were generated from which 50 μL was added to the each well. After 3 days of incubation at 37° C., a viability kit was used to measure the levels of intracellular ATP as an indirect estimate of the number of viable cells remaining. 100 μL of cell-titer go was directly added to the plates which were then placed on an orbital shaker for 10 minutes. Following this incubation, the luminescent signal was directly measured using an Envision plate reader. Dose-response curves were generated and EC50 values were obtained by sigmoidal fit non-linear regression using Graph Pad Prism software. Specific activities were determined by comparison of EC50 values with recombinant IFNα2b or pharmaceutical-grade Sylatron® (Peginterferon alfa-2b).

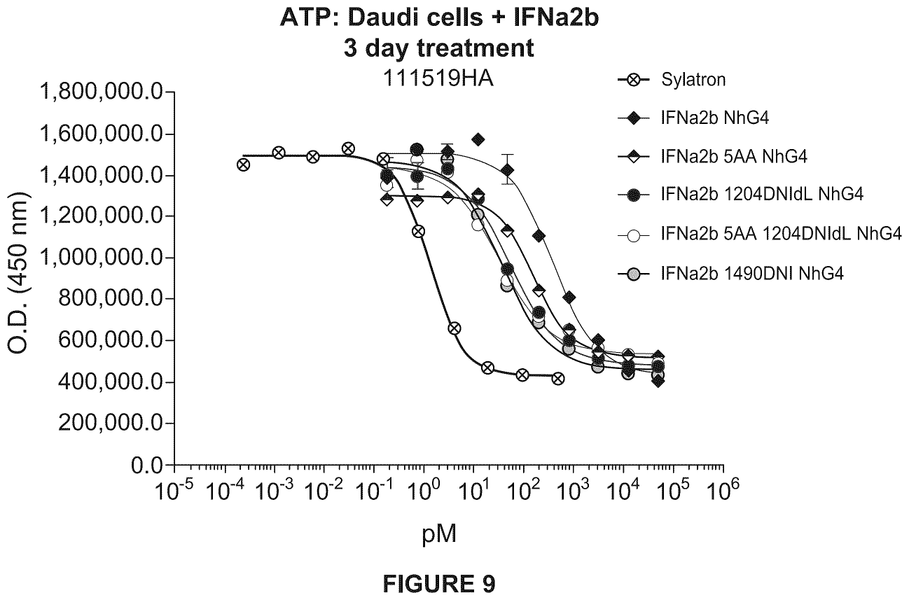

The anti-proliferative activity of IFNα-2b-containing ACCs in Daudi lymphoma cells indicated that the IFNα-2b activity of the uncleaved ACCs was decreased compared to Sylatron® (Peginterferon alfa-2b) (FIG. 9).

The data in FIGS. 10A-10B also shows that the activity of the (uncleaved) ACCs could be modulated by varying the length of the linker. The anti-proliferative effects of IFNa-2b-hIgG4 Fc fusion protein constructs with varying linker lengths or without a linker between the IFNa-2b and the hIgG4 Fc were tested in vitro using Daudi cells. The data indicate that the length of the flexible linkers and the length of the Linking Region (LR) between the cytokine and the Fc domain had an impact on the activity of the (uncleaved) ACCs. Constructs with zero linkers, or short linkers, and a correspondingly short LR display reduced cytokine activity, whereas constructs with longer linkers and thus a longer LR have a higher level of cytokine activity. The results are shown in FIGS. 10A-10B. The fusion protein constructs are the same as those described above in Example 2 with respect to FIGS. 8A and 8B.

### Example 4: Activity of Protease-Treated ACCs

Protease treated IFNα-2b-containing ACCs were tested for anti-proliferative responses in Daudi lympho cells and in the cell-based reporter assay to determine if the activity could be restored.

To cleave the dimerizing domain, IFNα-2b-containing ACCs were treated overnight at 37° C. with recombinant human proteases such at urokinase-type plasminogen activator (uPA), or matriptase (MT-SP1). A cocktail of protease inhibitors were added to neutralize the proteases prior to testing for activity as described in Example 2 and 3. The results from these assays indicate that the treatment of IFNα-2b-containing ACCs with proteases could restore activity to a level that is comparable to the recombinant cytokine. EC50 values for ACC IFNα-2b-1204DNIdl-hIgG4, ACC IFNα-2b-1204DNIdl-hIgG4+uPA, and Stem Cell IFNα-2b (human recombinant IFN-alpha 2b, available from StemCell Technologies, Catalog #78077.1) were computed from the Daudi apoptosis assay results, and are provided below in Table 3.

TABLE 3
EC50: Daudi Apoptosis Assay

| | IFNα-2b- | IFNα-2b- | |
|---|---|---|---|
| | 1204DNIdl-hIgG4 | 1204DNIdl-hIgG4 | Stem Cell |
| | (ACC) | (ACC) + uPA | IFNα-2b |
| EC50 | 131.8 | 0.5701 | 0.3664 |

EC50 values for ACC IFNα-2b-1204DNIdl-hIgG4, ACC IFNα-2b-1204DNIdl-hIgG4+uPA, and Stem Cell IFNα-2b were computed from the IFNα/β assay results, and are provided below in Table 4.

TABLE 4
EC50: IFNα/β Reporter Assay

| | | IFNα-2b- | | |
|---|---|---|---|---|
| | IFNα-2b- | 1204DNIdl- | | |
| | 1204DNIdl- | hIgG4 (ACC) + | | Commercial |
| | hIgG4 (ACC) | uPA | Sylatron ® | IFNα-2b |
| EC50 | 393.1 | 0.4611 | 3.019 | 1.280 |

These results show that without the presence of an activating protease, the activity of IFNα-2b-1204DNIdl-hIgG4 is significantly decreased relative to the IFNα-2b control.

### Example 5: Universal ProIFN

An ACC according to the present disclosure was prepared by recombinant methods having a universal interferon sequence (ProC859) (IFNaAD 0AA 1204DNIdL 0AA IgG4) having activity on both human and mouse cells. The 1^{st} and 2^{nd} monomer constructs of this ACC were identical, with each being a polypeptide having the amino acid sequence (SEQ ID NO: 323 and a signal sequence at its N-terminus). Each of the 1^{st} and 2^{nd} monomer constructs comprises, from N-terminus to C-terminus, a signal sequence, a mature cytokine protein that corresponds to a universal interferon molecule that is a hybrid of IFN alpha 1 and IFN alpha 2a (SEQ ID NO: 324), a cleavable moiety having the amino acid sequence of SEQ ID NO: 100, and a dimerization domain corresponding to human IgG Fc (SEQ ID NO: 3). The activity of the universal ProIFN was tested in vitro using IFN-responsive HEK293 cells and B16 mouse melanoma cells.

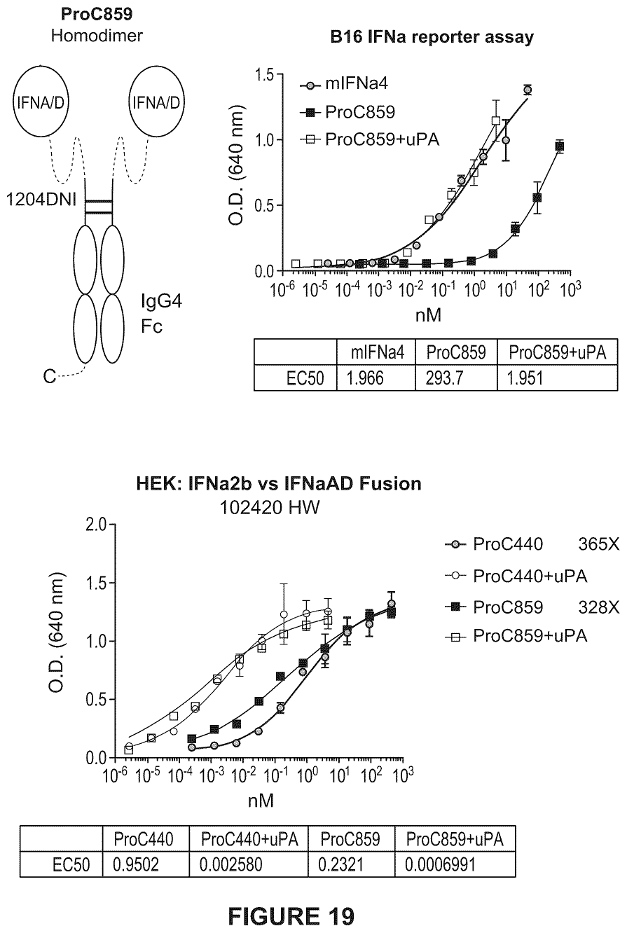

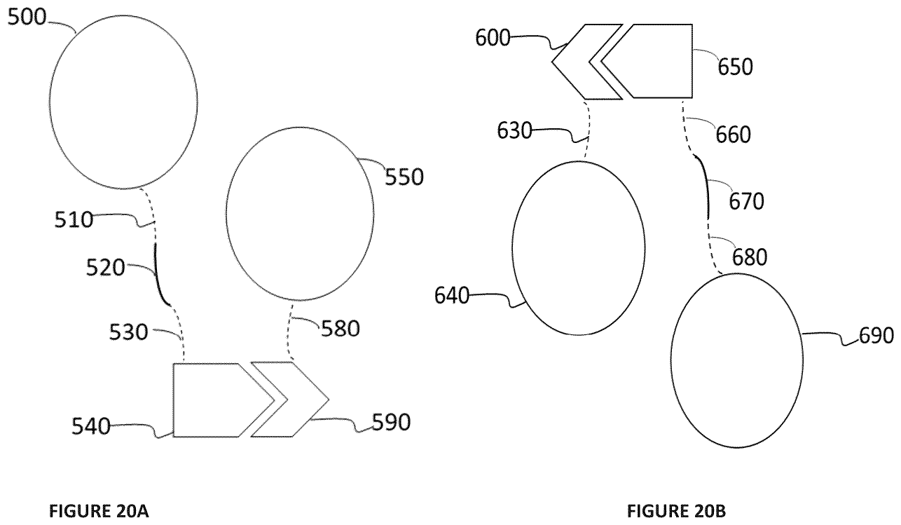

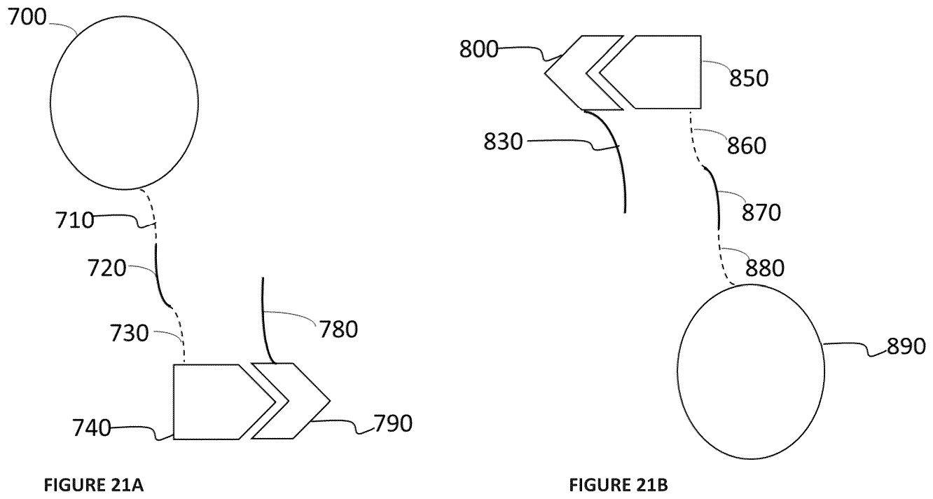

The activity of ProC859 was reduced at least 150× as compare to mouse IFNa4. Protease activation with uPa restored activity to a level that is comparable to mouse IFNa4 as shown in FIG. 19). EC50 values for ACC ProC859, ACC ProC859+uPA, and mouse IFNα4 were computed from the assay results and are provided in FIG. 19.

EC50: B16 IFNα/β Reporter Assay

| | ProC859 | ProC859 | |
|---|---|---|---|
| | (ACC) | (ACC) + uPA | IFNa4 |
| EC50 | 293.7 | 1.951 | 1.966 |

### Example 6: In Vitro Characterization of Lead ACC ProC440

Activatable cytokine construct ProC 440 (N IFNa2b 0 1204DNIdL 0AA Fc) was also prepared by recombinant methods. The 1^{st} and 2^{nd} monomer constructs of this ACC were identical, with each being a polypeptide having the amino acid sequence of SEQ ID NO: 313 and a signal sequence at its N-terminus. Each of the 1^{st} and 2^{nd} monomer constructs comprises, from N-terminus to C-terminus, a signal sequence, a mature cytokine protein that corresponds to human interferon alpha-2b (SEQ ID NO: 1), a cleavable moiety having the amino acid sequence of SEQ ID NO: 100, and a dimerization domain corresponding to human IgG Fc (SEQ ID NO: 3).

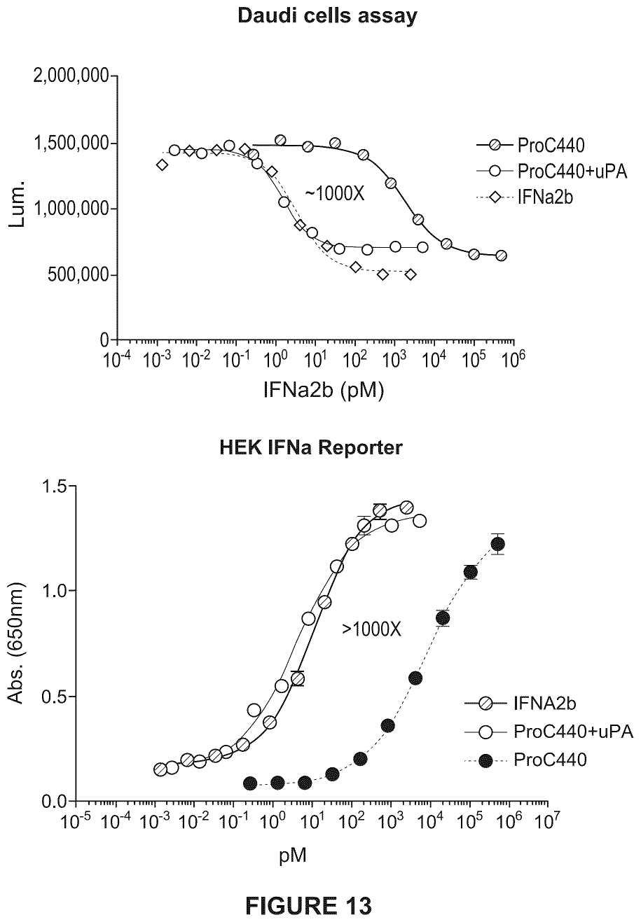

The activity of ProC440 was tested in vitro using IFN-responsive HEK293 cells and Daudi cells as previously described. In both assays, the activity of ProC440 was reduced at least 1,000× as compared to Stem Cell IFNα-2b (FIG. 13). Protease activation with uPa restored activity to a level that is comparable to the recombinant cytokine (IFNa2b as shown in FIG. 13). EC50 values for ACC ProC440, ACC ProC440+uPA, and Stem Cell IFNα-2b were computed from the IFNα/β assay results and are provided below in Table 5.

TABLE 5
EC50: IFNα/β Reporter Assay

| | ProC440 | ProC440 | Stem Cell |
|---|---|---|---|
| | (ACC) | (ACC) + uPA | IFNα-2b |
| EC50 | 7643 | 4.333 | 10.88 |

EC50 values for ACC ProC440, ACC ProC440+uPA, and Stem Cell IFNα-2b were computed from the Daudi apoptosis assay results and are provided below in Table 6.

TABLE 6
EC50: Daudi Apoptosis Assay

| | ProC440 | ProC440 | Stem Cell |
|---|---|---|---|
| | (ACC) | (ACC) + uPA | IFNα-2b |
| EC50 | 264.2 | 0.1842 | 0.3530 |

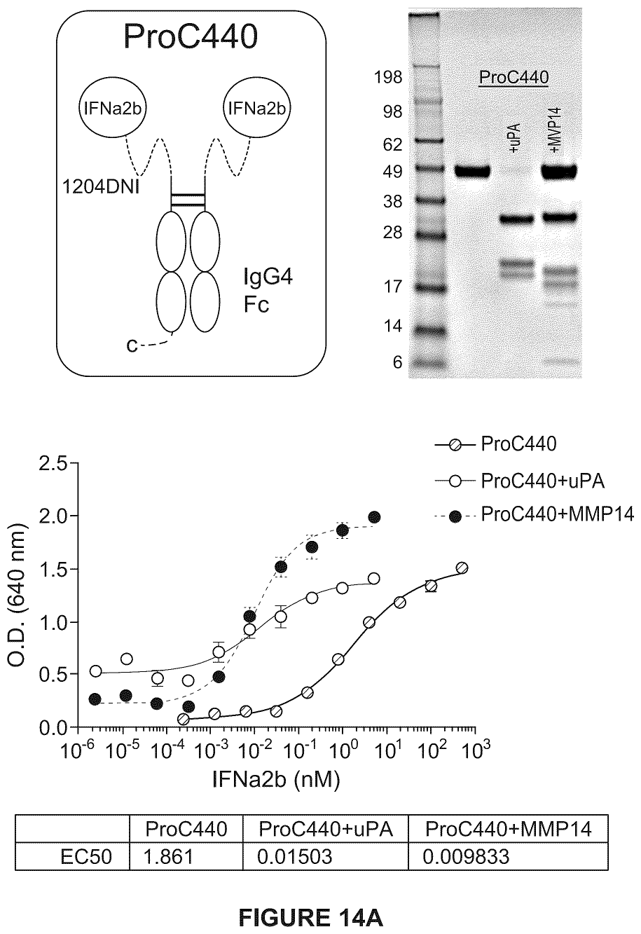

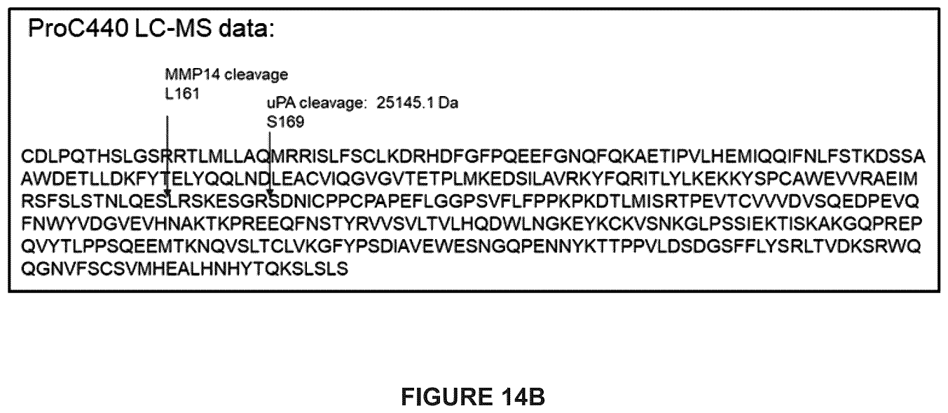

Cleavage with uPa at the expected site in the CM was confirmed by Mass spectrometry analysis (FIGS. 14A-14B). In addition to sensitivity to uPa activation, ProC440 is cleaved by MMP4 (FIGS. 14A-14B). Analysis by Mass spectrometry identified a MMP14 cleavage site at the C-terminal extremity of IFNa, near the cleavable moiety (FIG. 14B). Protease activation with MMP14 restored activity to a level that is comparable to the recombinant cytokine. All together, this indicates that ACC ProC440 can recover full activity after cleavage of intrinsic and engineered cleavable moieties by at least uPa and MMP14.

ACC ProC657 (N IFNa2b 0AA 1204DNIdL 0AA IgG4 KiHSS) was also prepared by recombinant methods. The 1^{st} monomer construct of this ACC is a polypeptide having the amino acid sequence of SEQ ID NO: 314 and a signal sequence at its N-terminus. The 1^{st} monomer construct of this ACC comprises, from N-terminus to C-terminus, a signal sequence, a mature cytokine protein that corresponds to human interferon alpha-2b (SEQ ID NO: 1), a cleavable moiety having the amino acid sequence of SEQ ID NO: 68, and a dimerization domain corresponding to human IgG Fc with a knob mutation (SEQ ID NO: 315). The 2^{nd} monomer construct of this ACC is a polypeptide having the amino acid sequence of SEQ ID NO: 322 and a signal sequence at its N-terminus. The 2^{nd} monomer construct has, from N-terminus to C-terminus, a signal sequence, a stub moiety (SEQ ID NO: 317), and a dimerization domain corresponding to human IgG Fc with a hole mutation (SEQ ID NO: 316).

The activity of ProC657 was tested in vitro using IFN-responsive HEK293 cells as previously described. The activity of ProC657 was reduced as compare to Stem Cell IFNα-2b or uPa-activated ProC440 but increased as compared to ProC440 (FIG. 15). Thus, the present disclosure provides different structures of ACCs that make it possible to modulate levels of reduction in activity in the ACCs.

### Example 7: In Vivo Antiproliferative Activity of ACCs

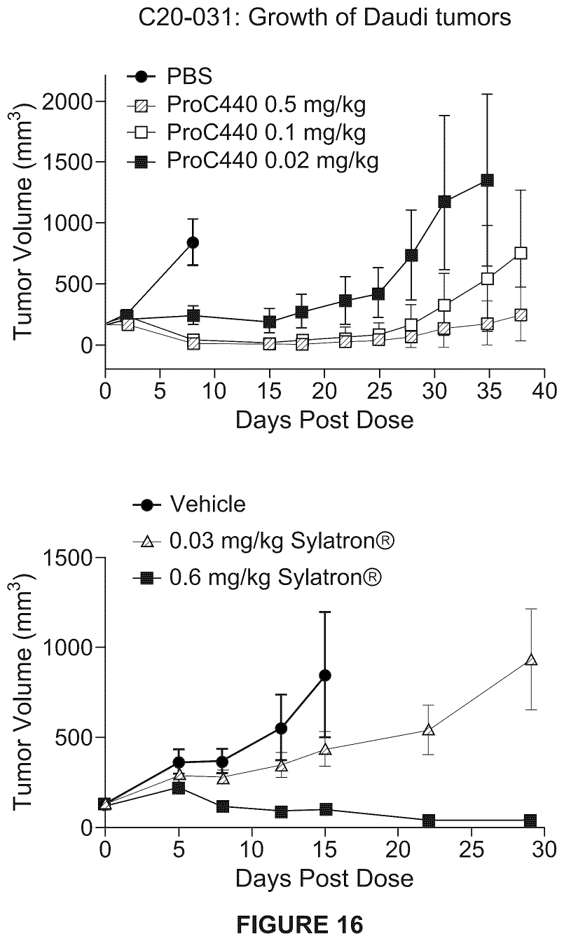

The anti-proliferative effects of IFNα-2b-containing ACC ProC440 was tested in vivo using the Daudi xenograft tumor model. Beige/SCID mice were implanted subcutaneously with 10×10⁶ Daudi cells in serum-free medium (1:1 Matrigel). When the average tumor volume reached ˜60-120 mm³, mice were randomized and dosed once a week for 5 weeks with ProC440. Body weights and tumor measurements were recorded twice weekly for the duration of the study. The data in FIG. 16 shows that IFNα-2b-containing ACC ProC440 induced complete tumor regression at a dose as low as 0.1 mg/kg and slowed-down tumor growth at a dose of 0.02 (top) and the anti-proliferative effects of Sylatron® are shown for comparison (bottom).

### Example 8: In Vivo Tolerability Activity of ACCs

Human IFNα-2b cross react with hamster IFNα receptor and has been previously shown to be active in Hamster (Altrock et al, Journal of Interferon Research, 1986). To assess the tolerability of IFNα-2b-containing ACC ProC440, Syrian Gold Hamsters were dosed with a starting dose of 0.4 mg/kg. Animals received one dose of test article and kept on study up to 7 days post dose, unless non tolerated toxicities (DLT means dose limiting toxicities) were identified. The starting dose (0.4 mg/kg (“mpk”)) represents an equivalent dose of INFα-con (recombinant interferon alpha, a non-naturally occurring type-I interferon manufactured by Amgen under the name Infergen®) expected to induce body weight loss, decreased food consumption and bone marrow suppression in a hamster (125 gr). (In cynomolgus monkeys (cyno), 0.1 mg/kg/day of INFα-con has been associated with body weight lost, decreased food consumption and bone marrow suppression (equal to 1.25-2.5×10\hat{ }7 U for a 125 gram hamster).) If the starting dose was tolerated, animals were moved up to a “medium dose” of 2 mg/kg and received three doses of test article unless not tolerated. If tolerated, animals were moved up to a “high dose” of 10 mg/kg and received three doses of test article unless not tolerated. If tolerated, animals were moved up to a “higher dose” of 15 mg/kg. At each stage, if the test dose was not tolerated, the animal was moved down to the next lower dose. If the starting dose was not tolerated, the animal was moved down to a “lower dose” of 0.08 mg/kg. Animals were dosed with an ACC having a N- to C-terminus structure of DD-CM-CP dimers (ProC286). As a negative control, animals were dosed with a human IgG4. The negative control did not induce any toxicity in the animals, as expected.

ProC286 (ChIgG4 5AA 1204DNIdL IFNa2b) was also prepared by recombinant methods. The 1^{st} and 2^{nd} monomer constructs were identical, with each being a polypeptide having the amino acid sequence of SEQ ID NO: 320 and a signal sequence at its N-terminus. Each of the 1^{st} and 2^{nd} monomer constructs comprises, from N-terminus to C-terminus, a signal sequence, a dimerization domain corresponding to human IgG Fc (SEQ ID NO: 3), a linker (SEQ ID NO: 321) a cleavable moiety having the amino acid sequence of SEQ ID NO: 100, a linker (SEQ ID NO: 2), and a mature cytokine protein that corresponds to human interferon alpha-2b (SEQ ID NO: 1).

ProC291 (NhIgG4 5AA 1204DNIdL IFNa2b) was also prepared by recombinant methods. The 1^{st} and 2^{nd} monomer constructs were identical. Each of the 1^{st} and 2^{nd} monomer constructs comprises, from N-terminus to C-terminus, a mature cytokine protein that corresponds to human interferon alpha-2b (SEQ ID NO: 1), a linker (SEQ ID NO: 321), a CM (SEQ ID NO: 100), a linker (GGGS), and a human IgG4 Fc region including the full hinge sequence (SEQ ID NO: 4).

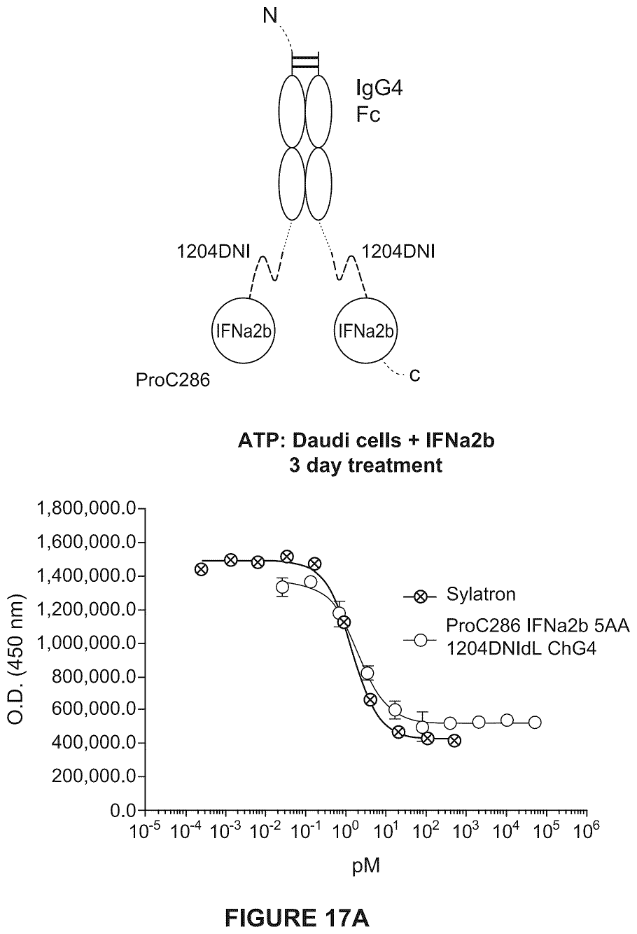

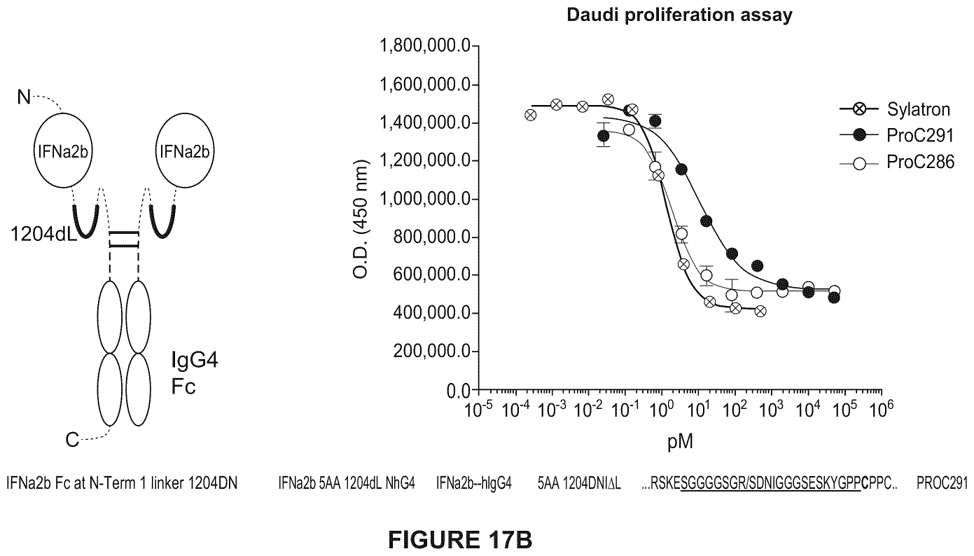

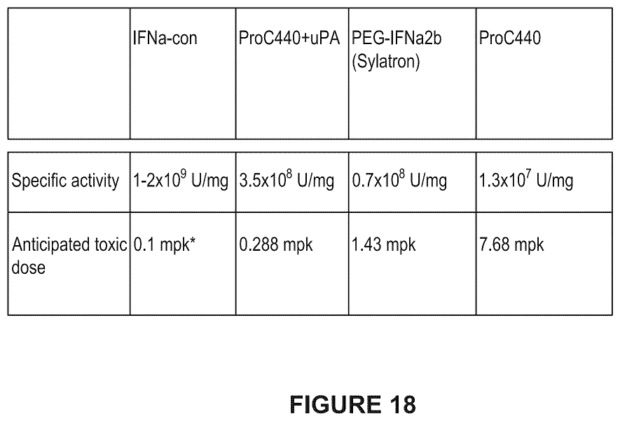

The activity of ProC286 and ProC291 were compared to the activity of Sylatron® (PEG-IFN-alpha2b) in the Daudi apotosis assay (FIGS. 17A-17B). In this assay, ProC286 and Sylatron® showed similar levels of activity as shown in FIG. 17A This indicates that ProC286 has similar activity to commercially-available pegylated IFN-alpha2b, and could be used as surrogate Sylatron® control to evaluate the tolerability of IFNα-2b in the hamster study. ProC291 showed reduced activity compared to ProC286 and Sylatron®, indicating that the structural orientation of the IFN N-terminal to the Fc was important for reduction in activity. That is, when the DD is a pair of Fc domains, positioning the cytokine N-terminal to the DD (as in ProC291) may provide greater reduction of cytokine activity than when the cytokine is positioned C-terminal to the DD (as in ProC286).

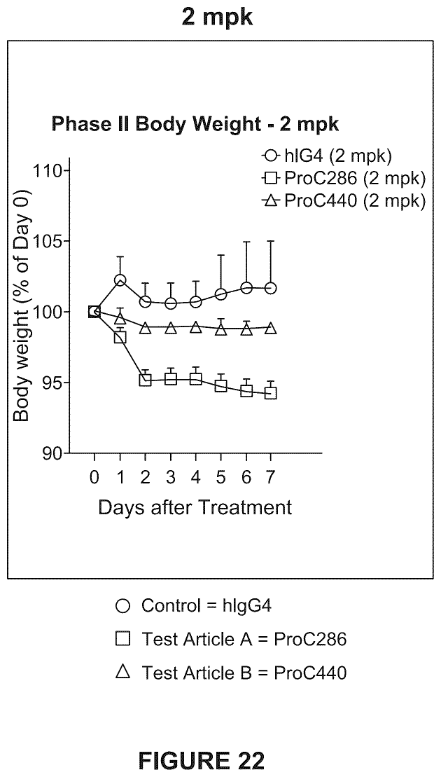

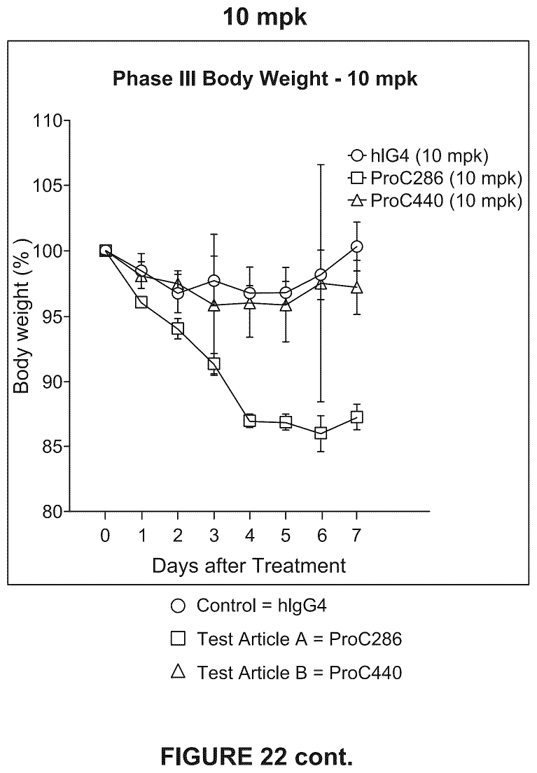

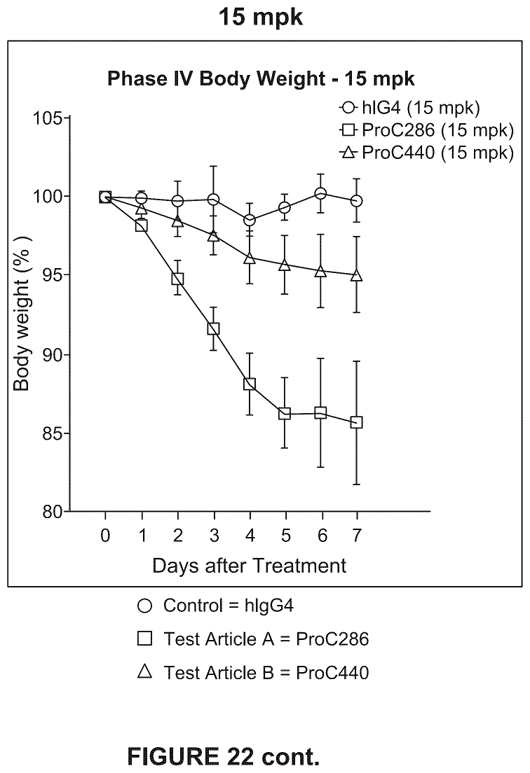

Animals were dosed on day 1 with the 0.4 mg/kg starting dose. Animals were kept on study for one week, unless a non-tolerated dose (DLT) was reached. Clinical observations, body weights & temperatures were measured prior to dosing, and at 6 h, 24 h, 72 h, and 7 d post-dose for each animal. Blood samples for Hematology and Chemistry analysis were collected at 72 h, 7 d post-dose for each animal. Hematology and Chemistry analysis were performed right after sampling. For the Hematology analysis, blood smear, differential white blood cell count, hematocrit, hemoglobin, mean corpuscular hemoglobin, mean corspucuar volume, platelet count, red blood cell (erythrocyte) count, red blood cell distribution width, reticulocyte count and white blood cell (leukocyte) count were evaluated. The clinical chemistry panel included measurement of alanine aminotransferase, albumin, albumin/globulin ratio, alakaline phosphatase, aspartate aminotransferase, calcium, chloride, cholesterol, creatine kinase, creatine, gamma glutamytransferase, globulin, glucose, inorganic phosphorus, potassium, sodium, total bilirubin, total protein, triglycerides, urea, nitrogen, and C-reactive protein. The evidence of toxicities in the tolerability study are summarized in FIGS. 22-24.

Overall, animals dosed with the unmasked ProC286 constructs showed on average 5% body weight loss at when dosed at 2mpk, and 15% body weight loss when dosed at 10mpk and 15mpk (FIG. 22). One animal dosed with ProC286 at 15mpk showed 20% body weight loss 7 days post-dose (end of study). This is considered a non-tolerated dose. In contrast, animals dosed with ProC440 at 2mpk and 10mpk did not show body weight loss.

Animals dosed with ProC440 at 15mpk showed on average 5% body weight loss (FIG. 22). This indicates that ACCs of the present disclosure with a dimerized structure of, starting at the N-terminus, CP-CM-DD unexpectedly limits IFNα-2b mediated bodyweight loss. Without wishing to be bound by theory, it is believed that positioning the interferon N-terminal of the DD and using a relatively short LR inhibits cytokine activity in the context of ProC440, reducing the toxicity of the interferon in comparison to PEGylated IFNα-2b (Sylatron®) or ProC286.

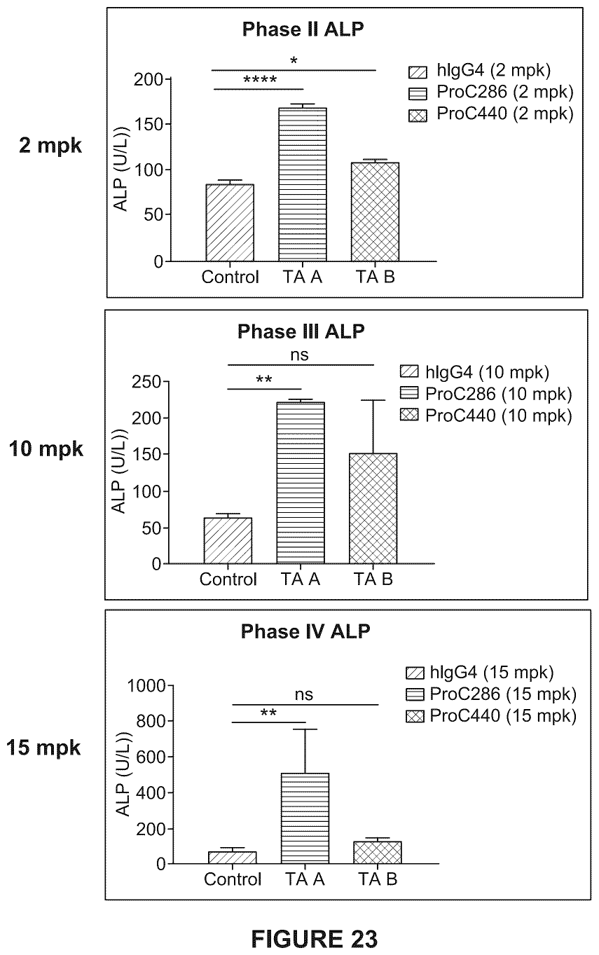

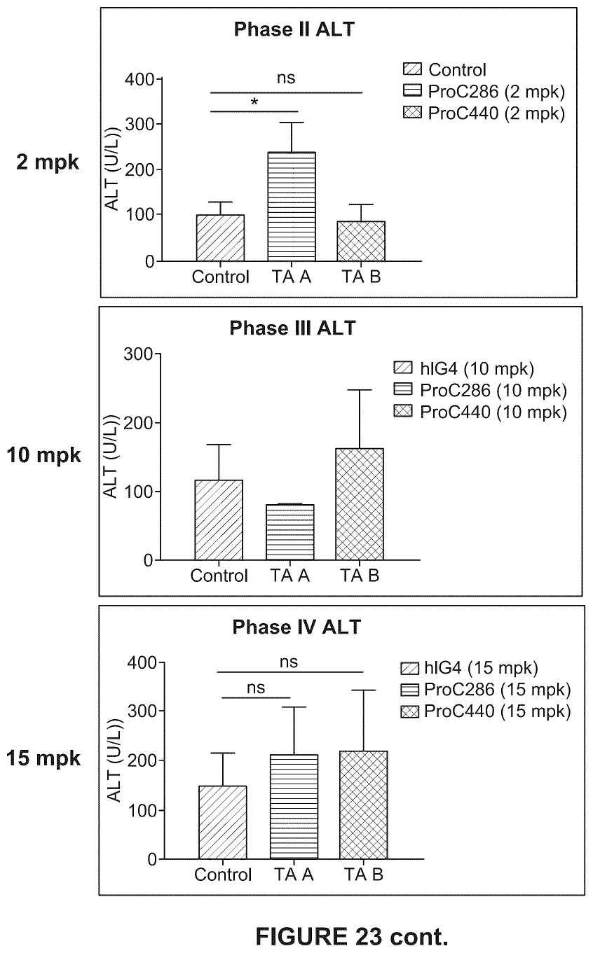

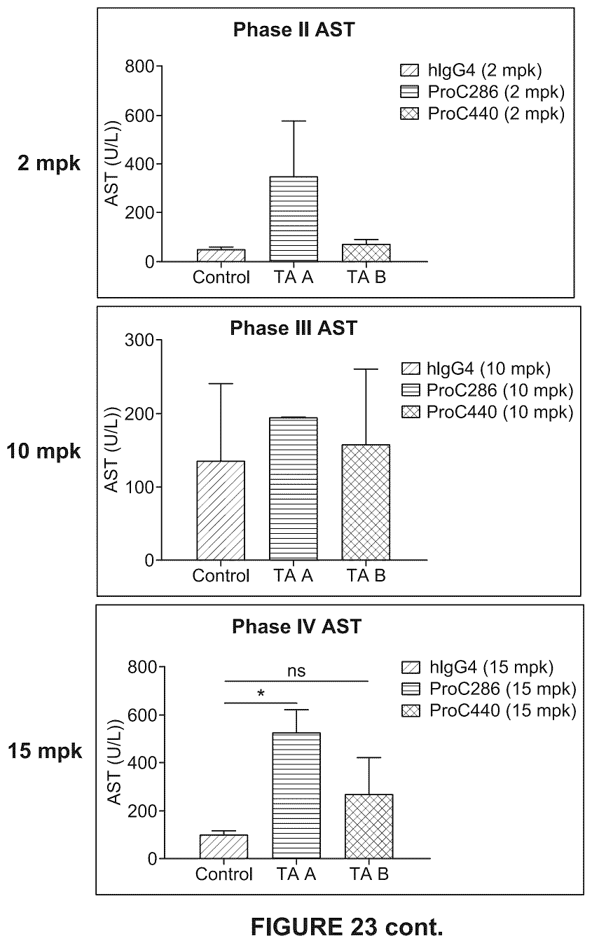

In terms of clinical chemistry, animals dosed with ProC286 showed significant elevation of Alkaline Phosphatase (ALP) at all doses (0.4mpk, 2mpk, 10 mpk and 15mpk), 7 days post-dose (end of study) (FIG. 23). No significant increase of ALP was measured when animals were dosed with 10 mpk or 15mpk of ProC440 (FIG. 23). Elevation of ALT is a marker of liver toxicity. IFNα-2b has been shown to induce liver toxicities. This indicates that ACCs of the present disclosure with a dimerized structure of, starting at the N-terminus, CP-CM-DD unexpectedly limits IFNa-2b mediated liver toxicities.

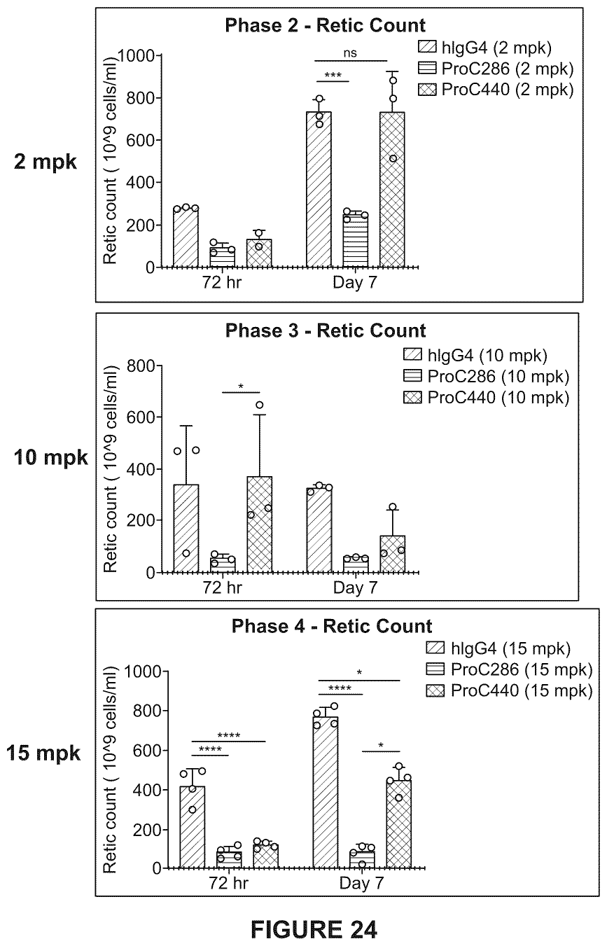

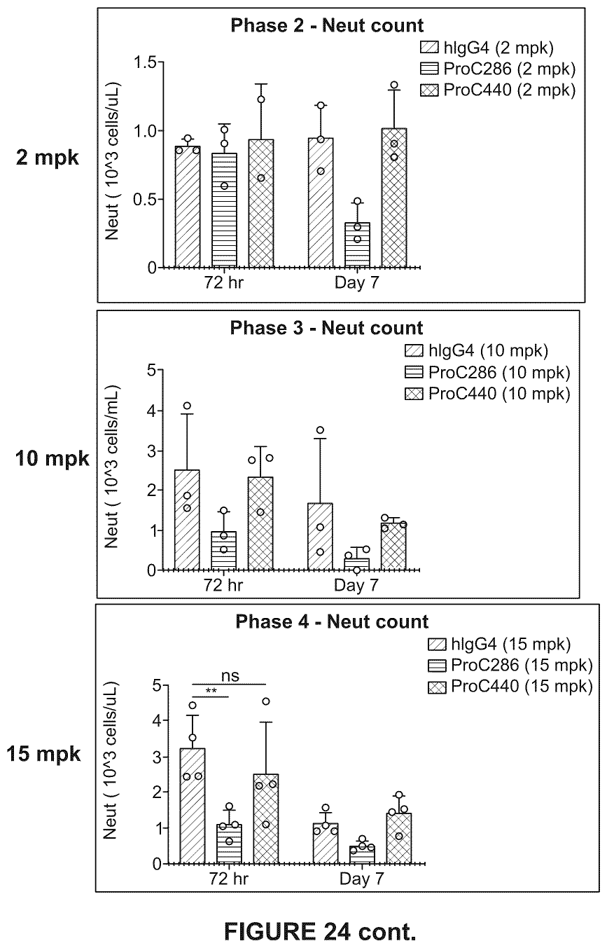

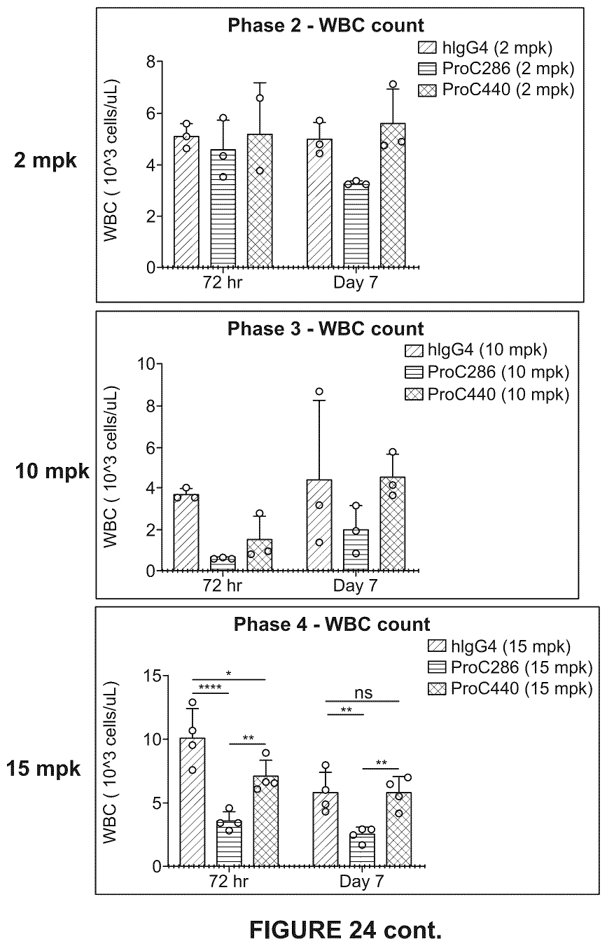

In terms of hematology, 3 days post-dose and 7 days post-dose (end of study), animals dosed with ProC286 at 2mpk, 10 mpk and 15mpk showed significant reduction level of Reticulocyte count, Neutrophil count and White Blood Cells (WBC) count (FIG. 24). These reductions are reminiscent of IFNa-2b mediated bone-marrow toxicities. Three days post-dose, animals dosed with ProC440 showed reduction level of Reticulocyte count, Neutrophil count and White Blood Cells (WBC) count (FIG. 24). Overall, the reduction level of hematopoetic cells observed in animals dosed with ProC440 is not as significant as the reduction levels observed in animals dosed with ProC286. At 7 days post-dose (end of study), in animals dosed with ProC440, the overall level of Reticulocyte count, Neutrophil count and White Blood Cells (WBC) count is back to normal levels, or to a similar level that what observed in animals dosed with the negative control IgG4 (FIG. 24). In animals dosed with ProC286, the level of Reticulocyte count, Neutrophil count and White Blood Cells (WBC) count remains low. This indicates that ACCs of the present disclosure with a dimerized structure of, starting at the N-terminus, CP-CM-DD unexpectedly limits IFNa-2b mediated bone marrow toxicities.

### Example 4. In Vitro Characterization of Additional Cytokine Constructs

Additional activatable cytokine constructs were also prepared by recombinant methods. The 1^{st} and 2^{nd} monomer constructs of these ACCs were identical. Each of the 1^{st} and 2^{nd} monomer constructs comprises, from N-terminus to C-terminus, a signal sequence from a mouse IgG kappa signal sequence (residues 1-20 of SEQ ID NO: 309), a mature cytokine protein that corresponds to human interferon alpha-2b (SEQ ID NO: 1), a cleavable moiety (CM) having the amino acid sequence of SEQ ID NO: 100, and a dimerization domain corresponding to human IgG4 S228P Fc (comprising SEQ ID NO: 3). In addition, these ACCs include or not a linker having the amino acid sequence SGGGG between the CP and the CM. These ACCs include or not a linker having the amino acid sequence GGGS between the CM and DD. These ACCs also contain or not portions of the hinge of the DD that are N-terminal to Cysteine 226. These additional activable cytokines constructs are described in Table 6 (see SEQ ID Nos: 336 to 342 and SEQ ID NO: 313).

TABLE 6
Activable cytokines having different lengths
of amino acid sequences between CP and
Cysteine 226 of human IgG

| | | | | Fc | |
|---|---|---|---|---|---|
| | | Link- | Link- | Hinge | LINK- |
| | | er be- | er be- | N-ter- | ING |
| | Alter- | tween | tween | minal | RE- |
| | native | CP and | CM and | resi- | GION |
| Name | Name | CM | DD | dues | LENGTH |
| ProC288 | IFNa2b | SGGGG | absent | absent | 12 |
| | 1204DNI | | | | |
| | 0AA Fc | | | | |
| ProC289 | IFNa2b | SGGGG | absent | GPP | 15 |
| | 1204DNI | | | | |
| | 3AA Fc | | | | |
| ProC290 | IFNa2b | SGGGG | absent | ESKYGPP | 19 |
| | 1204DNI | | | | |
| | 7AA Fc | | | | |
| ProC291 | IFNa2b | SGGGG | GGGS | ESKYGPP | 23 |
| | 1204DNI | | | | |
| | 11AA Fc | | | | |
| ProC440 | N IFNa2b 0 | absent | absent | absent | 7 |
| | 1204DNIdL | | | | |
| | 0AA Fc | | | | |
| ProC441 | N IFNa2b 0 | absent | absent | GPP | 10 |
| | 1204DNIdL | | | | |
| | 3AA Fc | | | | |
| ProC442 | N IFNa2b 0 | absent | absent | ESKYGPP | 14 |
| | 1204DNIdL | | | | |
| | 7AA Fc | | | | |
| ProC443 | N IFNa2b 0 | absent | GGGS | ESKYGPP | 18 |
| | 1204DNIdL | | | | |
| | 11AA Fc | | | | |

The activity of ProC440, an ACC with no flexible linker and an Fc region truncated to Cys226, and the activity of additional ACCs containing various linkers and Fc region sequences was tested in vitro using IFN-responsive HEK293 cells and Daudi cells as previously described. In both assays, the activity (e.g., anti-proliferative effects) of ProC440 was reduced as compared to all other ACCs containing various additional sequences between the cytokine and the first amino acid that binds the DD to the corresponding second monomer (i.e., Cys226). EC50 values for the ACCs were computed from the IFNα/β assay results and are provided below in Table 7.

TABLE 7
EC50: IFNα/β Reporter Assay

| | Pro | Pro | Pro | Pro | Pro | Pro | Pro | Pro |
|---|---|---|---|---|---|---|---|---|
| | C288 | C289 | C290 | C291 | C440 | C441 | C442 | C443 |
| EC50 | 34.34 | 17.93 | 10.33 | 8.743 | 41.37 | 6.28 | 6.637 | 1.687 |

EC50 values for the ACCs were computed from the Daudi apoptosis assay results and are provided below in Table 8.

TABLE 8
EC50: Daudi Apoptosis Assay

| | Pro | Pro | Pro | Pro | Pro | Pro | Pro | Pro |
|---|---|---|---|---|---|---|---|---|
| | C288 | C289 | C290 | C291 | C440 | C441 | C442 | C443 |
| EC50 | 112.8 | 64.55 | 23.04 | 13.39 | 2078 | 1053 | 642.9 | 478 |

The data in Tables 7-8 also shows that the activity of the (uncleaved) ACCs could be modulated by varying the length of the amino acid sequences between the cytokine and Cys226 of the DD.

Without wishing to be bound by theory, based on the results presented herein, the inventors envisage that positioning a cytokine N-terminal of the DD and using a relatively short LR inhibits cytokine activity for cytokines in addition to the interferon-alpha cytokines exemplified in the foregoing specific examples. As described above, the invention described herein encompasses activatable cytokine constructs that include various cytokine proteins discussed herein. As non-limiting examples, the CP used in the ACCs of the invention may be any of those listed in SEQ TD NOs: 101 to 209, and variants thereof. In particular, monomeric cytokines are suited to use in the ACCs described herein. Based on the results provided herein, it is believed that the ACCs of the invention will exhibit reduced cytokine activity relative to the corresponding wild type cytokine, and that upon cleavage of the ACC by the relevant protease(s), the cleavage product will recover cytokine activity similar to that of the corresponding wild type cytokine.

### Example Sequences

| SEQ | | |
|---|---|---|
| ID | | |
| NO. | NAME | SEQUENCE |
| 1 | Human Interferon- | CDLPQTHSLGSRRTLMLLAQMRRISLFSCLKDRHDFGF |
| | alpha-2b | PQEEFGNQFQKAETIPVLHEMIQQIFNLFSTKDSSAAW |
| | | DETLLDKFYTELYQQLNDLEACVIQGVGVTETPLMKED |
| | | SILAVRKYFQRITLYLKEKKYSPCAWEVVRAEIMRSFS |
| | | LSTNLQESLRSKE |
| 2 | Linker | GGGS |
| 3 | Human IgG4 Fc | CPPCPAPEFLGGPSVFLFPPKPKDTLMISRTPEVTCVV |
| | Region with S228P | VDVSQEDPEVQFNWYVDGVEVHNAKTKPREEQFNSTYR |
| | mutation, truncated | VVSVLTVLHQDWLNGKEYKCKVSNKGLPSSIEKTISKA |
| | to Cys226 | KGQPREPQVYTLPPSQEEMTKNQVSLTCLVKGFYPSDI |
| | | AVEWESNGQPENNYKTTPPVLDSDGSFFLYSRLTVDKS |
| | | RWQQGNVFSCSVMHEALHNHYTQKSLSLS |
| 4 | Human IgG4 Fc | ESKYGPPCPPCPAPEFLGGPSVFLFPPKPKDTLMISRT |
| | Region with S228P | PEVTCVVVDVSQEDPEVQFNWYVDGVEVHNAKTKPREE |
| | mutation and full | QFNSTYRVVSVLTVLHQDWLNGKEYKCKVSNKGLPSSI |
| | hinge region | EKTISKAKGQPREPQVYTLPPSQEEMTKNQVSLTCLVK |
| | | GFYPSDIAVEWESNGQPENNYKTTPPVLDSDGSFFLYS |
| | | RLTVDKSRWQQGNVFSCSVMHEALHNHYTQKSLSLS |
| 5 | CM | LSGRSDNH |
| 6 | CM | TGRGPSWV |
| 7 | CM | PLTGRSGG |
| 8 | CM | TARGPSFK |
| 9 | CM | NTLSGRSENHSG |
| 10 | CM | NTLSGRSGNHGS |
| 11 | CM | TSTSGRSANPRG |
| 12 | CM | TSGRSANP |
| 13 | CM | VHMPLGFLGP |
| 14 | CM | AVGLLAPP |
| 15 | CM | AQNLLGMV |
| 16 | CM | QNQALRMA |
| 17 | CM | LAAPLGLL |
| 18 | CM | STFPFGMF |
| 19 | CM | ISSGLLSS |
| 20 | CM | PAGLWLDP |
| 21 | CM | VAGRSMRP |
| 22 | CM | VVPEGRRS |
| 23 | CM | ILPRSPAF |
| 24 | CM | MVLGRSLL |
| 25 | CM | QGRAITFI |
| 26 | CM | SPRSIMLA |
| 27 | CM | SMLRSMPL |
| 28 | CM | ISSGLLSGRSDNH |
| 29 | CM | AVGLLAPPGGLSGRSDNH |
| 30 | CM | ISSGLLSSGGSGGSLSGRSDNH |
| 31 | CM | LSGRSGNH |
| 32 | CM | SGRSANPRG |
| 33 | CM | LSGRSDDH |
| 34 | CM | LSGRSDIH |
| 35 | CM | LSGRSDQH |
| 36 | CM | LSGRSDTH |
| 37 | CM | LSGRSDYH |
| 38 | CM | LSGRSDNP |
| 39 | CM | LSGRSANP |
| 40 | CM | LSGRSANI |
| 41 | CM | LSGRSDNI |
| 42 | CM | MIAPVAYR |
| 43 | CM | RPSPMWAY |
| 44 | CM | WATPRPMR |
| 45 | CM | FRLLDWQW |
| 46 | CM | ISSGL |
| 47 | CM | ISSGLLS |
| 48 | CM | ISSGLL |
| 49 | CM | ISSGLLSGRSANPRG |
| 50 | CM | AVGLLAPPTSGRSANPRG |
| 51 | CM | AVGLLAPPSGRSANPRG |
| 52 | CM | ISSGLLSGRSDDH |
| 53 | CM | ISSGLLSGRSDIH |
| 54 | CM | ISSGLLSGRSDQH |
| 55 | CM | ISSGLLSGRSDTH |
| 56 | CM | ISSGLLSGRSDYH |
| 57 | CM | ISSGLLSGRSDNP |
| 58 | CM | ISSGLLSGRSANP |
| 59 | CM | ISSGLLSGRSANI |
| 60 | CM | AVGLLAPPGGLSGRSDDH |
| 61 | CM | AVGLLAPPGGLSGRSDIH |
| 62 | CM | AVGLLAPPGGLSGRSDQH |
| 63 | CM | AVGLLAPPGGLSGRSDTH |
| 64 | CM | AVGLLAPPGGLSGRSDYH |
| 65 | CM | AVGLLAPPGGLSGRSDNP |
| 66 | CM | AVGLLAPPGGLSGRSANP |
| 67 | CM | AVGLLAPPGGLSGRSANI |
| 68 | CM | ISSGLLSGRSDNI |
| 69 | CM | AVGLLAPPGGLSGRSDNI |
| 70 | CM | GLSGRSDNHGGAVGLLAPP |
| 71 | CM | GLSGRSDNHGGVHMPLGFLGP |
| 72 | CM | LSGRSDNHGGVHMPLGFLGP |
| 73 | CM | ISSGLSS |
| 74 | CM | PVGYTSSL |
| 75 | CM | DWLYWPGI |
| 76 | CM | LKAAPRWA |
| 77 | CM | GPSHLVLT |
| 78 | CM | LPGGLSPW |
| 79 | CM | MGLFSEAG |
| 80 | CM | SPLPLRVP |
| 81 | CM | RMHLRSLG |
| 82 | CM | LLAPSHRA |
| 83 | CM | GPRSFGL |
| 84 | CM | GPRSFG |
| 85 | CM | SARGPSRW |
| 86 | CM | GGWHTGRN |
| 87 | CM | HTGRSGAL |
| 88 | CM | AARGPAIH |
| 89 | CM | RGPAFNPM |
| 90 | CM | SSRGPAYL |
| 91 | CM | RGPATPIM |
| 92 | CM | RGPA |
| 93 | CM | GGQPSGMWGW |
| 94 | CM | FPRPLGITGL |
| 95 | CM | SPLTGRSG |
| 96 | CM | SAGFSLPA |
| 97 | CM | LAPLGLQRR |
| 98 | CM | SGGPLGVR |
| 99 | CM | PLGL |
| 100 | CM | SGRSDNI |
| 101 | Human Interferon | CDLPQTHSLGSRRTLMLLAQMRKISLFSCLKDRHDFGF |
| | alpha-2a | PQEEFGNQFQKAETIPVLHEMIQQIFNLFSTKDSSAAW |
| | | DETLLDKFYTELYQQLNDLEACVIQGVGVTETPLMKED |
| | | SILAVRKYFQRITLYLKEKKYSPCAWEVVRAEIMRSFS |
| | | LSTNLQESLRSKE |
| 102 | Rat Interferon | CDLPHTHNLRNKRAFTLLAQMRRLSPVSCLKDRKDFGF |
| | alpha-2 | PLEKVDGQQIQKAQAIPVLHELTQQILSLFTSKESSTA |
| | | WDASLLDSFCNDLQQQLSGLQACLMQQVGVQESPLTQE |
| | | DSLLAVREYFHRITVYLREKKHSPCAWEVVRAEVWRAL |
| | | SSSANLLGRLREERNES |
| 103 | Mouse Interferon | CDLPHTYNLRNKRALKVLAQMRRLPFLSCLKDRQDFGF |
| | alpha-2 | PLEKVDNQQIQKAQAIPVLRDLTQQTLNLFTSKASSAA |
| | | WNATLLDSFCNDLHQQLNDLQTCLMQQVGVQEPPLTQE |
| | | DALLAVRKYFHRITVYLREKKHSPCAWEVVRAEVWRAL |
| | | SSSVNLLPRLSEEKE |
| 104 | Human Interferon | CDLPQTHSLGSRRTLMLLAQMRRISLFSCLKDRHDFGF |
| | Alpha-2b | PQEEFGNQFQKAETIPVLHEMIQQIFNLFSTKDSSAAW |
| | | DETLLDKFYTELYQQLNDLEACVIQGVGVTETPLMKED |
| | | SILAVRKYFQRITLYLKEKKYSPCAWEVVRAEIMRSFS |
| | | LSTNLQESLRSKE |
| 105 | Human Interferon | CDLPQTHSLGSRRTLMLLAQMRKISLFSCLKDRHDFGF |
| | Alpha-n3 | PQEEFGNQFQKAETIPVLHEMIQQIFNLFSTKDSSAAW |
| | | DETLLDKFYTELYQQLNDLEACVIQGVGVTETPLMKED |
| | | SILAVRKYFQRITLYLKEKKYSPCAWEVVRAEIMRSFS |
| | | LSTNLQESLRSKECDLPQTHSLGSRRTLMLLAQMRRIS |
| | | LFSCLKDRHDFGFPQEEFGNQFQKAETIPVLHEMIQQI |
| | | FNLFSTKDSSAAWDETLLDKFYTELYQQLNDLEACVIQ |
| | | GVGVTETPLMNEDSILAVRKYFQRITLYLKEKKYSPCA |
| | | WEVVRAEIMRSFSLSTNLQESLRSKECDLPQTHSLGSR |
| | | RTLMLLAQMRRISLFSCLKDRRDFGFPQEEFGNQFQKA |
| | | ETIPVLHEMIQQIFNLFSTKDSSAAWDETLLDKFYTEL |
| | | YQQLNDLEACVIQGVGVTETPLMNEDSILAVRKYFQRI |
| | | TLYLKEKKYSPCAWEVVRAEIMRSFSLSTNLQESLRSK |
| | | E |
| 106 | Human Interferon | MSYNLLGFLQRSSNFQCQKLLWQLNGRLEYCLKDRMNF |
| | beta-1a | DIPEEIKQLQQFQKEDAALTIYEMLQNIFAIFRQDSSS |
| | | TGWNETIVENLLANVYHQINHLKTVLEEKLEKEDFTRG |
| | | KLMSSLHLKRYYGRILHYLKAKEYSHCAWTIVRVEILR |
| | | NFYFINRLTGYLRN |
| 107 | Human Interferon | SYNLLGFLQRSSNFQSQKLLWQLNGRLEYCLKDRMNFD |
| | beta-1b | IPEEIKQLQQFQKEDAALTIYEMLQNIFAIFRQDSSST |
| | | GWNETIVENLLANVYHQINHLKTVLEEKLEKEDFTRGK |
| | | LMSSLHLKRYYGRILHYLKAKEYSHCAWTIVRVEILRN |
| | | FYFINRLTGYLRN |
| 108 | Mouse Interferon- | MNNRWILHAAFLLCFSTTALSINYKQLQLQERTNIRKC |
| | Beta | QELLEQLNGKINLTYRADFKIPMEMTEKMQKSYTAFAI |
| | | QEMLQNVFLVFRNNFSSTGWNETIVVRLLDELHQQTVF |
| | | LKTVLEEKQEERLTWEMSSTALHLKSYYWRVQRYLKLM |
| | | KYNSYAWMVVRAEIFRNFLIIRRLTRNFQN |
| 109 | Rat Interferon- | MANRWTLHIAFLLCFSTTALSIDYKQLQFRQSTSIRTC |
| | Beta | QKLLRQLNGRLNLSYRTDFKIPMEVMHPSQMEKSYTAF |
| | | AIQVMLQNVFLVFRSNFSSTGWNETIVESLLDELHQQT |
| | | ELLEIILKEKQEERLTWVTSTTTLGLKSYYWRVQRYLK |
| | | DKKYNSYAWMVVRAEVFRNFSIILRLNRNFQN |
| 110 | Human Interferon | MCDLPQNHGLLSRNTLVLLHQMRRISPFLCLKDRRDFR |
| | Omega | FPQEMVKGSQLQKAHVMSVLHEMLQQIFSLFHTERSSA |
| | | AWNMTLLDQLHTGLHQQLQHLETCLLQVVGEGESAGAI |
| | | SSPALTLRRYFQGIRVYLKEKKYSDCAWEVVRMEIMKS |
| | | LFLSTNMQERLRSKDRDLGSS |
| 111 | Human IL-1 alpha | MAKVPDMFEDLKNCYSENEEDSSSIDHLSLNQKSFYHV |
| | | SYGPLHEGCMDQSVSLSISETSKTSKLTFKESMVVVAT |
| | | NGKVLKKRRLSLSQSITDDDLEAIANDSEEEIIKPRSA |
| | | PFSFLSNVKYNFMRIIKYEFILNDALNQSIIRANDQYL |
| | | TAAALHNLDEAVKFDMGAYKSSKDDAKITVILRISKTQ |
| | | LYVTAQDEDQPVLLKEMPEIPKTITGSETNLLFFWETH |
| | | GTKNYFTSVAHPNLFIATKQDYWVCLAGGPPSITDFQI |
| | | LENQA |
| 112 | Mouse IL-1 alpha | MAKVPDLFEDLKNCYSENEDYSSAIDHLSLNQKSFYDA |
| | | SYGSLHETCTDQFVSLRTSETSKMSNFTFKESRVTVSA |
| | | TSSNGKILKKRRLSFSETFTEDDLQSITHDLEETIQPR |
| | | SAPYTYQSDLRYKLMKLVRQKFVMNDSLNQTIYQDVDK |
| | | HYLSTTWLNDLQQEVKFDMYAYSSGGDDSKYPVTLKIS |
| | | DSQLFVSAQGEDQPVLLKELPETPKLITGSETDLIFFW |
| | | KSINSKNYFTSAAYPELFIATKEQSRVHLARGLPSMTD |
| | | FQIS |
| 113 | Human IL-1 beta | MAEVPELASEMMAYYSGNEDDLFFEADGPKQMKCSFQD |
| | | LDLCPLDGGIQLRISDHHYSKGFRQAASVVVAMDKLRK |
| | | MLVPCPQTFQENDLSTFFPFIFEEEPIFFDTWDNEAYV |
| | | HDAPVRSLNCTLRDSQQKSLVMSGPYELKALHLQGQDM |
| | | EQQVVFSMSFVQGEESNDKIPVALGLKEKNLYLSCVLK |
| | | DDKPTLQLESVDPKNYPKKKMEKRFVFNKIEINNKLEF |
| | | ESAQFPNWYISTSQAENMPVFLGGTKGGQDITDFTMQF |
| | | VSS |
| 114 | Mouse IL-1 beta | MATVPELNCEMPPFDSDENDLFFEVDGPQKMKGCFQTF |
| | | DLGCPDESIQLQISQQHINKSFRQAVSLIVAVEKLWQL |
| | | PVSFPWTFQDEDMSTFFSFIFEEEPILCDSWDDDDNLL |
| | | VCDVPIRQLHYRLRDEQQKSLVLSDPYELKALHLNGQN |
| | | INQQVIFSMSFVQGEPSNDKIPVALGLKGKNLYLSCVM |
| | | KDGTPTLQLESVDPKQYPKKKMEKRFVFNKIEVKSKVE |
| | | FESAEFPNWYISTSQAEHKPVFLGNNSGQDIIDFTMES |
| | | VSS |
| 115 | Human IL-1RA | MEICRGLRSHLITLLLFLFHSETICRPSGRKSSKMQAF |
| | | RIWDVNQKTFYLRNNQLVAGYLQGPNVNLEEKIDVVPI |
| | | EPHALFLGIHGGKMCLSCVKSGDETRLQLEAVNITDLS |
| | | ENRKQDKRFAFIRSDSGPTTSFESAACPGWFLCTAMEA |
| | | DQPVSLTNMPDEGVMVTKFYFQEDE |
| 116 | Mouse IL-1RA | MEICWGPYSHLISLLLILLFHSEAACRPSGKRPCKMQA |
| | | FRIWDTNQKTFYLRNNQLIAGYLQGPNIKLEEKIDMVP |
| | | IDLHSVFLGIHGGKLCLSCAKSGDDIKLQLEEVNITDL |
| | | SKNKEEDKRFTFIRSEKGPTTSFESAACPGWFLCTTLE |
| | | ADRPVSLTNTPEEPLIVTKFYFQEDQ |
| 117 | Human IL-18 | MAAEPVEDNCINFVAMKFIDNTLYFIAEDDENLESDYF |
| | | GKLESKLSVIRNLNDQVLFIDQGNRPLFEDMTDSDCRD |
| | | NAPRTIFIISMYKDSQPRGMAVTISVKCEKISTLSCEN |
| | | KIISFKEMNPPDNIKDTKSDIIFFQRSVPGHDNKMQFE |
| | | SSSYEGYFLACEKERDLFKLILKKEDELGDRSIMFTVQ |
| | | NED |
| 118 | Mouse IL-18 | MAAMSEDSCVNFKEMMFIDNTLYFIPEENGDLESDNFG |
| | | RLHCTTAVIRNINDQVLFVDKRQPVFEDMTDIDQSASE |
| | | PQTRLIIYMYKDSEVRGLAVTLSVKDSKMSTLSCKNKI |
| | | ISFEEMDPPENIDDIQSDLIFFQKRVPGHNKMEFESSL |
| | | YEGHFLACQKEDDAFKLILKKKDENGDKSVMFTLTNLH |
| | | QS |
| 119 | Human IL-2 | MYRMQLLSCIALSLALVTNSAPTSSSTKKTQLQLEHLL |
| | | LDLQMILNGINNYKNPKLTRMLTFKFYMPKKATELKHL |
| | | QCLEEELKPLEEVLNLAQSKNFHLRPRDLISNINVIVL |
| | | ELKGSETTFMCEYADETATIVEFLNRWITFCQSIIS |
| 120 | Mouse IL-2 | MYSMQLASCVTLTLVLLVNSAPTSSSTSSSTAEAQQQQ |
| | | QQQQQQQQHLEQLLMDLQELLSRMENYRNLKLPRMLTF |
| | | KFYLPKQATELKDLQCLEDELGPLRHVLDLTQSKSFQL |
| | | EDAENFISNIRVTVVKLKGSDNTFECQFDDESATVVDF |
| | | LRRWIAFCQSIISTSPQ |
| 121 | Human IL-4 | MGLTSQLLPPLFFLLACAGNFVHGHKCDITLQEIIKTL |
| | | NSLTEQKTLCTELTVTDIFAASKNTTEKETFCRAATVL |
| | | RQFYSHHEKDTRCLGATAQQFHRHKQLIRFLKRLDRNL |
| | | WGLAGLNSCPVKEANQSTLENFLERLKTIMREKYSKCS |
| | | S |
| 122 | Mouse IL-4 | MGLNPQLVVILLFFLECTRSHIHGCDKNHLREIIGILN |
| | | EVTGEGTPCTEMDVPNVLTATKNTTESELVCRASKVLR |
| | | IFYLKHGKTPCLKKNSSVLMELQRLFRAFRCLDSSISC |
| | | TMNESKSTSLKDFLESLKSIMQMDYS |
| 123 | Human IL-7 | MFHVSFRYIFGLPPLILVLLPVASSDCDIEGKDGKQYE |
| | | SVLMVSIDQLLDSMKEIGSNCLNNEFNFFKRHICDANK |
| | | EGMFLFRAARKLRQFLKMNSTGDFDLHLLKVSEGTTIL |
| | | LNCTGQVKGRKPAALGEAQPTKSLEENKSLKEQKKLND |
| | | LCFLKRLLQEIKTCWNKILMGTKEH |
| 124 | Mouse IL-7 | MFHVSFRYIFGIPPLILVLLPVTSSECHIKDKEGKAYE |
| | | SVLMISIDELDKMTGTDSNCPNNEPNFFRKHVCDDTKE |
| | | AAFLNRAARKLKQFLKMNISEEFNVHLLTVSQGTQTLV |
| | | NCTSKEEKNVKEQKKNDACFLKRLLREIKTCWNKILKG |
| | | SI |
| 125 | Human IL-9 | MLLAMVLTSALLLCSVAGQGCPTLAGILDINFLINKMQ |
| | | EDPASKCHCSANVTSCLCLGIPSDNCTRPCFSERLSQM |
| | | TNTTMQTRYPLIFSRVKKSVEVLKNNKCPYFSCEQPCN |
| | | QTTAGNALTFLKSLLEIFQKEKMRGMRGKI |
| 126 | Mouse IL-9 | MLVTYILASVLLFSSVLGQRCSTTWGIRDTNYLIENLK |
| | | DDPPSKCSCSGNVTSCLCLSVPTDDCTTPCYREGLLQL |
| | | TNATQKSRLLPVFHRVKRIVEVLKNITCPSFSCEKPCN |
| | | QTMAGNTLSFLKSLLGTFQKTEMQRQKSRP |
| 127 | Human IL-13 | MHPLLNPLLLALGLMALLLTTVIALTCLGGFASPGPVP |
| | | PSTALRELIEELVNITQNQKAPLCNGSMVWSINLTAGM |
| | | YCAALESLINVSGCSAIEKTQRMLSGFCPHKVSAGQFS |
| | | SLHVRDTKIEVAQFVKDLLLHLKKLFREGRFN |
| 128 | Mouse IL-13 | MALWVTAVLALACLGGLAAPGPVPRSVSLPLTLKELIE |
| | | ELSNITQDQTPLCNGSMVWSVDLAAGGFCVALDSLTNI |
| | | SNCNAIYRTQRILHGLCNRKAPTTVSSLPDTKIEVAHF |
| | | ITKLLSYTKQLFRHGPF |
| 129 | Human IL-15 | MRISKPHLRSISIQCYLCLLLNSHFLTEAGIHVFILGC |
| | | FSAGLPKTEANWVNVISDLKKIEDLIQSMHIDATLYTE |
| | | SDVHPSCKVTAMKCFLLELQVISLESGDASIHDTVENL |
| | | IILANNSLSSNGNVTESGCKECEELEEKNIKEFLQSFV |
| | | HIVQMFINTS |
| 130 | Mouse IL-15 | MKILKPYMRNTSISCYLCFLLNSHFLTEAGIHVFILGC |
| | | VSVGLPKTEANWIDVRYDLEKIESLIQSIHIDTTLYTD |
| | | SDFHPSCKVTAMNCFLLELQVILHEYSNMTLNETVRNV |
| | | LYLANSTLSSNKNVAESGCKECEELEEKTFTEFLQSFI |
| | | RIVQMFINTS |
| 131 | Human IL-3 | MSRLPVLLLLQLLVRPGLQAPMTQTTPLKTSWVNCSNM |
| | | IDEIITHLKQPPLPLLDFNNLNGEDQDILMENNLRRPN |
| | | LEAFNRAVKSLQNASAIESILKNLLPCLPLATAAPTRH |
| | | PIHIKDGDWNEFRRKLTFYLKTLENAQAQQTTLSLAIF |
| 132 | Mouse IL-3 | MVLASSTTSIHTMLLLLLMLFHLGLQASISGRDTHRLT |
| | | RTLNCSSIVKEIIGKLPEPELKTDDEGPSLRNKSFRRV |
| | | NLSKFVESQGEVDPEDRYVIKSNLQKLNCCLPTSANDS |
| | | ALPGVFIRDLDDFRKKLRFYMVHLNDLETVLTSRPPQP |
| | | ASGSVSPNRGTVEC |
| 133 | Human IL-5 | MRMLLHLSLLALGAAYVYAIPTEIPTSALVKETLALLS |
| | | THRTLLIANETLRIPVPVHKNHQLCTEEIFQGIGTLES |
| | | QTVQGGTVERLFKNLSLIKKYIDGQKKKCGEERRRVNQ |
| | | FLDYLQEFLGVMNTEWIIES |
| 134 | Mouse IL-5 | MRRMLLHLSVLTLSCVWATAMEIPMSTVVKETLTQLSA |
| | | HRALLTSNETMRLPVPTHKNHQLCIGEIFQGLDILKNQ |
| | | TVRGGTVEMLFQNLSLIKKYIDRQKEKCGEERRRTRQF |
| | | LDYLQEFLGVMSTEWAMEG |
| 135 | Human GM-CSF | MWLQSLLLLGTVACSISAPARSPSPSTQPWEHVNAIQE |
| | | ARRLLNLSRDTAAEMNETVEVISEMFDLQEPTCLQTRL |
| | | ELYKQGLRGSLTKLKGPLTMMASHYKQHCPPTPETSCA |
| | | TQIITFESFKENLKDFLLVIPFDCWEPVQE |
| 136 | Mouse GM-CSF | MWLQNLLFLGIVVYSLSAPTRSPITVTRPWKHVEAIKE |
| | | ALNLLDDMPVTLNEEVEVVSNEFSFKKLTCVQTRLKIF |
| | | EQGLRGNFTKLKGALNMTASYYQTYCPPTPETDCETQV |
| | | TTYADFIDSLKTFLTDIPFECKKPGQK |
| 137 | Human IL-6 | MNSFSTSAFGPVAFSLGLLLVLPAAFPAPVPPGEDSKD |
| | | VAAPHRQPLTSSERIDKQIRYILDGISALRKETCNKSN |
| | | MCESSKEALAENNLNLPKMAEKDGCFQSGFNEETCLVK |
| | | IITGLLEFEVYLEYLQNRFESSEEQARAVQMSTKVLIQ |
| | | FLQKKAKNLDAITTPDPTTNASLLTKLQAQNQWLQDMT |
| | | THLILRSFKEFLQSSLRALRQM |
| 138 | Mouse IL-6 | MKFLSARDFHPVAFLGLMLVTTTAFPTSQVRRGDFTED |
| | | TTPNRPVYTTSQVGGLITHVLWEIVEMRKELCNGNSDC |
| | | MNNDDALAENNLKLPEIQRNDGCYQTGYNQEICLLKIS |
| | | SGLLEYHSYLEYMKNNLKDNKKDKARVLQRDTETLIHI |
| | | FNQEVKDLHKIVLPTPISNALLTDKLESQKEWLRTKTI |
| | | QFILKSLEEFLKVTLRSTRQT |
| 139 | Human IL-11 | MNCVCRLVLVVLSLWPDTAVAPGPPPGPPRVSPDPRAE |
| | | LDSTVLLTRSLLADTRQLAAQLRDKFPADGDHNLDSLP |
| | | TLAMSAGALGALQLPGVLTRLRADLLSYLRHVQWLRRA |
| | | GGSSLKTLEPELGTLQARLDRLLRRLQLLMSRLALPQP |
| | | PPDPPAPPLAPPSSAWGGIRAAHAILGGLHLTLDWAVR |
| | | GLLLLKTRL |
| 140 | Mouse IL-11 | MNCVCRLVLVVLSLWPDRVVAPGPPAGSPRVSSDPRAD |
| | | LDSAVLLTRSLLADTRQLAAQMRDKFPADGDHSLDSLP |
| | | TLAMSAGTLGSLQLPGVLTRLRVDLMSYLRHVQWLRRA |
| | | GGPSLKTLEPELGALQARLERLLRRLQLLMSRLALPQA |
| | | APDQPVIPLGPPASAWGSIRAAHAILGGLHLTLDWAVR |
| | | GLLLLKTRL |
| 141 | Human G-CSF | MAGPATQSPMKLMALQLLLWHSALWTVQEATPLGPASS |
| | | LPQSFLLKCLEQVRKIQGDGAALQEKLVSECATYKLCH |
| | | PEELVLLGHSLGIPWAPLSSCPSQALQLAGCLSQLHSG |
| | | LFLYQGLLQALEGISPELGPTLDTLQLDVADFATTIWQ |
| | | QMEELGMAPALQPTQGAMPAFASAFQRRAGGVLVASHL |
| | | QSFLEVSYRVLRHLAQP |
| 142 | Mouse G-CSF | MAQLSAQRRMKLMALQLLLWQSALWSGREAVPLVTVSA |
| | | LPPSLPLPRSFLLKSLEQVRKIQASGSVLLEQLCATYK |
| | | LCHPEELVLLGHSLGIPKASLSGCSSQALQQTQCLSQL |
| | | HSGLCLYQGLLQALSGISPALAPTLDLLQLDVANFATT |
| | | IWQQMENLGVAPTVQPTQSAMPAFTSAFQRRAGGVLAI |
| | | SYLQGFLETARLALHHLA |
| 143 | Human IL-12 alpha | MCPARSLLLVATLVLLDHLSLARNLPVATPDPGMFPCL |
| | | HHSQNLLRAVSNMLQKARQTLEFYPCTSEEIDHEDITK |
| | | DKTSTVEACLPLELTKNESCLNSRETSFITNGSCLASR |
| | | KTSFMMALCLSSIYEDLKMYQVEFKTMNAKLLMDPKRQ |
| | | IFLDQNMLAVIDELMQALNFNSETVPQKSSLEEPDFYK |
| | | TKIKLCILLHAFRIRAVTIDRVMSYLNAS |
| 144 | Human IL-12 beta | MCHQQLVISWFSLVFLASPLVAIWELKKDVYVVELDWY |
| | | PDAPGEMVVLTCDTPEEDGITWTLDQSSEVLGSGKTLT |
| | | IQVKEFGDAGQYTCHKGGEVLSHSLLLLHKKEDGIWST |
| | | DILKDQKEPKNKTFLRCEAKNYSGRFTCWWLTTISTDL |
| | | TFSVKSSRGSSDPQGVTCGAATLSAERVRGDNKEYEYS |
| | | VECQEDSACPAAEESLPIEVMVDAVHKLKYENYTSSFF |
| | | IRDIIKPDPPKNLQLKPLKNSRQVEVSWEYPDTWSTPH |
| | | SYFSLTFCVQVQGKSKREKKDRVFTDKTSATVICRKNA |
| | | SISVRAQDRYYSSSWSEWASVPCS |
| 145 | Mouse IL-12 beta | MCPQKLTISWFAIVLLVSPLMAMWELEKDVYVVEVDWT |
| | | PDAPGETVNLTCDTPEEDDITWTSDQRHGVIGSGKTLT |
| | | ITVKEFLDAGQYTCHKGGETLSHSHLLLHKKENGIWST |
| | | EILKNFKNKTFLKCEAPNYSGRFTCSWLVQRNMDLKFN |
| | | IKSSSSSPDSRAVTCGMASLSAEKVTLDQRDYEKYSVS |
| | | CQEDVTCPTAEETLPIELALEARQQNKYENYSTSFFIR |
| | | DIIKPDPPKNLQMKPLKNSQVEVSWEYPDSWSTPHSYF |
| | | SLKFFVRIQRKKEKMKETEEGCNQKGAFLVEKTSTEVQ |
| | | CKGGNVCVQAQDRYYNSSCSKWACVPCRVRS |
| 146 | Mouse IL-12 alpha | MCQSRYLLFLATLALLNHLSLARVIPVSGPARCLSQSR |
| | | NLLKTTDDMVKTAREKLKHYSCTAEDIDHEDITRDQTS |
| | | TLKTCLPLELHKNESCLATRETSSTTRGSCLPPQKTSL |
| | | MMTLCLGSIYEDLKMYQTEFQAINAALQNHNHQQIILD |
| | | KGMLVAIDELMQSLNHNGETLRQKPPVGEADPYRVKMK |
| | | LCILLHAFSTRVVTINRVMGYLSSA |
| 147 | Human LIF | MKVLAAGVVPLLLVLHWKHGAGSPLPITPVNATCAIRH |
| | | PCHNNLMNQIRSQLAQLNGSANALFILYYTAQGEPFPN |
| | | NLDKLCGPNVTDFPPFHANGTEKAKLVELYRIVVYLGT |
| | | SLGNITRDQKILNPSALSLHSKLNATADILRGLLSNVL |
| | | CRLCSKYHVGHVDVTYGPDTSGKDVFQKKKLGCQLLGK |
| | | YKQIIAVLAQAF |
| 148 | Mouse LIF | MKVLAAGIVPLLLLVLHWKHGAGSPLPITPVNATCAIR |
| | | HPCHGNLMNQIKNQLAQLNGSANALFISYYTAQGEPFP |
| | | NNVEKLCAPNMTDFPSFHGNGTEKTKLVELYRMVAYLS |
| | | ASLTNITRDQKVLNPTAVSLQVKLNATIDVMRGLLSNV |
| | | LCRLCNKYRVGHVDVPPVPDHSDKEAFQRKKLGCQLLG |
| | | TYKQVISVVVQAF |
| 149 | Human OSM | MGVLLTQRTLLSLVLALLFPSMASMAAIGSCSKEYRVL |
| | | LGQLQKQTDLMQDTSRLLDPYIRIQGLDVPKLREHCRE |
| | | RPGAFPSEETLRGLGRRGFLQTLNATLGCVLHRLADLE |
| | | QRLPKAQDLERSGLNIEDLEKLQMARPNILGLRNNIYC |
| | | MAQLLDNSDTAEPTKAGRGASQPPTPTPASDAFQRKLE |
| | | GCRFLHGYHRFMHSVGRVFSKWGESPNRSRRHSPHQAL |
| | | RKGVRRTRPSRKGKRLMTRGQLPR |
| 150 | Mouse OSM | MQTRLLRTLLSLTLSLLILSMALANRGCSNSSSQLLSQ |
| | | LQNQANLTGNTESLLEPYIRLQNLNTPDLRAACTQHSV |
| | | AFPSEDTLRQLSKPHFLSTVYTTLDRVLYQLDALRQKF |
| | | LKTPAFPKLDSARHNILGIRNNVFCMARLLNHSLEIPE |
| | | PTQTDSGASRSTTTPDVFNTKIGSCGFLWGYHRFMGSV |
| | | GRVFREWDDGSTRSRRQSPLRARRKGTRRIRVRHKGTR |
| | | RIRVRRKGTRRIWVRRKGSRKIRPSRSTQSPTTRA |
| 151 | Human IL-10 | MHSSALLCCLVLLTGVRASPGQGTQSENSCTHFPGNLP |
| | | NMLRDLRDAFSRVKTFFQMKDQLDNLLLKESLLEDFKG |
| | | YLGCQALSEMIQFYLEEVMPQAENQDPDIKAHVNSLGE |
| | | NLKTLRLRLRRCHRFLPCENKSKAVEQVKNAFNKLQEK |
| | | GIYKAMSEFDIFINYIEAYMTMKIRN |
| 152 | Mouse IL-10 | MPGSALLCCLLLLTGMRISRGQYSREDNNCTHFPVGQS |
| | | HMLLELRTAFSQVKTFFQTKDQLDNILLTDSLMQDFKG |
| | | YLGCQALSEMIQFYLVEVMPQAEKHGPEIKEHLNSLGE |
| | | KLKTLRMRLRRCHRFLPCENKSKAVEQVKSDFNKLQDQ |
| | | GVYKAMNEFDIFINCIEAYMMIKMKS |
| 153 | Human IL-20 | MKASSLAFSLLSAAFYLLWTPSTGLKTLNLGSCVIATN |
| | | LQEIRNGFSEIRGSVQAKDGNIDIRILRRTESLQDTKP |
| | | ANRCCLLRHLLRLYLDRVFKNYQTPDHYTLRKISSLAN |
| | | SFLTIKKDLRLCHAHMTCHCGEEAMKKYSQILSHFEKL |
| | | EPQAAVVKALGELDILLQWMEETE |
| 154 | Mouse IL-20 | MKGFGLAFGLFSAVGFLLWTPLTGLKTLHLGSCVITAN |
| | | LQAIQKEFSEIRDSVQAEDTNIDIRILRTTESLKDIKS |
| | | LDRCCFLRHLVRFYLDRVFKVYQTPDHHTLRKISSLAN |
| | | SFLIIKKDLSVCHSHMACHCGEEAMEKYNQILSHFIEL |
| | | ELQAAVVKALGELGILLRWMEEML |
| 155 | Human IL-14 | MKNQDKKNGAAKQSNPKSSPGQPEAGPEGAQERPSQAA |
| | | PAVEAEGPGSSQAPRKPEGAQARTAQSGALRDVSEELS |
| | | RQLEDILSTYCVDNNQGGPGEDGAQGEPAEPEDAEKSR |
| | | TYVARNGEPEPTPVVNGEKEPSKGDPNTEEIRQSDEVG |
| | | DRDHRRPQEKKKAKGLGKEITLLMQTLNTLSTPEEKLA |
| | | ALCKKYAELLEEHRNSQKQMKLLQKKQSQLVQEKDHLR |
| | | GEHSKAVLARSKLESLCRELQRHNRSLKEEGVQRAREE |
| | | EEKRKEVTSHFQVTLNDIQLQMEQHNERNSKLRQENME |
| | | LAERLKKLIEQYELREEHIDKVFKHKDLQQQLVDAKLQ |
| | | QAQEMLKEAEERHQREKDFLLKEAVESQRMCELMKQQE |
| | | THLKQQLALYTEKFEEFQNTLSKSSEVFTTFKQEMEKM |
| | | TKKIKKLEKETTMYRSRWESSNKALLEMAEEKTVRDKE |
| | | LEGLQVKIQRLEKLCRALQTERNDLNKRVQDLSAGGQG |
| | | SLTDSGPERRPEGPGAQAPSSPRVTEAPCYPGAPSTEA |
| | | SGQTGPQEPTSARA |
| 156 | Mouse IL-14 | MKNQDKKNGPAKHSNSKGSPGQREAGPEGAHGRPRQTA |
| | | PGAEAEGSTSQAPGKTEGARAKAAQPGALCDVSEELSR |
| | | QLEDILSTYCVDNNQGGPAEEGAQGEPTEPEDTEKSRT |
| | | YAARNGEPEPGIPVVNGEKETSKGEPGTEEIRASDEVG |
| | | DRDHRRPQEKKKAKGLGKEITLLMQTLNTLSTPEEKLA |
| | | ALCKKYAELLEEHRNSQKQMKLLQKKQSQLVQEKDHLR |
| | | GEHSKAVLARSKLESLCRELQRHNRSLKEEGVQRAREE |
| | | EEKRKEVTSHFQVTLNDIQLQMEQHNERNSKLRQENME |
| | | LAERLKKLIEQYELREEHIDKVFKHKDLQQQLVDAKLQ |
| | | QAQEMLKEAEERHQREKEFLLKEAVESQRMCELMKQQE |
| | | THLKQQLALYTEKFEEFQNTLSKSSEVFTTFKQEMEKM |
| | | TKKIKKLEKETTMYRSRWESSNKALLEMAEEKTVRDKE |
| | | LEGLQVKIQRLEKLCRALQTERNDLNKRVQDLTAGGIT |
| | | DIGSERRPEATTASKEQGVESPGAQPASSPRATDAPCC |
| | | SGAPSTGTAGQTGPGEPTPATA |
| 157 | Human IL-16 | MESHSRAGKSRKSAKFRSISRSLMLCNAKTSDDGSSPD |
| | | EKYPDPFEISLAQGKEGIFHSSVQLADTSEAGPSSVPD |
| | | LALASEAAQLQAAGNDRGKTCRRIFFMKESSTASSREK |
| | | PGKLEAQSSNFLFPKACHQRARSNSTSVNPYCTREIDF |
| | | PMTKKSAAPTDRQPYSLCSNRKSLSQQLDCPAGKAAGT |
| | | SRPTRSLSTAQLVQPSGGLQASVISNIVLMKGQAKGLG |
| | | FSIVGGKDSIYGPIGIYVKTIFAGGAAAADGRLQEGDE |
| | | ILELNGESMAGLTHQDALQKFKQAKKGLLTLTVRTRLT |
| | | APPSLCSHLSPPLCRSLSSSTCITKDSSSFALESPSAP |
| | | ISTAKPNYRIMVEVSLQKEAGVGLGIGLCSVPYFQCIS |
| | | GIFVHTLSPGSVAHLDGRLRCGDEIVEISDSPVHCLTL |
| | | NEVYTILSHCDPGPVPIIVSRHPDPQVSEQQLKEAVAQ |
| | | AVENTKFGKERHQWSLEGVKRLESSWHGRPTLEKEREK |
| | | NSAPPHRRAQKVMIRSSSDSSYMSGSPGGSPGSGSAEK |
| | | PSSDVDISTHSPSLPLAREPVVLSIASSRLPQESPPLP |
| | | ESRDSHPPLRLKKSFEIVRKPMSSKPKPPPRKYFKSDS |
| | | DPQKSLEERENSSCSSGHTPPTCGQEARELLPLLLPQE |
| | | DTAGRSPSASAGCPGPGIGPQTKSSTEGEPGWRRASPV |
| | | TQTSPIKHPLLKRQARMDYSFDTTAEDPWVRISDCIKN |
| | | LFSPIMSENHGHMPLQPNASLNEEEGTQGHPDGTPPKL |
| | | DTANGTPKVYKSADSSTVKKGPPVAPKPAWFRQSLKGL |
| | | RNRASDPRGLPDPALSTQPAPASREHLGSHIRASSSSS |
| | | SIRQRISSFETFGSSQLPDKGAQRLSLQPSSGEAAKPL |
| | | GKHEEGRFSGLLGRGAAPTLVPQQPEQVLSSGSPAASE |
| | | ARDPGVSESPPPGRQPNQKTLPPGPDPLLRLLSTQAEE |
| | | SQGPVLKMPSQRARSFPLTRSQSCETKLLDEKTSKLYS |
| | | ISSQVSSAVMKSLLCLPSSISCAQTPCIPKEGASPTSS |
| | | SNEDSAANGSAETSALDTGFSLNLSELREYTEGLTEAK |
| | | EDDDGDHSSLQSGQSVISLLSSEELKKLIEEVKVLDEA |
| | | TLKQLDGIHVTILHKEEGAGLGFSLAGGADLENKVITV |
| | | HRVFPNGLASQEGTIQKGNEVLSINGKSLKGTTHHDAL |
| | | AILRQAREPRQAVIVTRKLTPEAMPDLNSSTDSAASAS |
| | | AASDVSVESTAEATVCTVTLEKMSAGLGFSLEGGKGSL |
| | | HGDKPLTINRIFKGAASEQSETVQPGDEILQLGGTAMQ |
| | | GLTRFEAWNIIKALPDGPVTIVIRRKSLQSKETTAAGD |
| | | S |
| 158 | Mouse IL-16 | MEPHGHSGKSRKSTKFRSISRSLILCNAKTSDDGSSPD |
| | | EKYPDPFETSLCQGKEGFFHSSMQLADTFEAGLSNIPD |
| | | LALASDSAQLAAAGSDRGKHCRKMFFMKESSSTSSKEK |
| | | SGKPEAQSSSFLFPKACHQRTRSNSTSVNPYSAGEIDF |
| | | PMTKKSAAPTDRQPYSLCSNRKSLSQQLDYPILGTARP |
| | | TRSLSTAQLGQLSGGLQASVISNIVLMKGQAKGLGFSI |
| | | VGGKDSIYGPIGIYVKSIFAGGAAAADGRLQEGDEILE |
| | | LNGESMAGLTHQDALQKFKQAKKGLLTLTVRTRLTTPP |
| | | SLCSHLSPPLCRSLSSSTCGAQDSSPFSLESPASPAST |
| | | AKPNYRIMVEVSLKKEAGVGLGIGLCSIPYFQCISGIF |
| | | VHTLSPGSVAHLDGRLRCGDEIVEINDSPVHCLTLNEV |
| | | YTILSHCDPGPVPIIVSRHPDPQVSEQQLKEAVAQAVE |
| | | GVKFGKDRHQWSLEGVKRLESSWHGRPTLEKEREKHSA |
| | | PPHRRAQKIMVRSSSDSSYMSGSPGGSPCSAGAEPQPS |
| | | EREGSTHSPSLSPGEEQEPCPGVPSRPQQESPPLPESL |
| | | ERESHPPLRLKKSFEILVRKPTSSKPKPPPRKYFKNDS |
| | | EPQKKLEEKEKVTDPSGHTLPTCSQETRELLPLLLQED |
| | | TAGRAPCTAACCPGPAASTQTSSSTEGESRRSASPETP |
| | | ASPGKHPLLKRQARMDYSFDITAEDPWVRISDCIKNLF |
| | | SPIMSENHSHTPLQPNTSLGEEDGTQGCPEGGLSKMDA |
| | | ANGAPRVYKSADGSTVKKGPPVAPKPAWFRQSLKGLRN |
| | | RAPDPRRPPEVASAIQPTPVSRDPPGPQPQASSSIRQR |
| | | ISSFENFGSSQLPDRGVQRLSLQPSSGETTKFPGKQDG |
| | | GRFSGLLGQGATVTAKHRQTEVESMSTTFPNSSEVRDP |
| | | GLPESPPPGQRPSTKALSPDPLLRLLTTQSEDTQGPGL |
| | | KMPSQRARSFPLTRTQSCETKLLDEKASKLYSISSQLS |
| | | SAVMKSLLCLPSSVSCGQITCIPKERVSPKSPCNNSSA |
| | | AEGFGEAMASDTGFSLNLSELREYSEGLTEPGETEDRN |
| | | HCSSQAGQSVISLLSAEELEKLIEEVRVLDEATLKQLD |
| | | SIHVTILHKEEGAGLGFSLAGGADLENKVITVHRVFPN |
| | | GLASQEGTIQKGNEVLSINGKSLKGATHNDALAILRQA |
| | | RDPRQAVIVTRRTTVEATHDLNSSTDSAASASAASDIS |
| | | VESKEATVCTVTLEKTSAGLGFSLEGGKGSLHGDKPLT |
| | | INRIFKGTEQGEMVQPGDEILQLAGTAVQGLTRFEAWN |
| | | VIKALPDGPVTIVIRRTSLQCKQTTASADS |
| 159 | Human IL-17 | MTPGKTSLVSLLLLLSLEAIVKAGITIPRNPGCPNSED |
| | | KNFPRTVMVNLNIHNRNTNTNPKRSSDYYNRSTSPWNL |
| | | HRNEDPERYPSVIWEAKCRHLGCINADGNVDYHMNSVP |
| | | IQQEILVLRREPPHCPNSFRLEKILVSVGCTCVTPIVH |
| | | HVA |
| 160 | Mouse IL-17 | MSPGRASSVSLMLLLLLSLAATVKAAAIIPQSSACPNT |
| | | EAKDFLQNVKVNLKVFNSLGAKVSSRRPSDYLNRSTSP |
| | | WTLHRNEDPDRYPSVIWEAQCRHQRCVNAEGKLDHHMN |
| | | SVLIQQEILVLKREPESCPFTFRVEKMLVGVGCTCVAS |
| | | IVRQAA |
| 161 | Human CD154 | MIETYNQTSPRSAATGLPISMKIFMYLLTVFLITQMIG |
| | | SALFAVYLHRRLDKIEDERNLHEDFVFMKTIQRCNTGE |
| | | RSLSLLNCEEIKSQFEGFVKDIMLNKEETKKENSFEMQ |
| | | KGDQNPQIAAHVISEASSKTTSVLQWAEKGYYTMSNNL |
| | | VTLENGKQLTVKRQGLYYIYAQVTFCSNREASSQAPFI |
| | | ASLCLKSPGRFERILLRAANTHSSAKPCGQQSIHLGGV |
| | | FELQPGASVFVNVTDPSQVSHGTGFTSFGLLKL |
| 162 | Mouse CD154 | MIETYSQPSPRSVATGLPASMKIFMYLLTVFLITQMIG |
| | | SVLFAVYLHRRLDKVEEEVNLHEDFVFIKKLKRCNKGE |
| | | GSLSLLNCEEMRRQFEDLVKDITLNKEEKKENSFEMQR |
| | | GDEDPQIAAHVVSEANSNAASVLQWAKKGYYTMKSNLV |
| | | MLENGKQLTVKREGLYYVYTQVTFCSNREPSSQRPFIV |
| | | GLWLKPSSGSERILLKAANTHSSSQLCEQQSVHLGGVF |
| | | ELQAGASVFVNVTEASQVIHRVGFSSFGLLKL |
| 163 | Human LT-beta | MGALGLEGRGGRLQGRGSLLLAVAGATSLVTLLLAVPI |
| | | TVLAVLALVPQDQGGLVTETADPGAQAQQGLGFQKLPE |
| | | EEPETDLSPGLPAAHLIGAPLKGQGLGWETTKEQAFLT |
| | | SGTQFSDAEGLALPQDGLYYLYCLVGYRGRAPPGGGDP |
| | | QGRSVTLRSSLYRAGGAYGPGTPELLLEGAETVTPVLD |
| | | PARRQGYGPLWYTSVGFGGLVQLRRGERVYVNISHPDM |
| | | VDFARGKTFFGAVMVG |
| 164 | Mouse LT-beta | MGTRGLQGLGGRPQGRGCLLLAVAGATSLVTLLLAVPI |
| | | TVLAVLALVPQDQGRRVEKIIGSGAQAQKRLDDSKPSC |
| | | ILPSPSSLSETPDPRLHPQRSNASRNLASTSQGPVAQS |
| | | SREASAWMTILSPAADSTPDPGVQQLPKGEPETDLNPE |
| | | LPAAHLIGAWMSGQGLSWEASQEEAFLRSGAQFSPTHG |
| | | LALPQDGVYYLYCHVGYRGRTPPAGRSRARSLTLRSAL |
| | | YRAGGAYGRGSPELLLEGAETVTPVVDPIGYGSLWYTS |
| | | VGFGGLAQLRSGERVYVNISHPDMVDYRRGKTFFGAVM |
| | | VG |
| 165 | Human TNF-alpha | STESMIRDVELAEEALPKKTGGPQGSRRCLFLSLFSFL |
| | | IVAGATTLFCLLHFGVIGPQREEFPRDLSLISPLAQAV |
| | | RSSSRTPSDKPVAHVVANPQAEGQLQWLNRRANALLAN |
| | | GVELRDNQLVVPSEGLYLIYSQVLFKGQGCPSTHVLLT |
| | | HTISRIAVSYQTKVNLLSAIKSPCQRETPEGAEAKPWY |
| | | EPIYLGGVFQLEKGDRLSAEINRPDYLDFAESGQVYFG |
| | | IIAL |
| 166 | Mouse TNF-alpha | NHQVEEQLEWLSQRANALLANGMDLKDNQLVVPADGLY |
| | | LVYSQVLFKGQGCPDYVLLTHTVSRFAISYQEKVNLLS |
| | | AVKSPCPKDTPEGAELKPWYEPIYLGGVFQLEKGDQLS |
| | | AEVNLPKYLDFAESGQVYFGVIAL |
| 167 | Human TNF-beta | MTPPERLFLPRVCGTTLHLLLLGLLLVLLPGAQGLPGV |
| | | GLTPSAAQTARQHPKMHLAHSTLKPAAHLIGDPSKQNS |
| | | LLWRANTDRAFLQDGFSLSNNSLLVPTSGIYFVYSQVV |
| | | FSGKAYSPKATSSPLYLAHEVQLFSSQYPFHVPLLSSQ |
| | | KMVYPGLQEPWLHSMYHGAAFQLTQGDQLSTHTDGIPH |
| | | LVLSPSTVFFGAFAL |
| 168 | Human 4-1BBL | MEYASDASLDPEAPWPPAPRARACRVLPWALVAGLLLL |
| | | LLLAAACAVFLACPWAVSGARASPGSAASPRLREGPEL |
| | | SPDDPAGLLDLRQGMFAQLVAQNVLLIDGPLSWYSDPG |
| | | LAGVSLTGGLSYKEDTKELVVAKAGVYYVFFQLELRRV |
| | | VAGEGSGSVSLALHLQPLRSAAGAAALALTVDLPPASS |
| | | EARNSAFGFQGRLLHLSAGQRLGVHLHTEARARHAWQL |
| | | TQGATVLGLFRVTPEIPAGLPSPRSE |
| 169 | Mouse 4-1BBL | MDQHTLDVEDTADARHPAGTSCPSDAALLRDTGLLADA |
| | | ALLSDTVRPTNAALPTDAAYPAVNVRDREAAWPPALNF |
| | | CSRHPKLYGLVALVLLLLIAACVPIFTRTEPRPALTIT |
| | | TSPNLGTRENNADQVTPVSHIGCPNTTQQGSPVFAKLL |
| | | AKNQASLCNTTLNWHSQDGAGSSYLSQGLRYEEDKKEL |
| | | VVDSPGLYYVFLELKLSPTFTNTGHKVQGWVSLVLQAK |
| | | PQVDDFDNLALTVELFPCSMENKLVDRSWSQLLLLKAG |
| | | HRLSVGLRAYLHGAQDAYRDWELSYPNTTSFGLFLVKP |
| | | DNPWE |
| 170 | Human APRIL | AVLTQKQKKQHSVLHLVPINATSKDDSDVTEVMWQPAL |
| | | RRGRGLQAQGYGVRIQDAGVYLLYSQVLFQDVTFTMGQ |
| | | VVSREGQGRQETLFRCIRSMPSHPDRAYNSCYSAGVFH |
| | | LHQGDILSVIIPRARAKLNLSPHGTFLGFVKL |
| 171 | Mouse APRIL | MPASSPGHMGGSVREPALSVALWLSWGAVLGAVTCAVA |
| | | LLIQQTELQSLRREVSRLQRSGGPSQKQGERPWQSLWE |
| | | QSPDVLEAWKDGAKSRRRRAVLTQKHKKKHSVLHLVPV |
| | | NITSKADSDVTEVMWQPVLRRGRGLEAQGDIVRVWDTG |
| | | IYLLYSQVLFHDVTFTMGQVVSREGQGRRETLFRCIRS |
| | | MPSDPDRAYNSCYSAGVFHLHQGDIITVKIPRANAKLS |
| | | LSPHGTFLGFVKL |
| 172 | Human CD70 | MPEEGSGCSVRRRPYGCVLRAALVPLVAGLVICLVVCI |
| | | QRFAQAQQQLPLESLGWDVAELQLNHTGPQQDPRLYWQ |
| | | GGPALGRSFLHGPELDKGQLRIHRDGIYMVHIQVTLAI |
| | | CSSTTASRHHPTTLAVGICSPASRSISLLRLSFHQGCT |
| | | IASQRLTPLARGDTLCTNLTGTLLPSRNTDETFFGVQW |
| | | VRP |
| 173 | Mouse CD70 | MPEEGRPCPWVRWSGTAFQRQWPWLLLVVFITVFCCWF |
| | | HCSGLLSKQQQRLLEHPEPHTAELQLNLTVPRKDPTLR |
| | | WGAGPALGRSFTHGPELEEGHLRIHQDGLYRLHIQVTL |
| | | ANCSSPGSTLQHRATLAVGICSPAAHGISLLRGRFGQD |
| | | CTVALQRLTYLVHGDVLCTNLTLPLLPSRNADETFFGV |
| | | QWICP |
| 174 | Human CD153 | MDPGLQQALNGMAPPGDTAMHVPAGSVASHLGTTSRSY |
| | | FYLTTATLALCLVFTVATIMVLVVQRTDSIPNSPDNVP |
| | | LKGGNCSEDLLCILKRAPFKKSWAYLQVAKHLNKTKLS |
| | | WNKDGILHGVRYQDGNLVIQFPGLYFIICQLQFLVQCP |
| | | NNSVDLKLELLINKHIKKQALVTVCESGMQTKHVYQNL |
| | | SQFLLDYLQVNTTISVNVDTFQYIDTSTFPLENVLSIF |
| | | LYSNSD |
| 175 | Mouse CD153 | MEPGLQQAGSCGAPSPDPAMQVQPGSVASPWRSTRPWR |
| | | STSRSYFYLSTTALVCLVVAVAIILVLVVQKKDSTPNT |
| | | TEKAPLKGGNCSEDLFCTLKSTPSKKSWAYLQVSKHLN |
| | | NTKLSWNEDGTIHGLIYQDGNLIVQFPGLYFIVCQLQF |
| | | LVQCSNHSVDLTLQLLINSKIKKQTLVTVCESGVQSKN |
| | | IYQNLSQFLLHYLQVNSTISVRVDNFQYVDTNTFPLDN |
| | | VLSVFLYSSSD |
| 176 | Human CD178 | MQQPFNYPYPQIYWVDSSASSPWAPPGTVLPCPTSVPR |
| | | RPGQRRPPPPPPPPPLPPPPPPPPLPPLPLPPLKKRGN |
| | | HSTGLCLLVMFFMVLVALVGLGLGMFQLFHLQKELAEL |
| | | RESTSQMHTASSLEKQIGHPSPPPEKKELRKVAHLTGK |
| | | SNSRSMPLEWEDTYGIVLLSGVKYKKGGLVINETGLYF |
| | | VYSKVYFRGQSCNNLPLSHKVYMRNSKYPQDLVMMEGK |
| | | MMSYCTTGQMWARSSYLGAVFNLTSADHLYVNVSELSL |
| | | VNFEESQTFFGLYKL |
| 177 | Mouse CD178 | MQQPMNYPCPQIFWVDSSATSSWAPPGSVFPCPSCGPR |
| | | GPDQRRPPPPPPPVSPLPPPSQPLPLPPLTPLKKKDHN |
| | | TNLWLPVVFFMVLVALVGMGLGMYQLFHLQKELAELRE |
| | | FTNQSLKVSSFEKQIANPSTPSEKKEPRSVAHLTGNPH |
| | | SRSIPLEWEDTYGTALISGVKYKKGGLVINETGLYFVY |
| | | SKVYFRGQSCNNQPLNHKVYMRNSKYPEDLVLMEEKRL |
| | | NYCTTGQIWAHSSYLGAVFNLTSADHLYVNISQLSLIN |
| | | FEESKTFFGLYKL |
| 178 | Human GITRL | MTLHPSPITCEFLFSTALISPKMCLSHLENMPLSHSRT |
| | | QGAQRSSWKLWLFCSIVMLLFLCSFSWLIFIFLQLETA |
| | | KEPCMAKFGPLPSKWQMASSEPPCVNKVSDWKLEILQN |
| | | GLYLIYGQVAPNANYNDVAPFEVRLYKNKDMIQTLTNK |
| | | SKIQNVGGTYELHVGDTIDLIFNSEHQVLKNNTYWGII |
| | | LLANPQFIS |
| 179 | Mouse GITRL | MEEMPLRESSPQRAERCKKSWLLCIVALLLMLLCSLGT |
| | | LIYTSLKPTAIESCMVKFELSSSKWHMTSPKPHCVNTT |
| | | SDGKLKILQSGTYLIYGQVIPVDKKYIKDNAPFVVQIY |
| | | KKNDVLQTLMNDFQILPIGGVYELHAGDNIYLKFNSKD |
| | | HIQKTNTYWGIILMPDLPFIS |
| 180 | Human LIGHT | MEESVVRPSVFVVDGQTDIPFTRLGRSHRRQSCSVARV |
| | | GLGLLLLLMGAGLAVQGWFLLQLHWRLGEMVTRLPDGP |
| | | AGSWEQLIQERRSHEVNPAAHLTGANSSLTGSGGPLLW |
| | | ETQLGLAFLRGLSYHDGALVVTKAGYYYIYSKVQLGGV |
| | | GCPLGLASTITHGLYKRTPRYPEELELLVSQQSPCGRA |
| | | TSSSRVWWDSSFLGGVVHLEAGEKVVVRVLDERLVRLR |
| | | DGTRSYFGAFMV |
| 181 | Mouse LIGHT | MESVVQPSVFVVDGQTDIPFRRLEQNHRRRRCGTVQVS |
| | | LALVLLLGAGLATQGWFLLRLHQRLGDIVAHLPDGGKG |
| | | SWEKLIQDQRSHQANPAAHLTGANASLIGIGGPLLWET |
| | | RLGLAFLRGLTYHDGALVTMEPGYYYVYSKVQLSGVGC |
| | | PQGLANGLPITHGLYKRTSRYPKELELLVSRRSPCGRA |
| | | NSSRVWWDSSFLGGVVHLEAGEEVVVRVPGNRLVRPRD |
| | | GTRSYFGAFMV |
| 182 | Human OX40L | MERVQPLEENVGNAARPRFERNKLLLVASVIQGLGLLL |
| | | CFTYICLHFSALQVSHRYPRIQSIKVQFTEYKKEKGFI |
| | | LTSQKEDEIMKVQNNSVIINCDGFYLISLKGYFSQEVN |
| | | ISLHYQKDEEPLFQLKKVRSVNSLMVASLTYKDKVYLN |
| | | VTTDNTSLDDFHVNGGELILIHQNPGEFCVL |
| 183 | Mouse OX40L | MEGEGVQPLDENLENGSRPRFKWKKTLRLVVSGIKGAG |
| | | MLLCFIYVCLQLSSSPAKDPPIQRLRGAVTRCEDGQLF |
| | | ISSYKNEYQTMEVQNNSVVIKCDGLYIIYLKGSFFQEV |
| | | KIDLHFREDHNPISIPMLNDGRRIVFTVVASLAFKDKV |
| | | YLTVNAPDTLCEHLQINDGELIVVQLTPGYCAPEGSYH |
| | | STVNQVP |
| 184 | Human TALL-1 | MDDSTEREQSRLTSCLKKREEMKLKECVSILPRKESPS |
| | | VRSSKDGKLLAATLLLALLSCCLTVVSFYQVAALQGDL |
| | | ASLRAELQGHHAEKLPAGAGAPKAGLEEAPAVTAGLKI |
| | | FEPPAPGEGNSSQNSRNKRAVQGPEETVTQDCLQLIAD |
| | | SETPTIQKGSYTFVPWLLSFKRGSALEEKENKILVKET |
| | | GYFFIYGQVLYTDKTYAMGHLIQRKKVHVFGDELSLVT |
| | | LFRCIQNMPETLPNNSCYSAGIAKLEEGDELQLAIPRE |
| | | NAQISLDGDVTFFGALKLL |
| 185 | Mouse TALL-1 | MAMAFCPKDQYWDSSRKSCVSCALTCSQRSQRTCTDFC |
| | | KFINCRKEQGRYYDHLLGACVSCDSTCTQHPQQCAHFC |
| | | EKRPRSQANLQPELGRPQAGEVEVRSDNSGRHQGSEHG |
| | | PGLRLSSDQLTLYCTLGVCLCAIFCCFLVALASFLRRR |
| | | GEPLPSQPAGPRGSQANSPHAHRPVTEACDEVTASPQP |
| | | VETCSFCFPERSSPTQESAPRSLGIHGFAGTAAPQPCM |
| | | RATVGGLGVLRASTGDARPAT |
| 186 | Human TRAIL | MAMMEVQGGPSLGQTCVLIVIFTVLLQSLCVAVTYVYF |
| | | TNELKQMQDKYSKSGIACFLKEDDSYWDPNDEESMNSP |
| | | CWQVKWQLRQLVRKMILRTSEETISTVQEKQQNISPLV |
| | | RERGPQRVAAHITGTRGRSNTLSSPNSKNEKALGRKIN |
| | | SWESSRSGHSFLSNLHLRNGELVIHEKGFYYIYSQTYF |
| | | RFQEEIKENTKNDKQMVQYIYKYTSYPDPILLMKSARN |
| | | SCWSKDAEYGLYSIYQGGIFELKENDRIFVSVTNEHLI |
| | | DMDHEASFFGAFLVG |
| 187 | Mouse TRAIL | MPSSGALKDLSFSQHFRMMVICIVLLQVLLQAVSVAVT |
| | | YMYFTNEMKQLQDNYSKIGLACFSKTDEDFWDSTDGEI |
| | | LNRPCLQVKRQLYQLIEEVTLRTFQDTISTVPEKQLST |
| | | PPLPRGGRPQKVAAHITGITRRSNSALIPISKDGKTLG |
| | | QKIESWESSRKGHSFLNHVLFRNGELVIEQEGLYYIYS |
| | | QTYFRFQEAEDASKMVSKDKVRTKQLVQYIYKYTSYPD |
| | | PIVLMKSARNSCWSRDAEYGLYSIYQGGLFELKKNDRI |
| | | FVSVTNEHLMDLDQEASFFGAFLIN |
| 188 | Human TWEAK | MAARRSQRRRGRRGEPGTALLVPLALGLGLALACLGLL |
| | | LAVVSLGSRASLSAQEPAQEELVAEEDQDPSELNPQTE |
| | | ESQDPAPFLNRLVRPRRSAPKGRKTRARRAIAAHYEVH |
| | | PRPGQDGAQAGVDGTVSGWEEARINSSSPLRYNRQIGE |
| | | FIVTRAGLYYLYCQVHFDEGKAVYLKLDLLVDGVLALR |
| | | CLEEFSATAASSLGPQLRLCQVSGLLALRPGSSLRIRT |
| | | LPWAHLKAAPFLTYFGLFQVH |
| 189 | Mouse TWEAK | MASAWPRSLPQILVLGFGLVLMRAAAGEQAPGTSPCSS |
| | | GSSWSADLDKCMDCASCPARPHSDFCLGCAAAPPAHFR |
| | | LLWPILGGALSLVLVLALVSSFLVWRRCRRREKFTTPI |
| | | EETGGEGCPGVALIQ |
| 190 | Human TRANCE | MRRASRDYTKYLRGSEEMGGGPGAPHEGPLHAPPPPAP |
| | | HQPPAASRSMFVALLGLGLGQVVCSVALFFYFRAQMDP |
| | | NRISEDGTHCIYRILRLHENADFQDTTLESQDTKLIPD |
| | | SCRRIKQAFQGAVQKELQHIVGSQHIRAEKAMVDGSWL |
| | | DLAKRSKLEAQPFAHLTINATDIPSGSHKVSLSSWYHD |
| | | RGWAKISNMTFSNGKLIVNQDGFYYLYANICFRHHETS |
| | | GDLATEYLQLMVYVTKTSIKIPSSHTLMKGGSTKYWSG |
| | | NSEFHFYSINVGGFFKLRSGEEISIEVSNPSLLDPDQD |
| | | ATYFGAFKVRDID |
| 191 | Mouse TRANCE | MRRASRDYGKYLRSSEEMGSGPGVPHEGPLHPAPSAPA |
| | | PAPPPAASRSMFLALLGLGLGQVVCSIALFLYFRAQMD |
| | | PNRISEDSTHCFYRILRLHENADLQDSTLESEDTLPDS |
| | | CRRMKQAFQGAVQKELQHIVGPQRFSGAPAMMEGSWLD |
| | | VAQRGKPEAQPFAHLTINAASIPSGSHKVTLSSWYHDR |
| | | GWAKISNMTLSNGKLRVNQDGFYYLYANICFRHHETSG |
| | | SVPTDYLQLMVYVVKTSIKIPSSHNLMKGGSTKNWSGN |
| | | SEFHFYSINVGGFFKLRAGEEISIQVSNPSLLDPDQDA |
| | | TYFGAFKVQDID |
| 192 | Human TGF-beta1 | MPPSGLRLLLLLLPLLWLLVLTPGRPAAGLSTCKTIDM |
| | | ELVKRKRIEAIRGQILSKLRLASPPSQGEVPPGPLPEA |
| | | VLALYNSTRDRVAGESAEPEPEPEADYYAKEVTRVLMV |
| | | ETHNEIYDKFKQSTHSIYMFFNTSELREAVPEPVLLSR |
| | | AELRLLRLKLKVEQHVELYQKYSNNSWRYLSNRLLAPS |
| | | DSPEWLSFDVTGVVRQWLSRGGEIEGFRLSAHCSCDSR |
| | | DNTLQVDINGFTTGRRGDLATIHGMNRPFLLLMATPLE |
| | | RAQHLQSSRHRRALDTNYCFSSTEKNCCVRQLYIDFRK |
| | | DLGWKWIHEPKGYHANFCLGPCPYIWSLDTQYSKVLAL |
| | | YNQHNPGASAAPCCVPQALEPLPIVYYVGRKPKVEQLS |
| | | NMIVRSCKCS |
| 193 | Mouse TGF-beta1 | MPPSGLRLLPLLLPLPWLLVLTPGRPAAGLSTCKTIDM |
| | | ELVKRKRIEAIRGQILSKLRLASPPSQGEVPPGPLPEA |
| | | VLALYNSTRDRVAGESADPEPEPEADYYAKEVTRVLMV |
| | | DRNNAIYEKTKDISHSIYMFFNTSDIREAVPEPPLLSR |
| | | AELRLQRLKSSVEQHVELYQKYSNNSWRYLGNRLLTPT |
| | | DTPEWLSFDVTGVVRQWLNQGDGIQGFRFSAHCSCDSK |
| | | DNKLHVEINGISPKRRGDLGTIHDMNRPFLLLMATPLE |
| | | RAQHLHSSRHRRALDTNYCFSSTEKNCCVRQLYIDFRK |
| | | DLGWKWIHEPKGYHANFCLGPCPYIWSLDTQYSKVLAL |
| | | YNQHNPGASASPCCVPQALEPLPIVYYVGRKPKVEQLS |
| | | NMIVRSCKCS |
| 194 | Human TGF-beta2 | MHYCVLSAFLILHLVTVALSLSTCSTLDMDQFMRKRIE |
| | | AIRGQILSKLKLTSPPEDYPEPEEVPPEVISIYNSTRD |
| | | LLQEKASRRAAACERERSDEEYYAKEVYKIDMPPFFPS |
| | | ENAIPPTFYRPYFRIVRFDVSAMEKNASNLVKAEFRVF |
| | | RLQNPKARVPEQRIELYQILKSKDLTSPTQRYIDSKVV |
| | | KTRAEGEWLSFDVTDAVHEWLHHKDRNLGFKISLHCPC |
| | | CTFVPSNNYIIPNKSEELEARFAGIDGTSTYTSGDQKT |
| | | IKSTRKKNSGKTPHLLLMLLPSYRLESQQTNRRKKRAL |
| | | DAAYCFRNVQDNCCLRPLYIDFKRDLGWKWIHEPKGYN |
| | | ANFCAGACPYLWSSDTQHSRVLSLYNTINPEASASPCC |
| | | VSQDLEPLTILYYIGKTPKIEQLSNMIVKSCKCS |
| 195 | Mouse TGF-beta2 | MHYCVLSTFLLLHLVPVALSLSTCSTLDMDQFMRKRIE |
| | | AIRGQILSKLKLTSPPEDYPEPDEVPPEVISIYNSTRD |
| | | LLQEKASRRAAACERERSDEEYYAKEVYKIDMPSHLPS |
| | | ENAIPPTFYRPYFRIVRFDVSTMEKNASNLVKAEFRVF |
| | | RLQNPKARVAEQRIELYQILKSKDLTSPTQRYIDSKVV |
| | | KTRAEGEWLSFDVTDAVQEWLHHKDRNLGFKISLHCPC |
| | | CTFVPSNNYIIPNKSEELEARFAGIDGTSTYASGDQKT |
| | | IKSTRKKTSGKTPHLLLMLLPSYRLESQQSSRRKKRAL |
| | | DAAYCFRNVQDNCCLRPLYIDFKRDLGWKWIHEPKGYN |
| | | ANFCAGACPYLWSSDTQHTKVLSLYNTINPEASASPCC |
| | | VSQDLEPLTILYYIGNTPKIEQLSNMIVKSCKCS |
| 196 | Human TGF-beta3 | MKMHLQRALVVLALLNFATVSLSLSTCTTLDFGHIKKK |
| | | RVEAIRGQILSKLRLTSPPEPTVMTHVPYQVLALYNST |
| | | RELLEEMHGEREEGCTQENTESEYYAKEIHKFDMIQGL |
| | | AEHNELAVCPKGITSKVFRFNVSSVEKNRTNLFRAEFR |
| | | VLRVPNPSSKRNEQRIELFQILRPDEHIAKQRYIGGKN |
| | | LPTRGTAEWLSFDVTDTVREWLLRRESNLGLEISIHCP |
| | | CHTFQPNGDILENIHEVMEIKFKGVDNEDDHGRGDLGR |
| | | LKKQKDHHNPHLILMMIPPHRLDNPGQGGQRKKRALDT |
| | | NYCFRNLEENCCVRPLYIDFRQDLGWKWVHEPKGYYAN |
| | | FCSGPCPYLRSADTTHSTVLGLYNTLNPEASASPCCVP |
| | | QDLEPLTILYYVGRTPKVEQLSNMVVKSCKCS |
| 197 | Mouse TGF-beta3 | MHLQRALVVLALLNLATISLSLSTCTTLDFGHIKKKRV |
| | | EAIRGQILSKLRLTSPPEPSVMTHVPYQVLALYNSTRE |
| | | LLEEMHGEREEGCTQETSESEYYAKEIHKFDMIQGLAE |
| | | HNELAVCPKGITSKVFRFNVSSVEKNGTNLFRAEFRVL |
| | | RVPNPSSKRTEQRIELFQILRPDEHIAKQRYIGGKNLP |
| | | TRGTAEWLSFDVTDTVREWLLRRESNLGLEISIHCPCH |
| | | TFQPNGDILENVHEVMEIKFKGVDNEDDHGRGDLGRLK |
| | | KQKDHHNPHLILMMIPPHRLDSPGQGSQRKKRALDTNY |
| | | CFRNLEENCCVRPLYIDFRQDLGWKWVHEPKGYYANFC |
| | | SGPCPYLRSADTTHSTVLGLYNTLNPEASASPCCVPQD |
| | | LEPLTILYYVGRTPKVEQLSNMVVKSCKCS |
| 198 | Human EPO | MGVHECPAWLWLLLSLLSLPLGLPVLGAPPRLICDSRV |
| | | LERYLLEAKEAENITTGCAEHCSLNENITVPDTKVNFY |
| | | AWKRMEVGQQAVEVWQGLALLSEAVLRGQALLVNSSQP |
| | | WEPLQLHVDKAVSGLRSLTTLLRALGAQKEAISPPDAA |
| | | SAAPLRTITADTFRKLFRVYSNFLRGKLKLYTGEACRT |
| | | GDR |
| 199 | Mouse EPO | MGVPERPTLLLLLSLLLIPLGLPVLCAPPRLICDSRVL |
| | | ERYILEAKEAENVTMGCAEGPRLSENITVPDTKVNFYA |
| | | WKRMEVEEQAIEVWQGLSLLSEAILQAQALLANSSQPP |
| | | ETLQLHIDKAISGLRSLTSLLRVLGAQKELMSPPDTTP |
| | | PAPLRTLTVDTFCKLFRVYANFLRGKLKLYTGEVCRRG |
| | | DR |
| 200 | Human TPO | MELTELLLVVMLLLTARLTLSSPAPPACDLRVLSKLLR |
| | | DSHVLHSRLSQCPEVHPLPTPVLLPAVDFSLGEWKTQM |
| | | EETKAQDILGAVTLLLEGVMAARGQLGPTCLSSLLGQL |
| | | SGQVRLLLGALQSLLGTQLPPQGRTTAHKDPNAIFLSF |
| | | QHLLRGKVRFLMLVGGSTLCVRRAPPTTAVPSRTSLVL |
| | | TLNELPNRTSGLLETNFTASARTTGSGLLKWQQGFRAK |
| | | IPGLLNQTSRSLDQIPGYLNRIHELLNGTRGLFPGPSR |
| | | RTLGAPDISSGTSDTGSLPPNLQPGYSPSPTHPPTGQY |
| | | TLFPLPPTLPTPVVQLHPLLPDPSAPTPTPTSPLLNTS |
| | | YTHSQNLSQEG |
| 201 | Mouse TPO | MELTDLLLAAMLLAVARLTLSSPVAPACDPRLLNKLLR |
| | | DSHLLHSRLSQCPDVDPLSIPVLLPAVDFSLGEWKTQT |
| | | EQSKAQDILGAVSLLLEGVMAARGQLEPSCLSSLLGQL |
| | | SGQVRLLLGALQGLLGTQLPLQGRTTAHKDPNALFLSL |
| | | QQLLRGKVRFLLLVEGPTLCVRRTLPTTAVPSSTSQLL |
| | | TLNKFPNRTSGLLETNFSVTARTAGPGLLSRLQGFRVK |
| | | ITPGQLNQTSRSPVQISGYLNRTHGPVNGTHGLFAGTS |
| | | LQTLEASDISPGAFNKGSLAFNLQGGLPPSPSLAPDGH |
| | | TPFPPSPALPTTHGSPPQLHPLFPDPSTTMPNSTAPHP |
| | | VTMYPHPRNLSQET |
| 202 | Human FLT-3L | MTVLAPAWSPTTYLLLLLLLSSGLSGTQDCSFQHSPIS |
| | | SDFAVKIRELSDYLLQDYPVTVASNLQDEELCGGLWRL |
| | | VLAQRWMERLKTVAGSKMQGLLERVNTEIHFVTKCAFQ |
| | | PPPSCLRFVQTNISRLLQETSEQLVALKPWITRQNFSR |
| | | CLELQCQPDSSTLPPPWSPRPLEATAPTAPQPPLLLLL |
| | | LLPVGLLLLAAAWCLHWQRTRRRTPRPGEQVPPVPSPQ |
| | | DLLLVEH |
| 203 | Mouse FLT-3L | MTVLAPAWSPNSSLLLLLLLLSPCLRGTPDCYFSHSPI |
| | | SSNFKVKFRELTDHLLKDYPVTVAVNLQDEKHCKALWS |
| | | LFLAQRWIEQLKTVAGSKMQTLLEDVNTEIHFVTSCTF |
| | | QPLPECLRFVQTNISHLLKDTCTQLLALKPCIGKACQN |
| | | FSRCLEVQCQPDSSTLLPPRSPIALEATELPEPRPRQL |
| | | LLLLLLLLPLTLVLLAAAWGLRWQRARRRGELHPGVPL |
| | | PSHP |
| 204 | Human SCF | MKKTQTWILTCIYLQLLLFNPLVKTEGICRNRVTNNVK |
| | | DVTKLVANLPKDYMITLKYVPGMDVLPSHCWISEMVVQ |
| | | LSDSLTDLLDKFSNISEGLSNYSIIDKLVNIVDDLVEC |
| | | VKENSSKDLKKSFKSPEPRLFTPEEFFRIFNRSIDAFK |
| | | DFVVASETSDCVVSSTLSPEKDSRVSVTKPFMLPPVAA |
| | | SSLRNDSSSSNRKAKNPPGDSSLHWAAMALPALFSLII |
| | | GFAFGALYWKKRQPSLTRAVENIQINEEDNEISMLQEK |
| | | EREFQEV |
| 205 | Mouse SCF | MKKTQTWIITCIYLQLLLFNPLVKTKEICGNPVTDNVK |
| | | DITKLVANLPNDYMITLNYVAGMDVLPSHCWLRDMVIQ |
| | | LSLSLTTLLDKFSNISEGLSNYSIIDKLGKIVDDLVLC |
| | | MEENAPKNIKESPKRPETRSFTPEEFFSIFNRSIDAFK |
| | | DFMVASDTSDCVLSSTLGPEKDSRVSVTKPFMLPPVAA |
| | | SSLRNDSSSSNRKAAKAPEDSGLQWTAMALPALISLVI |
| | | GFAFGALYWKKKQSSLTRAVENIQINEEDNEISMLQQK |
| | | EREFQEV |
| 206 | Human M-CSF | MTAPGAAGRCPPTTWLGSLLLLVCLLASRSITEEVSEY |
| | | CSHMIGSGHLQSLQRLIDSQMETSCQrTFEFVDQEQLK |
| | | DPVCYLKKAFLLVQDIMEDTMRFRDNTPNAIAIVQLQE |
| | | LSLRLKSCFTKDYEEHDKACVRTFYETPLQLLEKVKNV |
| | | FNETKNLLDKDWNIFSKNCNNSFAECSSQDVVTKPDCN |
| | | CLYPKAIPSSDPASVSPHQPLAPSMAPVAGLTWEDSEG |
| | | TEGSSLLPGEQPLHTVDPGSAKQRPPRSTCQSFEPPET |
| | | PVVKDSTIGGSPQPRPSVGAFNPGMEDILDSAMGTNWV |
| | | PEEASGEASEIPVPQGTELSPSRPGGGSMQTEPARPSN |
| | | FLSASSPLPASAKGQQPADVTGTALPRVGPVRPTGQDW |
| | | NHTPQKTDHPSALLRDPPEPGSPRISSLRPQGLSNPST |
| | | LSAQPQLSRSHSSGSVLPLGELEGRRSTRDRRSPAEPE |
| | | GGPASEGAARPLPRFNSVPLTDTGHERQSEGSFSPQLQ |
| | | ESVFHLLVPSVILVLLAVGGLLFYRWRRRSHQEPQRAD |
| | | SPLEQPEGSPLTQDDRQVELPV |
| 207 | Mouse M-CSF | MTARGAAGRCPSSTWLGSRLLLVCLLMSRSIAKEVSEH |
| | | CSHMIGNGHLKVLQQLIDSQMETSCQIAFEFVDQEQLD |
| | | DPVCYLKKAFFLVQDIIDETMRFKDNTPNANATERLQE |
| | | LSNNLNSCFTKDYEEQNKACVRTFHETPLQLLEKIKNF |
| | | FNETKNLLEKDWNIFTKNCNNSFAKCSSRDVVTKPDCN |
| | | CLYPKATPSSDPASASPHQPPAPSMAPLAGLAWDDSQR |
| | | TEGSSLLPSELPLRIEDPGSAKQRPPRSTCQTLESTEQ |
| | | PNHGDRLTEDSQPHPSAGGPVPGVEDILESSLGTNWVL |
| | | EEASGEASEGFLTQEAKFSPSTPVGGSIQAETDRPRAL |
| | | SASPFPKSTEDQKPVDITDRPLTEVNPMRPIGQTQNNT |
| | | PEKTDGTSTLREDHQEPGSPHIATPNPQRVSNSATPVA |
| | | QLLLPKSHSWGIVLPLGELEGKRSTRDRRSPAELEGGS |
| | | ASEGAARPVARFNSIPLTDTGHVEQHEGSSDPQIPESV |
| | | FHLLVPGIILVLLTVGGLLFYKWKWRSHRDPQTLDSSV |
| | | GRPEDSSLTQDEDRQVELPV |
| 208 | Human MSP | MGWLPLLLLLTQCLGVPGQRSPLNDFQVLRGTELQHLL |
| | | HAVVPGPWQEDVADAEECAGRCGPLMDCRAFHYNVSSH |
| | | GCQLLPWTQHSPHTRLRRSGRCDLFQKKDYVRTCIMNN |
| | | GVGYRGTMATTVGGLPCQAWSHKFPNDHKYTPTLRNGL |
| | | EENFCRNPDGDPGGPWCYTTDPAVRFQSCGIKSCREAA |
| | | CVWCNGEEYRGAVDRTESGRECQRWDLQHPHQHPFEPG |
| | | KFLDQGLDDNYCRNPDGSERPWCYTTDPQIEREFCDLP |
| | | RCGSEAQPRQEATTVSCFRGKGEGYRGTANTTTAGVPC |
| | | QRWDAQIPHQHRFTPEKYACKDLRENFCRNPDGSEAPW |
| | | CFTLRPGMRAAFCYQIRRCTDDVRPQDCYHGAGEQYRG |
| | | TVSKTRKGVQCQRWSAETPHKPQFTFTSEPHAQLEENF |
| | | CRNPDGDSHGPWCYTMDPRTPFDYCALRRCADDQPPSI |
| | | LDPPDQVQFEKCGKRVDRLDQRRSKLRVVGGHPGNSPW |
| | | TVSLRNRQGQHFCGGSLVKEQWILTARQCFSSCHMPLT |
| | | GYEVWLGTLFQNPQHGEPSLQRVPVAKMVCGPSGSQLV |
| | | LLKLERSVTLNQRVALICLPPEWYVVPPGTKCEIAGWG |
| | | ETKGTGNDTVLNVALLNVISNQECNIKHRGRVRESEMC |
| | | TEGLLAPVGACEGDYGGPLACFTHNCWVLEGIIIPNRV |
| | | CARSRWPAVFTRVSVFVDWIHKVMRLG |
| 209 | Mouse MSP | MGLPLPLLQSSLLLMLLLRLSAASTNLNWQCPRIPYAA |
| | | SRDFSVKYVVPSFSAGGRVQATAAYEDSTNSAVFVATR |
| | | NHLHVLGPDLQFIENLTTGPIGNPGCQTCASCGPGPHG |
| | | PPKDTDTLVLVMEPGLPALVSCGSTLQGRCFLHELEPR |
| | | GKALHLAAPACLFSANNNKPEACTDCVASPLGTRVTVV |
| | | EQGHASYFYVASSLDPELAASFSPRSVSIRRLKSDTSG |
| | | FQPGFPSLSVLPKYLASYLIKYVYSFHSGDFVYFLTVQ |
| | | PISVTSPPSALHTRLVRLNAVEPEIGDYRELVLDCHFA |
| | | PKRRRRGAPEGTQPYPVLQAAHSAPVDAKLAVELSISE |
| | | GQEVLFGVFVTVKDGGSGMGPNSVVCAFPIYHLNILIE |
| | | EGVEYCCHSSNSSSLLSRGLDFFQTPSFCPNPPGGEAS |
| | | GPSSRCHYFPLMVHASFTRVDLFNGLLGSVKVTALHVT |
| | | RLGNVTVAHMGTVDGRVLQVEIARSLNYLLYVSNFSLG |
| | | SSGQPVHRDVSRLGNDLLFASGDQVFKVPIQGPGCRHF |
| | | LTCWRCLRAQRFMGCGWCGDRCDRQKECPGSWQQDHCP |
| | | PEISEFYPHSGPLRGTTRLTLCGSNFYLRPDDVVPEGT |
| | | HQITVGQSPCRLLPKDSSSPRPGSLKEFIQELECELEP |
| | | LVTQAVGTTNISLVITNMPAGKHFRVEGISVQEGFSFV |
| | | EPVLTSIKPDFGPRAGGTYLTLEGQSLSVGTSRAVLVN |
| | | GTQCRLEQVNEEQILCVTPPGAGTARVPLHLQIGGAEV |
| | | PGSWTFHYKEDPIVLDISPKCGYSGSHIMIHGQHLTSA |
| | | WHFTLSFHDGQSTVESRCAGQFVEQQQRRCRLPEYVVR |
| | | NPQGWATGNLSVWGDGAAGFTLPGFRFLPPPSPLRAGL |
| | | VELKPEEHSVKVEYVGLGAVADCVTVNMTVGGEVCQHE |
| | | LRGDVVICPLPPSLQLGKDGVPLQVCVDGGCHILSQVV |
| | | RSSPGRASQRILLIALLVLILLVAVLAVALIFNSRRRK |
| | | KQLGAHSLSPTTLSDINDTASGAPNHEESSESRDGTSV |
| | | PLLRTESIRLQDLDRMLLAEVKDVLIPHEQVVIHTDQV |
| | | IGKGHFGVVYHGEYTDGAQNQTHCAIKSLSRITEVQEV |
| | | EAFLREGLLMRGLHHPNILALIGIMLPPEGLPRVLLPY |
| | | MRHGDLLHFIRSPQRNPTVKDLVSFGLQVACGMEYLAE |
| | | QKFVHRDLAARNCMLDESFTVKVADFGLARGVLDKEYY |
| | | SVRQHRHARLPVKWMALESLQTYRFTTKSDVWSFGVLL |
| | | WELLTRGAPPYPHIDPFDLSHFLAQGRRLPQPEYCPDS |
| | | LYHVMLRCWEADPAARPTFRALVLEVKQVVASLLGDHY |
| | | VQLTAAYVNVGPRAVDDGSVPPEQVQPSPQHCRSTSKP |
| | | RPLSEPPLPT |
| 210 | Linker | GSSGGSGGSGG |
| 211 | Linker | GGGSGGGS |
| 212 | Linker | GGGSGGGSGGGS |
| 213 | Linker | GGGGSGGGGSGGGGS |
| 214 | Linker | GGGGSGGGGSGGGGSGGGGSGGGGS |
| 215 | Linker | GGGGSGGGGS |
| 216 | Linker | (GGGGS)n |
| 217 | Linker | GGGGSGS |
| 218 | Linker | GGGGSGGGGSGGGGSGS |
| 219 | Linker | GGSLDPKGGGGS |
| 220 | Linker | PKSCDKTHTCPPCPAPELLG |
| 221 | Linker | SKYGPPCPPCPAPEFLG |
| 222 | Linker | GKSSGSGSESKS |
| 223 | Linker | GSTSGSGKSSEGKG |
| 224 | Linker | GSTSGSGKSSEGSGSTKG |
| 225 | Linker | GSTSGSGKPGSGEGSTKG |
| 226 | Linker | GSTSGSGKPGSSEGST |
| 227 | Linker | (GSGGS)n |
| 228 | Linker | (GGGS)n |
| 229 | Linker | GGSG |
| 230 | Linker | GGSGG |
| 231 | Linker | GSGSG |
| 232 | Linker | GSGGG |
| 233 | Linker | GGGSG |
| 234 | Linker | GSSSG |
| 235 | (Intentionally | (Intentionally Omitted) |
| to | Omitted) | |
| 244 | | |
| 245 | Linker | GPQGTAGQ |
| 246 | (Intentionally | (Intentionally Omitted) |
| to | Omitted) | |
| 249 | | |
| 250 | Linker | YGAGLGW |
| 251 | (Intentionally | (Intentionally Omitted) |
| to | Omitted) | |
| 262 | | |
| 263 | CM | APRSALAHGLF |
| 264 | CM | AQNLLGMY |
| 265 | CM | LSGRSDNHGGAVGLLAPP |
| 266 | CM | VHMPLGFLGPGGLSGRSDNH |
| 267 | CM | LSGRSDNHGGVHMPLGFLGP |
| 268 | CM | LSGRSDNHGGSGGSISSGLLSS |
| 269 | CM | ISSGLLSSGGSGGSLSGRSGNH |
| 270 | CM | LSGRSDNHGGSGGSQNQALRMA |
| 271 | CM | QNQALRMAGGSGGSLSGRSDNH |
| 272 | CM | LSGRSGNHGGSGGSQNQALRMA |
| 273 | CM | QNQALRMAGGSGGSLSGRSGNH |
| 274 | CM | ISSGLLSGRSGNH |
| 275 | CM | AVGLLAPPGGTSTSGRSANPRG |
| 276 | CM | TSTSGRSANPRGGGAVGLLAPP |
| 277 | CM | VHMPLGFLGPGGTSTSGRSANPRG |
| 278 | CM | TSTSGRSANPRGGGVHMPLGFLGP |
| 279 | CM | LSGRSGNHGGSGGSISSGLLSS |
| 280 | Cleavable Sequence | PRFKIIGG |
| 281 | Cleavable Sequence | PRFRIIGG |
| 282 | Cleavable Sequence | SSRHRRALD |
| 283 | Cleavable Sequence | RKSSIIIRMRDVVL |
| 284 | Cleavable Sequence | SSSFDKGKYKKGDDA |
| 285 | Cleavable Sequence | SSSFDKGKYKRGDDA |
| 286 | Cleavable Sequence | IEGR |
| 287 | Cleavable Sequence | IDGR |
| 288 | Cleavable Sequence | GGSIDGR |
| 289 | Cleavable Sequence | PLGLWA |
| 290 | Cleavable Sequence | GPQGIAGQ |
| 291 | Cleavable Sequence | GPQGLLGA |
| 292 | Cleavable Sequence | GIAGQ |
| 293 | Cleavable Sequence | GPLGIAGI |
| 294 | Cleavable Sequence | GPEGLRVG |
| 295 | Cleavable Sequence | YGAGLGVV |
| 296 | Cleavable Sequence | AGLGVVER |
| 297 | Cleavable Sequence | AGLGISST |
| 298 | Cleavable Sequence | EPQALAMS |
| 299 | Cleavable Sequence | QALAMSAI |
| 300 | Cleavable Sequence | AAYHLVSQ |
| 301 | Cleavable Sequence | MDAFLESS |
| 302 | Cleavable Sequence | ESLPVVAV |
| 303 | Cleavable Sequence | SAPAVESE |
| 304 | Cleavable Sequence | DVAQFVLT |
| 305 | Cleavable Sequence | VAQFVLT |
| 306 | Cleavable Sequence | VAQFVLTE |
| 307 | Cleavable Sequence | AQFVLTEG |
| 308 | Cleavable Sequence | PVQPIGPQ |
| 309 | IFN-α2b-1204dL- | METDTLLLWVLLLWVPGSTGCDLPQTHSLGSRRTLMLL |
| | hIgG4 | AQMRRISLFSCLKDRHDFGFPQEEFGNQFQKAETIPVL |
| | | HEMIQQIFNLFSTKDSSAAWDETLLDKFYTELYQQLND |
| | | LEACVIQGVGVTETPLMKEDSILAVRKYFQRITLYLKE |
| | | KKYSPCAWEVVRAEIMRSFSLSTNLQESLRSKESGRSD |
| | | NIGGGSESKYGPPCPPCPAPEFLGGPSVFLFPPKPKDT |
| | | LMISRTPEVTCVVVDVSQEDPEVQFNWYVDGVEVHNAK |
| | | TKPREEQFNSTYRVVSVLTVLHQDWLNGKEYKCKVSNK |
| | | GLPSSIEKTISKAKGQPREPQVYTLPPSQEEMTKNQVS |
| | | LTCLVKGFYPSDIAVEWESNGQPENNYKTTPPVLDSDG |
| | | SFFLYSRLTVDKSRWQQGNVFSCSVMHEALHNHYTQKS |
| | | LSLS |
| 310 | IFN-α-1204dL- | ATGGAAACCGACACACTGCTGCTGTGGGTGCTGCTTTT |
| | hIgG4 | GTGGGTGCCAGGATCCACAGGCTGTGATCTGCCTCAAA |
| | (polynucleotide) | CGCATTCATTGGGGTCCAGGCGCACGCTTATGTTGCTT |
| | | GCACAGATGAGGAGAATATCACTTTTCTCTTGCTTGAA |
| | | GGACCGCCACGATTTTGGCTTTCCGCAGGAAGAGTTCG |
| | | GTAACCAGTTCCAAAAGGCAGAGACAATCCCCGTTTTG |
| | | CATGAGATGATCCAACAGATCTTTAACCTGTTTTCAAC |
| | | CAAGGATAGCAGCGCAGCGTGGGATGAGACACTGCTTG |
| | | ACAAGTTTTACACCGAGCTCTATCAGCAACTTAATGAT |
| | | CTCGAAGCCTGCGTAATTCAAGGAGTAGGCGTTACAGA |
| | | GACACCTTTGATGAAGGAGGATTCCATCCTTGCAGTAA |
| | | GAAAATACTTCCAGAGGATCACCCTCTACCTCAAAGAA |
| | | AAGAAATACTCCCCATGCGCGTGGGAAGTAGTGCGAGC |
| | | TGAAATAATGCGGAGCTTTTCTTTGTCAACTAATCTCC |
| | | AAGAATCTCTGAGAAGCAAGGAGTCAGGTAGGTCTGAT |
| | | AATATCGGGGGAGGTTCTGAATCTAAGTACGGCCCTCC |
| | | TTGTCCTCCATGTCCTGCTCCAGAGTTTCTCGGAGGCC |
| | | CCTCCGTGTTCCTGTTTCCTCCAAAGCCTAAGGACACC |
| | | CTGATGATCAGCAGAACCCCTGAAGTGACCTGCGTGGT |
| | | GGTCGACGTTTCACAAGAGGACCCCGAGGTGCAGTTCA |
| | | ATTGGTACGTGGACGGCGTGGAAGTGCACAACGCCAAG |
| | | ACCAAGCCTAGAGAGGAACAGTTCAACAGCACCTACAG |
| | | AGTGGTGTCCGTGCTGACCGTGCTGCACCAGGATTGGC |
| | | TGAACGGCAAAGAGTACAAGTGCAAGGTGTCCAACAAG |
| | | GGCCTGCCTAGCAGCATCGAGAAAACCATCAGCAAGGC |
| | | CAAGGGCCAGCCAAGGGAACCCCAGGTTTACACACTGC |
| | | CACCTAGCCAAGAGGAAATGACCAAGAACCAGGTGTCC |
| | | CTGACCTGCCTGGTCAAGGGCTTTTACCCCTCCGATAT |
| | | CGCCGTGGAATGGGAGAGCAATGGCCAGCCTGAGAACA |
| | | ACTACAAGACCACACCTCCTGTGCTGGACAGCGACGGC |
| | | TCATTCTTCCTGTACAGCAGACTGACCGTGGACAAGAG |
| | | CAGATGGCAGCAGGGCAACGTGTTCAGCTGCAGCGTGA |
| | | TGCACGAGGCCCTGCACAACCACTACACCCAGAAGTCT |
| | | CTGAGCCTGAGCTGA |
| 311 | IFN-α2b-1490DNI- | METDTLLLWVLLLWVPGSTGCDLPQTHSLGSRRTLMLL |
| | hIgG4 | AQMRRISLFSCLKDRHDFGFPQEEFGNQFQKAETIPVL |
| | | HEMIQQIFNLFSTKDSSAAWDETLLDKFYTELYQQLND |
| | | LEACVIQGVGVTETPLMKEDSILAVRKYFQRITLYLKE |
| | | KKYSPCAWEVVRAEIMRSFSLSTNLQESLRSKEISSGL |
| | | LSGRSDNIGGGSESKYGPPCPPCPAPEFLGGPSVFLFP |
| | | PKPKDTLMISRTPEVTCVVVDVSQEDPEVQFNWYVDGV |
| | | EVHNAKTKPREEQFNSTYRVVSVLTVLHQDWLNGKEYK |
| | | CKVSNKGLPSSIEKTISKAKGQPREPQVYTLPPSQEEM |
| | | TKNQVSLTCLVKGFYPSDIAVEWESNGQPENNYKTTPP |
| | | VLDSDGSFFLYSRLTVDKSRWQQGNVFSCSVMHEALHN |
| | | HYTQKSLSLS |
| 312 | IFN-α2b-1490DNI- | ATGGAAACCGACACACTGCTGCTGTGGGTGCTGCTTTT |
| | hIgG4 | GTGGGTGCCAGGATCCACAGGCTGTGATCTGCCTCAAA |
| | (polynucleotide) | CGCATTCATTGGGGTCCAGGCGCACGCTTATGTTGCTT |
| | | GCACAGATGAGGAGAATATCACTTTTCTCTTGCTTGAA |
| | | GGACCGCCACGATTTTGGCTTTCCGCAGGAAGAGTTCG |
| | | GTAACCAGTTCCAAAAGGCAGAGACAATCCCCGTTTTG |
| | | CATGAGATGATCCAACAGATCTTTAACCTGTTTTCAAC |
| | | CAAGGATAGCAGCGCAGCGTGGGATGAGACACTGCTTG |
| | | ACAAGTTTTACACCGAGCTCTATCAGCAACTTAATGAT |
| | | CTCGAAGCCTGCGTAATTCAAGGAGTAGGCGTTACAGA |
| | | GACACCTTTGATGAAGGAGGATTCCATCCTTGCAGTAA |
| | | GAAAATACTTCCAGAGGATCACCCTCTACCTCAAAGAA |
| | | AAGAAATACTCCCCATGCGCGTGGGAAGTAGTGCGAGC |
| | | TGAAATAATGCGGAGCTTTTCTTTGTCAACTAATCTCC |
| | | AAGAATCTCTGAGAAGCAAGGAGATTAGTTCTGGCCTG |
| | | CTGTCAGGTAGGTCTGATAATATCGGGGGAGGTTCTGA |
| | | ATCTAAGTACGGCCCTCCTTGTCCTCCATGTCCTGCTC |
| | | CAGAGTTTCTCGGAGGCCCCTCCGTGTTCCTGTTTCCT |
| | | CCAAAGCCTAAGGACACCCTGATGATCAGCAGAACCCC |
| | | TGAAGTGACCTGCGTGGTGGTCGACGTTTCACAAGAGG |
| | | ACCCCGAGGTGCAGTTCAATTGGTACGTGGACGGCGTG |
| | | GAAGTGCACAACGCCAAGACCAAGCCTAGAGAGGAACA |
| | | GTTCAACAGCACCTACAGAGTGGTGTCCGTGCTGACCG |
| | | TGCTGCACCAGGATTGGCTGAACGGCAAAGAGTACAAG |
| | | TGCAAGGTGTCCAACAAGGGCCTGCCTAGCAGCATCGA |
| | | GAAAACCATCAGCAAGGCCAAGGGCCAGCCAAGGGAAC |
| | | CCCAGGTTTACACACTGCCACCTAGCCAAGAGGAAATG |
| | | ACCAAGAACCAGGTGTCCCTGACCTGCCTGGTCAAGGG |
| | | CTTTTACCCCTCCGATATCGCCGTGGAATGGGAGAGCA |
| | | ATGGCCAGCCTGAGAACAACTACAAGACCACACCTCCT |
| | | GTGCTGGACAGCGACGGCTCATTCTTCCTGTACAGCAG |
| | | ACTGACCGTGGACAAGAGCAGATGGCAGCAGGGCAACG |
| | | TGTTCAGCTGCAGCGTGATGCACGAGGCCCTGCACAAC |
| | | CACTACACCCAGAAGTCTCTGAGCCTGAGCTGA |
| 313 | ProC440 without | CDLPQTHSLGSRRTLMLLAQMRRISLFSCLKDRHDFGF |
| | signal sequence | PQEEFGNQFQKAETIPVLHEMIQQIFNLFSTKDSSAAW |
| | | DETLLDKFYTELYQQLNDLEACVIQGVGVTETPLMKED |
| | | SILAVRKYFQRITLYLKEKKYSPCAWEVVRAEIMRSFS |
| | | LSTNLQESLRSKESGRSDNICPPCPAPEFLGGPSVFLF |
| | | PPKPKDTLMISRTPEVTCVVVDVSQEDPEVQFNWYVDG |
| | | VEVHNAKTKPREEQFNSTYRVVSVLTVLHQDWLNGKEY |
| | | KCKVSNKGLPSSIEKTISKAKGQPREPQVYTLPPSQEE |
| | | MTKNQVSLTCLVKGFYPSDIAVEWESNGQPENNYKTTP |
| | | PVLDSDGSFFLYSRLTVDKSRWQQGNVFSCSVMHEALH |
| | | NHYTQKSLSLS |
| 314 | PROC657 first | CDLPQTHSLGSRRTLMLLAQMRRISLFSCLKDRHDFGF |
| | monomer (knob | PQEEFGNQFQKAETIPVLHEMIQQIFNLFSTKDSSAAW |
| | mutation) without | DETLLDKFYTELYQQLNDLEACVIQGVGVTETPLMKED |
| | signal sequence | SILAVRKYFQRITLYLKEKKYSPCAWEVVRAEIMRSFS |
| | | LSTNLQESLRSKESGRSDNICPPCPAPEFEGGPSVFLF |
| | | PPKPKDTLMISRTPEVTCVVVDVSQEDPEVQFNWYVDG |
| | | VEVHNAKTKPREEQFNSTYRVVSVLTVLHQDWLNGKEY |
| | | KCKVSNKGLPSSIEKTISKAKGQPREPQVYTLPPCQEE |
| | | MTKNQVSLWCLVKGFYPSDIAVEWESNGQPENNYKTTP |
| | | PVLDSDGSFFLYSRLTVDKSRWQEGNVFSCSVMHEALH |
| | | NHYTQKSLSLSLG |
| 315 | human IgG Fc with | CPPCPAPEFEGGPSVFLFPPKPKDTLMISRTPEVTCVV |
| | a knob mutation | VDVSQEDPEVQFNWYVDGVEVHNAKTKPREEQFNSTYR |
| | | VVSVLTVLHQDWLNGKEYKCKVSNKGLPSSIEKTISKA |
| | | KGQPREPQVYTLPPCQEEMTKNQVSLWCLVKGFYPSDI |
| | | AVEWESNGQPENNYKTTPPVLDSDGSFFLYSRLTVDKS |
| | | RWQEGNVFSCSVMHEALHNHYTQKSLSLSLG |
| 316 | human IgG Fc with | CPPCPAPEFEGGPSVFLFPPKPKDTLMISRTPEVTCVV |
| | a hole mutation | VDVSQEDPEVQFNWYVDGVEVHNAKTKPREEQFNSTYR |
| | | VVSVLTVLHQDWLNGKEYKCKVSNKGLPSSIEKTISKA |
| | | KGQPREPQVCTLPPSQEEMTKNQVSLSCAVKGFYPSDI |
| | | AVEWESNGQPENNYKTTPPVLDSDGSFFLVSRLTVDKS |
| | | RWQEGNVFSCSVMHEALHNRFTQKSLSLSLG |
| 317 | stub moiety | SDNI |
| 318 | Linker | GSSGGS |
| 319 | Linker | ESKY |
| 320 | ProC286 without | ESKYGPPCPPCPAPEFLGGPSVFLFPPKPKDTLMISRT |
| | signal sequence | PEVTCVVVDVSQEDPEVQFNWYVDGVEVHNAKTKPREE |
| | | QFNSTYRVVSVLTVLHQDWLNGKEYKCKVSNKGLPSSI |
| | | EKTISKAKGQPREPQVYTLPPSQEEMTKNQVSLTCLVK |
| | | GFYPSDIAVEWESNGQPENNYKTTPPVLDSDGSFFLYS |
| | | RLTVDKSRWQQGNVFSCSVMHEALHNHYTQKSLSLSSG |
| | | GGGSGRSDNIGGGSCDLPQTHSLGSRRTLMLLAQMRRI |
| | | SLFSCLKDRHDFGFPQEEFGNQFQKAETIPVLHEMIQQ |
| | | IFNLFSTKDSSAAWDETLLDKFYTELYQQLNDLEACVI |
| | | QGVGVTETPLMKEDSILAVRKYFQRITLYLKEKKYSPC |
| | | AWEVVRAEIMRSFSLSTNLQESLRSKE |
| 321 | Linker | SGGG |
| 322 | PROC657 second | SDNICPPCPAPEFEGGPSVFLFPPKPKDTLMISRTPEV |
| | monomer (hole | TCVVVDVSQEDPEVQFNWYVDGVEVHNAKTKPREEQFN |
| | mutation) without | STYRVVSVLTVLHQDWLNGKEYKCKVSNKGLPSSIEKT |
| | signal sequence | ISKAKGQPREPQVCTLPPSQEEMTKNQVSLSCAVKGFY |
| | | PSDIAVEWESNGQPENNYKTTPPVLDSDGSFFLVSRLT |
| | | VDKSRWQEGNVFSCSVMHEALHNRFTQKSLSLSLG |
| 323 | PR0859 sequence | CDLPQTHSLGSRRTLMLLAQMRKISLFSCLKDRHDFGF |
| | without signal | PQEEFGNQFQKAETIPVLHEMIQQIFNLFTTKDSSAAW |
| | sequence | DEDLLDKFCTELYQQLNDLEACVMQEERVGETPLMNVD |
| | | SILAVKKYFRRITLYLTEKKYSPCAWEVVRAEIMRSLS |
| | | LSTNLQERLRRKELSGRSDNICPPCPAPEFLGGPSVFL |
| | | FPPKPKDTLMISRTPEVTCVVVDVSQEDPEVQFNWYVD |
| | | GVEVHNAKTKPREEQFNSTYRVVSVLTVLHQDWLNGKE |
| | | YKCKVSNKGLPSSIEKTISKAKGQPREPQVYTLPPSQE |
| | | EMTKNQVSLTCLVKGFYPSDIAVEWESNGQPENNYKTT |
| | | PPVLDSDGSFFLYSRLTVDKSRWQQGNVFSCSVMHEAL |
| | | HNHYTQKSLSLS |
| 324 | Universal IFN- | CDLPQTHSLGSRRTLMLLAQMRKISLFSCLKDRHDFGF |
| | alpha A/D sequence | PQEEFGNQFQKAETIPVLHEMIQQIFNLFTTKDSSAAW |
| | | DEDLLDKFCTELYQQLNDLEACVMQEERVGETPLMNVD |
| | | SILAVKKYFRRITLYLTEKKYSPCAWEVVRAEIMRSLS |
| | | LSTNLQERLRRKE |
| 325 | Interferon beta, | MSYNLLGFLQRSSNFQCQKLLWQLNGRLEYCLKDRMNF |
| | Chain A, human | DIPEEIKQLQQFQKEDAALTIYEMLQNIFAIFRQDSSS |
| | (1AU1) | TGWNETIVENLLANVYHQINHLKTVLEEKLEKEDFTRG |
| | | KLMSSLHLKRYYGRILHYLKAKEYSHCAWTIVRVEILR |
| | | NFYFINRLTGYLRN |
| 326 | IFNB_CHICK | MTANHQSPGMHSILLLLLLPALTTTFSCNHLRHQDANF |
| | Q90873.1 | SWKSLQLLQNTAPPPPQPCPQQDVTFPFPETLLKSKDK |
| | | KQAAITTLRILQHLFNMLSSPHTPKHWIDRTRHSLLNQ |
| | | IQHYIHHLEQCFVNQGTRSQRRGPRNAHLSINKYFRSI |
| | | HNFLQHNNYSACTWDHVRLQARDCFRHVDTLIQWMKSR |
| | | APLTASSKRLNTQ |
| 327 | IFNA3_CANLF | MALPCSFSVALVLLSCHSLCCLACHLPDTHSLRNWRVL |
| | 097945.1 | TLLGQMRRLSASSCDHYTTDFAFPKELFDGQRLQEAQA |
| | | LSVVHVMTQKVFHLFCTNTSSAPWNMTLLEELCSGLSE |
| | | QLDDLDACPLQEAGLAETPLMHEDSTLRTYFQRISLYL |
| | | QDRNHSPCAWEMVRAEIGRSFFSLTILQERVRRRK |
| 328 | IFN_ANAPL | MPGPSAPPPPAIYSALALLLLLTPPANAFSCSPLRLHD |
| | P51526.1 | SAFAWDSLQLLRNMAPSPTQPCPQQHAPCSFPDTLLDT |
| | | NDTQQAAHTALHLLQHLFDTLSSPSTPAHWLHTARHDL |
| | | LNQLQHHIHHLERCFPADAARLHRRGPRNLHLSINKYF |
| | | GCIQHFLQNHTYSPCAWDHVRLEAHACFQRIHRLTRTM |
| | | R |
| 329 | IFNAH_BOVIN | MAPAWSFLLALLLLSCNAICSLGCHLPHTHSLPNRRVL |
| | P49878.1 | TLLRQLRRVSPSSCLQDRNDFAFPQEALGGSQLQKAQA |
| | | ISVLHEVTQHTFQLFSTEGSAAAWDESLLDKLRAALDQ |
| | | QLTDLQACLRQEEGLRGAPLLKEDASLAVRKYFHRLTL |
| | | YLREKRHNPCAWEVVRAEVMRAFSSSTNLQERFRRKD |
| 330 | IFNA1_CHICK | MAVPASPQHPRGYGILLLTLLLKALATTASACNHLRPQ |
| | P42165.1 | DATFSHDSLQLLRDMAPTLPQLCPQHNASCSFNDTILD |
| | | TSNTRQADKTTHDILQHLFKILSSPSTPAHWNDSQRQS |
| | | LLNRIHRYTQHLEQCLDSSDTRSRTRWPRNLHLTIKKH |
| | | FSCLHTFLQDNDYSACAWEHVRLQARAWFLHIHNLTGN |
| | | TRT |
| 331 | IFNA_FELCA | MALPSSFLVALVALGCNSVCSLGCDLPQTHGLLNRRAL |
| | P35849.1 | TLLGQMRRLPASSCQKDRNDFAFPQDVFGGDQSHKAQA |
| | | LSVVHVTNQKIFHFFCTEASSSAAWNTTLLEEFCTGLD |
| | | RQLTRLEACVLQEVEEGEAPLTNEDIHPEDSILRNYFQ |
| | | RLSLYLQEKKYSPCAWEIVRAEIMRSLYYSSTALQKRL |
| | | RSEK |
| 332 | interferon-beta-1 | MANKCILQIALLMCFSTTALSMSYDVLRYQQRSSNLAC |
| | [Sus scrofa] | QKLLGQLPGTPQYCLEDRMNFEVPEEIMQPPQFQKEDA |
| | AAA31056.1 | VLIIHEMLQQIFGILRRNFSSTGWNETVIKTILVELDG |
| | | QMDDLETILEEIMEEENFPRGDMTILHLKKYYLSILQY |
| | | LKSKEYRSCAWTVVQVEILRNFSFLNRLTDYLRN |
| 333 | IFNB2_BOVIN | MTHRCLLQMVLLLCFSTTALSRSYSLLRFQQRRSLALC |
| | P01576.1 | QKLLRQLPSTPQHCLEARMDFQMPEEMKQAQQFQKEDA |
| | | ILVIYEMLQQIFNILTRDFSSTGWSETIIEDLLEELYE |
| | | QMNHLEPIQKEIMQKQNSTMGDTTVLHLRKYYFNLVQY |
| | | LKSKEYNRCAWTVVRVQILRNFSFLTRLTGYLRE |
| 334 | A Chain A, | CDLPQTHSLGSRRTLMLLAQMRRISLFSCLKDRHDFGF |
| | INTERFERON- | PQEEFGNQFQKAETIPVLHEMIQQIFNLFSTKDSSAAW |
| | ALPHA 2B 1RH2 | DETLLDKFYTELYQQLNDLEACVIQGVGVTETPLMNED |
| | | SILAVRKYFQRITLYLKEKKYSPCAWEVVRAEIMRSFS |
| | | LSTNLQESLRSKE |
| 335 | Linker | SGGGG |
| 336 | ProC288 without | CDLPQTHSLGSRRTLMLLAQMRRISLFSCLKDRHDFGF |
| | signal sequence | PQEEFGNQFQKAETIPVLHEMIQQIFNLFSTKDSSAAW |
| | | DETLLDKFYTELYQQLNDLEACVIQGVGVTETPLMKED |
| | | SILAVRKYFQRITLYLKEKKYSPCAWEVVRAEIMRSFS |
| | | LSTNLQESLRSKESGGGGSGRSDNICPPCPAPEFLGGP |
| | | SVFLFPPKPKDTLMISRTPEVTCVVVDVSQEDPEVQFN |
| | | WYVDGVEVHNAKTKPREEQFNSTYRVVSVLTVLHQDWL |
| | | NGKEYKCKVSNKGLPSSIEKTISKAKGQPREPQVYTLP |
| | | PSQEEMTKNQVSLTCLVKGFYPSDIAVEWESNGQPENN |
| | | YKTTPPVLDSDGSFFLYSRLTVDKSRWQQGNVFSCSVM |
| | | HEALHNHYTQKSLSLS |
| 337 | ProC289 without | CDLPQTHSLGSRRTLMLLAQMRRISLFSCLKDRHDFGF |
| | signal sequence | PQEEFGNQFQKAETIPVLHEMIQQIFNLFSTKDSSAAW |
| | | DETLLDKFYTELYQQLNDLEACVIQGVGVTETPLMKED |
| | | SILAVRKYFQRITLYLKEKKYSPCAWEVVRAEIMRSFS |
| | | LSTNLQESLRSKESGGGGSGRSDNIGPPCPPCPAPEFL |
| | | GGPSVFLFPPKPKDTLMISRTPEVTCVVVDVSQEDPEV |
| | | QFNWYVDGVEVHNAKTKPREEQFNSTYRVVSVLTVLHQ |
| | | DWLNGKEYKCKVSNKGLPSSIEKTISKAKGQPREPQVY |
| | | TLPPSQEEMTKNQVSLTCLVKGFYPSDIAVEWESNGQP |
| | | ENNYKTTPPVLDSDGSFFLYSRLTVDKSRWQQGNVFSC |
| | | SVMHEALHNHYTQKSLSLS |
| 338 | ProC290 without | CDLPQTHSLGSRRTLMLLAQMRRISLFSCLKDRHDFGF |
| | signal sequence | PQEEFGNQFQKAETIPVLHEMIQQIFNLFSTKDSSAAW |
| | | DETLLDKFYTELYQQLNDLEACVIQGVGVTETPLMKED |
| | | SILAVRKYFQRITLYLKEKKYSPCAWEVVRAEIMRSFS |
| | | LSTNLQESLRSKESGGGGSGRSDNIESKYGPPCPPCPA |
| | | PEFLGGPSVFLFPPKPKDTLMISRTPEVTCVVVDVSQE |
| | | DPEVQFNWYVDGVEVHNAKTKPREEQFNSTYRVVSVLT |
| | | VLHQDWLNGKEYKCKVSNKGLPSSIEKTISKAKGQPRE |
| | | PQVYTLPPSQEEMTKNQVSLTCLVKGFYPSDIAVEWES |
| | | NGQPENNYKTTPPVLDSDGSFFLYSRLTVDKSRWQQGN |
| | | VFSCSVMHEALHNHYTQKSLSLS |
| 339 | ProC291 without | CDLPQTHSLGSRRTLMLLAQMRRISLFSCLKDRHDFGF |
| | signal sequence | PQEEFGNQFQKAETIPVLHEMIQQIFNLFSTKDSSAAW |
| | | DETLLDKFYTELYQQLNDLEACVIQGVGVTETPLMKED |
| | | SILAVRKYFQRITLYLKEKKYSPCAWEVVRAEIMRSFS |
| | | LSTNLQESLRSKESGGGGSGRSDNIGGGSESKYGPPCP |
| | | PCPAPEFLGGPSVFLFPPKPKDTLMISRTPEVTCVVVD |
| | | VSQEDPEVQFNWYVDGVEVHNAKTKPREEQFNSTYRVV |
| | | SVLTVLHQDWLNGKEYKCKVSNKGLPSSIEKTISKAKG |
| | | QPREPQVYTLPPSQEEMTKNQVSLTCLVKGFYPSDIAV |
| | | EWESNGQPENNYKTTPPVLDSDGSFFLYSRLTVDKSRW |
| | | QQGNVFSCSVMHEALHNHYTQKSLSLS |
| 340 | ProC441 without | CDLPQTHSLGSRRTLMLLAQMRRISLFSCLKDRHDFGF |
| | signal sequence | PQEEFGNQFQKAETIPVLHEMIQQIFNLFSTKDSSAAW |
| | | DETLLDKFYTELYQQLNDLEACVIQGVGVTETPLMKED |
| | | SILAVRKYFQRITLYLKEKKYSPCAWEVVRAEIMRSFS |
| | | LSTNLQESLRSKESGRSDNIGPPCPPCPAPEFLGGPSV |
| | | FLFPPKPKDTLMISRTPEVTCVVVDVSQEDPEVQFNWY |
| | | VDGVEVHNA |
| 341 | ProC442 without | CDLPQTHSLGSRRTLMLLAQMRRISLFSCLKDRHDFGF |
| | signal sequence | PQEEFGNQFQKAETIPVLHEMIQQIFNLFSTKDSSAAW |
| | | DETLLDKFYTELYQQLNDLEACVIQGVGVTETPLMKED |
| | | SILAVRKYFQRITLYLKEKKYSPCAWEVVRAEIMRSFS |
| | | LSTNLQESLRSKESGRSDNIESKYGPPCPPCPAPEFLG |
| | | GPSVFLFPPKPKDTLMISRTPEVTCVVVDVSQEDPEVQ |
| | | FNWYVDGVEVHNAKTKPREEQFNSTYRVVSVLTVLHQD |
| | | WLNGKEYKCKVSNKGLPSSIEKTISKAKGQPREPQVYT |
| | | LPPSQEEMTKNQVSLTCLVKGFYPSDIAVEWESNGQPE |
| | | NNYKTTPPVLDSDGSFFLYSRLTVDKSRWQQGNVFSCS |
| | | VMHEALHNHYTQKSLSLS |
| 342 | ProC443 without | CDLPQTHSLGSRRTLMLLAQMRRISLFSCLKDRHDFGF |
| | signal sequence | PQEEFGNQFQKAETIPVLHEMIQQIFNLFSTKDSSAAW |
| | | DETLLDKFYTELYQQLNDLEACVIQGVGVTETPLMKED |
| | | SILAVRKYFQRITLYLKEKKYSPCAWEVVRAEIMRSFS |
| | | LSTNLQESLRSKESGRSDNIGGGSESKYGPPCPPCPAP |
| | | EFLGGPSVFLFPPKPKDTLMISRTPEVTCVVVDVSQED |
| | | PEVQFNWYVDGVEVHNAKTKPREEQFNSTYRVVSVLTV |
| | | LHQDWLNGKEYKCKVSNKGLPSSIEKTISKAKGQPREP |
| | | QVYTLPPSQEEMTKNQVSLTCLVKGFYPSDIAVEWESN |
| | | GQPENNYKTTPPVLDSDGSFFLYSRLTVDKSRWQQGNV |
| | | FSCSVMHEALHNHYTQKSLSLS |
| 343 | Signal sequence | MRAWIFFLLCLAGRALA |
| 344 | Signal sequence | MALTFALLVALLVLSCKSSCSVG |
| 345 | Signal sequence | METDTLLLWVLLLWVPGSTG |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. (canceled)

2. A composition comprising an activatable cytokine construct (ACC) comprising a first monomer construct and a second monomer construct, wherein:
(a) the first monomer construct comprises a first mature cytokine protein (CP1), a first cleavable moiety (CM1), and a first dimerization domain (DD1);
(b) the second monomer construct comprises a second mature cytokine protein (CP2), a second cleavable moiety (CM2), and a second dimerization domain (DD2);
(c) the first monomer construct is a polypeptide comprising, in an N- to C-terminal direction, the CP1, the CM1, and the DD1 and the first monomer construct is characterized in that the CP1 and the DD1 are linked by a Linking Region of no more than 18 amino acids such that the Linking Region of no more than 18 amino acids includes the CM1:
(d) further wherein:
(i) the second monomer construct is the same as the first monomer construct, and
(ii) the DD1 and the DD2 are a pair of IgG Fc domains;
(e) the DD1 and the DD2 are covalently bound to each other via at least one disulfide bond thereby forming a dimer of the first monomer construct and the second monomer construct; and
(f) the ACC is characterized by having a reduced level of cytokine activity as compared to a corresponding control cytokine; and
a pharmaceutically acceptable carrier.

3. The composition of claim 2, wherein the CP1 comprises a sequence that is at least 95% identical to a sequence selected from the group consisting of SEQ ID NOs: 1, 111, 113, 115, 117, 119, 121, 123, 125, 127, 129, 131, 133, 137, 139, 143, 144, 151, 153, 155, 157, and 159.

4. The composition of claim 2, wherein the CP1 is a mature interferon or a mature interleukin.

5. The composition of claim 2, wherein the CP1 is IL-15.

6. The pharmaceutical composition of claim 2, wherein the CP1 comprises a sequence that is at least 95% identical to SEQ ID NO: 1.

7. The pharmaceutical composition of claim 2, wherein the CP1 comprises the sequence of SEQ ID NO: 1.

8. The pharmaceutical composition of claim 2, wherein the CP1 comprises a sequence that is at least 95% identical to SEQ ID NO: 129.

9. The pharmaceutical composition of claim 2, wherein the DD1 and the DD2 each comprises a sequence that is at least 95% identical to SEQ ID NO: 3, 315, or 316.

10. The pharmaceutical composition of claim 2, wherein the DD1 and the DD2 each comprises the sequence of SEQ ID NO: 3, 315, or 316.

11. The pharmaceutical composition of claim 2, wherein each of the first and second monomer constructs comprises SEQ ID NO: 313.

12. The composition of claim 2, wherein:
the CP1 has a sequence selected from the group consisting of SEQ ID NOs: 1, 111, 113, 115, 117, 119, 121, 123, 125, 127, 129, 131, 133, 137, 139, 143, 144, 151, 153, 155, 157, and 159.

13. The composition of claim 2, wherein:
(a) the CP1 and the CM1 directly abut each other and the CM1 and the DD1 directly abut each other;
(b) the CP2 and the CM2 directly abut each other and the CM2 and the DD2 directly abut each other;
(c) wherein the CP1 has a sequence at least 95% identical to a sequence selected from the group consisting of SEQ ID NOs: 1, 111, 113, 115, 117, 119, 121, 123, 125, 127, 129, 131, 133, 137, 139, 143, 144, 151, 153, 155, 157, and 159,
(d) further wherein the DD1 and the DD2 are a pair of IgG1 or IgG4 Fc domains.

14. A composition comprising an activatable cytokine construct (ACC) that includes a first monomer construct and a second monomer construct, wherein:
(a) the first monomer construct comprises a first mature cytokine protein (CP1), a first cleavable moiety (CM1), and a first dimerization domain (DD1),
wherein the CM1 is positioned between the CP1 and the DD1 and the first monomer construct is characterized in that the CP1 and the DD1 are linked by a Linking Region of no more than 18 amino acids such that the Linking Region of no more than 18 amino acids includes the CM1; and
(b) the second monomer construct comprises a second mature cytokine protein (CP2), a second cleavable moiety (CM2), and a second dimerization domain (DD2),
wherein the CM2 is positioned between the CP2 and the DD2 and the second monomer construct is characterized in that the CP2 and the DD2 are linked by a Linking Region of no more than 18 amino acids such that the Linking Region of no more than 18 amino acids includes the CM2; or
(a) the first monomer construct comprises a first mature cytokine protein (CP1), a first dimerization domain (DD1), and
(b) the second monomer construct comprises a second mature cytokine protein (CP2), a cleavable moiety (CM), and a second dimerization domain (DD2), wherein the CM is positioned between the CP2 and the DD2, wherein the CM functions as a substrate for a protease and the second monomer construct is characterized in that the CP2 and the DD2 are linked by a Linking Region of no more than 18 amino acids such that the Linking Region of no more than 18 amino acids includes the CM2; or
(a) the first monomer construct comprises a first mature cytokine protein (CP1), a cleavable moiety (CM), and a first dimerization domain (DD1), wherein the CM is positioned between the CP1 and the DD1 and the first monomer construct is characterized in that the CP1 and the DD1 are linked by a Linking Region of no more than 18 amino acids such that the Linking Region of no more than 18 amino acids includes the CM1, and
(b) the second monomer construct comprises a second mature cytokine protein (CP2), and a second dimerization domain (DD2),
wherein the CM functions as a substrate for a protease; or
(a) the first monomer construct comprises a first mature cytokine protein (CP1), and a first dimerization domain (DD1), and
(b) the second monomer construct comprises a second mature cytokine protein (CP2), and a second dimerization domain (DD2), wherein the CP1, the CP2, or both CP1 and CP2 include(s) an amino acid sequence that functions as a substrate for a protease and the first monomer construct is characterized in that the CP1 and the DD1 are linked by a Linking Region of no more than 18 amino acids and the second monomer construct is characterized in that the CP2 and the DD2 are linked by a Linking Region of no more than 18 amino acids;
wherein CP1 and CP2 each have a sequence at least 95% identical to a sequence selected from the group consisting of SEQ ID NOs: 1, 111, 113, 115, 117, 119, 121, 123, 125, 127, 129, 131, 133, 137, 139, 143, 144, 151, 153, 155, 157, and 159;
wherein the DD1 and the DD2 bind each other thereby forming a dimer of the first monomer construct and the second monomer construct;
wherein the ACC is characterized by having a reduced level of CP1 or CP2 activity as compared to a control level of the CP1 or CP2 activity, and
wherein the first monomer construct and the second monomer construct have a structure, wherein CP1 and CP2 are N-terminal to DD1 and DD2, respectively, in each construct; and
a pharmaceutically acceptable carrier.

15. A composition comprising:
a polypeptide comprising a first mature cytokine protein (CP1), a first cleavable moiety (CM1), and a first dimerization domain (DD1),
wherein the polypeptide comprises, in an N- to C-terminal direction, the CP1, the CM1, and the DD1 and the first monomer construct is characterized in that the CP1 and the DD1 are linked by a Linking Region of no more than 18 amino acids such that the Linking Region of no more than 18 amino acids includes the CM1,
wherein the polypeptide is characterized by having a reduced level of cytokine activity as compared to a corresponding control cytokine; and
a pharmaceutically acceptable carrier.

16. A composition comprising:
a polypeptide comprising a first mature cytokine protein (CP1), a first cleavable moiety (CM1), and a first dimerization domain (DD1),
wherein the polypeptide comprises, in an N- to C-terminal direction, the CP1, the CM1, and the DD1 and the polypeptide is characterized in that the CP1 and the DD1 are linked by a Linking Region of no more than 18 amino acids such that the Linking Region of no more than 18 amino acids includes the CM1,
wherein the DD1 is an IgG Fc domain; and
a second dimerization domain (DD2), wherein the DD2 is an IgG Fc domain;
the DD1 and the DD2 are covalently bound to each other via at least one disulfide bond thereby forming a dimer,
wherein the dimer is characterized by having a reduced level of cytokine activity as compared to a corresponding control cytokine; and
a pharmaceutically acceptable carrier.

17. A method of treating a subject in need thereof comprising administering the pharmaceutical composition of claim 2 to the subject.

18. A pair of polynucleotides comprising a first polynucleotide and a second polynucleotide, wherein the first polynucleotide encodes the first monomer construct and the second polynucleotide encodes the second monomer construct of claim 2.

19. A cell comprising the pair of polynucleotides of claim 18.

20. A method of producing an activatable cytokine construct (ACC) comprising:
a) culturing the cell of claim 19 in a liquid culture medium to produce the ACC;
b) recovering the ACC from the cell or the liquid culture medium; and
c) isolating the ACC recovered from the cell or the liquid culture medium.

21. A mammalian cell comprising the first polynucleotide and/or the second polynucleotide of claim 18.

22. A method of manufacturing an activatable cytokine construct (ACC), the method comprising:
a) expressing the ACC in the mammalian cell of claim 21; and
b) purifying the expressed ACC.
